SEQUENCE LISTING (384 sequences)

SEQ ID NO: 1 - protein, 9 aa, Artificial sequence
  short peptide
YMDGTMSQV

SEQ ID NO: 2 - protein, 9 aa, Artificial sequence
  short peptide
YLLPAIVHI

SEQ ID NO: 3 - protein, 19 aa, Artificial sequence
  short peptide
TTAGCAAAGCATACCATCA

SEQ ID NO: 4 - protein, 19 aa, Artificial sequence
  short peptide
CCAGACAAAGAGGTCATAA

SEQ ID NO: 5 - protein, 18 aa, Artificial sequence
  short peptide
AGGCTGCAATAAGAGATA

SEQ ID NO: 6 - protein, 18 aa, Artificial sequence
  short peptide
TTCGCTGACAAGTTTTAC

SEQ ID NO: 7 - protein, 10 aa, Artificial sequence
  short peptide
EAAGIGILTV

SEQ ID NO: 8 - protein, 163 aa, homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 164 aa, homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 10 - protein, 123 aa, homo sapiens
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 11 - protein, 101 aa, homo sapiens
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 12 - protein, 220 aa, homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 13 - protein, 255 aa, homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 14 - protein, 199 aa, homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 15 - protein, 277 aa, homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 16 - protein, 184 aa, homo sapiens
MAPLLPIRTLPLILILLALLSPGAADFNISSLSGLLSPALTESLLVALPPCHLTGGNATLMVRRANDSKVVTSSFVVPPCRGRRELVSVVDSGAGFTVTRLSAYQVTNLVPGTKFYISYLVKKGTATESSREIPMSTLPRRNMESIGLGMARTGGMVVITVLLSVAMFLLVLGFIIALALGSRK

SEQ ID NO: 17 - protein, 273 aa, homo sapiens
MASAAAAEAEKGSPVVVGLLVVGNIIILLSGLSLFAETIWVTADQYRVYPLMGVSGKDDVFAGAWIAIFCGFSFFMVASFGVGAALCRRRSMVLTYLVLMLIVYIFECASCITSYTHRDYMVSNPSLITKQMLTFYSADTDQGQELTRLWDRVMIEQECCGTSGPMDWVNFTSAFRAATPEVVFPWPPLCCRRTGNFIPLNEEGCRLGHMDYLFTKAGVQWHNLSSLQRLPPGFKGFSHLSFQSSWDYRAASNTSATPSTATRGVSRGLGLPS

SEQ ID NO: 18 - protein, 258 aa, homo sapiens
MASAAAAEAEKGSPVVVGLLVVGNIIILLSGLSLFAETIWVTADQYRVYPLMGVSGKDDVFAGAWIAIFCGFSFFMVASFGVGAALCRRRSMVLTYLVLMLIVYIFECASCITSYTHRDYMVSNPSLITKQMLTFYSADTDQGQELTRLWDRVMIEQECCGTSGPMDWVNFTSAFRAATPEVVFPWPPLCCRRTGNFIPLNEEGCRLGHMDYLFTKGCFEHIGHAIDSYTWGISWFGFAILMWTLPVMLIAMYFYTML

SEQ ID NO: 19 - protein, 245 aa, homo sapiens [sequence omitted]

SEQ ID NO: 20 - protein, 114 aa, homo sapiens
MAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 21 - protein, 386 aa, homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 22 - protein, 418 aa, homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQVLKVIFAVAFCLISAVLMVLLFIHIRRGLCWQRESYGNI

SEQ ID NO: 23 - protein, 353 aa, homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 24 - protein, 335 aa, homo sapiens
MPRPRLLAALCGALLCAPSLLVALDICSKNPCHNGGLCEEISQEVRGDVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQIFPGNWDNHSHKKNLFETPILARYVRILPVAWHNRIALRLELLGC

SEQ ID NO: 25 - protein, 264 aa, homo sapiens
MTPGTQSPFFLLLLLTVLTATTAPKPATVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 26 - protein, 203 aa, homo sapiens
MTPGTQSPFFLLLLLTVLTVVTGSGHASSTPGGEKETSATQRSSVPSSTEKNAIPAPTTTKSCRETFLKCFCRFINKGVFWASPILSSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 27 - protein, 159 aa, homo sapiens
MTPGTQSPFFLLLLLTVLTATTAPKPATVVTGSGHASSTPGGEKETSATQRSSVPSSTEKNAFNSSLEDPSTDYYQELQRDISEMAVCQCRRKNYGQLDIFPARDTYHPMSEYPTYHTHGRYVPPSSTDRSPYEKVSAGNGGSSLSYTNPAVAATSANL

SEQ ID NO: 28 - protein, 475 aa, homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 29 - protein, 282 aa, homo sapiens
MTPGTQSPFFLLLLLTVLTATTAPKPATVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 30 - protein, 238 aa, homo sapiens
MTPGTQSPFFLLLLLTVLTATTAPKPATVVTGSGHASSTPGGEKETSATQRSSVPSSTEKNAIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 31 - protein, 241 aa, homo sapiens
MTPGTQSPFFLLLLLTVLTATTAPKPATVVTGSGHASSTPGGEKETSATQRSSVPSSTEKNAFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 32 - protein, 198 aa, homo sapiens
MTPGTQSPFFLLLLLTVLTVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSGCLSVPPKELRAAGHLSSPGYLPSYERVPHLPHPWALCAP

SEQ ID NO: 33 - protein, 189 aa, homo sapiens
MTPGTQSPFFLLLLLTVLTATTAPKPATVVTGSGHASSTPGGEKETSATQRSSVPSSTEKNAFNSSLEDPSTDYYQELQRDISEMSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 34 - protein, 177 aa, homo sapiens
MTPGTQSPFFLLLLLTVLTATTAPKPATVVTGSGHASSTPGGEKETSATQRSSVPSSTEKNALSTGVSFFFLSFHISNLQFNSSLEDPSTDYYQELQRDISEMAVCQCRRKNYGQLDIFPARDTYHPMSEYPTYHTHGRYVPPSSTDRSPYEKVSAGNGGSSLSYTNPAVAATSANL

SEQ ID NO: 35 - protein, 255 aa, homo sapiens
MTPGTQSPFFLLLLLTVLTVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 36 - protein, 150 aa, homo sapiens
MTPGTQSPFFLLLLLTVLTVVTGSGHASSTPGGEKETSATQRSSVPSSTEKNAFNSSLEDPSTDYYQELQRDISEMAVCQCRRKNYGQLDIFPARDTYHPMSEYPTYHTHGRYVPPSSTDRSPYEKVSAGNGGSSLSYTNPAVAATSANL

SEQ ID NO: 37 - protein, 158 aa, homo sapiens
MTPGTQSPFFLLLLLTVLTVVTGSGHASSTPGGEKETSATQRSSVPSSTEKNAIPAPTTTKSCRETFLKCFCRFINKGVFWASPILSSVWGWGARLGHRAAGAGLCSGCAGHCLSHCLGCLSVPPKELRAAGHLSSPGYLPSYERVPHLPHPWALCAP

SEQ ID NO: 38 - protein, 484 aa, homo sapiens
MTPGTQSPFFLLLLLTVLTATTAPKPATVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 39 - protein, 219 aa, homo sapiens
MTPGTQSPFFLLLLLTVLTATTAPKPATVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVWGWGARLGHRAAGAGLCSGCAGHCLSHCLGCLSVPPKELRAAGHLSSPGYLPSYERVPHLPHPWALCAP

SEQ ID NO: 40 - protein, 217 aa, homo sapiens
MTPGTQSPFFLLLLLTVLTGGEKETSATQRSSVPSSTEKNAIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 41 - protein, 239 aa, homo sapiens
MTPGTQSPFFLLLLLTVLTATTAPKPATVVTGSGHASSTPGGEKETSATQRSSVPSSTEKNAIPAPTTTKSCRETFLKWPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 42 - protein, 230 aa, homo sapiens
MTPGTQSPFFLLLLLTVLTVVTGSGHASSTPGGEKETSATQRSSVPSSTEKNAIPAPTTTKSCRETFLKWPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 43 - protein, 212 aa, homo sapiens
MTPGTQSPFFLLLLLTVLTATTAPKPATVVTGSGHASSTPGGEKETSATQRSSVPSSTEKNAIPAPTTTKSCRETFLKCFCRFINKGVFWASPILSSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 44 - protein, 273 aa, homo sapiens
MTPGTQSPFFLLLLLTVLTVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 45 - protein, 575 aa, homo sapiens
MDLVLKRCLLHLAVIGALLAVGATKGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 46 - protein, 661 aa, homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 47 - protein, 668 aa, homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPVPGILLTGQEAGLGQVPLIVGILLVLMAVVLASLIYRRRLMKQDFSVPQLPHSSSHWLRLPRIFCSCPIGENSPLLSGQQV

SEQ ID NO: 48 - protein, 118 aa, homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 49 - protein, 1069 aa, homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 50 - protein, 1132 aa, homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 51 - protein, 353 aa, homo sapiens
MAHFPGFGQSLLFGYPVYVFGDCVQGDWCPISGGLCSARLHRHALLATCPEHQITWDPIDGRVIGSALQFLIPRLPSFPTQRTSKTLKVLTPPITHTTPNIPPSFLQAMRKYSPFRNGYMEPTLGQHLPTLSFPDPGLRPQNLYTLWGGSVVCMYLYQLSPPITWPLLPHVIFCHPGQLGAFLTNVPYKRIEKLLYKISLTTGALIILPEDCLPTTLFQPARAPVTLTAWQNGLLPFHSTLTTPGLIWTFTDGTPMISGPCPKDGQPSLVLQSSSFIFHKFQTKAYHPSFLLSHGLIQYSSFHNLHLLFEEYTNIPISLLFNEKEADDNDHEPQISPGGLEPLSEKHFRETEV

SEQ ID NO: 52 - protein, 345 aa, homo sapiens
MAHFPGFGQSLLYGYPVYVFGDCVQADWCPISGGLCSPRLHRHALLATCPEHQITWDPIDGRVVGSPLQYLIPRLPSFPTQRTSKTLKVLTPPTTPVTPKVPPSFFQSVRRHSPYRNGCLETTLGEQLPSLAFPEPGLRPQNVYTIWGKTIVCLYIYQLSPPMTWPLIPHVIFCNPRQLGAFLSNVPPKRLEELLYKLYLHTGAIIILPEDALPTTLFQPVRAPCVQTTWNTGLLPYQPNLTTPGLIWTFNDGSPMISGPCPKAGQPSLVVQSSLLIFERFQTKAYHPSYLLSHQLIQYSSFHHLYLLFDEYTTIPFSLLFKEKEGDDRDNDPLPGATASPQGQN

SEQ ID NO: 53 - protein, 180 aa, homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 54 - protein, 309 aa, homo sapiens
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 55 - protein, 314 aa, homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 56 - protein, 1225 aa, homo sapiens
MKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 57 - protein, 1240 aa, homo sapiens
MPRGSWKPQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 58 - protein, 1055 aa, homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRNM

SEQ ID NO: 59 - protein, 603 aa, homo sapiens
MKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHS

SEQ ID NO: 60 - protein, 1255 aa, homo sapiens [sequence omitted]

SEQ ID NO: 61 - protein, 781 aa, homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 62 - protein, 781 aa, homo sapiens; identical to SEQ ID NO: 61

SEQ ID NO: 63 - protein, 781 aa, homo sapiens; identical to SEQ ID NO: 61

SEQ ID NO: 64 - protein, 529 aa, homo sapiens [sequence omitted]

SEQ ID NO: 65 - protein, 693 aa, homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEIT

SEQ ID NO: 66 - protein, 464 aa, homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 67 - protein, 538 aa, homo sapiens
MEGGRRARVVIESKRNFFLGAFPTPFPAEHVELGRLGDSETAMVPGKGGADYILLPFKKMDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 68 - protein, 496 aa, homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRFHFCRMSWAEANSQCQTQSVPFWRRVDHLLIRVMLYQISEEVSRSELRSFKFLLQEEISKCKLDDDMNLLDIFIEMEKRVILGEGKLDILKRVCAQINKSLLKIINDYEEFSKGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 69 - protein, 479 aa, homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 70 - protein, 464 aa, homo sapiens; identical to SEQ ID NO: 66

SEQ ID NO: 71 - protein, 235 aa, homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNDFGQSLPNEKQTSGILSDHQQSQFCKSTGESAQTSQH

SEQ ID NO: 72 - protein, 9 aa, Artificial sequence
  short peptide
ILKEPVHGV

SEQ ID NO: 73 - protein, 9 aa, Artificial sequence
  short peptide
SLYNTVATL

SEQ ID NO: 74 - protein, 8 aa, Artificial sequence
  short peptide
ILEPVHGV

SEQ ID NO: 75 - protein, 9 aa, Artificial sequence
  short peptide
GILGFVFTL

SEQ ID NO: 76 - protein, 252 aa, Influenza A virus
MSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSAGALASCMGLIYNRMGAVTTEVAFAVVCATCEQIADSQHRSHRQMVTTTNPLIRHENRMVLASTTAKAMEQMAGSSEQAAEAMEVASQARQMVQAMRAIGTPPSSSAGLKDDLLENLQAYQKRMGVQMQRFK

SEQ ID NO: 77 - protein, 584 aa, Influenza A virus
MKAIIVLLMVVTSNADRICTGITSSNSPHVVKTATQGEVNVTGVIPLTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNYLSELEVKNLQRLSGAMNELHDEILELDEKVDDLRADTISSQIELAVLLSNEGIINSEDEHLLALERKLKKMLGPSAVEIGNGCFETKHKCNQTCLDRIAAGTFNAGDFSLPTFDSLNITAASLNDDGLDNHTILLYYSTAASSLAVTLMIAIFIVYMVSRDNVSCSICL

SEQ ID NO: 78 - protein, 566 aa, Influenza A virus
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSNNSFFSRLNWLTHLKFKYPALNVTMPNNEKFDKLYIWGVHHPGTDNDQISLYAQASGRITVSTKRSQQTVIPSIGSRPRIRDVPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 79 - protein, 466 aa, Influenza B virus
MLPSTVQTLTLLLTSGGVLLSLYVSASLSYLLYSDVLLKFSSTKTTAPTMSLECTNASNAQTVNHSATKEMTFPPPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 80 - protein, 565 aa, Influenza C virus
MSDRRQNRKTPDEQRKANALIINENIEAYIAICKEVGLNGDEMLILENGIAIEKAIRICCDGKYQEKREKKAREAQRADSNFNADSIGIRLVKRAGSGTNITYHAVVELTSRSRIVQILKSHWGNELNRAKIAGKRLGFSALFASNLEAIIYQRGRNAARRNGSAELFTLTQGAGIETRYKWIMEKHIGIGVLIADAKGLINGKREGKRGVDANVKLRAGTTGSPLERAMQGIEKKAFPGPLRALARRVVKANYNDAREALNVIAEASLLLKPQITNKMTMPWCMWLAARLTLKDEFANFCAYAGRRAFEVFNIAMEKIGICSFQGTIMNDDEIESIEDKAQVLMMACFGLAYEDFSLVSAMVSHPLKLRNRMKIGNFRVGEKVSTVLSPLLRFTRWAEFAQRFALQANTSREGAQISNSAVFAVERKITTDVQRVEELLNKVQAHEDEPLQTLYKKVREQISIIGRNKSEIKEFLGSSMYDLNDQEKQNPINFRSGAHPFFFEFDPDYNPIRVKRPKKPIAKRNSNISRLEEEGMDENSEIGQAKKMKPLDQLTSTSSNIPGKN

SEQ ID NO: 81 - protein, 498 aa, Influenza A virus
MASQGTKRSYEQMETDGERQNATEIRASVGKMIDGIGRFYIQMCTELKLSDYEGRLIQNSLTIERMVLSAFDERRNKYLEEHPSAGKDPKKTGGPIYKRVDGKWMRELVLYDKEEIRRIWRQANNGDDATAGLTHMMIWHSNLNDTTYQRTRALVRTGMDPRMCSLMQGSTLPRRSGAAGAAVKGVGTMVMELIRMIKRGINDRNFWRGENGRKTRSAYERMCNILKGKFQTAAQRAMMDQVRESRNPGNAEIEDLIFLARSALILRGSVAHKSCLPACVYGPAIASGYNFEKEGYSLVGIDPFKLLQNSQVYSLIRPNENPAHKSQLVWMACNSAAFEDLRVLSFIRGTKVSPRGKLSTRGVQIASNENMDTMESSTLELRSRYWAIRTRSGGNTNQQRASAGQISVQPAFSVQRNLPFDKPTIMAAFTGNTEGRTSDMRAEIIRMMEGAKPEEMSFQGRGVFELSDEKATNPIVPSFDMSNEGSYFFGDNAEEYDN

SEQ ID NO: 82 - protein, 498 aa, Influenza A virus
MASQGTKRSYEQMETGGERQNATEIRASVGRMVGGIGRFYVQMCTELKLSDQEGRLIQNSITIERMVLSAFDERRNRYLEEHPSAGKDPKKTGGPIYRRRDGKWVRELILYDKEEIRRIWRQANNGEDATAGLTHMMIWHSNLNDATYQRTRALVRTGMDPRMCSLMQGSTLPRRSGAAGAAIKGVGTMVMELIRMIKRGINDRNFWRGDNGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVIPRGQLSTRGVQIASNENVEAMDSSTLELRSRYWAIRTRSGGNTNQQRASAGQISVQPTFSVQRNLPFERPTIMAAFKGNTEGRTSDMRTEIIRMMESARPEDVSFQGRGVFELSDEKATNPIVPSFDMSNEGSYFFGDNAEEYDN

SEQ ID NO: 83 - protein, 560 aa, Influenza B virus
MSNMDIDSINTGTIDKTPEELTPGTSGATRPIIKPATLAPPSNKRTRNPSPERTTTSSETDIGRKIQKKQTPTEIKKSVYKMVVKLGEFYNQMMVKAGLNDDMERNLIQNAQAVERILLAATDDKKTEYQKKRNARDVKEGKEEIDHNKTGGTFYKMVRDDKTIYFSPIKITFLKEEVKTMYKTTMGSDGFSGLNHIMIGHSQMNDVCFQRSKGLKRVGLDPSLISTFAGSTLPRRSGTTGVAIKGGGTLVDEAIRFIGRAMADRGLLRDIKAKTAYEKILLNLKNKCSAPQQKALVDQVIGSRNPGIADIEDLTLLARSMVVVRPSVASKVVLPISIYAKIPQLGFNTEEYSMVGYEAMALYNMATPVSILRMGDDAKDKSQLFFMSCFGAAYEDLRVLSALTGTEFKPRSALKCKGFHVPAKEQVEGMGAALMSIKLQFWAPMTRSGGNEVSGEGGSGQISCSPVFAVERPIALSKQAVRRMLSMNVEGRDADVKGNLLKMMNDSMAKKTSGNAFIGKKMFQISDKNKVNPIEIPIKQTIPNFFFGRDTAEDYDDLDY

SEQ ID NO: 84 - protein, 248 aa, Influenza B virus
MSLFGDTIAYLLSLIEDGEGKAELAEKLHCWFGGKEFDLDSALEWIKNKRCLTDIQKALIGASICFLKPKDQERKRRFITEPLSGMGTTATKKKGLILAERKMRRCVSFHEAFEIAEGHESSALLYCLMVMYLNPENYSMQVKLGTLCALCEKQASHSHRAHSRAARSSVPGVRREMQMVSAMNTAKTMNGMGKGEDVQKLAEELQNNIGVLRSLGASQKNGEGIAKDVMEVLKQSSMGNSALVRKYL

SEQ ID NO: 85 - protein, 97 aa, Influenza A virus
MSLLTEVETPIRNEWGCRCNGSSDPLAIAANIIGILHLILWILDRLFFKCIYRRFKYGLKGGPSTEGVPKSMREEYRKEQQSAVDADDGHFVSIELE

SEQ ID NO: 86 - protein, 281 aa, Influenza B virus
MADNMTTTQIEVGPGATNATINFEAGILECYERFSWQRALDYPGQDRLHRLKRKLESRIKTHNKSEPENKRMSLEERKAIGVKMMKVLLFMDPSAGIEGFEPYCVKNPSTSKCPNYDWTDYPPTPGKYLDDIEEEPENVDHPIEVVLRDMNNKDARQKIKDEVNTQKEGKFRLTIKRDIRNVLSLRVLVNGTFLKHPNGDKSLSTLHRLNAYDQNGGLVAKLVATDDRTVEDEKDGHRILNSLFERFDEGHSKPIRAAETAVGVLSQFGQEHRLSPEEGDN

SEQ ID NO: 87 - protein, 122 aa, Influenza B virus
MADNMTTTQIEWRMKKMAIGSSTHSSSVLMKDIQSQFEQLKLRWESYPNLVKSTDYHQKRETIRLATEELYLLSKRIDDSILFHKTVIANSSIIADMIVSLSLLETLYEMKDVVEVYSRQCL

SEQ ID NO: 88 - protein, 716 aa, Influenza A virus
MEDFVRQCFNPMIVELAEKAMKEYGEDLKIETNKFAAICTHLEVCFMYSDFHFINEQGESIIVEPEDPNALLKHRFEIIEGRDRTMAWTVVNSICNTTGAEKPKFLPDLYDYKENRFIEIGVTRREVHIYYLEKANKIKSEKTHIHIFSFTGEEMATKADYTLDEESRARIKTRLFTIRQEMASRGLWDSFRQSERGEETIEERFEITGTMRRLADQSLPPNFSCLENFRAYVDGFEPNGYIEGKLSQMSKEVNARIEPFLRTTPRPIRLPDGPPCFQRSKFLLMDSLKLSIEDPSHEGEGIPLYDAIKCMRTFFGWKEPSVVKPHGKGINPNYLLSWKQVLAELQDIESEEKIPRTKNMKKTSQLKWALGENMAPEKVDFDDCKDISDLKQYDSDEPELRSLSSWIQNEFNKACELTDSIWIELDEIGEDVAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLLRSAIGQVSRPMFLYVRTNGTSKIKMKWGMEMRRCLLQSLQQIESMIEAESSVKEKDMTKEFFENRSETWPIGESPKGVEEGSIGKVCRTLLAKSVFNSLYASPQLEGFSAESRKLLLIVQALRDNLEPGTFDLGGLYEAIEECLINDPWVLLNASWFNSFLTHALR

SEQ ID NO: 89 - protein, 752 aa, Influenza B virus
MNINPYFLFIDVPIQAAISTTFPYTGVPPYSHGTGTGYTIDTVIRTHEYSNKGKQYISDVTGCVMVDPTNGPLPEDNEPSAYAQLDCVLEALDRMDEEHPGLFQAGSQNAMEALMVTTVDKLTQGRQTFDWTVCRNQPAATALNTTITSFRLNDLNGADKGGLVPFCQDIIDSLDKPEMIFFTVKNIKKKLPAKNRKGFLIKRIPMKVKDRITRVEYIKRALSLNTMTKDAERGKLKRRAIATAGIQIRGFVLVVENLAKNICENLEQSGLPVGGNEKKAKLSNAVAKMLSNCPPGGISMTVTGDNTKWNECLNPRIFLAMTERITRDSPIWFRDFCSIAPVLFSNKIARLGKGFMITSKTKRLKAQIPCPDLFNIPLERYNEETRAKLKKLKPFFNEEGTASLSPGMMMGMFNMLSTVLGVAALGIKNIGNKEYLWDGLQSSDDFALFVNAKDEETCMEGINDFYRTCKLLGINMSKKKSYCNETGMFEFTSMFYRDGFVSNFAMELPSFGVAGVNESADMAIGMTIIKNNMINNGMGPATAQTAIQLFIADYRYTYKCHRGDSKVEGKRMKIIKELWENTKGRDGLLVADGGPNLYNLRNLHIPEIILKYNIMDPEYKGRLLHPQNPFVGHLSIEGIKEADITPAHGPIKKMDYDAVSGTHSWRTKRNRSILNTDQRNMILEEQCYAKCCNLFEACFNSASYRKPVGQHSMLEAMAHRLRMDARLDYESGRMSKEDFEKAMAHLGEIGYM

SEQ ID NO: 90 - protein, 759 aa, Influenza A virus
MERIKELRNLMSQSRTREILTKTTVDHMAIIKKYTSGRQEKNPALRMKWMMAMKYPITADKRITEMIPERNEQGQTLWSKMNDAGSDRVMVSPLAVTWWNRNGPMTNTVHYPKIYKTYFERVERLKHGTFGPVHFRNQVKIRRRVDINPGHADLSAKEAQDVIMEVVFPNEVGARILTSESQLTITKEKKEELQDCKISPLMVAYMLERELVRKTRFLPVAGGTSSVYIEVLHLTQGTCWEQMYTPGGEVKNDDVDQSLIIAARNIVRRAAVSADPLASLLEMCHSTQIGGIRMVDILKQNPTEEQAVGICKAAMGLRISSSFSFGGFTFKRTSGSSVKREEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGVEPIDNVMGMIGILPDMTPSIEMSMRGVRISKMGVDEYSSTERVVVSIDRFLRVRDQRGNVLLSPEEVSETQGTEKLTITYSSSMMWEINGPESVLVNTYQWIIRNWETVKIQWSQNPTMLYNKMEFEPFQSLVPKAIRGQYSGFVRTLFQQMRDVLGTFDTAQIIKLLPFAAAPPKQSRMQFSSFTVNVRGSGMRILVRGNSPVFNYNKATKRLTVLGKDAGTLTEDPDEGTAGVESAVLRGFLILGKEDRRYGPALSINELSNLAKGEKANVLIGQGDVVLVMKRKRDSSILTDSQTATKRIRMAIN

SEQ ID NO: 91 - protein, 109 aa, Influenza B virus
MLEPLQILSICSFILSALHFMAWTIGHLNQIKRGVNLKIQIRNPNKEAINREVSILRHNYQKEIQAKETMKKILSDNMEVLGDHIVVEGLSTDEIIKMGETVLEVEELQ

SEQ ID NO: 92 - protein, 100 aa, Influenza B virus
MNNATFNCTNINPITHIRGSIIITICVSLIVILIVFGCIAKIFINKNNCTNNVIRVHKRIKCPDCEPFCNKRDDISTPRAGVDIPSFILPGLNLSEGTPN

SEQ ID NO: 93 - protein, 498 aa, Influenza A virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRDPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVVPRGKLSTRGVQIASNENMETMESSTLELRSRYWAIRTRSGGNTNQQRASAGQISIQPTFSVQRNLPFDRTTVMAAFTGNTEGRTSDMRTEIIRMMESARPEDVSFQGRGVFELSDEKAASPIVPSFDMSNEGSYFFGDNAEEYDN

SEQ ID NO: 94 - protein, 548 aa, Human herpesvirus 5
MESRGRRCPEMISVLGPISGHVLKAVFSRGDTPVLPHETRLLQTGIHVRVSQPSLILVSQYTPDSTPCHRGDNQLQVQHTYFTGSEVENVSVNEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTSGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 95 - protein, 561 aa, Human herpesvirus 5
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTSGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 96 - protein, 551 aa, Human herpesvirus 5
MASVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 97 - protein, 551 aa, Human herpesvirus 5; identical to SEQ ID NO: 96

SEQ ID NO: 98 - protein, 353 aa, Human T-lymphotropic virus 1]; identical to SEQ ID NO: 51

SEQ ID NO: 99 - protein, 345 aa, Human T-lymphotropic virus 1]; identical to SEQ ID NO: 52

SEQ ID NO: 100 - skipped

SEQ ID NO: 101 - protein, 9 aa, Artificial sequence
  short peptide
STNRQSGRQ

SEQ ID NO: 102 - protein, 9 aa, Artificial sequence
  short peptide
LLFGYPVYV

SEQ ID NO: 103 - protein, 10 aa, Artificial sequence
  short peptide
GLSPTVWLSV

SEQ ID NO: 104 - protein, 9 aa, Artificial sequence
  short peptide
AMDGTMSQV

SEQ ID NO: 105 - protein, 9 aa, Artificial sequence
  short peptide
YADGTMSQV

SEQ ID NO: 106 - protein, 9 aa, Artificial sequence
  short peptide
YMAGTMSQV

SEQ ID NO: 107 - protein, 9 aa, Artificial sequence
  short peptide
YMDATMSQV

SEQ ID NO: 108 - protein, 9 aa, Artificial sequence
  short peptide
YMDGAMSQV

SEQ ID NO: 109 - protein, 9 aa, Artificial sequence
  short peptide
YMDGTASQV

SEQ ID NO: 110 - protein, 9 aa, Artificial sequence
  short peptide
YMDGTMAQV

SEQ ID NO: 111 - protein, 9 aa, Artificial sequence
  short peptide
YMDGTMSAV

SEQ ID NO: 112 - protein, 9 aa, Artificial sequence
  short peptide
YMDGTMSQA

SEQ ID NO: 113 - protein, 9 aa, Artificial sequence
  short peptide
YMDGTMSQV

SEQ ID NO: 114 - protein, 9 aa, Artificial sequence
  short peptide
YMNGTMSQV

SEQ ID NO: 115 - protein, 9 aa, Artificial sequence
  short peptide
YMDNVMSEV

SEQ ID NO: 116 - protein, 9 aa, Artificial sequence
  short peptide
VMDSKIVQV

SEQ ID NO: 117 - protein, 9 aa, Artificial sequence
  short peptide
LMNGTLKQV

SEQ ID NO: 118 - protein, 9 aa, Artificial sequence
  short peptide
SQDGTRSQV

SEQ ID NO: 119 - protein, 9 aa, Artificial sequence
  short peptide
VMDTTKSQV

SEQ ID NO: 120 - protein, 9 aa, Artificial sequence
  short peptide
GMDGTQQQI

SEQ ID NO: 121 - protein, 9 aa, Artificial sequence
  short peptide
GMVGTMTEV

SEQ ID NO: 122 - protein, 9 aa, Artificial sequence
  short peptide
MMDATFSAV

SEQ ID NO: 123 - protein, 9 aa, Artificial sequence
  short peptide
QMDPTGSQL

SEQ ID NO: 124 - protein, 9 aa, Artificial sequence
  short peptide
SMDGSMRTV

SEQ ID NO: 125 - protein, 9 aa, Artificial sequence
  short peptide
WMDGIASQI

SEQ ID NO: 126 - protein, 9 aa, Artificial sequence
  short peptide
YLEGILSQV

SEQ ID NO: 127 - protein, 9 aa, Artificial sequence
  short peptide
YMAIKMSQL

SEQ ID NO: 128 - protein, 9 aa, Artificial sequence
  short peptide
YMDAVVSLV

SEQ ID NO: 129 - protein, 9 aa, Artificial sequence
  short peptide
YMDGTNRRI

SEQ ID NO: 130 - protein, 9 aa, Artificial sequence
  short peptide
YMDPSTYQV

SEQ ID NO: 131 - protein, 9 aa, Artificial sequence
  short peptide
YMLGTNHQL

SEQ ID NO: 132 - protein, 9 aa, Artificial sequence
  short peptide
YMPGTASLI

SEQ ID NO: 133 - protein, 9 aa, Artificial sequence
  short peptide
YMRETRSQL

SEQ ID NO: 134 - protein, 9 aa, Artificial sequence
  short peptide
MMDGAMGYV

SEQ ID NO: 135 - protein, 9 aa, Artificial sequence
  short peptide
NMDSFMAQV

SEQ ID NO: 136 - protein, 9 aa, Artificial sequence
  short peptide
QMDFIMSCV

SEQ ID NO: 137 - protein, 9 aa, Artificial sequence
  short peptide
YEDLKMYQV

SEQ ID NO: 138 - protein, 9 aa, Artificial sequence
  short peptide
YMDTIMELV

SEQ ID NO: 139 - protein, 9 aa, Artificial sequence
  short peptide
YTDLAMSTV

SEQ ID NO: 140 - protein, 9 aa, Artificial sequence
  short peptide
YVDFVMSSV

SEQ ID NO: 141 - protein, 9 aa, Artificial sequence
  short peptide
RMFPNAPYL

SEQ ID NO: 142 - protein, 9 aa, Artificial sequence
  short peptide
LDFPNLPYL

SEQ ID NO: 143 - protein, 9 aa, Artificial sequence
  short peptide
RCFPNCPFL

SEQ ID NO: 144 - protein, 9 aa, Artificial sequence
  short peptide
LMFENAAYL

SEQ ID NO: 145 - protein, 9 aa, Artificial sequence
  short peptide
RMFPNKYSL

SEQ ID NO: 146 - protein, 9 aa, Artificial sequence
  short peptide
RLFPNAKFL

SEQ ID NO: 147 - protein, 9 aa, Artificial sequence
  short peptide
RLFPNLPEL

SEQ ID NO: 148 - protein, 9 aa, Artificial sequence
  short peptide
RMFPTPPSL

SEQ ID NO: 149 - protein, 9 aa, Artificial sequence
  short peptide
RMVPRAVYL

SEQ ID NO: 150 - protein, 9 aa, Artificial sequence
  short peptide
RMFFNGRYI

SEQ ID NO: 151 - protein, 9 aa, Artificial sequence
  short peptide
RMLPHAPGV

SEQ ID NO: 152 - protein, 9 aa, Artificial sequence
  short peptide
YMFPNAPYL

SEQ ID NO: 153 - protein, 9 aa, Artificial sequence
  short peptide
AMDPNAAYV

SEQ ID NO: 154 - protein, 9 aa, Artificial sequence
  short peptide
ICFPNAPKV

SEQ ID NO: 155 - protein, 9 aa, Artificial sequence
  short peptide
NMFENGCYL

SEQ ID NO: 156 - protein, 9 aa, Artificial sequence
  short peptide
NMPPNFPYI

SEQ ID NO: 157 - protein, 9 aa, Artificial sequence
  short peptide
REMTQAPYL

SEQ ID NO: 158 - protein, 9 aa, Artificial sequence
  short peptide
RMAPRAPWI

SEQ ID NO: 159 - protein, 9 aa, Artificial sequence
  short peptide
RMEPRAPWI

SEQ ID NO: 160 - protein, 9 aa, Artificial sequence
  short peptide
RMEPRAPWV

SEQ ID NO: 161 - protein, 9 aa, Artificial sequence
  short peptide
RMFLNNPSI

SEQ ID NO: 162 - protein, 9 aa, Artificial sequence
  short peptide
RMFQQTFYL

SEQ ID NO: 163 - protein, 9 aa, Artificial sequence
  short peptide
RMNPNSPSI

SEQ ID NO: 164 - protein, 9 aa, Artificial sequence
  short peptide
RQFPNASLI

SEQ ID NO: 165 - protein, 9 aa, Artificial sequence
  short peptide
RQFPNKDAL

SEQ ID NO: 166 - protein, 9 aa, Artificial sequence
  short peptide
RVFPWASSL

SEQ ID NO: 167 - protein, 9 aa, Artificial sequence
  short peptide
RLFPWGNKL

SEQ ID NO: 168 - protein, 9 aa, Artificial sequence
  short peptide
AMFPNAPYL

SEQ ID NO: 169 - protein, 9 aa, Artificial sequence
  short peptide
RAFPNAPYL

SEQ ID NO: 170 - protein, 9 aa, Artificial sequence
  short peptide
RMAPNAPYL

SEQ ID NO: 171 - protein, 9 aa, Artificial sequence
  short peptide
RMFANAPYL

SEQ ID NO: 172 - protein, 9 aa, Artificial sequence
  short peptide
RMFPAAPYL

SEQ ID NO: 173 - protein, 9 aa, Artificial sequence
  short peptide
RMFPNAAYL

SEQ ID NO: 174 - protein, 9 aa, Artificial sequence
  short peptide
RMFPNAPAL

SEQ ID NO: 175 - protein, 9 aa, Artificial sequence
  short peptide
RMFPNAPYA

SEQ ID NO: 176 - protein, 10 aa, Artificial sequence
  short peptide
GVYDGREHTV

SEQ ID NO: 177 - protein, 10 aa, Artificial sequence
  short peptide
GLADGRTHTV

SEQ ID NO: 178 - protein, 10 aa, Artificial sequence
  short peptide
GVSDGRWHSV

SEQ ID NO: 179 - protein, 10 aa, Artificial sequence
  short peptide
GVYDGEEHSV

SEQ ID NO: 180 - protein, 9 aa, Artificial sequence
  short peptide
GLYDGMEHL

SEQ ID NO: 181 - protein, 10 aa, Artificial sequence
  short peptide
GVSDGQWHTV

SEQ ID NO: 182 - protein, 10 aa, Artificial sequence
  short peptide
GVYAGREHFL

SEQ ID NO: 183 - protein, 10 aa, Artificial sequence
  short peptide
GLYDGMEHLI

SEQ ID NO: 184 - protein, 10 aa, Artificial sequence
  short peptide
ASYDGTEVTV

SEQ ID NO: 185 - protein, 10 aa, Artificial sequence
  short peptide
AVLDGRELRV

SEQ ID NO: 186 - protein, 10 aa, Artificial sequence
  short peptide
GLYDGIEHFM

SEQ ID NO: 187 - protein, 10 aa, Artificial sequence
  short peptide
GLYDGPVHEV

SEQ ID NO: 188 - protein, 10 aa, Artificial sequence
  short peptide
GVCAGREHFI

SEQ ID NO: 189 - protein, 10 aa, Artificial sequence
  short peptide
GVYAGRPLSV

SEQ ID NO: 190 - protein, 10 aa, Artificial sequence
  short peptide
TVYDLREQSV

SEQ ID NO: 191 - protein, 10 aa, Artificial sequence
  short peptide
VVDDGVEHTI

SEQ ID NO: 192 - protein, 9 aa, Artificial sequence
  short peptide
GVFDGLHTV

SEQ ID NO: 193 - protein, 10 aa, Artificial sequence
  short peptide
AVYDGREHTV

SEQ ID NO: 194 - protein, 10 aa, Artificial sequence
  short peptide
GAYDGREHTV

SEQ ID NO: 195 - protein, 10 aa, Artificial sequence
  short peptide
GVADGREHTV

SEQ ID NO: 196 - protein, 10 aa, Artificial sequence
  short peptide
GVYAGREHTV

SEQ ID NO: 197 - protein, 10 aa, Artificial sequence
  short peptide
GVYDAREHTV

SEQ ID NO: 198 - protein, 10 aa, Artificial sequence
  short peptide
GVYDGAEHTV

SEQ ID NO: 199 - protein, 10 aa, Artificial sequence
  short peptide
GVYDGRAHTV

SEQ ID NO: 200 - protein, 10 aa, Artificial sequence
  short peptide
GVYDGREATV

SEQ ID NO: 201 - protein, 10 aa, Artificial sequence
  short peptide
GVYDGREHAV

SEQ ID NO: 202 - protein, 10 aa, Artificial sequence
  short peptide
GVYDGREHTA

SEQ ID NO: 203 - protein, 9 aa, Artificial sequence
  short peptide
ALSVMGVYV

SEQ ID NO: 204 - protein, 9 aa, Artificial sequence
  short peptide
ALSVLGVMV

SEQ ID NO: 205 - protein, 9 aa, Artificial sequence
  short peptide
ALSRKGIYV

SEQ ID NO: 206 - protein, 9 aa, Artificial sequence
  short peptide
ALSVMYSYL

SEQ ID NO: 207 - protein, 9 aa, Artificial sequence
  short peptide
AVSHMGVLV

SEQ ID NO: 208 - protein, 9 aa, Artificial sequence
  short peptide
LLSLMGVLV

SEQ ID NO: 209 - protein, 9 aa, Artificial sequence
  short peptide
VLSIMGVYA

SEQ ID NO: 210 - protein, 9 aa, Artificial sequence
  short peptide
ALQVRKVYV

SEQ ID NO: 211 - protein, 9 aa, Artificial sequence
  short peptide
ALQVYGVEV

SEQ ID NO: 212 - protein, 9 aa, Artificial sequence
  short peptide
ALSVAGGFV

SEQ ID NO: 213 - protein, 9 aa, Artificial sequence
  short peptide
ALSVLGKVV

SEQ ID NO: 214 - protein, 9 aa, Artificial sequence
  short peptide
ALSVMIPAV

SEQ ID NO: 215 - protein, 9 aa, Artificial sequence
  short peptide
DLSVCSVYV

SEQ ID NO: 216 - protein, 9 aa, Artificial sequence
  short peptide
ILGVMGVDV

SEQ ID NO: 217 - protein, 9 aa, Artificial sequence
  short peptide
LLSVNGVSV

SEQ ID NO: 218 - protein, 9 aa, Artificial sequence
  short peptide
SLSPMGRYV

SEQ ID NO: 219 - protein, 10 aa, Artificial sequence
  short peptide
ALSAVMGVTL

SEQ ID NO: 220 - protein, 10 aa, Artificial sequence
  short peptide
AILLVMGVDV

SEQ ID NO: 221 - protein, 9 aa, Artificial sequence
  short peptide
ALSDHHVYL

SEQ ID NO: 222 - protein, 9 aa, Artificial sequence
  short peptide
AASVMGVYV

SEQ ID NO: 223 - protein, 9 aa, Artificial sequence
  short peptide
ALAVMGVYV

SEQ ID NO: 224 - protein, 9 aa, Artificial sequence
  short peptide
ALSAMGVYV

SEQ ID NO: 225 - protein, 9 aa, Artificial sequence
  short peptide
ALSVAGVYV

SEQ ID NO: 226 - protein, 9 aa, Artificial sequence
  short peptide
ALSVMAVYV

SEQ ID NO: 227 - protein, 9 aa, Artificial sequence
  short peptide
ALSVMGAYV

SEQ ID NO: 228 - protein, 9 aa, Artificial sequence
  short peptide
ALSVMGVAV

SEQ ID NO: 229 - protein, 9 aa, Artificial sequence
  short peptide
ALSVMGVYA

SEQ ID NO: 230 - protein, 9 aa, Artificial sequence
  short peptide
TLMSAMTNL

SEQ ID NO: 231 - protein, 9 aa, Artificial sequence
  short peptide
TLMSAEANL

SEQ ID NO: 232 - protein, 9 aa, Artificial sequence
  short peptide
QLCSAMTQL

SEQ ID NO: 233 - protein, 9 aa, Artificial sequence
  short peptide
RLMSALTQL

SEQ ID NO: 234 - protein, 9 aa, Artificial sequence
  short peptide
GLMSLTTNL

SEQ ID NO: 235 - protein, 9 aa, Artificial sequence
  short peptide
GLMSMATNL

SEQ ID NO: 236 - protein, 9 aa, Artificial sequence
  short peptide
GLMSMTTNL

SEQ ID NO: 237 - protein, 9 aa, Artificial sequence
  short peptide
LLMSISTNL

SEQ ID NO: 238 - protein, 9 aa, Artificial sequence
  short peptide
QLPSTMTNL

SEQ ID NO: 239 - protein, 9 aa, Artificial sequence
  short peptide
TLASSMGNL

SEQ ID NO: 240 - protein, 9 aa, Artificial sequence
  short peptide
TLFSALTGL

SEQ ID NO: 241 - protein, 9 aa, Artificial sequence
  short peptide
TLGSATTEL

SEQ ID NO: 242 - protein, 9 aa, Artificial sequence
  short peptide
TLMRAMTDC

SEQ ID NO: 243 - protein, 9 aa, Artificial sequence
  short peptide
TLMSMVANL

SEQ ID NO: 244 - protein, 9 aa, Artificial sequence
  short peptide
TLPSAETAL

SEQ ID NO: 245 - protein, 9 aa, Artificial sequence
  short peptide
TLPSRMTVL

SEQ ID NO: 246 - protein, 9 aa, Artificial sequence
  short peptide
RLMSALTQV

SEQ ID NO: 247 - protein, 9 aa, Artificial sequence
  short peptide
SIHSQMTNL

SEQ ID NO: 248 - protein, 9 aa, Artificial sequence
  short peptide
SIMFAMTPL

SEQ ID NO: 249 - protein, 9 aa, Artificial sequence
  short peptide
TIVAAMSNL

SEQ ID NO: 250 - protein, 9 aa, Artificial sequence
  short peptide
TLITAMEQL

SEQ ID NO: 251 - protein, 9 aa, Artificial sequence
  short peptide
TLTSNMSQL

SEQ ID NO: 252 - protein, 9 aa, Artificial sequence
  short peptide
ALMSAMTNL

SEQ ID NO: 253 - protein, 9 aa, Artificial sequence
  short peptide
TLASAMTNL

SEQ ID NO: 254 - protein, 9 aa, Artificial sequence
  short peptide
TLMAAMTNL

SEQ ID NO: 255 - protein, 9 aa, Artificial sequence
  short peptide
TLMSAATNL

SEQ ID NO: 256 - protein, 9 aa, Artificial sequence
  short peptide
TLMSAMANL

SEQ ID NO: 257 - protein, 9 aa, Artificial sequence
  short peptide
TLMSAMTAL

SEQ ID NO: 258 - protein, 9 aa, Artificial sequence
  short peptide
TLMSAMTNA

SEQ ID NO: 259 - protein, 9 aa, Artificial sequence
  short peptide
VMDSKIVQV

SEQ ID NO: 260 - protein, 9 aa, Artificial sequence
  short peptide
RMLPHAPGV

SEQ ID NO: 261 - protein, 9 aa, Artificial sequence
  short peptide
AMDPNAAYV

SEQ ID NO: 262 - protein, 9 aa, Artificial sequence
  short peptide
RMNPNSPSI

SEQ ID NO: 263 - protein, 10 aa, Artificial sequence
  short peptide
GLADGRTHTV

SEQ ID NO: 264 - protein, 10 aa, Artificial sequence
  short peptide
GLYDGPVHEV

SEQ ID NO: 265 - protein, 9 aa, Artificial sequence
  short peptide
GVFDGLHTV

SEQ ID NO: 266 - protein, 9 aa, Artificial sequence
  short peptide
ALSDHHVYL

SEQ ID NO: 267 - protein, 9 aa, Artificial sequence
  short peptide
RLMSALTQL

SEQ ID NO: 268 - protein, 9 aa, Artificial sequence
  short peptide
RLMSALTQV

SEQ ID NO: 269 - protein, 10 aa, Artificial sequence
  short peptide
ELAGIGILTV

SEQ ID NO: 270 - protein, 9 aa, Artificial sequence
  short peptide
NLVPMVATV

SEQ ID NO: 271 - protein, 9 aa, Artificial sequence
  short peptide
SLYNTVATL

SEQ ID NO: 272 - protein, 9 aa, Artificial sequence
  short peptide
YMDGTMSQV

SEQ ID NO: 273 - protein, 9 aa, Artificial sequence
  short peptide
RMFPNAPYL

SEQ ID NO: 274 - protein, 10 aa, Artificial sequence
  short peptide
GVYDGREHTV

SEQ ID NO: 275 - protein, 9 aa, Artificial sequence
  short peptide
TLMSAMTNL

SEQ ID NO: 276 - protein, 9 aa, Artificial sequence
  short peptide
ALSVMGVYV

SEQ ID NO: 277 - protein, 9 aa, Artificial sequence
  short peptide
KASEKIFYV

SEQ ID NO: 278 - protein, 9 aa, Artificial sequence
  short peptide
SLLMWITQC

SEQ ID NO: 279 - protein, 9 aa, Artificial sequence
  short peptide
TLFDYEVRL

SEQ ID NO: 280 - DNA, 702 bp, Artificial sequence
  Nucleic Acid sequence of D11 light chain
atgagaccgtctattcagttcctggggctcttgttgttctggcttcatggtgctcagtgtgacatccagatgacacagtctccatcctcactgtctgcatctctgggaggcaaagtcaccatcacatgcaaggcaagccaagacattcacaactatatagcttggtaccaacacaagcctgtaaaaggtcctaggctgctcatacattacacatctacattacagccaggcaccccatcaaggttcagtggaagtgggtctgggagagattattccttcagcatcagcaacctggagcctgaagatattgcaacttattattgtctacagtatgataatctgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 281 - protein, 233 aa, Artificial sequence
  Amino Acid sequence of D11 light chain
MRPSIQFLGLLLFWLHGAQCDIQMTQSPSSLSASLGGKVTITCKASQDIHNYIAWYQHKPVKGPRLLIHYTSTLQPGTPSRFSGSGSGRDYSFSISNLEPEDIATYYCLQYDNLWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 282 - DNA, 1398 bp, Artificial sequence
  Nucleic Acid sequence of D11 heavy chain
atggacaggcttacttcctcattcctgctgctgattgtccctgcatatgtcctttcccaggtaactctgaaagagtctggccctgggatattgcagccctcccagaccctcagtctgacttgttctttctctgggttttcactgaccacttctggtatgggtgtgagctggattcgtcagccttcaggaaagggtctggagtggctggcacacatttactgggatgatgacaagcgctataacccatccctgaagagccgactcacaatctccaaggatacctccagaaaccaggtattcctcaagatcaccagtgtggacgctgcagatactgccacatactactgtgctcgaaaggactacggtagtagcttctatgctatgcactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 283 - protein, 465 aa, Artificial sequence
  Amino Acid sequence of D11 heavy chain
MDRLTSSFLLLIVPAYVLSQVTLKESGPGILQPSQTLSLTCSFSGFSLTTSGMGVSWIRQPSGKGLEWLAHIYWDDDKRYNPSLKSRLTISKDTSRNQVFLKITSVDAADTATYYCARKDYGSSFYAMHYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 284 - DNA, 33 bp, Artificial sequence
  Nucleic Acid sequence of D11 light chain CDR
aaggcaagccaagacattcacaactatatagct

SEQ ID NO: 285 - DNA, 21 bp, Artificial sequence
  Nucleic Acid sequence of D11 light chain CDR
tacacatctacattacagcca

SEQ ID NO: 286 - DNA, 24 bp, Artificial sequence
  Nucleic Acid sequence of D11 light chain CDR
ctacagtatgataatctgtggacg

SEQ ID NO: 287 - protein, 11 aa, Artificial sequence
  Amino Acid sequence of D11 light chain CDR
KASQDIHNYIA

SEQ ID NO: 288 - protein, 7 aa, Artificial sequence
  Amino Acid sequence of D11 light chain CDR
YTSTLQP

SEQ ID NO: 289 - protein, 8 aa, Artificial sequence
  Amino Acid sequence of D11 light chain CDR
LQYDNLWT

SEQ ID NO: 290 - DNA, 21 bp, Artificial sequence
  Nucleic Acid sequence of D11 heavy chain CDR
acttctggtatgggtgtgagc

SEQ ID NO: 291 - DNA, 48 bp, Artificial sequence
  Nucleic Acid sequence of D11 heavy chain CDR
cacatttactgggatgatgacaagcgctataacccatccctgaagagc

SEQ ID NO: 292 - DNA, 36 bp, Artificial sequence
  Nucleic Acid sequence of D11 heavy chain CDR
aaggactacggtagtagcttctatgctatgcactac

SEQ ID NO: 293 - protein, 7 aa, Artificial sequence
  Amino Acid sequence of D11 heavy chain CDR
TSGMGVS

SEQ ID NO: 294 - protein, 16 aa, Artificial sequence
  Amino Acid sequence of D11 heavy chain CDR
HIYWDDDKRYNPSLKS

SEQ ID NO: 295 - protein, 12 aa, Artificial sequence
  Amino Acid sequence of D11 heavy chain CDR
KDYGSSFYAMHY

SEQ ID NO: 296 - DNA, 705 bp, Artificial sequence
  Nucleic Acid sequence of D7 light chain
atgagtgtgcccactcaggtcctggggttgctgctgctgtggcttacagatgccagatgtgacatccagatgactcagtctccagcctccctatctgtatctgtgggagaaactgtcaccatcacatgtcgagcaagtgatattatttacagtaatttagcatggtatcagcagaaacagggaaaatctcctcagctcctggtctatgctgcaacaaacttagcagctggtgtgccatcaaggttcagtggcagtggatcaggcacacagtattccctcaagatcaatagcctgcagtctgaagattttgggacttattactgtcaacatttttggggtagttcaatctcgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 297 - protein, 234 aa, Artificial sequence
  Amino Acid sequence of D7 light chain
MSVPTQVLGLLLLWLTDARCDIQMTQSPASLSVSVGETVTITCRASDIIYSNLAWYQQKQGKSPQLLVYAATNLAAGVPSRFSGSGSGTQYSLKINSLQSEDFGTYYCQHFWGSSISFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 298 - DNA, 1380 bp, Artificial sequence
  Nucleic Acid sequence of D7 heavy chain
atggctgtcctggtgctgttcctctgcctggttgcatttccaagctgtgtcctgtcccaggtgcaactgaaggaatcaggacctggtctggtggcgccctcacagagcctgtccatcacttgcactgtctctgggttttcattaaccagctatggtgtacactgggttcgccagcctccaggaaagggtctggagtggctgggagtaatatgggctggtggaaccacaaattataattcggctctcatgtccagactgagcatcagcagagacaactccaagagccaagttttcttagaaatgaacagtctgcaaactgatgacacagccatttactactgtgccagagatggtcacttccactttgacttctggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 299 - protein, 459 aa, Artificial sequence
  Amino Acid sequence of D7 heavy chain
MAVLVLFLCLVAFPSCVLSQVQLKESGPGLVAPSQSLSITCTVSGFSLTSYGVHWVRQPPGKGLEWLGVIWAGGTTNYNSALMSRLSISRDNSKSQVFLEMNSLQTDDTAIYYCARDGHFHFDFWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 300 - DNA, 33 bp, Artificial sequence
  Nucleic Acid sequence of D7 light chain CDR
cgagcaagtgatattatttacagtaatttagca

SEQ ID NO: 301 - DNA, 21 bp, Artificial sequence
  Nucleic Acid sequence of D7 light chain CDR
gctgcaacaaacttagcagct

SEQ ID NO: 302 - DNA, 27 bp, Artificial sequence
  Nucleic Acid sequence of D7 light chain CDR
caacatttttggggtagttcaatctcg

SEQ ID NO: 303 - protein, 11 aa, Artificial sequence
  Amino Acid sequence of D7 light chain CDR
RASDIIYSNLA

SEQ ID NO: 304 - protein, 7 aa, Artificial sequence
  Amino Acid sequence of D7 light chain CDR
AATNLAA

SEQ ID NO: 305 - protein, 9 aa, Artificial sequence
  Amino Acid sequence of D7 light chain CDR
QHFWGSSIS

SEQ ID NO: 306 - DNA, 15 bp, Artificial sequence
  Nucleic Acid sequence of D7 heavy chain CDR
agctatggtgtacac

SEQ ID NO: 307 - DNA, 48 bp, Artificial sequence
  Nucleic Acid sequence of D7 heavy chain CDR
gtaatatgggctggtggaaccacaaattataattcggctctcatgtcc

SEQ ID NO: 308 - DNA, 24 bp, Artificial sequence
  Nucleic Acid sequence of D7 heavy chain CDR
gatggtcacttccactttgacttc

SEQ ID NO: 309 - protein, 5 aa, Artificial sequence
  Amino Acid sequence of D7 heavy chain CDR
SYGVH

SEQ ID NO: 310 - protein, 16 aa, Artificial sequence
  Amino Acid sequence of D7 heavy chain CDR
VIWAGGTTNYNSALMS

SEQ ID NO: 311 - protein, 8 aa, Artificial sequence
  Amino Acid sequence of D7 heavy chain CDR
DGHFHFDF

SEQ ID NO: 312 - DNA, 642 bp, Artificial sequence
  Nucleic Acid sequence of B47B6 light chain
gatattgtgctcactcagtctccagccaccctgtctgtgagtccaggagatagcgtcagtctttcctgcagggccagccaaagtattagcaacagcctacactggtatcaacaaaaatcacatgagtctccaaggcttctcatcaagtatgcttcccagtccatctctggaatcccctctaggttcagtggcagtggatcagggacagatttcactctcagtatcaacagtgtggagactgaagattttggaatgtatttctgtcaacagagttacagctggcctctcacgttcggtgctgggtccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 313 - protein, 214 aa, Artificial sequence
  Amino Acid sequence of B47B6 light chain
DIVLTQSPATLSVSPGDSVSLSCRASQSISNSLHWYQQKSHESPRLLIKYASQSISGIPSRFSGSGSGTDFTLSINSVETEDFGMYFCQQSYSWPLTFGAGSKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 314 - DNA, 1323 bp, Artificial sequence
  Nucleic Acid sequence of B47B6 heavy chain
gaagtgcagttggtggagtcggggggaggcttagtgaagcctggagggtccctgaaactctcctgtgcagcctctggattcgttttcagtagctatgacatgtcttgggttcgccaggctcaggagaagaggctggagtgggtcgcatacatgagtagtggtggcggcacctactatccagacactgtgaagggccgattcaccatctccagagacaatgccaagaacaccctgcacctgcaaatgagcagcctgaagtctgaggacacagccatgtattactgtgcaagacatgatgagattactaactttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 315 - protein, 441 aa, Artificial sequence
  Amino Acid sequence of B47B6 heavy chain
EVQLVESGGGLVKPGGSLKLSCAASGFVFSSYDMSWVRQAQEKRLEWVAYMSSGGGTYYPDTVKGRFTISRDNAKNTLHLQMSSLKSEDTAMYYCARHDEITNFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 316 - DNA, 33 bp, Artificial sequence
  Nucleic Acid sequence of B47B6 light chain CDR
agggccagccaaagtattagcaacagcctacac

SEQ ID NO: 317 - DNA, 21 bp, Artificial sequence
  Nucleic Acid sequence of B47B6 light chain CDR
tatgcttcccagtccatctct

SEQ ID NO: 318 - DNA, 27 bp, Artificial sequence
  Nucleic Acid sequence of B47B6 light chain CDR
caacagagttacagctggcctctcacg

SEQ ID NO: 319 - protein, 11 aa, Artificial sequence
  Amino Acid sequence of B47B6 light chain CDR
RASQSISNSLH

SEQ ID NO: 320 - protein, 7 aa, Artificial sequence
  Amino Acid sequence of B47B6 light chain CDR
YASQSIS

SEQ ID NO: 321 - protein, 9 aa, Artificial sequence
  Amino Acid sequence of B47B6 light chain CDR
QQSYSWPLT

SEQ ID NO: 322 - DNA, 15 bp, Artificial sequence
  Nucleic Acid sequence of B47B6  heavy chain CDR
agctatgacatgtct

SEQ ID NO: 323 - DNA, 48 bp, Artificial sequence
  Nucleic Acid sequence of B47B6  heavy chain CDR
tacatgagtagtggtggcggcacctactatccagacactgtgaagggc

SEQ ID NO: 324 - DNA, 27 bp, Artificial sequence
  Nucleic Acid sequence of B47B6  heavy chain CDR
catgatgagattactaactttgactac

SEQ ID NO: 325 - protein, 5 aa, Artificial sequence
  Amino Acid sequence of B47B6  heavy chain CDR
SYDMS

SEQ ID NO: 326 - protein, 16 aa, Artificial sequence
  Amino Acid sequence of B47B6 heavy chain CDR
YMSSGGGTYYPDTVKG

SEQ ID NO: 327 - protein, 9 aa, Artificial sequence
  Amino Acid sequence of B47B6 heavy chain CDR
HDEITNFDY

SEQ ID NO: 328 - DNA, 639 bp, Artificial sequence
  Nucleic Acid sequence of C106B9 light chain
caaattgttctcacccagtctccagcaatcatgtctgcatctccaggggagaaggtcaccataacctgcagtgtcagctcaagtgtagattacattcactggttccagcagaagccaggcacttctcccaaattctggatttatagcacatccatcctggcttctggagtccctgctcgcttcagtggcagtggatctgggacctcttactctctcacaatcagccgaatggaggctgaagatgctgccacttattactgccagcaaaggagtagttacccacccacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 329 - protein, 213 aa, Artificial sequence
  Amino Acid sequence of C106B9 light chain
QIVLTQSPAIMSASPGEKVTITCSVSSSVDYIHWFQQKPGTSPKFWIYSTSILASGVPARFSGSGSGTSYSLTISRMEAEDAATYYCQQRSSYPPTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 330 - DNA, 1320 bp, Artificial sequence
  Nucleic Acid sequence of C106B9 heavy chain
caggttcaactgcagcagtctggaggtgaggtgatgaagcctggggcctcagtgaagctttcctgcaaggctactggctacacattcactggctactggatagagtggataaaacagaggcctggacatggccttgagtggattggagagattttacctggaagtggtggtactaactacaatgagaaattcaagggcaaggccacattcactgcacatacatcctccaacacagcctacatgcaactcagcagcctgacaactgaggactctgccatctattactgtgcaagggatagtaactcctttacttactggggccaagggactctggtcactgtctcttcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 331 - protein, 440 aa, Artificial sequence
  Amino Acid sequence of C106B9 heavy chain
QVQLQQSGGEVMKPGASVKLSCKATGYTFTGYWIEWIKQRPGHGLEWIGEILPGSGGTNYNEKFKGKATFTAHTSSNTAYMQLSSLTTEDSAIYYCARDSNSFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 332 - DNA, 30 bp, Artificial sequence
  Nucleic Acid sequence of C106B9 light chain CDR
agtgtcagctcaagtgtagattacattcac

SEQ ID NO: 333 - DNA, 21 bp, Artificial sequence
  Nucleic Acid sequence of C106B9 light chain CDR
agcacatccatcctggcttct

SEQ ID NO: 334 - DNA, 27 bp, Artificial sequence
  Nucleic Acid sequence of C106B9 light chain CDR
cagcaaaggagtagttacccacccacg

SEQ ID NO: 335 - protein, 10 aa, Artificial sequence
  Amino Acid sequence of C106B9 light chain CDR
SVSSSVDYIH

SEQ ID NO: 336 - protein, 7 aa, Artificial sequence
  Amino Acid sequence of C106B9 light chain CDR
STSILAS

SEQ ID NO: 337 - protein, 9 aa, Artificial sequence
  Amino Acid sequence of C106B9 light chain CDR
QQRSSYPPT

SEQ ID NO: 338 - DNA, 15 bp, Artificial sequence
  Nucleic Acid sequence of C106B9 heavy chain CDR
ggctactggatagag

SEQ ID NO: 339 - DNA, 51 bp, Artificial sequence
  Nucleic Acid sequence of C106B9 heavy chain CDR
gagattttacctggaagtggtggtactaactacaatgagaaattcaagggc

SEQ ID NO: 340 - DNA, 21 bp, Artificial sequence
  Nucleic Acid sequence of C106B9 heavy chain CDR
gatagtaactcctttacttac

SEQ ID NO: 341 - protein, 5 aa, Artificial sequence
  Amino Acid sequence of C106B9 heavy chain CDR
GYWIE

SEQ ID NO: 342 - protein, 17 aa, Artificial sequence
  Amino Acid sequence of C106B9 heavy chain CDR
EILPGSGGTNYNEKFKG

SEQ ID NO: 343 - protein, 7 aa, Artificial sequence
  Amino Acid sequence of C106B9 heavy chain CDR
DSNSFTY

SEQ ID NO: 344 - DNA, 642 bp, Artificial sequence
  Nucleic Acid sequence of F184C7 light chain
gacatccagatgactcagtctccagcctccctatctgtatctgtgggagaaactgtcaccatcacatgtcgagcaagtgagaatatttacagaaatttagcatggtatcagcagaaacagggaaaatctcctcaactcctggtccatgctgcaacaaacttagcagatggtgtgccatcaaggttcagtggcagtggatcagacacacagtattccctcaagatcaacagcctgcagtctgaagattttgggaattattactgtcaacatttttgggggactccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 345 - protein, 214 aa, Artificial sequence
  Amino Acid sequence of F184C7 light chain
DIQMTQSPASLSVSVGETVTITCRASENIYRNLAWYQQKQGKSPQLLVHAATNLADGVPSRFSGSGSDTQYSLKINSLQSEDFGNYYCQHFWGTPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 346 - DNA, 1338 bp, Artificial sequence
  Nucleic Acid sequence of F184C7 heavy chain
caggttcagctgcagcagtctggacctgagatggtgaagcctggggcctcagtgaagattccctgcaaggcttctggctacgcattcagtagctcctggatgaactgggtgaagcagaggcctggaaagggtcttgagtggattggacggatttatcctggagatggagatactaactacaatgagaagttcaagggcaaggccacactgactgtagacaaatcctccagcacagtctacatgcaactcagcagcctgacatctgaggactctgcggtctacttctgtgcaagagaggctactacggtagtggccccgtactactttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 347 - protein, 446 aa, Artificial sequence
  Amino Acid sequence of F184C7 heavy chain
QVQLQQSGPEMVKPGASVKIPCKASGYAFSSSWMNWVKQRPGKGLEWIGRIYPGDGDTNYNEKFKGKATLTVDKSSSTVYMQLSSLTSEDSAVYFCAREATTVVAPYYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 348 - DNA, 33 bp, Artificial sequence
  Nucleic Acid sequence of F184C7 light chain CDR
cgagcaagtgagaatatttacagaaatttagca

SEQ ID NO: 349 - DNA, 21 bp, Artificial sequence
  Nucleic Acid sequence of F184C7 light chain CDR
gctgcaacaaacttagcagat

SEQ ID NO: 350 - DNA, 27 bp, Artificial sequence
  Nucleic Acid sequence of F184C7 light chain CDR
caacatttttgggggactccgctcacg

SEQ ID NO: 351 - protein, 11 aa, Artificial sequence
  Amino Acid sequence of F184C7 light chain CDR
RASENIYRNLA

SEQ ID NO: 352 - protein, 7 aa, Artificial sequence
  Amino Acid sequence of F184C7 light chain CDR
AATNLAD

SEQ ID NO: 353 - protein, 9 aa, Artificial sequence
  Amino Acid sequence of F184C7 light chain CDR
QHFWGTPLT

SEQ ID NO: 354 - DNA, 22 bp, Artificial sequence
  Nucleic Acid sequence of F184C7 heavy chain CDR
attcagtagctcctggatgaac

SEQ ID NO: 355 - DNA, 51 bp, Artificial sequence
  Nucleic Acid sequence of F184C7 heavy chain CDR
cggatttatcctggagatggagatactaactacaatgagaagttcaagggc

SEQ ID NO: 356 - DNA, 39 bp, Artificial sequence
  Nucleic Acid sequence of F184C7 heavy chain CDR
gaggctactacggtagtggccccgtactactttgactac

SEQ ID NO: 357 - protein, 7 aa, Artificial sequence
  Amino Acid sequence of F184C7 heavy chain CDR
FSSSWMN

SEQ ID NO: 358 - protein, 17 aa, Artificial sequence
  Amino Acid sequence of F184C7 heavy chain CDR
RIYPGDGDTNYNEKFKG

SEQ ID NO: 359 - protein, 13 aa, Artificial sequence
  Amino Acid sequence of F184C7 heavy chain CDR
EATTVVAPYYFDY

SEQ ID NO: 360 - DNA, 642 bp, Artificial sequence
  Nucleic Acid sequence of D10A3 light chain
aatattgtgctgacccagactcccaaattcctgcttgtatcagcaggagacagggtttccataacctgcaaggccagtcagcgtgtgaataatgatgtagcttggtaccaacagaagccagggcagtctcctaaactgctgatatactatgcatccaatcgctacactggagtccctgatcgcttcactggcagtggatatgggacggatttcactttcaccatcagcactgtgcaggctgaagacctggcagtttatttctgtcagcaggattatagctctccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 361 - protein, 214 aa, Artificial sequence
  Amino Acid sequence of D10A3 light chain
NIVLTQTPKFLLVSAGDRVSITCKASQRVNNDVAWYQQKPGQSPKLLIYYASNRYTGVPDRFTGSGYGTDFTFTISTVQAEDLAVYFCQQDYSSPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 362 - DNA, 1332 bp, Artificial sequence
  Nucleic Acid sequence of D10A3 heavy chain
gaggtccagctgcaacagtttggaactgagctggtgaagcctggggcttcagtgaagatatcctgcaaggcttctggctacacattcactgactacaacatggactgggtgaagcagagccatggaaagagccttgagtggattggagatattaatcctaactatgatactactacctacaaccagaagttcaagggaaaggccacattgactgtagacaagtcctccagcacagcctacatggagctccgcagcctgacttctgaggacactgcagtcttttactgtgcaagaaggaactatggtaactacgtggggtttgacttctggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 363 - protein, 444 aa, Artificial sequence
  Amino Acid sequence of D10A3 heavy chain
EVQLQQFGTELVKPGASVKISCKASGYTFTDYNMDWVKQSHGKSLEWIGDINPNYDTTTYNQKFKGKATLTVDKSSSTAYMELRSLTSEDTAVFYCARRNYGNYVGFDFWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 364 - DNA, 33 bp, Artificial sequence
  Nucleic Acid sequence of D10A3 light chain CDR
aaggccagtcagcgtgtgaataatgatgtagct

SEQ ID NO: 365 - DNA, 21 bp, Artificial sequence
  Nucleic Acid sequence of D10A3 light chain CDR
tatgcatccaatcgctacact

SEQ ID NO: 366 - DNA, 27 bp, Artificial sequence
  Nucleic Acid sequence of D10A3 light chain CDR
cagcaggattatagctctccattcacg

SEQ ID NO: 367 - protein, 11 aa, Artificial sequence
  Amino Acid sequence of D10A3 light chain CDR
KASQRVNNDVA

SEQ ID NO: 368 - protein, 7 aa, Artificial sequence
  Amino Acid sequence of D10A3 light chain CDR
YASNRYT

SEQ ID NO: 369 - protein, 9 aa, Artificial sequence
  Amino Acid sequence of D10A3 light chain CDR
QQDYSSPFT

SEQ ID NO: 370 - DNA, 15 bp, Artificial sequence
  Nucleic Acid sequence of D10A3 heavy chain CDR
gactacaacatggac

SEQ ID NO: 371 - DNA, 51 bp, Artificial sequence
  Nucleic Acid sequence of D10A3 heavy chain CDR
gatattaatcctaactatgatactactacctacaaccagaagttcaaggga

SEQ ID NO: 372 - DNA, 33 bp, Artificial sequence
  Nucleic Acid sequence of D10A3 heavy chain CDR
aggaactatggtaactacgtggggtttgacttc

SEQ ID NO: 373 - protein, 5 aa, Artificial sequence
  Amino Acid sequence of D10A3 heavy chain CDR
DYNMD

SEQ ID NO: 374 - protein, 17 aa, Artificial sequence
  Amino Acid sequence of D10A3 heavy chain CDR
DINPNYDTTTYNQKFKG

SEQ ID NO: 375 - protein, 11 aa, Artificial sequence
  Amino Acid sequence of D10A3 heavy chain CDR
RNYGNYVGFDF

SEQ ID NO: 376 - protein, 163 aa, homo sapiens; identical to SEQ ID NO: 8

SEQ ID NO: 377 - protein, 164 aa, homo sapiens; identical to SEQ ID NO: 9

SEQ ID NO: 378 - protein, 123 aa, homo sapiens; identical to SEQ ID NO: 10

SEQ ID NO: 379 - protein, 101 aa, homo sapiens; identical to SEQ ID NO: 11

SEQ ID NO: 380 - protein, 220 aa, homo sapiens; identical to SEQ ID NO: 12

SEQ ID NO: 381 - protein, 255 aa, homo sapiens; identical to SEQ ID NO: 13

SEQ ID NO: 382 - protein, 199 aa, homo sapiens; identical to SEQ ID NO: 14

SEQ ID NO: 383 - protein, 277 aa, homo sapiens; identical to SEQ ID NO: 15

SEQ ID NO: 384 - protein, 9 aa, Artificial Sequence
  short peptide
DLMGYIPLV

(19) United States
(12) Patent Application Publication
(10) Pub. No.: US 2018/0179283 A1
(43) Pub. Date: Jun. 28, 2018

(54) T CELL RECEPTOR LIKE ANTIBODIES HAVING FINE SPECIFICITY

(71) Applicant: Adicet Bio Inc., Menlo Park, CA (US)

(72) Inventors: Mira PELED KAMAR, Herzlia (IL); Galit DENKBERG, Nofit (IL); Yoram REITER, Haifa (IL); Ilan BEER, Haifa (IL); Keren SINIK, Ramat-Yishai (IL); Yael TEBOUL (ELBAZ), Haifa (IL); Yael SHPERBER (SERY), Kfar-Vradim (IL); Reut EREL SEGAL, Haifa (IL); Ravit OREN, Haifa (IL); Dror Shmuel ALISHEKEVITZ, Kiryat-Tivon (IL)

(21) Appl. No.: 15/579,974

(22) PCT Filed: Jun. 8, 2016
(86) PCT No.: PCT/IL2016/050599

§ 371 Date: Dec. 6, 2017

Related U.S. Application Data
(60) Provisional application No. 62/172,264, filed on Jun. 8, 2015.

(51) Int. Cl. C07K 16/28; C07K 16/40; C07K 16/30; G01N 33/566

(52) U.S. Cl. CPC C07K 16/2833; C07K 16/40; C07K 16/3053; A61K 2039/505; C07K 2317/31; C07K 2317/55; G01N 33/566

(57) ABSTRACT

An antibody capable of binding, with a human major histocompatibility complex (MHC)-restricted specificity, a MHC being complexed with an HLA-restricted peptide antigen is provided. The antibody having a binding specificity dictated by at least 4 amino acid residues in said HLA-restricted peptide such that at least 70% reduction in binding of said antibody to said complex is observed when each of said at least 4 amino acid residues is substituted as determined by FACS of cells loaded with said HLA-restricted peptide comprising said substitution, said at least 4 amino acid residues not being anchor residues.

### FIELD AND BACKGROUND OF THE INVENTION

The present invention, in some embodiments thereof, relates to T Cell Receptor like antibodies having fine specificity.

Major histocompatibility complex (MHC) class I molecules are key in the immune response against malignant cells by shaping the T-cell repertoire and presenting peptides from endogenous antigens to CD8+ cytotoxic T cells. Because of their unique specificity, MHC-peptide complexes are a desirable target for novel immunotherapeutic approaches. These complexes can be targeted by recombinant T-cell receptors (TCRs). However, most TCRs produced thus far have affinities which are too low for target detection under normal assay conditions, and limited stability (due to their generation in a single-chain version). Developing high-affinity soluble antibody molecules endowed with a TCR-like specificity toward tumor epitopes, termed TCR-like antibodies, addresses the low affinity of TCRs. These high affinity TCR-like antibodies are being developed as a new immunotherapeutic class for targeting tumor cells and mediating their specific killing. In addition, these antibodies are valuable research reagents enabling the study of human class I peptide-MHC ligand-presentation and TCR-peptide-MHC interactions.

The generation of high affinity TCR-like antibodies has been the focus of many research laboratories and pharmaceutical companies. A key for successful production of TCR-like antibodies is having an MHC-peptide complex folded in a native conformation that is recognized by the T cell (reviewed in Cohen and Reiter Antibodies 1013 2:517-534). Once such a complex is at hand various technologies may be implemented towards the generation and/or selection of high affinity TCR-like antibodies, including phage display libraries (e.g., native libraries or immunized libraries) as well as the hybridoma technology.

However, one of the most critical challenges for TCR-like antibodies is the risk of treatment-induced toxicity. Such a situation might arise through non-specific binding of the introduced antibody with off-target peptides.

There is thus a need for screening assays that would result in a novel class of TCR-like antibodies devoid of off-target toxicity.

Additional background art includes:

WO2008/120202

Cameron et al. 2013 Sci. Transl. Med. 6:197ra103; and

Cohen and Reiter 2013 Antibodies 2:517-534

### SUMMARY OF THE INVENTION

According to an aspect of some embodiments of the present invention there is provided an antibody capable of binding, with a human major histocompatibility complex (MHC)-restricted specificity, a MHC being complexed with an HLA-restricted peptide antigen, the antibody having a binding specificity dictated by at least 4 amino acid residues in the HLA-restricted peptide such that at least 70% reduction in binding of the antibody to the complex is observed when each of the at least 4 amino acid residues is substituted as determined by FACS of cells loaded with the HLA-restricted peptide comprising the substitution, the at least 4 amino acid residues not being anchor residues.

According to an aspect of some embodiments of the present invention there is provided an antibody capable of binding, with a human major histocompatibility complex (MHC)-restricted specificity, a HLA-A2/TyrD369-377 peptide complex, the antibody having a binding specificity dictated by at least 4 amino acid residues in the TyrD369-377 peptide such that at least 70% reduction in binding of the antibody to the complex is observed when each of the at least 4 amino acid residues is substituted as determined by FACS of cells loaded with the peptide comprising the substitution, the at least 4 amino acid residues not being anchor residues.

According to some embodiments of the invention, the at least 4 amino acid residues are selected from X1, X3, X4, X6 and X7 of TyrD 369-377.

According to some embodiments of the invention, the at least 4 amino acid residues are selected from X3, X4, X6 and X7 of TyrD369-377.

According to some embodiments of the invention, the at least 4 amino acid residues are selected from X1, X3, X4 and X6 of TyrD369-377.

According to some embodiments of the invention, the at least 70% reduction in binding is at least 90% reduction in binding observed when at least 1 amino acid residue of the at least 4 amino acid residues comprises the substitution.

According to some embodiments of the invention, the at least 70% reduction in binding is at least 90% reduction in binding observed when each of at least 2 amino acid residues of the at least 4 amino acid residues comprise the substitutions.

According to an aspect of some embodiments of the present invention there is provided an antibody capable of binding, with a human major histocompatibility complex (MHC)-restricted specificity, a MHC being complexed with an HLA-restricted peptide antigen, wherein the antibody does not bind to any HLA-presented peptides, which are present in essential tissues as determined by FACS analysis of cells loaded with the HLA-presented peptides, the HLA-presented peptides having at least one amino acid substitution as compared to the HLA-restricted peptide antigen in an amino acid residue not critical for binding the peptide antigen, as determined by alanine scanning of the HLA-restricted peptide antigen.

According to some embodiments of the invention, the antibody has a binding affinity below 20 nanomolar to a single chain human major histocompatibility complex (MHC) complexed with the HLA-restricted peptide antigen, as determined by surface plasmon resonance assay.

According to some embodiments of the invention, the antibody has a binding affinity below 10 nanomolar to a single chain human major histocompatibility complex (MHC) complexed with the HLA-restricted peptide antigen, as determined by surface plasmon resonance assay.

According to some embodiments of the invention, the antibody is capable of binding the HLA-restricted peptide antigen when naturally presented on cells, as determined by FACS.

According to some embodiments of the invention, the cells are cancer cells.

According to some embodiments of the invention, the antibody is of an IgG1 or

IgG4 subclass.

According to some embodiments of the invention, the antibody comprises a therapeutic moiety.

According to some embodiments of the invention, the antibody comprises an identifiable moiety.

According to some embodiments of the invention, the antibody does not bind an in-silico predicted HLA-restricted peptide.

According to some embodiments of the invention, the HLA-restricted peptide antigen is selected from the group consisting of a tumor HLA-restricted peptide antigen, a viral HLA-restricted peptide antigen and an autoimmune HLA-restricted peptide antigen.

According to some embodiments of the invention, the MHC is class I MHC.

According to some embodiments of the invention, the antibody comprises a therapeutic moiety for use in treating a cancer.

According to some embodiments of the invention, the therapeutic moiety comprises CD3.

According to some embodiments of the invention, the antibody is a bispecific antibody.

According to some embodiments of the invention, the antibody is soluble.

According to some embodiments of the invention, the antibody is insoluble.

According to some embodiments of the invention, the antibody forms a CAR.

According to some embodiments of the invention, the HLA-restricted peptide antigen is derived from tyrosinase.

According to some embodiments of the invention, the HLA-restricted peptide antigen is derived from WT1.

According to some embodiments of the invention, the at least one amino acid substitution comprises 1-4 amino acid substitutions.

According to an aspect of some embodiments of the present invention there is provided an isolated polynucleotide comprising a nucleic acid sequence encoding the antibody.

According to an aspect of some embodiments of the present invention there is provided n expression vector comprising the polynucleotide operably linked to a cis-acting regulatory element.

According to an aspect of some embodiments of the present invention there is provided a cell comprising the polynucleotide or the expression vector.

According to an aspect of some embodiments of the present invention there is provided a method for antibody qualification for TCRL therapy, the method comprising:

(a) providing an antibody capable of binding, with a human major histocompatibility complex (MHC)-restricted specificity, a MHC being complexed with an HLA-restricted peptide antigen, wherein the binding is with a predetermined affinity;
(b) providing HLA-presented peptides which are present on at least one essential tissue, the HLA-presented peptides having at least one amino acid substitution as compared to the HLA-restricted peptide antigen in an amino acid residue not critical for binding the peptide antigen, as determined by alanine scanning of the HLA-restricted peptide antigen;
(c) determining binding of the antibody to the HLA-presented peptides by FACS analysis of cells loaded with the HLA-presented peptides, the antibody being qualified for TCRL therapy if the binding is undetectable by the FACS analysis.

According to an aspect of some embodiments of the present invention there is provided an antibody capable of binding, with a human major histocompatibility complex (MHC)-restricted specificity, a MHC being complexed with an HLA-restricted peptide tumor antigen or autoimmune antigen, the antibody having a binding specificity dictated by at least 4 amino acid residues in the HLA-restricted peptide such that at least 70% reduction in binding of the antibody to the complex is observed when each of the at least 4 amino acid residues is substituted by alanine as determined by FACS of cells loaded with the HLA-restricted peptide comprising the substitution, the at least 4 amino acid residues not being anchor residues.

According to an aspect of some embodiments of the present invention there is provided an antibody capable of binding, with a human major histocompatibility complex (MHC)-restricted specificity, a MHC being complexed with an HLA-restricted peptide antigen, the antibody having a binding specificity dictated by at least 4 amino acid residues in the HLA-restricted peptide such that at least 70% reduction in binding of the antibody to the complex is observed when each of the at least 4 amino acid residues is substituted by alanine as determined by FACS of cells loaded with the HLA-restricted peptide comprising the substitution, the at least 4 amino acid residues not being anchor residues and further wherein the antibody does not bind HLA-restricted peptide antigens presented on normal essential tissues, wherein the HLA-restricted peptide antigen is not from LMP-2A polypeptide.

According to an aspect of some embodiments of the present invention there is provided an antibody capable of binding, with a human major histocompatibility complex (MHC)-restricted specificity, a HLA-A2/TyrD369-377 peptide complex, the antibody having a binding specificity dictated by at least 4 amino acid residues in the TyrD369-377 peptide such that at least 70% reduction in binding of the antibody to the complex is observed when each of the at least 4 amino acid residues is substituted by alanine as determined by FACS of cells loaded with the peptide comprising the substitution, the at least 4 amino acid residues not being anchor residues.

According to some embodiments of the invention, the at least 4 amino acid residues are selected from X1, X3, X4, X6 and X7 of TyrD 369-377.

According to some embodiments of the invention, the at least 4 amino acid residues are selected from X3, X4, X6 and X7 of TyrD369-377.

According to some embodiments of the invention, the at least 4 amino acid residues are selected from X1, X3, X4 and X6 of TyrD369-377.

According to some embodiments of the invention, the at least 70% reduction in binding is at least 90% reduction in binding observed when at least 1 amino acid residue of the at least 4 amino acid residues comprises the substitution.

According to some embodiments of the invention, the at least 70% reduction in binding is at least 90% reduction in binding observed when each of at least 2 amino acid residues of the at least 4 amino acid residues comprise the substitutions.

According to some embodiments of the invention, the at least 4 amino acid residues comprise 5 amino acid residues such that at least 70% reduction in binding of the antibody to the complex is observed when each of at least 4 amino acid residues of the 5 amino acids residues is substituted by alanine and wherein at least 30% reduction in binding of the antibody to the complex is observed when a fifth amino acid of the 5 amino acids is substituted by alanine.

According to some embodiments of the invention, the antibody does not bind to HLA-presented peptides, which are present in essential tissues as determined by FACS analysis of cells loaded with the HLA-presented peptides, the HLA-presented peptides having at least one amino acid substitution as compared to the HLA-restricted peptide antigen in an amino acid residue which is not one of the at least 4 amino acid residues.

According to an aspect of some embodiments of the present invention there is provided an antibody capable of binding, with a human major histocompatibility complex (MHC)-restricted specificity, a MHC being complexed with an HLA-restricted peptide antigen, wherein the antibody does not bind to HLA-presented peptides, which are present in essential tissues as determined by FACS analysis of cells loaded with the HLA-presented peptides, the HLA-presented peptides having at least one amino acid substitution as compared to the HLA-restricted peptide antigen in an amino acid residue not critical for binding the peptide antigen, as determined by as determined by FACS of cells loaded with the HLA-restricted peptide comprising an alanine substitution.

According to some embodiments of the invention, the HLA-restricted peptide antigen is not from LMP-2A polypeptide.

According to some embodiments of the invention, the antibody has a binding affinity below 20 nanomolar to a single chain human major histocompatibility complex (MHC) complexed with the HLA-restricted peptide antigen, as determined by surface plasmon resonance assay.

According to some embodiments of the invention, has a binding affinity below 10 nanomolar to a single chain human major histocompatibility complex (MHC) complexed with the HLA-restricted peptide antigen, as determined by surface plasmon resonance assay.

According to some embodiments of the invention, the antibody is capable of binding the HLA-restricted peptide antigen when naturally presented on cells, as determined by FACS.

According to some embodiments of the invention, the cells are cancer cells.

According to some embodiments of the invention, the antibody is of an IgG1 or IgG4 subclass.

According to some embodiments of the invention, the antibody comprises to a therapeutic moiety.

According to some embodiments of the invention, the antibody comprises an identifiable moiety.

According to some embodiments of the invention, the antibody does not bind an in-silico predicted HLA-restricted peptide.

According to some embodiments of the invention, the HLA-restricted peptide antigen is selected from the group consisting of a tumor HLA-restricted peptide antigen, a viral HLA-restricted peptide antigen and an autoimmune HLA-restricted peptide antigen.

According to some embodiments of the invention, the MHC is class I MHC.

According to some embodiments of the invention, the antibody comprises a therapeutic moiety for use in treating a cancer.

According to some embodiments of the invention, the therapeutic moiety comprises a CD3 engaging molecule.

According to some embodiments of the invention, the antibody is a bispecific antibody.

According to some embodiments of the invention, the antibody is soluble.

According to some embodiments of the invention, the antibody is insoluble.

According to some embodiments of the invention, the antibody forms a CAR.

According to some embodiments of the invention, the HLA-restricted peptide antigen is derived from tyrosinase.

According to some embodiments of the invention, the HLA-restricted peptide antigen is derived from WT1.

According to some embodiments of the invention, the HLA-restricted peptide antigen is derived from MAGE-A4.

According to some embodiments of the invention, the HLA-restricted peptide antigen is derived from MAGE-A9.

According to some embodiments of the invention, the HLA-restricted peptide antigen is derived from PAP.

According to some embodiments of the invention, the at least one amino acid substitution comprises 1-4 amino acid substitutions.

According to an aspect of some embodiments of the present invention there is provided an isolated polynucleotide comprising a nucleic acid sequence encoding the antibody.

According to an aspect of some embodiments of the present invention there is provided an expression vector comprising the polynucleotide is operably linked to a cis-acting regulatory element.

According to an aspect of some embodiments of the present invention there is provided a cell comprising the polynucleotide or the expression vector.

According to an aspect of some embodiments of the present invention there is provided a method for selecting highly selective TCR-like antibody with the optimal fine specificity towards a specific MHC being complexed with an HLA-restricted peptide antigen, the method comprising:

(a) providing an antibody capable of binding, with a human major histocompatibility complex (MHC)-restricted specificity, a MHC being complexed with an HLA-restricted peptide antigen, wherein the binding is with a predetermined affinity;
(b) determining binding of the antibody to peptides similar to the HLA-restricted peptide antigen having been mutated with alanine/glycine/valine/leucine in amino acids other than anchor residues so as to identify amino acids which are critical for binding of the antibody to the HLA-restricted peptide;
(c) determining binding of the antibody to in silico-predicted and/or validated HLA-presented peptides which are present on at least one normal essential tissue, the HLA-presented peptides comprising 1-4 amino acid substitutions as compared to the HLA-restricted peptide;
wherein the determining binding of the antibody to the peptides of (a) and (b) is by FACS analysis of cells loaded with the peptides or by functional assay, the antibody being qualified if the binding of (b) is undetectable by the FACS analysis.

According to some embodiments of the invention, the HLA-presented peptides which are present on at least one essential tissue have at least one amino acid substitution as compared to the HLA-restricted peptide antigen in an amino acid residue not critical for binding the peptide antigen.

According to some embodiments of the invention, the method further comprises determining binding of the antibody to normal cells, which do not present the HLA-restricted peptide but are positive for the HLA.

According to an aspect of some embodiments of the present invention there is provided an antibody which qualifies the above criteria.

According to some embodiments of the invention, the predetermined affinity is below 20 nM.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1: Apparent binding affinity determination of TCR-like antibodies targeting HLA-A2/Tyrosinase complexes. Purified IgGs were immobilized indirectly to the SPR sensor chip with anti-mouse or human IgG. Analyte was purified recombinant single-chain HLA-A2/Tyrosinase complexes generated by in vitro refolding of E. coli expressed scHLA-A2 complexes.

FIG. 2: Epitope specificity determination of TCR-like antibodies by Alanine scanning. The Tyrosinase peptide sequence was substituted with Alanine at positions 1, 2, 3, 4, 5, 6, 7, and 8. The Ala mutated peptides were synthesized and loaded onto T2 cells APCs at a concentration of 10⁻⁴-10⁻⁵M for 12 hrs at 37° C. Binding of TCR-like antibodies at a concentration of 10 μg/ml was accessed by flow cytometry and binding intensity as measured by mean fluorescence intensity was measured and compared with the binding intensity to WT native Tyrosinase peptide. The relative effect of each position Ala substitution was evaluated as percentage to the binding to WT peptide.

FIG. 3: Binding of D11 and D7 TCR-like antibodies to T2 APCs loaded with tyrosinase peptide and control HLA-A2 restricted peptides. T2 cells were loaded with Tyrosinase peptide and indicated peptides at a concentration of 10⁻⁴-10⁻⁵M for 12 hrs at 37° C. Binding was monitored by flow cytometry using secondary PE-labeled anti-mouse IgG. MAb BB7.2 was used to monitor expression of HLA-A2. Mean fluorescence intensity (MFI) is indicated.

FIG. 4: Binding of D11 and D7 TCR-like antibodies to T2 APCs loaded with tyrosinase peptide and control HLA-A2 restricted peptides. T2 cells were loaded with Tyrosinase peptide and indicated peptides at a concentration of 10⁻⁴-10⁻⁵M for 12 hrs at 37° C. Binding was monitored by flow cytometry using secondary PE-labeled anti-mouse IgG. MAb BB7.2 was used to monitor expression of HLA-A2. Mean fluorescence intensity (MFI) is indicated.

FIG. 5: Binding of D11 TCR-like antibody to T2 APCs loaded with tyrosinase peptide and control HLA-A2 restricted peptides. T2 cells were loaded with Tyrosinase peptide and indicated peptides at a concentration of 10⁴-10⁻⁵M for 12 hrs at 37° C. Binding was monitored by flow cytometry using secondary PE-labeled anti-mouse IgG. MAb BB7.2 was used to monitor expression of HLA-A2. Mean fluorescence intensity (MFI) is indicated.

FIG. 6: Binding of D7 TCR-like antibody to T2 APCs loaded with tyrosinase peptide and control HLA-A2 restricted peptides. T2 cells were loaded with Tyrosinase peptide and indicated peptides at a concentration of 10⁴-10⁻⁵M for 12 hrs at 37° C. Binding was monitored by flow cytometry using secondary PE-labeled anti-mouse IgG. MAb BB7.2 was used to monitor expression of HLA-A2. Mean fluorescence intensity (MFI) is indicated.

FIG. 7: Binding of MC1 TCR-like antibody to T2 APCs loaded with tyrosinase peptide and control HLA-A2 restricted peptides. T2 cells were loaded with Tyrosinase peptide and indicated peptides at a concentration of 10⁻⁴-10⁻⁵M for 12 hrs at 37° C. Binding was monitored by flow cytometry using secondary PE-labeled anti-mouse IgG. MAb BB7.2 was used to monitor expression of HLA-A2. Mean fluorescence intensity (MFI) is indicated.

FIG. 8: Binding of MC1 TCR-like antibody to melanoma cells that express HLA-A2 and Tyrosinase. Melanoma cells were monitored by flow cytometry for binding of TCR-like antibody MC1 using secondary PE-labeled anti-human IgG. MAb BB7.2 was used to monitor expression of HLA-A2. Mean fluorescence intensity (MFI) is indicated.

FIG. 9: Binding of MC1 TCR-like antibody to HLA-A2+ and Tyrosinase antigen positive or negative cells. Tumor cells that express HLA-A2 and are positive or negative for Tyrosinase were monitored by flow cytometry for binding of TCR-like antibody MC1 using secondary PE-labeled anti-human IgG. MAb BB7.2 was used to monitor expression of HLA-A2. Mean fluorescence intensity (MFI) is indicated.

FIG. 10: Binding of D11 and D7 TCR-like antibodies to HLA-A2+ and Tyrosinase antigen positive or negative cells. Tumor cells that express HLA-A2 and are positive or negative for Tyrosinase were monitored by flow cytometry for binding of TCR-like antibody MC1 using secondary PE-labeled anti-mouse IgG. MAb BB7.2 was used to monitor expression of HLA-A2. Mean fluorescence intensity (MFI) is indicated.

FIG. 11: Binding of D11 and D7 TCR-like antibodies to HLA-A2+ and Tyrosinase negative cells. Tumor cells that express HLA-A2 and are negative for Tyrosinase were monitored by flow cytometry for binding of TCR-like antibody MC1 using secondary PE-labeled anti-mouse IgG. MAb BB7.2 was used to monitor expression of HLA-A2. Mean fluorescence intensity (MFI) is indicated.

FIG. 12: Comparative Binding of D11, D7, and MC1 TCR-like antibodies to HLA-A2+ and Tyrosinase positive or negative cells. Tumor cells that express HLA-A2 and are positive or negative for Tyrosinase were monitored by flow cytometry for binding of TCR-like antibody D11, D7, and MC1 using secondary PE-labeled anti-mouse IgG.

FIG. 13: Binding of D11 TCR-like antibody to HLA-A2+/Tyrosinase negative normal primary cells. Primary normal cells of histological origin as indicated that express HLA-A2 and are negative for Tyrosinase were monitored by flow cytometry for binding of TCR-like antibody D11, using secondary PE-labeled anti-mouse IgG. MAb BB7.2 was used to monitor expression of HLA-A2.

FIG. 14: Binding of D11 TCR-like antibody to HLA-A2+/Tyrosinase negative normal primary cells. Primary normal cells of histological origin as indicated that express HLA-A2 and are negative for Tyrosinase were monitored by flow cytometry for binding of TCR-like antibody D11, using secondary PE-labeled anti-mouse IgG.

FIG. 15: Binding of D7 TCR-like antibody to HLA-A2+/Tyrosinase negative normal primary cells. Primary normal cells of histological origin as indicated that express HLA-A2 and are negative for Tyrosinase were monitored by flow cytometry for binding of TCR-like antibody D7, using secondary PE-labeled anti-mouse IgG.

FIG. 16: Binding of BB7.2 to normal primary cells. Primary normal cells of histological origin were monitored by flow cytometry for expression of HLA-A2 using MAb BB7.2 and secondary PE-labeled anti-mouse IgG.

FIG. 17: Binding of MC1, D11 and D7 TCR-like antibodies to normal PBMCs. PBMCs were characterized for HLA-A2 homo or heterozygosity by PCR. Binding of TCR-like antibodies was monitored by PE-labeled secondary anti-mouse IgG.

FIG. 18: Summary of D11 TCR-like antibody selectivity. Binding of D11 TCR-like antibodies to HLA-A2+ antigen positive and negative cells was monitored by using PE-labeled anti-mouse IgG. +/− indicate tyrosinase mRNA gene expression as measured by PCR. HLA-A2 expression was monitored with MAb BB7.2.

FIG. 19: Summary of D7 TCR-like antibody selectivity. Binding of D7 TCR-like antibodies to HLA-A2+ antigen positive and negative cells was monitored by using PE-labeled anti-mouse IgG. +/− indicate tyrosinase mRNA gene expression as measured by PCR. HLA-A2 expression was monitored with MAb BB7.2.

FIG. 20: Binding of MC1, D11, and D7 TCR-like antibodies to T2 APCs loaded with tyrosinase peptide and tyrosinase similar HLA-A2 restricted peptides. T2 cells were loaded with Tyrosinase peptide and indicated peptides at a concentration of 10⁻⁴M for 12 hrs at 37° C. Binding was monitored by flow cytometry using secondary PE-labeled anti-mouse IgG.

FIG. 21: Binding of D11 TCR-like antibody to T2 APCs loaded with tyrosinase peptide similar HLA-A2 restricted peptides. T2 cells were loaded with Tyrosinase peptide and indicated peptides at a concentration of 10⁻⁵M for 12 hrs at 37° C. Binding was monitored by flow cytometry using secondary PE-labeled anti-mouse IgG. Binding of MAb BB7.2 ensured measurement of peptide loading efficiency.

FIG. 22: Binding of D11 TCR-like antibody to T2 APCs loaded with tyrosinase peptide similar HLA-A2 restricted peptides. T2 cells were loaded with Tyrosinase peptide and indicated peptides at a concentration of 10⁻⁵M for 12 hrs at 37° C. Binding was monitored by flow cytometry using secondary PE-labeled anti-mouse IgG. Binding of MAb BB7.2 ensured measurement of peptide loading efficiency.

FIG. 23: Binding of D11 TCR-like antibody to T2 APCs loaded with tyrosinase peptide similar HLA-A2 restricted peptides. T2 cells were loaded with Tyrosinase peptide and indicated peptides at a concentration of 10⁻⁵M for 12 hrs at 37° C. Binding was monitored by flow cytometry using secondary PE-labeled anti-mouse IgG. Binding of MAb BB7.2 ensured measurement of peptide loading efficiency.

FIG. 24: Binding of D11 TCR-like antibody to T2 APCs loaded with tyrosinase similar HLA-A2 restricted peptides. T2 cells were loaded with Tyrosinase peptide and indicated peptides at a concentration of −10⁻⁵M for 12 hrs at 37° C. Binding was monitored by flow cytometry using secondary PE-labeled anti-mouse IgG. Binding of MAb BB7.2 ensured measurement of peptide loading efficiency.

FIG. 25: Binding of D7 TCR-like antibody to T2 APCs loaded with tyrosinase similar HLA-A2 restricted peptides. T2 cells were loaded with Tyrosinase peptide and indicated peptides at a concentration of 10⁻⁵M for 12 hrs at 37° C. Binding was monitored by flow cytometry using secondary PE-labeled anti-mouse IgG. Binding of MAb BB7.2 ensured measurement of peptide loading efficiency.

FIG. 26: Binding of D7 TCR-like antibody to T2 APCs loaded with tyrosinase similar HLA-A2 restricted peptides. T2 cells were loaded with Tyrosinase peptide and indicated peptides at a concentration of 10⁻⁵M for 12 hrs at 37° C. Binding was monitored by flow cytometry using secondary PE-labeled anti-mouse IgG. Binding of MAb BB7.2 ensured measurement of peptide loading efficiency.

FIG. 27: Binding of D7 TCR-like antibody to T2 APCs loaded with tyrosinase similar HLA-A2 restricted peptides. T2 cells were loaded with Tyrosinase peptide and indicated peptides at a concentration of 10⁻⁵M for 12 hrs at 37° C. Binding was monitored by flow cytometry using secondary PE-labeled anti-mouse IgG. Binding of MAb BB7.2 ensured measurement of peptide loading efficiency.

FIG. 28: Binding of D7 TCR-like antibody to T2 APCs loaded with tyrosinase similar HLA-A2 restricted peptides identified after alanine scanning. T2 cells were loaded with Tyrosinase peptide and indicated peptides which were selected according to epitope recognition specificity of by D7 of Ala mutated peptides at a concentration of 10⁻⁵M for 12 hrs at 37° C. Binding was monitored by flow cytometry using secondary PE-labeled anti-mouse IgG. Binding of MAb BB7.2 ensured measurement of peptide loading efficiency.

FIG. 29: Apparent binding affinity determination of TCR-like antibody B47B6 targeting HLA-A2/WT1 complexes. Purified IgGs were immobilized indirectly to the SPR sensor chip with anti-mouse. Analyte was purified recombinant single-chain HLA-A2/WT1 complexes generated by in vitro refolding of E. coli expressed scHLA-A2 complexes.

FIG. 30: Binding of B47 and ESK1 TCR-like antibodies to T2 APCs loaded with WT1 HLA-A2 restricted peptide. T2 cells were loaded with WT1 at a concentration of 10⁻⁴-10⁻⁵M for 12 hrs at 37° C. Binding was monitored by flow cytometry using secondary PE-labeled anti-mouse IgG (for B47) or human IgG (for ESK1). MAb BB7.2 was used to monitor expression of HLA-A2. Mean fluorescence intensity (MFI) is indicated.

FIG. 31: Binding of B47 and ESK1 TCR-like antibodies to T2 APCs loaded with WT1 peptide and control HLA-A2 restricted peptides. T2 cells were loaded with WT1 peptide and indicated peptides at a concentration of 10⁻⁴M for 12 hrs at 37° C. Binding was monitored by flow cytometry using secondary PE-labeled anti-mouse IgG (for B47) or human IgG (for ESK1). MAb BB7.2 was used to monitor expression of HLA-A2. Mean fluorescence intensity (MFI) is indicated.

FIG. 32: Binding of B47 and ESK1 TCR-like antibodies to T2 APCs loaded with WT1 similar HLA-A2 restricted peptides. T2 cells were loaded with WT1 peptide and indicated peptides at a concentration of 10⁻⁴-10⁻⁵M for 12 hrs at 37° C. Binding was monitored by flow cytometry using secondary PE-labeled anti-mouse IgG (for B47) or human IgG (for ESK1). Binding of MAb BB7.2 ensured measurement of peptide loading efficiency.

FIG. 33: Binding of B47 TCR-like antibody to T2 APCs loaded with WT1 peptide or control HLA-A2 restricted peptides. T2 cells were loaded with WT1 peptide and indicated peptides at a concentration of 10⁻⁴M for 12 hrs at 37° C. Binding was monitored by flow cytometry using secondary PE-labeled anti-mouse IgG. Binding of MAb BB7.2 ensured measurement of peptide loading efficiency.

FIG. 34: Binding of B47 TCR-like antibody to T2 APCs loaded with WT1 similar HLA-A2 restricted peptides. T2 cells were loaded with WT1 peptide and indicated peptides at a concentration of 10⁻⁴-10⁻⁵M for 12 hrs at 37° C. Binding was monitored by flow cytometry using secondary PE-labeled anti-mouse IgG. Binding of MAb BB7.2 ensured measurement of peptide loading efficiency.

FIG. 35: Binding of B47 and ESK1 TCR-like antibodies to HLA-A2 positive cells that express or not express WT1. Binding was monitored by flow cytometry using secondary PE-labeled anti-mouse IgG (for B47) or human IgG (for ESK1). Expression of HLA-A2 was assessed with MAb BB7.2.

FIG. 36: Summary of B47 TCR-like antibody selectivity. Binding of B47 TCR-like antibodies to HLA-A2+ antigen positive and negative cells was monitored by using PE-labeled anti-mouse IgG. +/− indicate WT1 mRNA gene expression as measured by PCR. HLA-A2 expression was monitored with MAb BB7.2.

FIG. 37: Epitope specificity determination of TCR-like antibodies by Alanine scanning. The WT1 peptide sequence was substituted with Alanine at positions 1, 3, 4, 5, 7, and 8. The Ala mutated peptides were synthesized and loaded APCs Binding of TCR-like antibody ESK1 was accessed by flow cytometry and binding intensity as measured by mean fluorescence intensity was measured and compared with the binding intensity to WT native WT1 peptide. The relative effect of each position Ala substitution was evaluated as percentage to the binding to WT peptide. Data from Dao et al. Sci Transl Med 5, 176ra33 (2013).

FIG. 38: Binding of D11, D7, and biotinylated MC1 to T2 APCs loaded with Tyrosinase peptide and Tyrosinase similar HLA-A2 restricted peptides. S17-S23 are Alanine-based similar peptides. T2 cells were loaded with Tyrosinase and indicated peptides at a concentration of 10⁻⁵M for 12 hrs at 37° C. Cells were stained with TCRL antibodies at a concentration of 10 μg/ml followed by secondary PE-labeled streptavidin/anti-mouse antibody and analyzed by flow cytometry Mean fluorescence intensity (MFI) is indicated.

FIG. 39: Binding of D11, D7 and MC1 TCR-like antibodies to T2 APCs loaded with Tyrosinase peptide and Tyrosinase similar HLA-A2 restricted peptides. KIAA0355, S7, S17-S23 are Alanine-based similar peptides. T2 cells were loaded with Tyrosinase and indicated peptides at a concentration of 10⁻⁵ M for 12 hrs at 37° C. Cells were stained with TCRL antibodies at a concentration of 10 μg/ml followed by secondary PE-labeled streptavidin/anti-mouse antibody and analyzed by flow cytometry Mean fluorescence intensity (MFI) is indicated.

FIGS. 40A-C: Binding of D11 (FIG. 40A), D7 (FIG. 40B) and biotinylated MC1 (FIG. 40C) TCR-like antibodies to HLA-A2+, Tyrosinase antigen positive or negative cells. Tumor and normal primary cells that express HLA-A2 were tested by qPCR for Tyrosinase mRNA expression. Tumor cells were stained with the indicated TCR-like antibodies at a concentration of 10 μg/ml followed by secondary PE-labeled streptavidin/anti-mouse antibody and analyzed by flow cytometry. Mean fluorescence intensity (MFI) is indicated.

FIG. 41: Killing of HLA-A2+/Tyrosinase+(positive) and HLA-A2+/Tyrosinase− (negative) cell lines by bi-specific (BS) TCRL having an anti CD-3 arm and a D11 arm, termed Tyr D11 BS TCRL. Tyr D11 BS TCRL was incubated with melanoma HLA-A2+/Tyrosinase+ cells and control tumor cells that are HLA-A2+/Tyrosinase-. Cells were incubated for 24 hrs with the Tyr D11 BS TCRL and with naïve PBMCs isolated from healthy individuals at 10:1 E:T ratio (10:1 effector:target ratio). Cytotoxicity determined by lactate dehydrogenase (LDH) release assay.

FIG. 42: Killing of HLA-A2+/Tyrosinase− normal primary cells by Tyr D11. BS D11 was incubated with melanoma HLA-A2+/Tyrosinase+ cells as control and normal primary cells that are HLA-A2+/Tyrosinase-. Cells were incubated for 24 hrs with the D11 BS TCRL and with naïve PBMCs isolated from healthy individuals at 10:1 E:T ratio.

FIG. 43: Killing of HLA-A2+/Tyrosinase+ and HLA-A2+/Tyrosinase− cell lines by Tyr D7 BS TCRL. D7 BS was incubated with melanoma HLA-A2+/Tyrosinase+ cells and control tumor cells that are HLA-A2+/Tyrosinase-. Cells were incubated for 24 hrs with the D7 BS and with naïve PBMCs isolated from healthy individuals at 10:1 E:T ratio.

FIG. 44: Killing of HLA-A2+/Tyrosinase− normal primary cells by D7 BS. D7 BS was incubated with melanoma HLA-A2+/Tyrosinase+ cells as control and normal primary cells that are HLA-A2+/Tyrosinase-. Cells were incubated for 24 hrs with the D7 BS and with naïve PBMCs isolated from healthy individuals at 10:1 E:T ratio.

FIG. 45 In vivo efficacy of D7 BS in preventing an S.C. 501A melanoma tumor formation in NOD/SCID mice.

FIG. 46: Binding of biotinylated ESK1 and B47B6 TCR-like antibodies to T2 APCs loaded with WT1 peptide and other HLA-A2 restricted peptides. T2 cells were loaded with WT1 peptide and indicated peptides at a concentration of 10⁻⁵M for 12 hrs at 37° C. Cells were stained with ESK1 or B47B6 TCRL antibodies at a concentration of 10 μg/ml followed by secondary PE-labeled streptavidin/anti-mouse antibody and analyzed by flow cytometry Mean fluorescence intensity (MFI) is indicated.

FIG. 47: Binding of ESK1 and B47B6 TCR-like antibodies to T2 APCs loaded with WT1 peptide and WT1 similar HLA-A2 restricted peptides. S2, S6 and S7 are Alanine-based similar peptides. S11 is a heteroclitic peptide. T2 cells were loaded with WT1 peptide and indicated peptides at a concentration of 10⁻⁵ M for 12 hrs at 37° C. Cells were stained with ESK1 or B47B6 TCRL antibodies at a concentration of 10 μg/ml followed by secondary PE-labeled streptavidin/anti-mouse antibody and analyzed by flow cytometry Mean fluorescence intensity (MFI) is indicated.

FIG. 48: Affinity by SPR—Apparent binding affinity determination of ESK1 and B47B6 TCR-like antibodies targeting HLA-A2/WT1 complexes. Purified recombinant biotinylated single-chain HLA-A2/WT1 complex generated by in vitro refolding of E. coli expressed scHLA-A2 complexes, was immobilized indirectly to the SPR sensor chip with NeutrAvidin. Purified ESK1 and B47B6 TCRL Fabs served as analytes.

FIG. 49: Epitope specificity determination by Alanine scanning mutagenesis. The mutant WT1 peptides with Alanine substitutions at positions 1, 2, 3, 4, 5, 7, 8 and 9 were synthesized and loaded onto T2 cells APCs at a concentration of 10⁻⁵M for 12 hrs at 37° C. Cells were stained with the B47B6 TCR-like antibody at a concentration of 10 μg/ml and analyzed by flow cytometry. The relative effect of Ala substitution at each position was expressed as percentage of the binding to wild-type peptide.

FIG. 50: Binding of ESK1 and B47B6 TCR-like antibodies to HLA-A2+ and WT1 mRNA positive or negative cells. Tumor cells that express HLA-A2 were tested by qPCR for WT1 mRNA expression. Tumor cells were stained with biotinylated ESK1 and B47B6 TCRL antibodies at 10 μg/ml followed by secondary PE-labeled streptavidin. Mean fluorescence intensity (MFI) is indicated. Also shown are mRNA expression data and cell killing with the bispecific forms (with anti-CD3) of the antibodies, as described herein.

FIG. 51A: Killing of HLA-A2+/WT1+ and HLA-A2+/WT1− normal primary cells by B47B6 BS vs ESK1 BS. B47B6 BS and ESK1 BS were incubated with normal primary cells that are HLA-A2+/WT1+ or HLA-A2+/WT1-. Cells were incubated for 24 hrs with the B47B6 BS or ESK1 BS and with naïve PBMCs isolated from healthy individuals at 10:1 E:T ratio. Cytotoxicity was determined by LDH release assay.

FIG. 51B: Killing of HLA-A2+/WT1+ and HLA-A2+/WT1− cell lines by B47B6 BS vs ESK1 BS. B47B6 BS and ESK1 BS were incubated with tumor cells that are HLA-A2+/WT1+ or HLA-A2+/WT1-. Cells were incubated for 24 hrs with the B47B6 BS or ESK1 BS and with naïve PBMCs isolated from healthy individuals at 10:1 E:T ratio (#F3-Format—in which the anti-CD3 scFv fragment was fused to the VLCL of the Fab),

FIG. 52: Binding of C106B9 TCR-like antibody to T2 APCs loaded with MAGE-A4₂₃₀₋₂₃₉ (also referred to as MAGE-A4 peptide) peptide and other HLA-A2 restricted peptides. T2 cells were loaded with MAGE-A4 and indicated peptides at a concentration of 10⁻⁵ M for 12 hrs at 37° C. Cells were stained with C106B9 TCRL antibody at 10 μg/ml followed by secondary PE-labeled anti-mouse antibody and analyzed by flow cytometry. Mean fluorescence intensity (MFI) is indicated.

FIG. 53: Binding of C106B9 TCR-like antibody to T2 APCs loaded with MAGE-A4 peptide and MAGE-A4 similar HLA-A2 restricted peptides. T2 cells were loaded with MAGE-A4 and indicated peptides at a concentration of 10⁻⁵M for 12 hrs at 37° C. Cells were stained with C106B9 TCRL antibody at 10 μg/ml followed by secondary PE-labeled anti-mouse antibody and analyzed by flow cytometry.

FIG. 54: Affinity by SPR—Apparent binding affinity determination of C106B9 TCR-like antibody targeting HLA-A2/MAGE-A4 complexes. Purified recombinant biotinylated single-chain HLA-A2/MAGE-A4 complex generated by in vitro refolding of E. coli expressed scHLA-A2 complexes, was immobilized indirectly to the SPR sensor chip with NeutrAvidin. Purified C106B9 TCRL Fab was used as the analyte.

FIG. 55: Epitope specificity determination by Alanine scanning mutagenesis. The mutant MAGE-A4 peptides with alanine substitutions at positions 1, 2, 3, 4, 5, 6, 7, 8 and 9 were synthesized. Possible anchor positions are shown by a gray star. The native and mutant MAGE-A4 peptides were loaded onto T2 cells APCs at a concentration of 10⁻⁵ M for 12 hrs at 37° C. Cells were stained with C106B9 TCR-like antibody at a concentration of 10 μg/ml and analyzed by flow cytometry. MFI values for cells loaded with mutant and wild type peptides were compared. The relative effect of each Ala substitution was expressed as percentage of the binding to native wild-type peptide.

FIG. 56: Binding of C106B9 TCR-like antibody to HLA-A2+ and MAGE-A4 antigen positive or negative cells. Expression of MAGE-A4 mRNA in the cells was confirmed by qPCR. Tumor cells were stained with C106B9 at 10 μg/ml followed by secondary PE-labeled anti-mouse antibody and analyzed by flow cytometry. Mean fluorescence intensity (MFI) is indicated. Also shown are mRNA expression data and cell killing with the bispecific forms (with anti-CD3) of the antibodies, as described herein.

FIG. 57: Killing of HLA-A2+/MAGE-A4+ and HLA-A2+/MAGE-A4− cell lines by C106B9 BS. C106B9 BS was incubated with tumor cells that are HLA-A2+/MAGE-A4+ cells and control tumor cells that are HLA-A2+/MAGE-A4-. Cells were incubated for 24 hrs with the C106B9 BS and with naïve PBMCs isolated from healthy individuals at 10:1 E:T ratio.

FIG. 58: Killing of HLA-A2+/MAGE-A4− normal primary cells by C106B9 BS. C106B9 BS was incubated with normal primary cells that are HLA-A2+/MAGE-A4-. Cells were incubated for 24 hrs with the C106B9 BS and with naïve PBMCs isolated from healthy individuals at 10:1 E:T ratio.

FIG. 59: In vivo efficacy of MAGE-A4 BS C106B9 BS in prevention of S.C. melanoma tumor formation in NOD/SCID mice.

FIG. 60: Binding of F184C7 TCR-like antibody to T2 APCs loaded with MAGE-A9₂₂₃₋₂₃₁ peptide (also referred to as MAGE-A9 peptide) and other HLA-A2 restricted peptides. T2 cells were loaded with MAGE-A9 peptide and indicated peptides at a concentration of 10⁻⁵ M for 12 hrs at 37° C. Cells were stained with F184C7 TCRL antibody at 10 μg/ml followed by secondary PE-labeled anti-mouse antibody and analyzed by flow cytometry. Mean fluorescence intensity (MFI) is indicated.

FIG. 61: Binding of F184C7 TCR-like antibodies to T2 APCs loaded with MAGE-A9 peptide and MAGE-A9 similar HLA-A2 restricted peptides. S8 is an Alanine-based similar peptide. T2 cells were loaded with MAGE-A9 peptide and indicated peptides at a concentration of 10⁻⁵M for 12 hrs at 37° C. Cells were stained with F184C7 TCRL antibody at 10 μg/ml followed by secondary PE-labeled anti-mouse antibody and analyzed by flow cytometry.

FIG. 62: Epitope specificity determination by Alanine scanning mutagenesis. The mutant MAGE-A9 peptides with alanine substitutions at positions 2, 3, 4, 5, 6, 7, 8 and 9 were synthesized. The Ala mutant and native peptides were loaded onto T2 cells APCs at a concentration of 10⁻⁵M for 12 hrs at 37° C. Cells were stained with F184C7 TCR-like antibody at a concentration of 10 μg/ml and analyzed by flow cytometry. MFI values for cells loaded with mutant and wild type peptides were compared. The relative effect of each Ala substitution was expressed as percentage of the binding to native peptide.

FIG. 63: Binding of F184C7 TCR-like antibody to HLA-A2+ normal primary cells. Normal primary cells were stained with F184C7 TCRL antibody at 10 μg/ml followed by secondary PE-labeled anti-mouse antibody. Mean fluorescence intensity (MFI) is indicated.

FIG. 64: Binding of D10A3 TCR-like antibody to T2 APCs loaded with PAP₁₁₂₋₁₂₀ peptide (also referred to as PAP peptide) and other HLA-A2 restricted peptides. T2 cells were loaded with PAP and indicated peptides at a concentration of 10⁻⁵ M for 12 hrs at 37° C. Cells were stained with D10A3 TCRL antibody at 10 μg/ml followed by secondary PE-labeled anti-mouse antibody. Mean fluorescence intensity (MFI) is indicated.

FIG. 65: Binding of D10A3 TCR-like antibodies to T2 APCs loaded with PAP peptide and PAP similar HLA-A2 restricted peptides. T2 cells were loaded with PAP and indicated peptides at a concentration of 10⁻⁵ M for 12 hrs at 37° C. Cells were stained with D10A3 TCRL antibody at 10 μg/ml followed by secondary PE-labeled anti-mouse antibody. Mean fluorescence intensity (MFI) is indicated.

FIG. 66: Epitope specificity determination by Alanine scanning mutagenesis. The mutant PAP peptides with Alanine substitutions at positions 1, 3, 4, 6, 7, 8 and 9 were synthesized and loaded onto T2 cells APCs at a concentration of 10⁻⁵M for 12 hrs at 37° C. Cells were stained with D10A3 TCR-like antibody at a concentration of 10 μg/ml. MFI values for cells loaded with mutant and wild type peptides were compared. The relative effect of each Ala substitution was expressed as percentage of the binding to WT peptide.

FIG. 67: Binding of D10A3 TCR-like antibody to HLA-A2+ normal primary cells. Normal primary cells were stained with D10A3 TCRL antibody at 10 μg/ml followed by secondary PE-labeled anti-mouse antibody. Mean fluorescence intensity (MFI) is indicated.

FIG. 68: Amino acids and nucleic acids of D11 antibody (SEQ ID NOs: 280-295).

FIG. 69: Amino acids and nucleic acids of D7 antibody (SEQ ID NOs: 296-311).

FIG. 70: Amino acids and nucleic acids of B47B6 antibody (SEQ ID NOs: 312-327).

FIG. 71: Amino acids and nucleic acids of C106B9 antibody (SEQ ID NOs: 328-343).

FIG. 72: Amino acids and nucleic acids of F184C7 antibody (SEQ ID NOs: 344-359).

FIG. 73: Amino acids and nucleic acids of D10A3 antibody (SEQ ID NOs: 360-375).

### DESCRIPTION OF SPECIFIC EMBODIMENTS OF THE INVENTION

The present invention, in some embodiments thereof, relates to T Cell Receptor like antibodies having fine specificity.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Antibodies with MHC-restricted specificity of T cells are rare and have been difficult to generate. The last few years have witnessed a major progress in the development of such antibodies as several groups were able to generate occasionally in a reproducible manner, T cell receptor (TCR)-like antibodies directed against a growing repertoire of tumor and viral T-cell epitopes.

Despite a major progress in the isolation of TCR-like antibodies having considerably high affinity (e.g., K_{D} below 10 nM), the issue of off-target specificity has been neglected thus resulting in antibodies which may be endowed with treatment-induced toxicity.

While conceiving embodiments of the present invention and reducing it to practice, the present inventors have assembled a set of criteria that can be used to evaluate the selectivity and fine specificity of TCR-like antibodies (hereinafter “antibodies”, “TCRLs” or “TCRL antibodies”).

Once an antibody of a predetermined affinity to a MHC-being complexed with a HLA-restricted peptide antigen is obtained, the binding of the antibody towards the specific peptide epitope is evaluated in comparison to a set of control HLA restricted peptides (irrelevant control peptides) by FACS of peptide-loaded cells.

The binding of the antibody is evaluated towards a large panel of cells from various histological origins including normal primary cells from essential and non-essential tissues. Evaluation strategy can be flow cytometry when presentation level on the cell surface is sufficiently high with cut off to flow cytometry detection of ˜100 complexes per cell. When presentation level is low (below 100 complexes per cell) functional assays can be used to evaluate specificity of binding. For example, a TCR-like antibody is labeled with a high sensitive marker or a TCR-like antibody is armed with a potent effector (therapeutic) moiety such as toxin, drug, or CD3 bi-specific arm.

The antibody is evaluated for binding to a panel of similar peptides which are naturally presented (as determined by MS databases of peptides eluted from cells or other databases of HLA peptidome) or similar in silico produced peptides.

The selection of similar peptides which are naturally presented is further described hereinbelow. It will be appreciated that the selection of these peptides much depends on determining the amino acids in the peptide, which are critical for the antibody binding. This is effected by evaluation of binding of the antibody towards a panel of alanine mutated (alanine scanning) peptides of the target peptide antigen. This peptide epitope specificity determination measures the residues in the target peptide antigen that are sensitive to Ala mutation in decreasing the binding of the TCR-like antibody to the target antigen when presented on loaded cells. This tool enables to determine which are the critical positions for binding by the TCR-like antibody and are extremely useful in determining the selectivity and fine specificity level of the TCR-like antibody. It enables a filtering tool to select the best candidate among several TCR-like antibodies for further evaluation to obtain the most optimal highly selective TCR-like antibody with the best optimal fine specificity towards the specific HLA-A2/peptide complex. The more sensitive Ala positions identified the more selective and specific the TCR-like antibody is. As exemplified herein, TCR-like antibodies with 4-5 sensitive Ala positions exhibited superior selectivity and specificity patterns compared to TCR-like antibodies that exhibited a single or 3 sensitive position in the peptide. These epitope specificity determination also enables selecting additional similar peptides for further evaluation of selectivity and fine specificity of the TCR-like antibody candidate.

The robustness of the present selection method identifies those TCRLs which are of clinically relevant specificity or in other words, allows for the selection of highly selective TCR-like antibody with the optimal fine specificity towards a specific MHC being complexed with an HLA-restricted peptide antigen. The present inventors were able to prove that even those TCRLs which are allegedly identified in the art as therapeutics are endowed with poor specificity and therefore may incur tissue toxicity. For example, the MC1 TCRL targeting MHC-presented tyrosinase peptide exhibits clinically relevant affinity having a KD of below than 5 nM. However, upon studying its specificity, the present inventors have realized that since it hasn't been subjected to the strict selection rules taught herein, such an antibody can't be used in the efficacious treatment of melanoma for the risk of toxicity. Similarly, the ESK TCRL targeting MHC-presented WT1 was found to exhibit not only poor affinity but also low specificity.

In sharp contrast, by applying the present selection rules the present inventors were able to identify a novel class of TCRLs which are of fine specificity, where as much as 6 or 7 out of the 9 residues (or 10 residues in the case of MAGE-A4) making the HLA-restricted peptide target are critical for binding the TCRL. Such antibodies which exhibit high affinity (below 5 nM) are expected to advance the entire field of TCRLs and in fact any immunomodulation which is based on TCR-like recognition including adoptive cell therapy e.g., CAR technology.

As used herein a “T Cell Receptor-like antibody” or “TCRL” refers to an antibody which binds an MHC being complexed with an HLA-restricted peptide antigen. Binding of the TCRL to its target is with an MHC-restricted specificity. The TCRL antibody does not bind said MHC in the absence of said complexed peptide, and the antibody does not bind said peptide in an absence of said MHC.

As used herein “binding” or “binds” refers to an antibody-antigen mode of binding, which is generally, in the case of clinically relevant TCRLs, in the range of K_{D} below 20 nM, as determined by Surface Plasmon Resonance assay (SPR).

The affinity of the antigen binding domain to its antigen is determined using the soluble form of the antibody from which the CDRs of the antigen binding domain of the antibody are derived. For affinity evaluation, the antigen is used in its soluble form e.g., as a single chain MHC-peptide complex as further described hereinbelow.

As used herein the term “K_{D}” refers to the equilibrium dissociation constant between the antigen binding domain and its respective antigen.

According to an embodiment of this aspect of the present invention, the antibody binds to the MHC-peptide complex with a K_{D} less than 50 nM. According to another embodiment the K_{D} is less that 20 nM. According to a further embodiment, the antibody binds to the MHC-peptide complex with a K_{D} less than 10 nM. According to a further embodiment, the antibody binds to the MHC-peptide complex with a K_{D} less than 5 nM. According to a further embodiment, the antibody binds to the MHC-peptide complex with a K_{D} less than 1 nM. According to a further embodiment, the antibody binds to the MHC-peptide complex with a K_{D} in the range of 0.1-50 nM. According to a further embodiment, the antibody binds to the MHC-peptide complex with a K_{D} in the range of 0.1-20 nM. According to a further embodiment, the antibody binds to the MHC-peptide complex with a K_{D} in the range of 0.1-10 nM. According to a further embodiment, the antibody binds to the MHC-peptide complex with a K_{D} in the range of 0.1-5 nM. According to a further embodiment, the antibody binds to the MHC-peptide complex with a K_{D} in the range of 0.1-1 nM. According to a further embodiment, the antibody binds to the MHC-peptide complex with a K_{D} in the range of 1-50 nM. According to a further embodiment, the antibody binds to the MHC-peptide complex with a K_{D} in the range of 1-20 nM. According to a further embodiment, the antibody binds to the MHC-peptide complex with a K_{D} in the range of 1-10 nM. According to a further embodiment, the antibody binds to the MHC-peptide complex with a K_{D} in the range of 1-5 nM. According to a further embodiment, the antibody binds to the MHC-peptide complex with a K_{D} in the range of 1-4 nM.

Higher affinities are also contemplated e.g., 50-100 nM, 100 nM-1 μM, 200 nM-1 μM, 300 nM-1 μM, 500 nM-1 μM, 800 nM-1 μM, 100-500 nM, 100-400 nM, 100-600 nM.

As used herein, the phrase “major histocompatibility complex (MHC)” refers to a complex of antigens encoded by a group of linked loci, which are collectively termed H-2 in the mouse and HLA in humans. The two principal classes of the MHC antigens, class I and class II, each comprise a set of cell surface glycoproteins which play a role in determining tissue type and transplant compatibility. In transplantation reactions, cytotoxic T-cells (CTLs) respond mainly against foreign class I glycoproteins, while helper T-cells respond mainly against foreign class II glycoproteins. According to a specific embodiment, the MHC is a human MHC.

According to a specific embodiment, the MHC is a class I MHC.

Major histocompatibility complex (MHC) class I molecules are expressed on the surface of nearly all cells. These molecules function in presenting peptides which are mainly derived from endogenously synthesized proteins to CD8+ T cells via an interaction with the αβ T-cell receptor. The class I MHC molecule is a heterodimer composed of a 46-kDa heavy chain which is non-covalently associated with the 12-kDa light chain β-2 microglobulin. In humans, there are several MHC haplotypes, such as, for example, HLA-A2, HLA-A1, HLA-A3, HLA-A24, HLA-A28, HLA-A31, HLA-A33, HLA-A34, HLA-B7, HLA-B45 and HLA-Cw8, their sequences can be found at the kabbat database, at htexttransferprotocol://immuno.bme.nwu.edu. Further information concerning MHC haplotypes can be found in Paul, B. Fundamental Immunology Lippincott-Rven Press. According to a specific embodiment, the MHC haplotype is HLA-A2.

Based on accumulated experimental data, it is nowadays possible to predict which of the peptides of a protein will bind to MHC, class I. The HLA-A2 MHC class I has been so far characterized better than other HLA haplotypes, yet predictive and/or sporadic data is available for all other haplotypes.

With respect to HLA-A2 binding peptides, assume the following positions (P1-P9) in a 9-mer peptide:
P1 P2 P3 P4 P5 P6 P7 P8 P9

The P2 and P9 positions include the anchor residues which are the main residues participating in binding to MHC molecules. Amino acid resides engaging positions P2 and P9 are hydrophilic aliphatic non-charged natural amino (examples being Ala, Val, Leu, Ile, Gln, Thr, Ser, Cys, preferably Val and Leu) or of a non-natural hydrophilic aliphatic non-charged amino acid (examples being norleucine (Nle), norvaline (Nva), α-aminobutyric acid).

It will be appreciated that in case of a 10 mer peptide, the P3 and P10 are the anchor residues.

While a more detailed description is provided herein with respect to MHC class I, the present invention also concerns MHC class II.

According to a specific embodiment, the MHC is a class II MHC.

The term “antibody” as used in this invention includes intact molecules as well as functional fragments thereof, such as Fab, F(ab′)2, Fv, scFv, dsFv, or single domain molecules such as VH and VL that are capable of binding to an epitope of an antigen in an MHC restricted manner. As a more general statement the term “antibody” aims to encompass any affinity binding entity which binds a cell surface presented molecule with an MHC restricted specificity. Thus, CDRs of the antibodies of some embodiments of the present invention may be implanted in artificial molecules such as T cell receptors or CARs as further described hereinbelow.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′)2.

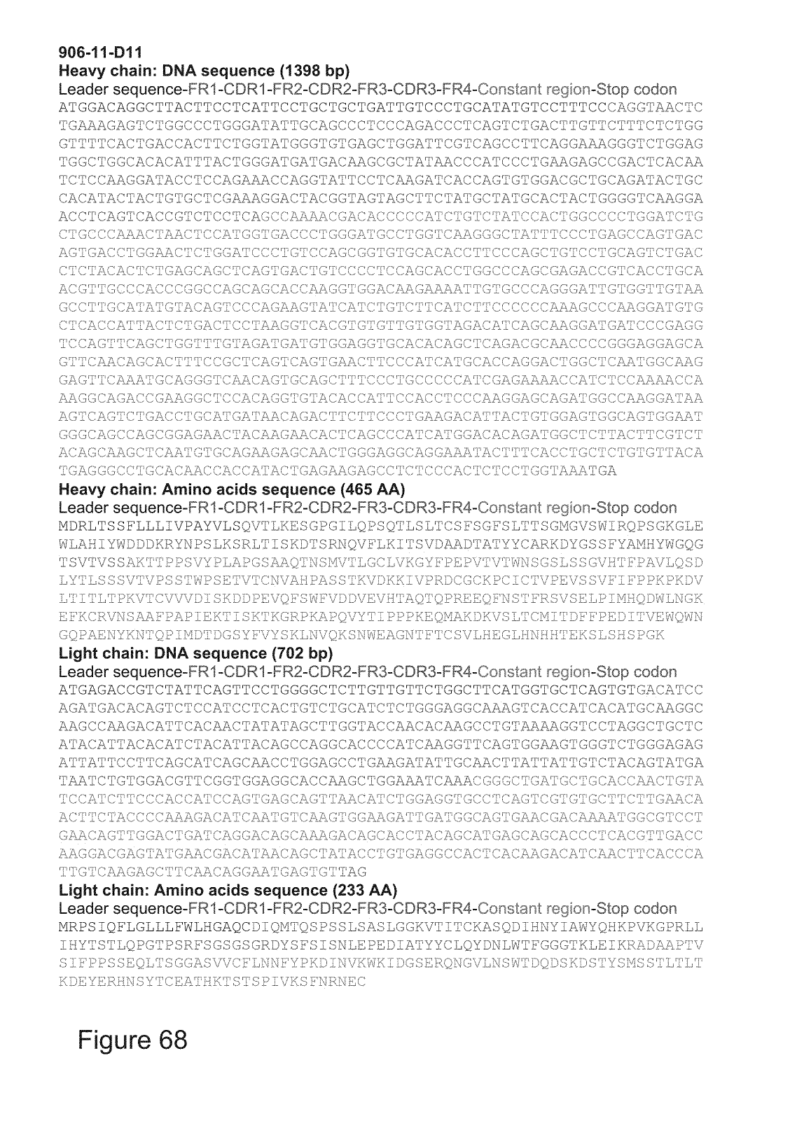

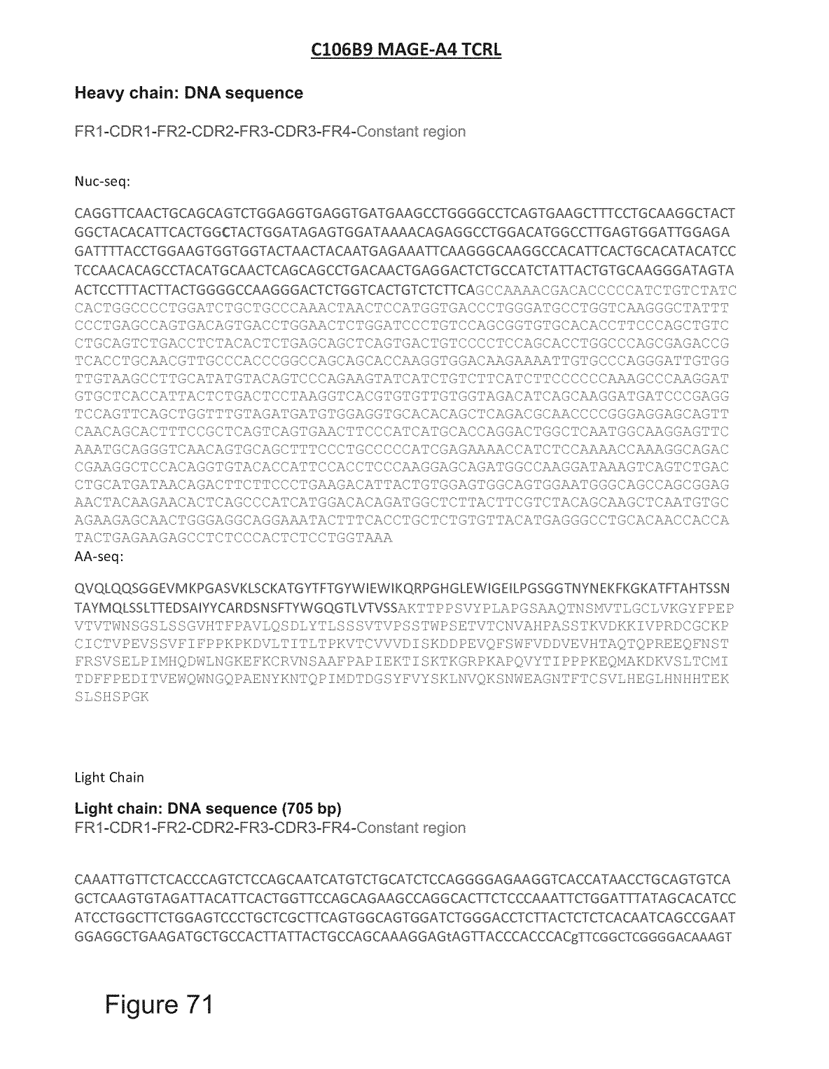

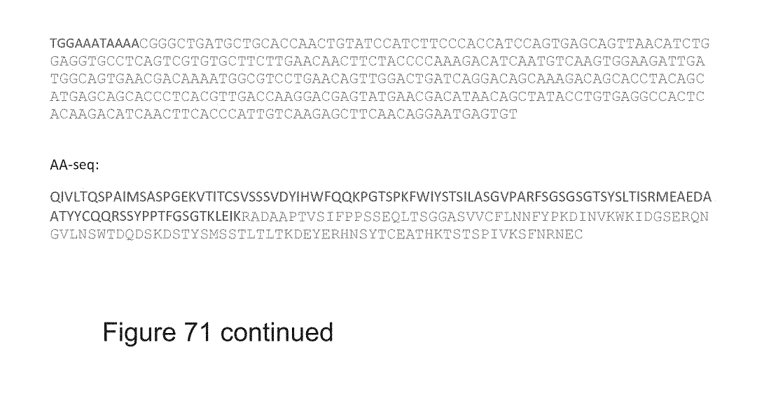

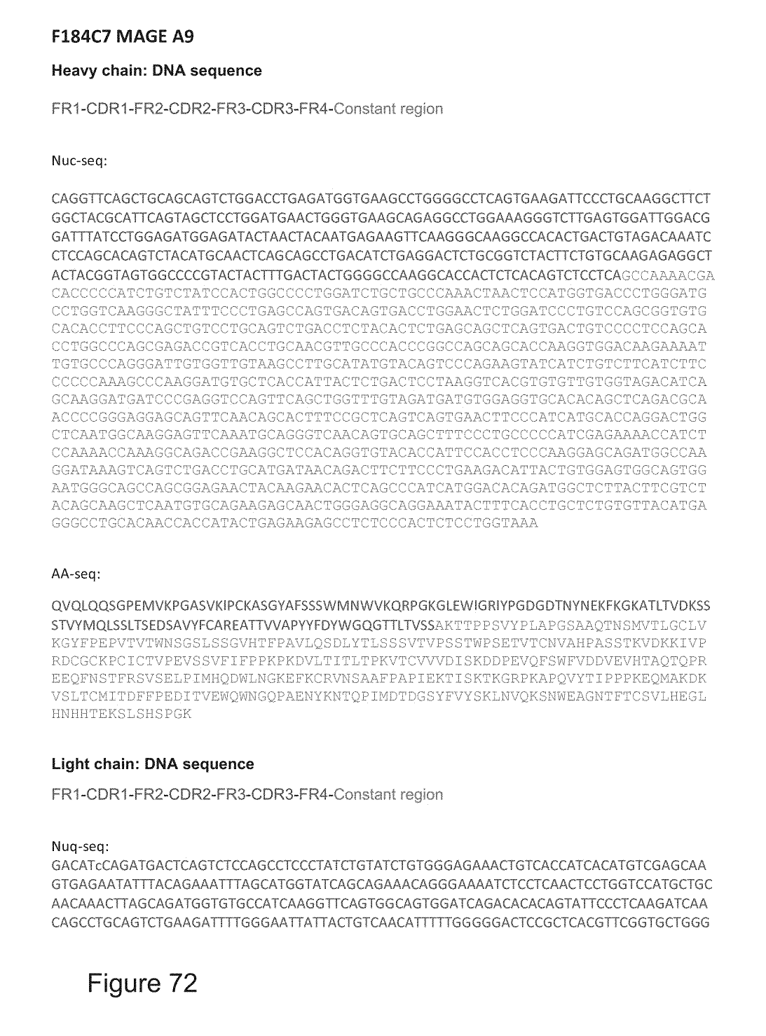

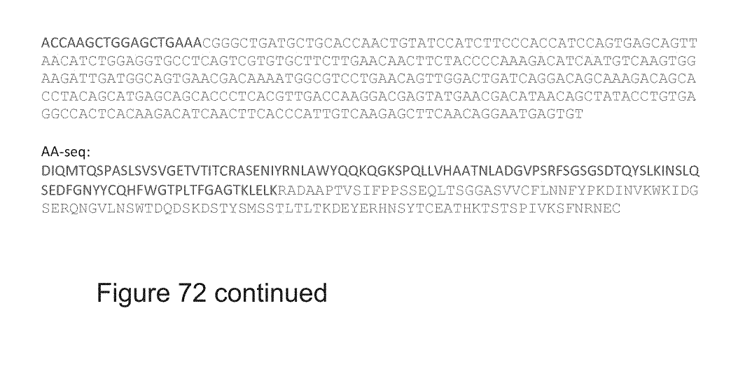

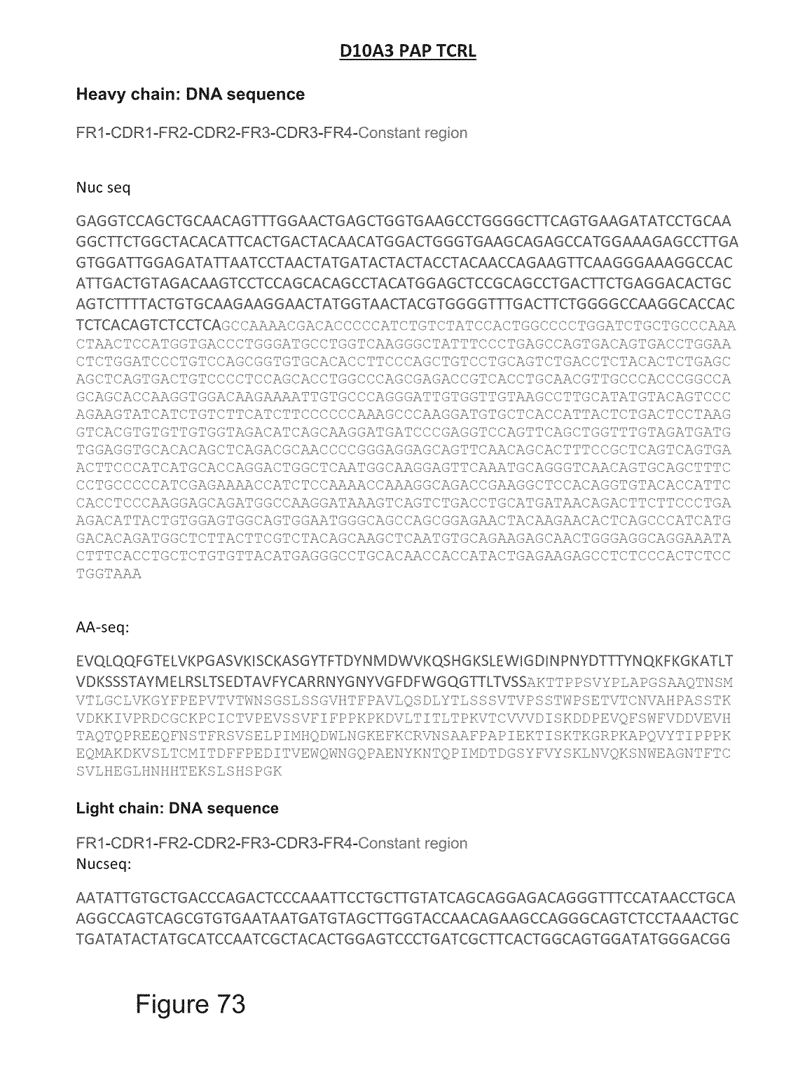

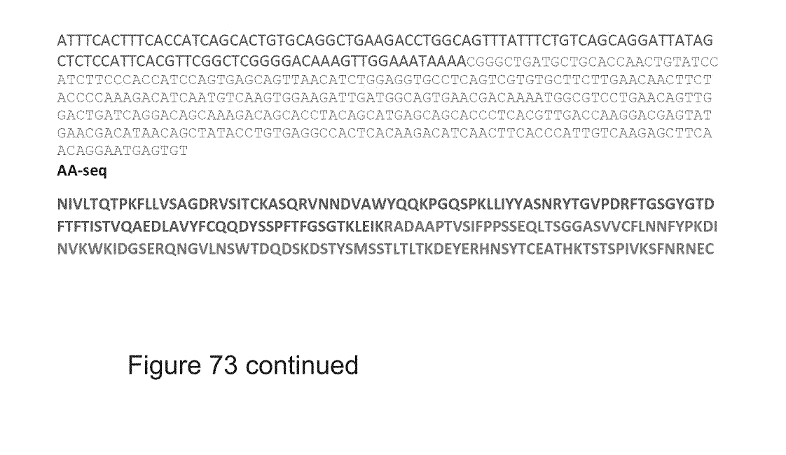

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3). Examples of such CDR sequences are provided for D7 and D11—TCRLs produced according to Example I below. Additional examples include, WT1 B47B6, MAGE-A4 C106B9, MAGE-A9 F184C7, PAP D10A3 (shown in FIGS. 68-73).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www.bioinf-org.uk/abs), available complex crystal structures as defined by the contact definition (see MacCallum et al., J. Mol. Biol. 262:732-745, 1996), the “conformational definition” (see, e.g., Makabe et al., Journal of Biological Chemistry, 283:1156-1166, 2008) and IMGT [Lefranc M P, et al. (2003) IMGT unique numbering for immunoglobulin and T cell receptor variable domains and Ig superfamily V-like domains. Dev Comp Immunol 27: 55-77].

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:

(i) Fv, defined as a genetically engineered fragment consisting of the variable region of the light chain (VL) and the variable region of the heavy chain (VH) expressed as two chains;

(ii) single chain Fv (“scFv”), a genetically engineered single chain molecule including the variable region of the light chain and the variable region of the heavy chain, linked by a suitable polypeptide linker as a genetically fused single chain molecule.

(iii) disulfide-stabilized Fv (“dsFv”), a genetically engineered antibody including the variable region of the light chain and the variable region of the heavy chain, linked by a genetically engineered disulfide bond.

(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;

(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);

(vi) F(ab′) 2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and

(vii) Single domain antibodies or nanobodies are composed of a single VH or VL domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Methods of producing polyclonal and monoclonal antibodies as well as fragments thereof are well known in the art (See for example, Harlow and Lane,

Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

In an embodiment in which the antibody is a full length antibody, the heavy and light chains of an antibody of the invention may be full-length (e.g., an antibody can include at least one, and preferably two, complete heavy chains, and at least one, or two, complete light chains) or may include an antigen-binding portion (a Fab, F(ab′).sub.2, Fv or a single chain Fv fragment (“scFv”)). In other embodiments, the antibody heavy chain constant region is chosen from, e.g., IgG1, IgG2, IgG3, IgG4, IgM, IgA1, IgA2, IgD, and IgE. In some embodiments, the immunoglobulin isotype is selected from IgG1, IgG2, IgG3, and IgG4, more particularly, IgG1 (e.g., human IgG1) or IgG4 (e.g., human IgG4). The choice of antibody type will depend on the immune effector function that the antibody is designed to elicit.

Bispecific configurations of antibodies are also contemplated herein. A bispecific monoclonal antibody (BsMAb, BsAb) is an artificial protein that is composed of fragments of two different monoclonal antibodies and consequently binds to two different types of antigen. According to a specific embodiment the BsMAb is engineered to simultaneously bind to a cytotoxic cell (e.g., using a receptor like CD3) and a target like a tumour cell to be destroyed (further described hereinbelow).

As used herein the phrase “chimeric antigen receptor (CAR)” refers to a recombinant or synthetic molecule which combines antibody-based specificity for a desired antigen with a T cell receptor-activating intracellular domain to generate a chimeric protein that exhibits cellular immune activity to the specific antigen.

As used herein the phrase “MHC (or HLA)-restricted peptide” refers to a peptide which is potentially presented on an MHC molecule. Such peptides may be identified by “wet” laboratory procedures such as Mass-Spectrometry or by in-silico analysis. An MHC (or HLA)-presented peptide refers to a peptide which is confirmed in vitro or in vivo as being presented by an MHC molecule.

As used herein, the term “HLA-restricted antigen” refers to a peptide capable of specifically binding an antigen-binding groove of an MHC. Such an antigen is commonly referred to in the art as being “restricted” by such an MHC. A typical antigen, such as a pathogen-derived antigen, tumor antigen or autoantigen, is typically generated in a human cell by intracellular processing of a larger polypeptide such as derived from the pathogen or the tumor. The antigen generally has a characteristic dimension and/or chemical composition—for example, a characteristic amino acid length and set of anchor residues, respectively, in the case of a peptide antigen—enabling it to specifically bind the antigen-binding groove of a particular MHC haplotype so as to form an MHC/antigen complex therewith having an antigen presenting portion capable of specifically binding a variable region of a cognate TCR. For HLA-A2, ALA-A3, HLA-A*6801, HLA-B7 and HLA-B27, for example, the anchoring positions are P2 and P9. For HLA-B*08 the anchor positions P5 and P9. For

HLA-C*14:02 the anchor positions P2 and P3.

HLA restricted peptide antigens may be from a tumor antigen (e.g., tumor specific antigen or a tumor associated antigen), a viral protein antigen, or an autoimmune associated antigen (e.g., a “self” antigen).

According to a specific embodiment, the peptide has an MHC-restricted viral antigen structure.

According to a specific embodiment, the peptide does not have an MHC-restricted viral antigen structure.

Following are non-limiting sequences of HLA class I-restricted tumor antigens which can bind to the antigen binding domain of the TCRL-antibody of the invention.

TABLE 1

| | | | SEQ ID | |
|---|---|---|---|---|
| | | GenBank Accession | NO: of | |
| | | No. of the | the tumor | |
| Cancer | TAA/Marker | tumor antigens | antigens | HLA |
| Transitional | Uroplakin II | NP_006751.1 | 16 | HLA-A2 |
| cell | (UPKII) | | | |
| carcinoma | | | | |
| Transitional | Uroplakin Ia | NP_001268372.1; NP_008931.1 | 17, 18 | HLA-A2 |
| cell | (UPK1A) | | | |
| carcinoma | | | | |
| Carcinoma | prostate specific | AAO16090.1 | 19 | HLA-A2 |
| of the | antigen (NPSA) | | | |
| prostate | | | | |
| Carcinoma | prostate specific | NP_005663.2 | 20 | HLA-A2 |
| of the | membrane | | | |
| prostate | antigen (PSCA) | | | |
| Carcinoma | prostate acid | NP_001090.2; NP_001127666.1; | 21-23 | HLA-A2 |
| of the | phosphatase | NP_001278966.1 | | |
| prostate | (ACPP) | | | |
| Breast cancer | BA-46 | NP_001108086.1; NP_005919.2; | 24 | HLA-A2 |
| | MFGE8 milk fat | | | |
| | globule-EGF | | | |
| | factor 8 protein | | | |
| | [lactadherin] | | | |
| Breast cancer | Mucin 1 | NP_001018016.1; NP_001018017.1; | 25-44 | HLA-A2 |
| | (MUC1) | NP_001037855.1; NP_001037856.1; | | |
| | | NP_001037857.1; NP_001037858.1; | | |
| | | NP_001191214.1; NP_001191215.1; | | |
| | | NP_001191216.1; NP_001191217.1; | | |
| | | NP_001191218.1; NP_001191219.1; | | |
| | | NP_001191220.1; NP_001191221.1; | | |
| | | NP_001191222.1; NP_001191223.1; | | |
| | | NP_001191224.1; NP_001191225.1; | | |
| | | NP_001191226.1; NP_002447.4 | | |
| Melanoma | premelanosome | NP_001186982.1; NP_001186983.1; | 45-47 | HLA-A2 |
| | protein (PMEL; | NP_008859.1 | | |
| | also known as | | | |
| | Gp100) | | | |
| Melanoma | melan-A | NP_005502.1; | 48 | HLA-A2 |
| | (MLANA; also | | | |
| | known as | | | |
| | MART1) | | | |
| All tumors | telomerase | NP_001180305.1; NP_937983.2 | 49-50 | HLA-A2 |
| | reverse | | | |
| | transcriptase | | | |
| | (TERT) | | | |
| Leukemia and | TAX | NP_057864.1; YP_002455788.1 | 51-52 | HLA-A2 |
| Burkitts | tax p40 [Human | | | |
| Lymphoma | T-lymphotropic | | | |
| | virus 1] and | | | |
| | Tax [Human T- | | | |
| | lymphotropic | | | |
| | virus 4]; | | | |
| Carcinomas | NY-ESO | NP_001318.1 | 53 | HLA-A2 |
| | cancer/testis | | | |
| | antigen 1B | | | |
| | (CTAG1B) | | | |
| Melanoma | Melanoma | NP_004979.3 | 54 | HLA-A2 |
| | antigen family | | | |
| | A1 (MAGEA1) | | | |
| Melanoma | Melanoma | NP_005353.1 | 55 | HLA-A24 |
| | antigen family | | | |
| | A3 (MAGEA3, | | | |
| | MAGE-A3) | | | |
| Carcinomas | HER2; erb-b2 | NP_001005862.1; NP_001276865.1; | 56-60 | HLA-A2 |
| | receptor | NP_001276866.1; NP_001276867.1; | | |
| | tyrosine kinase | NP_004439.2; | | |
| | 2 (ERBB2) | | | |
| Melanoma | Beta-catenine; | NP_001091679.1; NP_001091680.1; | 61-63 | HLA-A24 |
| | catenin | NP_001895.1; | | |
| | (cadherin- | | | |
| | associated | | | |
| | protein), beta 1, | | | |
| | 88 kDa | | | |
| | (CTNNB1) | | | |
| Melanoma | Tyrosinase | NP_000363.1 | 64 | HLA- |
| | (TYR) | | | DRB1 |
| Leukemia | Bcr-ab1 | AAA35594.1 | 65 | HLA-A2 |
| Head and neck | caspase 8, | NP_001073593.1; NP_001073594.1; | 66-71 | HLA- |
| | apoptosis- | NP_001219.2; NP_203519.1; | | B35 |
| | related cysteine | NP_203520.1; NP_203522.1 | | |
| | peptidase | | | |
| | (CASP8) | | | |

According to some embodiments of the invention, the tumor associated antigen comprises the WT1 protein.

The Wilms' tumor oncogene protein (WT1) is an attractive target for immunotherapy for most leukemias and a wide range of cancers. WT1 is a zinc finger transcription factor that is normally expressed in mesodermal tissues during embryogenesis. In normal adult tissue, WT1 expression is limited to low levels in CD34⁺ hematopoietic stem cells but is over-expressed in leukemias of multiple lineages and a wide range of solid tumors. WT1 expression has been reported to be a marker of minimal residual disease. Increasing transcript levels in patients with acute myeloid leukemia (AML) in morphologic remission have been predictive of overt clinical relapse. Furthermore, antibodies to WT1 are detected in patients with hematopoietic malignancies and solid tumors, indicating that WT1 is a highly immunogenic antigen.

WT1 presentation is associated with cancers, including for example, breast cancer, ovarian cancer, prostate cancer, chronic myelocytic leukemia, multiple myeloma, acute lymphoblastic leukemia (ALL), acute myeloid/myelogenous leukemia (AML) and myelodysplastic syndrome (MDS).

According to some embodiments of the invention, the MHC-restricted tumor associated antigen is the WT1₁₂₆₋₁₃₄ peptide set forth in RMFPNAPYL.

According to some embodiments of the invention, the tumor associated antigen comprises the tyrosinase protein.

Tyrosinase peptides that bind to class I MHC molecules (also referred to herein interchangeably as HLA-restricted tyrosinase epitopes, HLA-restricted tyrosinase epitopes and MHC-restricted tyrosinase antigens) are derived from the tyrosinase enzyme (Genebank Accession No: NP_000363.1) and are typically 8-10 amino acids long, bind to the heavy chain α1-α2 groove via two or three anchor residues that interact with corresponding binding pockets in the MHC molecule.

Tyrosinase is a membrane-associated N-linked glycoprotein and it is the key enzyme in melanin synthesis. It is expressed in all healthy melanocytes and in nearly all melanoma tumor samples (H. Takeuchi, et al., 2003; S. Reinke, et al., 2005). Peptides derived from this enzyme are presented on MHC class I molecules and are recognized by autologous cytolytic T lymphocytes in melanoma patients [T. Wolfel, et al., 1994; Brichard, et al., 1993; Renkvist et al, Cancer immunology immunotherapy 2001 50:3-15; Novellino L, et al., March 2004 update. Cancer Immunol Immunotherapy. 54:187-207, 2005]. Additional tumor tyrosinase HLA-restricted peptides derived from tumor associated antigens (TAA) can be found at the website of the Istituto Nazionale per lo Studio e la Cura dei Tumori at hypertexttransferprotocol://worldwideweb.istitutotumori.mi.it.

Non-limiting examples of MHC class I restricted tyrosinase antigenic peptides are provided in WO2008/120202, which is fully incorporated herein by reference in its entirety, e.g., in Table 139 of WO2008/120202.

According to some embodiments of the invention, the tyrosinase antigenic peptide is the TyrD369-377 peptide [YMDGTMSQV, SEQ ID NO: 1].

According to some embodiments of the invention, the MART-1 antigenic peptide is the peptide set forth by EAAGIGILTV, SEQ ID NO: 7.

Following are non-limiting sequences of HLA class I-restricted viral antigens which can bind to the antigen binding domain of the TCRL-antibody of the invention (Table 2 below).

According to some embodiments of the invention, the viral antigens include viral epitopes from a polypeptide selected from the group consisting of: human T cell lymphotropic virus type I (HTLV-1) transcription factor (TAX), influenza matrix protein epitope, Epstein-Bar virus (EBV)-derived epitope, HIV-1 RT, HIV Gag, HIV Pol, influenza membrane protein M 1, influenza hemagglutinin, influenza neuraminidase, influenza nucleoprotein, influenza nucleoprotein, influenza matrix protein (M1), influenza ion channel (M2), influenza non-structural protein NS-1, influenza non-structural protein NS-2, influenza PA, influenza PB1, influenza PB2, influenza BM2 protein, influenza NB protein, influenza nucleocapsid protein, Cytomegalovirus (CMV) phosphorylated matrix protein (pp65), TAX, hepatitis C virus (HCV), HBV pre-S protein 85-66, HTLV-1 tax 11-19, HBV surface antigen 185-194.

TABLE 2

| | | GenBank Accession | | |
|---|---|---|---|---|
| | | Nos. of the | SEQ ID | |
| | | viral antigens; | NOs: of | |
| | | or the peptide | the viral | |
| Disease | Viral antigen | sequence | antigens | HLA |
| AIDS (HTLV-1) | HIV-1 RT 476-484 | | 72 | HLA-A2 |
| Gag (HIV) | Gag 77-85 | SLYNTVATL | 73 | |
| Pol (HIV) | Pol 476-484 | ILEPVHGV | 74 | |
| Influenza | | GILGFVFTL | 75 | HLA-A2 |
| Influenza | Membrane protein | YP_308854.1 | 76 | |
| | M1 of influenza A | | | |
| | virus | | | |
| | A/Korea/426/68(H2N2) | | | |
| Influenza | hemagglutinin of | NP_056660.1; | 77-78 | |
| | influenza B virus; | YP_308839.1 | | |
| | hemagglutinin of | | | |
| | influenza A virus | | | |
| | (A/New | | | |
| | York/392/2004(H | | | |
| | 3N2) | | | |
| Influenza | neuraminidase of | NP_056663.1 | 79 | |
| | influenza B virus | | | |
| Influenza | nucleoprotein of | YP_089656.1 | 80 | |
| | influenza C virus; | | | |
| Influenza | nucleoprotein of | YP_308871.1; | 81-82 | |
| | influenza A virus | YP_581749.1; | | |
| | such as the | | | |
| | A/Korea/426/68(H | | | |
| | 2N2) strain; or the | | | |
| | A/Hong | | | |
| | Kong/1073/99(H9 | | | |
| | N2) strain | | | |
| Influenza | nucleoprotein of | NP_056661.1; | 83 | |
| | influenza B virus | | | |
| Influenza | matrix protein | NP_056664.1 | 84 | |
| | (M1) of influenza | | | |
| | B virus | | | |
| Influenza | ion channel (M2) | NP_040979.2 | 85 | |
| | of influenza A | | | |
| | virus A/Puerto | | | |
| | Rico/8/34(H1N1) | | | |
| | strain | | | |
| Influenza | non-structural | NP_056666.1 | 86 | |
| | protein NS-1 of | | | |
| | influenza B virus | | | |
| Influenza | non-structural | NP_056665.1 | 87 | |
| | protein NS-2 of | | | |
| | influenza B virus | | | |
| Influenza | PA of influenza A | AAL60433 | 88 | |
| | virus | | | |
| | A/Charlottesville/ | | | |
| | 28/95(H1N1) | | | |
| Influenza | PB1 of influenza | NP_056657.1 | 89 | |
| | B virus | | | |
| Influenza | PB2 of influenza | NP_040987.1 | 90 | |
| | A virus (A/Puerto | | | |
| | Rico/8/34(H1N1) | | | |
| Influenza | BM2 protein of | YP_419283.1 | 91 | |
| | influenza B virus | | | |
| Influenza | NB protein of | NP_056662.1 | 92 | |
| | influenza B virus | | | |
| Influenza | nucleocapsid | NP_040982.1 | 93 | |
| | protein of | | | |
| | influenza A virus | | | |
| | A/Puerto | | | |
| | Rico/8/34(H1N1) | | | |
| CMV disease | CMV | AAA45996; | 94 | HLA-A2 |
| | phosphorylated | P06725; | | |
| | matrix protein | AAA45994.1; | | |
| | (pp65) [Human | P18139 | | |
| | herpesvirus 5] | | | |
| Leukemia and | TAX tax p40 [Human | NP_057864.1; | 95 | HLA-A2 |
| Burkitts | T-lymphotropic | YP_002455788.1 | | |
| Lymphoma | virus 1] and | | | |
| | Tax [Human | | | |
| | T-lymphotropic virus 4]; | | | |
| Hepatitis C | HCV | | 100, 384 | HLA-A2 |
| Hepatitis B | HBV pre-S protein | STNRQSGRQ | 101 | HLA-A2 |
| | 85-66 | | | |
| HTLV-1 | HTLV-1 tax 11-19 | LLFGYPVYV | 102 | HLA-A2 |
| Leukemia | | | | |
| Hepatitis | HBV surface | GLSPTVWLSV | 103 | HLA-A2 |
| | antigen 185-194 | | | |

Cytomegalovirus (CMV) belongs to the human herpesviruses. There are several known strains of CMV, including strains 1042, 119, 2387, 4654, 5035, 5040, 5160, 5508, AD169, Eisenhardt, Merlin, PT, Toledo and Towne. During viral infection, the expressed viral proteins, e.g., pp65 of the CMV AD169 strain [GenBank Accession No. AAA45996.1; or GenBank Accession No. P06725] pp64 of the CMV Towne strain [GenBank Accession No. AAA45994.1 for amino acids; or GenBank Accession No. P18139] are subject to proteasomal degradation and the MHC-restricted peptides bind to the MHC molecules [e.g., MHC class I or MHC class II] and are further presented therewith on the cell surface. The pp65 (561 amino acids in length) and pp64 (551 amino acids in length) proteins of the CMV AD169 and Towne strains, respectively, are 99% identical proteins and share the same amino acid sequence from position 3-551 of pp64 and 13-561 of pp65.

According to some embodiments of the invention, the MHC-restricted CMV antigenic peptide is the antigenic peptide derived from the pp65 or pp64 proteins and described in Table 137 of WO2008/120203, which is fully incorporated herein by reference in its entirety.

According to some embodiments of the invention, the antigen is an autoantigen associated with an autoimmune disease.

The term “autoimmune disease” as used herein is defined as a disorder that results from an autoimmune response. An autoimmune disease is the result of an inappropriately excessive response to a self-antigen.

Examples of autoimmune diseases include but are not limited to, Addision's disease, alopecia greata, ankylosing spondylitis, autoimmune hepatitis, autoimmune parotitis, Crohn's disease, diabetes (Type 1), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein the phrase “autoantigenic peptide” refers to an antigen derived from an endogenous (i.e., self protein) or a consumed protein (e.g., by food) against which an inflammatory response is elicited as part of an autoimmune inflammatory response.

It should be noted that the phrases “endogenous”, “self” are relative expressions referring to the individual in which the autoimmune response is elicited. Auto-antigens comprise, but are not limited to, cellular proteins, phosphoproteins, cellular surface proteins, cellular lipids, nucleic acids, glycoproteins, including cell surface receptors.

It should be noted that presentation of an autoantigenic peptide on antigen presenting cells (APCs) can result in recognition of the MHC-autoantigenic peptides by specific T cells, and consequently generation of an inflammatory response that can activate and recruit T cell and B cell responses against the APCs cells.

A common basis for several autoimmune diseases, including Multiple Sclerosis (MS), Type 1 Diabetes (T1D) and Rheumatoid Arthritis (RA), is the strong linkage between HLA genotype and susceptibility to the disease (Nepom, 1991; Sawcer, 2005; McDaniel, 1989). While some alleles are tightly linked to certain diseases, others confer protection and are extremely rare in patients. This linkage is not surprising due to the involvement of T-cells in the progression of these diseases. Activation or disregulation of CD4+ T-cells directed to self organ-specific proteins, combined with yet-undefined events, may contribute to the pathogenesis of a variety of human autoimmune diseases.

In order to generate TCRL-antibodies, a purified MHC-peptide complex folded in a native conformation that is recognized by a T cell, is generated. Such complexes are typically formed using recombinant DNA technology. The skilled artisan is well aware of methods of producing such complexes, where the general theme is to bring the peptide target to native complexation with the soluble MHC in solution.

For example, each of the MHC heavy chain and β2m are individually produced in E. coli. The peptide-MHC complexes are in vitro refolded from inclusion bodies produced in E. Coli (Chames et al. 2000 PNAS 97(14):7969-7974).

Alternatively, MHC-peptide complexes are generated by expressing the extracellular domains of the MHC heavy chain and f32m (e.g., as a single chain e.g., wherein the (32m is translationally fused N-terminally of the heavy chain e.g., via a peptide linker) in inclusion bodies in E. coli followed by in vitro refolding in the presence of the desired HLA-restricted peptide (Altman et al. 1996 Science 274:94-96, Denkberg et al. Eur. J. Immunol. 2000 30:3522-3532).

The refolded peptide-MHC complexes are purified typically in a monomeric form and may be further biotinylated in a site-specific manner, a feature that is utilized for the in-vitro selection procedures of TCRL isolation.

Once the complex is at hand, TCRL antibodies can be produced against the complex using a standard hybridoma approach or selected by employing immunized or non-immunized (e.g., phage) antibody libraries of full length antibodies or antibody fragments such as Fabs. TCRLs thus produced are described e.g., Reiter, U.S. Patent App. Pub. No. 2004/0191260 A1, filed Mar. 26, 2003; Andersen et al., U.S. Patent App. Pub. No. 2002/0150914 A1, filed Sep. 19, 2001; Hoogenboom et al., U.S. Patent App. Pub. No. 2003/0223994, filed Feb. 20, 2003; and Reiter et al., PCT App. Pub. No. WO 03/068201, filed Feb. 11, 2003, WO2008/120202 filed Mar. 27, 2008). Another approach, for generating TCR-like antibodies is described in U.S. Pat. Appl. 20120294874 that teaches a method of producing a T cell receptor-like antibody, the method comprising the steps of forming an immunogen comprising a monomeric MHC-peptide complex; administering an effective amount of the immunogen to a host-for eliciting an immune response to the peptide within the MHC-peptide complex; selecting a B cell specific to the peptide in the MHC-peptide complex; forming a hybridoma by fusing the B cell with an immortalized cell; and isolating an antibody produced by the hybridoma.

Regardless of the mode of production, once positive clones are at hand a series of assays may be employed to characterize the clones. In the case where hybridoma technique is used to generate the antibodies, these assays can even be done at the hybridoma supernatant level, i.e., before cloning of the TCRL coding sequence. Such a selection typically increases the robustness (high throughput) of screening. The assays may include, but are not limited to:

Binding of the antibody to the specific MHC-peptide complex against which the antibody was produced or selected against (hereinafter “the specific complex”).

This is typically effected by Enzyme Linked Immunosorbent Assay (ELISA): The method involves fixation of the complex (or non-relevant MHC-peptide complexes which are used as negative control) to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Differential ELISA for those TCRL-antibodies which exhibit a positive binding in ELISA (positive clones) is determined by analyzing specific versus non-specific (non-relevant) MHC-peptide complexes. For example, a gp100 complex may be used as an irrelevant complex when screening for anti tyrosinase TCRLs. Alternatively, different HLA-restricted peptides from the same target may be used as an irrelevant complex.

Typically, the binding of the TCRL antibody to the non-relevant MHC-peptide complex does not exceed background level (e.g., unloaded cells stained with a secondary antibody).

Further analysis is done by SPR analysis of the TCRL-antibody (e.g., of the supernatant) to the specific complex, affinity range will typically be in the K_{D} range of pM to nM (e.g., below 20 nM).

In order to get insight on the binding of the TCRL-antibody to cells, the binding is determined to peptide loaded cells (i.e., loaded with the specific MHC-restricted peptide) versus binding to cells loaded with irrelevant MHC-restricted peptides. Cells used in this assay are antigen presenting cells (APC) such as JY cells or T2 cells for MHC class I presentation. Measures are taken to use an APC which presents the same HLA for which the HLA-restricted peptide is restricted (e.g., HLA-A2). According to a specific embodiment, the loading assay uses the T2 cells. MHC class I antigen presentation and cell surface expression depend primarily on peptide transport into the endoplasmic reticulum or Golgi by the transporter associated with antigen transport (TAP). T2 cell lines are deficient in TAP but still express low amounts of MHC class I on the surface of the cells. T2 cells were established by PEG-mediated fusion of the B-lymphoblastoid cell line (B-LCL) LCL 721.174 with an 8-azaguanine and ouabain-resistant variant of the T-LCL CEM (CEMR.3); the cells were described to synthesize HLA-A2 and -B5 RNA, but express only small amounts of A2 and no B5 antigen on the cell surface. In the presence of the loaded peptide, the HLA-A2 is induced and 10⁴-10⁵ of complexes are presented dependent on the affinity of the peptide to the HLA-A2. Affinity of the TCRL-antibody is analyzed also after cloning of the TCRL where the end product has a predetermined affinity characterized by the above described K_{D} range (e.g., 0.1 nM to 50 nM).

By “cloning” the invention refers to post-fusion cloning of the TCRL coding sequence or in the case of phage-display screening, cloning of the antibody fragment to a full-length antibody.

The specificity of the TCRL is further analyzed versus a list of in silico-predicted or naturally presented MHC-restricted peptides.

Similar Peptides

An important criterion for the selection of potential target peptides for TCRL development is the presentation of other peptides that are similar in sequence to the target peptide. A similar peptide that is actually presented on an HLA molecule might be undesirably targeted by the TCRL against the candidate target. The underlying assumption in this case is that sequence similarity might infer TCRL non-specific binding. Any candidate target will have similar peptides to consider and the higher the number of similar peptides a peptide candidate possess the more extensive evaluation of binding of TCR-like antibodies to these peptides should be performed and subjected to careful evaluation of selectivity. This may be regarded as target qualification. To assess this evaluation and filtering process, a search of human protein libraries such as Uniprot (www.uniprot.org) and NCBI-nr (ftp://ftp.ncbi.nih.gov/blast/db/FASTA/nr.gz) is performed for peptides with sequences similarity to the target peptide according to the following criteria:

(i) A difference of up to four amino acids

(ii) The peptide length is 9-11 amino acids

(iii) The predicted binding score (affinity) to HLA-A2 is above 2 based on the bimas algorithm www-bimas.cit.nih.gov/molbio/hla_bind to include only peptides that can bind to HLA-A2.

The search is extended to peptides that are similar to the target peptide when considering amino acids that have similar characteristics as equivalent. The equivalent classes are [AG], [DE], [ILV], [NQ] and [ST] (e.g., A and G are considered as equivalent). To these peptides the same criteria is applied as above.

The criteria indicated above generates a list of similar peptides from which a subset is selected for further processing and evaluation according to the following criteria:
(1) Highest priority is assigned to peptides that are found in mass spectrometry analysis of various cells or tissues from which HLA restricted peptides have been eluted and peptide sequence was verified as HLA binder, peptides that are known in the literature or present in HLA peptide data bases;
(2) Peptides most similar to the target peptide are selected having the potential of being presented by the same HLA as the peptide of interest (up to 4 amino acid substitutions);
(3) Peptides derived from genes that are ubiquitous in essential tissues are also assigned high priority.

It is important and critical to note that the actual existence of a peptide in the protein libraries does not indicate that the peptide is naturally presented by HLA. Hence, according to some embodiments of the invention, validation for the true representation of in-silico predicted peptides (e.g., by MS analysis of essential tissues) is effected.

Thus, according to some embodiments of the invention, selected similar peptides are chemically synthesized and used for several further analyses.

According to a specific embodiment, similar peptides are those that preserve the critical residues for antibody binding (as determined by alanine/glycine scanning as described below) as well as the anchor residues. Thus, a round of similar peptides selection is performed when Alanine/Glycine scanning data are available as described below for a particular TCR-like antibody. Based on alanine scanning the contribution of each amino acid residue in the peptide antigen to TCRL binding is measured and evaluated, such as FACS analysis of peptide loaded cells. Similar peptides that preserve the critical positions are identified by the below described tools and are assigned higher priority. These peptides are synthesized and used for fine specificity evaluation as described below.

The strategy described here combines in silico analysis of peptide sequence similarity combined with Mass spectroscopy analysis of eluted HLA peptides, peptide data bases and alanine scanning provides a tool box to fully control peptide search parameters, more than other tools such as BLAST or ScanProsite provide. Additional parameters are employed including the range of allowed peptide lengths, the maximum allowed number or differences in sequence, and the requirement for HLA binding score. The tool also applies the ability to define certain amino acids as equivalent. Most important is the ability to highlight peptides that have been found by mass spectrometry or from database data or literature.

A myriad of bioinformatic tools are known in the art for compiling lists of similar peptides which may have the potential of being MHC-restricted.

See for instance:

1. www.mhc-pathway.net/2.

### 2. NetChop 3.1 (20S 3.0)

### 3. MAPPP

### 4. PaProC

### 5. IEDB Analysis Resource

6. BIMAS—scoring peptide presentation potential: /www-bimas.cit.nih.gov/molbio/hla_bind/.

Databases or otherwise collections of naturally presented peptides (or as used herein “HLA-presented peptides”) are readily available such as a result of mass-spectrometry (MS) analysis of peptides from human tissues.

See e.g., SYFPEITHI:

www.syfpeithi.de/bin/MHCServer.dll/FindYourMotif.htm

As well as the following patent applications listing HLA-presented peptides. US 2009/041794, U.S. Pat. No. 7,396,904, EP 2113253 B, US 2010/158929, US 2009/123489, US 2009/136528, U.S. Pat. No. 7,833,969, US 2010/158931, US 2011/0002963, US 2009/148400, US 2010/021441, US 2010/029571, US 2010/029571, US 2009/317428, US 2009/221509, WO 2011/073215, US 2011150849, US 2011/0117117, US 2011/10229505, US 2011/10229504, US 2010/0003718, US 2010/0003718, US 2009/0226474, US 2009/0062512, US 2007/0099182, US 2007/0026433, WO 2010/111467, US 2010/068186, US 2008/292549, US 2009/214551, US 2011/0014169, WO 2011/001152, US 2008/292602, EP 1771727A, WO 2010/106535, WO 2009/090651, WO 02/094981, U.S. Pat. No. 7,488,793, U.S. Pat. No. 7,351,409, WO 97/15597.

In order to augment the specificity selection of the TCRL-antibody, the present invention provides for a method of qualifying a TCR-like antibody (which may be useful for therapy), the method comprising:

(a) providing a TCRL-antibody, as mentioned, an antibody capable of binding, with a human major histocompatibility complex (MHC)-restricted specificity, a MHC being complexed with an HLA-restricted peptide antigen of interest, wherein the binding is with a predetermined affinity;
(b) providing HLA-presented peptides which are present on at least one essential tissue, the HLA-presented peptides having at least one amino acid substitution as compared to the HLA-restricted peptide antigen in an amino acid residue not critical for binding the peptide antigen, as determined by alanine scanning of the HLA-restricted peptide antigen; and
(c) determining binding of the antibody to the HLA-presented peptides by

FACS analysis of cells loaded with the HLA-presented peptides, the antibody being qualified for TCRL therapy if the binding is undetectable by the FACS analysis.

According to an additional or an alternative aspect there is provided a method for selecting highly selective TCR-like antibody with the optimal fine specificity towards a specific MHC being complexed with an HLA-restricted peptide antigen, the method comprising:

(a) providing an antibody capable of binding, with a human major histocompatibility complex (MHC)-restricted specificity, a MHC being complexed with an HLA-restricted peptide antigen, wherein the binding is with a predetermined affinity;

(b) determining binding of the antibody to peptides similar to the HLA-restricted peptide antigen having been mutated with alanine/glycine/valine/leucine in amino acids other than anchor residues so as to identify amino acids which are critical for binding of the antibody;

(c) determining binding of the antibody to in silico-predicted and/or validated HLA-presented peptides which are present on at least one normal essential tissue, the HLA-presented peptides comprising 1-4 amino acid substitutions as compared to the HLA-restricted peptide;

wherein the determining binding of the antibody to the peptides of (a) and (b) is by FACS analysis of cells loaded with the peptides or by functional assay, the antibody being qualified for TCRL therapy if the binding of (b) is undetectable by the FACS analysis.

According to some embodiments of the invention, the HLA-presented peptides which are present on at least one essential tissue have at least one amino acid substitution as compared to the HLA-restricted peptide antigen in an amino acid residue not critical for binding the peptide antigen.

According to some embodiments of the invention, the method further comprises determining binding of the antibody to normal cells, which do not present the HLA-restricted peptide but are positive for the same HLA type.

According to an aspect of the invention, there is provided an antibody having been selected according to the methods described herein or which qualifies to the criteria of selection described herein.

According to a specific embodiment, the predetermined affinity is below 20 nM.

As used herein “an essential tissue” refers to one of the following tissue of the brain, heart, kidneys, liver, pancreas, colon, stomach and lung systems. According to a specific embodiment the essential tissue is a normal tissue not affected by a disease (e.g., cancer, autoimmunity or pathogen-related diseases e.g., viral infected). Hence the HLA-presented peptides screened are not associated with a disease.

Normal cells are cells not affected by a disease (e.g., cancer, autoimmunity or pathogen-related diseases e.g., viral infected).

Hence, the binding specificity of the TCRL is determined with respect to a series of peptides in which typically a single amino acid is mutated with respect to the naïve HLA-restricted peptide against which the TCRL has been produced.

For systematic analysis, the peptide is subject to an Alanine/Glycine scanning analysis. Alanine/Glycine scanning is a technique used to determine the contribution of a specific residue to the stability or function of given protein. According to an embodiment of the invention the general art term “alanine scanning” refers to substitutions by alanine, glycine, valine or leucine. These are used because of their non-bulky, chemically inert, methyl functional group that nevertheless mimics the secondary structure preferences that many of the other amino acids possess.

Of note, as any HLA-restricted peptide comprises anchor residues which anchor the peptide to the MHC complex, the mutations are not in those anchor residues. Typically these residues are P2 and P9 for HLA-A2. Hence the mutations are typically in P1, P3, P4, P5, P6, P7 and P8 in this haplotype.

In 10 amino acids peptides these residues are P2 or P3 and P9 or P10 for HLA-A2.

Such peptides are then loaded to antigen presenting cells and binding of the TCRL is determined by FACS.

For complexes which are presented at levels below 100 per cell functional assays should be employed such as described above.

It is considered that FACS can detect as low as 40-80 peptide-HLA (e.g., A2) complexes per cell when antibody affinity is in the low nM range (i.e., below 10 nM).

A critical binding site is determined as a site in which a substitution mutation reduces binding of the TCRL to the mutated peptide loaded cell by at least 70% as compared to binding of the TCRL to the native peptide loaded cells, as determined by FACS.

According to a specific embodiment, a critical binding site is determined as a site in which a substitution mutation reduces binding of the TCRL to the mutated peptide loaded cell by at least 75% as compared to binding of the TCRL to the native peptide loaded cells, as determined by FACS.

According to a specific embodiment, a critical binding site is determined as a site in which a substitution mutation reduces binding of the TCRL to the mutated peptide loaded cell by at least 80% as compared to binding of the TCRL to the native peptide loaded cells, as determined by FACS.

According to a specific embodiment, a critical binding site is determined as a site in which a substitution mutation reduces binding of the TCRL to the mutated peptide loaded cell by at least 85% as compared to binding of the TCRL to the native peptide loaded cells, as determined by FACS.

According to a specific embodiment, a critical binding site is determined as a site in which a substitution mutation reduces binding of the TCRL to the mutated peptide loaded cell by at least 90% as compared to binding of the TCRL to the native peptide loaded cells, as determined by FACS.

According to a specific embodiment, a critical binding site is determined as a site in which a substitution mutation reduces binding of the TCRL to the mutated peptide loaded cell by at least 95% as compared to binding of the TCRL to the native peptide loaded cells, as determined by FACS.

According to a specific embodiment, a critical binding site is determined as a site in which a substitution mutation reduces binding of the TCRL to the mutated peptide loaded cell by 100% (completely abrogated binding) as compared to binding of the TCRL to the native peptide loaded cells, as determined by FACS.

Once the amino acids, which are critical for binding, are determined [typically for a qualified TCRL, according to the present teachings, at least 4-5 such residues (not including the anchor residues)], HLA-presented peptides which are present on at least one essential tissue having at least one amino acid substitution as compared to the HLA-restricted peptide antigen in an amino acid not critical for binding the peptide antigen (also referred to as similar peptides) are synthesized. Typically, these amino acid substitutions are in 1, 2 or 3 positions in the HLA-restricted peptide (those amino acids which are not critical for binding as determined above).

The presence of such HLA-presented peptides i.e., natural presentation, in essential tissues is typically determined by mass-spectrometry. When such peptides are identified, these peptides are synthesized and the binding of the TCRL is determined on peptide-loaded cells by FACS. The TCRL is qualified as specific and suitable for therapy when binding to these similar peptides is undetectable in the FACS analysis (e.g., FACS detection threshold is mentioned above).

It will be appreciated that the TCRLs may be further qualified with respect to similar HLA-peptide complexes which are found to be presented not necessarily on essential tissues. Similar may include 1, 2, 3, 4 or 5 alterations in the sequence of the HLA restricted peptide (not in anchor positions). See for instance qualification on similar peptides done for the D7, D11 and B47 in the Examples section which follows.

TCRL-antibodies selected according to the present teachings are characterized by unprecedented specificity.

Thus according to an aspect of the invention there is provided an antibody capable of binding, with a human major histocompatibility complex (MHC)-restricted specificity, a MHC being complexed with an HLA-restricted peptide antigen, the antibody having a binding specificity dictated by at least 4 amino acid residues in the HLA-restricted peptide such that at least 70% reduction in binding of the antibody to the complex is observed when each of the at least 4 amino acid residues is substituted as determined by FACS of cells loaded with the HLA-restricted peptide comprising the substitution, the at least 4 amino acid residues not being anchor residues.

FACS assays are well known in the art. Measures are taken to employ controls (e.g., positive control of the binding peptide), same antigen presenting cells [e.g., dendritic cells (DC), T2 cells, Epstein Barr virus-transformed B cell lines (e.g., JY and TK-6] and the same level of presentation between the assayed peptides.

Thus substitution in each of the at least 4 amino acid residues (which are not anchor residues) causes a loss of binding by at least 70% according to the above parameters. The present inventors have realized that a collection of as much as of at least 4 residues, 5 or 6 which are critical for binding imparts the antibody with fine specificity and mitigates toxicity concerns.

According to a specific embodiment, the residues which are critical for binding include X1, X3, X4, X5.

According to a specific embodiment, the residues which are critical for binding include X3, X4, X5, X6.

According to a specific embodiment, the residues which are critical for binding include X4, X5, X6, X7.

According to a specific embodiment, the residues which are critical for binding include X5, X6, X7, X8.

According to a specific embodiment, the residues which are critical for binding include X1, X3, X5, X6.

According to a specific embodiment, the residues which are critical for binding include X1, X3, X6, X7.

According to a specific embodiment, the residues which are critical for binding include X1, X3, X7, X8.

According to a specific embodiment, the residues which are critical for binding include X1, X3, X4, X6.

According to a specific embodiment, the residues which are critical for binding include X1, X3, X4, X7.

According to a specific embodiment, the residues which are critical for binding include X1, X3, X4, X8.

According to a specific embodiment, the residues which are critical for binding include X3, X4, X6, X8.

According to a specific embodiment, the residues which are critical for binding include X4, X6, X7, X8.

According to a further aspect of the invention there is provided an antibody capable of binding, with a human major histocompatibility complex (MHC)-restricted specificity, a HLA-A2/TyrD369-377 peptide complex, the antibody having a binding specificity dictated by at least 4 amino acid residues in the TyrD369-377 peptide such that at least 70% reduction in binding of the antibody to the complex is observed when each of the at least 4 amino acid residues is substituted as determined by FACS of cells loaded with the peptide comprising the substitution, the at least 4 amino acid residues not being anchor residues.

According to an embodiment of this aspect of the invention, the at least 4 amino acid residues are selected from X1, X3, X4, X6 and X7 of TyrD369-377.

According to an embodiment of this aspect of the invention, the at least 4 amino acid residues are selected from X1, X3, X4, X6 and X7 of TyrD369-377.

According to an embodiment of this aspect of the invention, the at least 4 amino acid residues are selected from X3, X4, X6 and X7 of TyrD369-377 (D7).

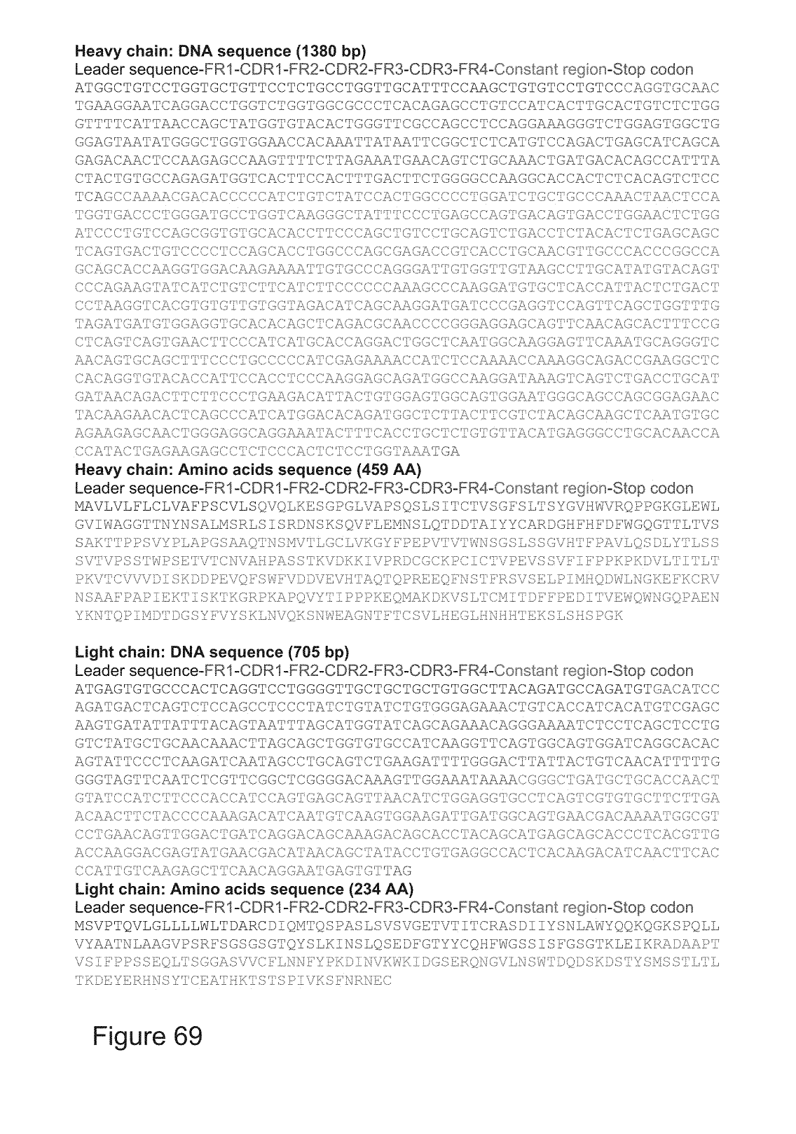

The D7 antibody comprises the CDR sequences (HC-heavy chain; LC-light chain, the CDRs are ordered N to C), as shown in FIG. 69.

According to an embodiment of this aspect of the invention, the at least 4 amino acid residues are selected from X1, X3, X4 and X6 of TyrD369-377 (D11).

The D11 antibody comprises the following CDR sequences (HC-heavy chain; LC-light chain, the CDRs are ordered N to C) as shown in FIG. 68.

According to a specific embodiment, the at least 4 amino acid residues comprise 5 amino acid residues such that at least 70% reduction in binding of the antibody to the complex is observed when each of at least 4 amino acid residues of the 5 amino acids residues is substituted by alanine and wherein at least 30% reduction in binding of the antibody to the complex is observed when a fifth amino acid of the 5 amino acids is substituted by alanine.

According to some embodiments of the invention the at least 70% reduction in binding is at least 90% reduction in binding observed when at least 1 amino acid residue of the at least 4 amino acid residues comprises the substitution.

According to some embodiments of the invention the at least 70% reduction in binding is at least 90% reduction in binding observed when each of at least 2 amino acid residues of the at least 4 amino acid residues comprise the substitutions.

Alternatively or additionally there is provided an antibody capable of binding, with a human major histocompatibility complex (MHC)-restricted specificity, a MHC being complexed with an HLA-restricted peptide antigen, wherein said antibody does not bind to any HLA-presented peptides, which are present in essential tissues as determined by FACS analysis of cells loaded with said HLA-presented peptides, the HLA-presented peptides having at least one amino acid substitution as compared to said HLA-restricted peptide antigen in an amino acid residue not critical for binding said peptide antigen, as determined by alanine scanning of said HLA-restricted peptide antigen.

The amino acid substitutions described herein may be conservative, semi conservative or radical. A conservative substitution relates to a change to an amino acid with similar physiochemical properties. Following is a canonic classification of amino acids into groups, though other conservative substitutions may be done involving also synthetic amino acids. As the MHC-peptide epitope is not a simple epitope some amino acids may be substituted conservatively while others may be substituted semi-conservatively or radically.

Aliphatic amino acids. The side chains of glycine, alanine, valine, leucine, and isoleucine, contain saturated carbon-carbon and carbon-hydrogen bonds only.

Aromatic amino acids. Phenylalanine, tyrosine, and tryptophan contain ring systems.

Ionizable basic amino acids. Histidine, lysine, and arginine each have a nitrogen atom which, unlike the nitrogen of tryptophan, is ionized at the pH ranges found in the cell.

Carboxyllate-containing amino acids. Aspartic acid and asparagine have four carbons; glutamic acid and glutamine have five carbons in all.

Hydroxyl and sulfur-containing side chains Serine and cysteine can be thought of as being related to alanine. Serine is alanine with a hydroxyl (—OH) group and cysteine is alanine with a sulfhydryl (—SH) group. Threonine has four carbons, with a hydroxyl group on the beta carbon. Methionine has a methyl group on its sulfur.

It will be appreciated that the substitution mutation may be a conservative substitution or non-conservative substitution (e.g., glycine/alanine, dependent of course on the amino acid identity at the selected position), though for a rougher selection a non-conservative substitution is employed.

According to a specific embodiment, the TCRL antibody thus selected has a binding affinity below 10 nanomolar to a soluble (e.g., single chain human) major histocompatibility complex (MHC) complexed with said HLA-restricted peptide antigen, as determined by surface plasmon resonance assay.

According to a specific embodiment, the TCRL antibody thus selected is capable of binding the HLA-restricted peptide antigen when naturally presented on cells, as determined by FACS.

According to a specific embodiment, the cells are cancer cells.

TCRL antibodies of the present invention may be produced using recombinant DNA technology.

Thus according to an aspect of the invention there is provided an isolated polynucleotide comprising a nucleic acid sequence encoding the antibody as described herein.

Also provided is an expression vector, comprising the polynucleotide operably linked to a cis-acting regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the antibody from a host cell in which it is placed. Preferably the signal sequence for this purpose is a mammalian signal sequence.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of TCRL mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Also provided are cells which comprise the polynucleotides/expression vectors as described herein.

Such cells are typically selected for high expression of recombinant proteins (e.g., bacterial, plant or eukaryotic cells e.g., CHO, HEK-293 cells), but may also be host cells having a specific immune effector activity (e.g., T cells or NK cells) when for instance the CDRs of the TCRL are implanted in a T Cell Receptor or CAR transduced in said cells which are used in adoptive cell therapy as further described hereinbelow.

The high specificity of the TCRLs renders them particularly suitable for diagnostic and therapeutic applications.

Thus, according to an aspect of the present invention, there is provided a method of detecting a cell presenting an HLA-restricted peptide antigen of interest. The method comprises contacting the cell with the TCRL of the present invention having specificity to the HLA-restricted peptide antigen of interest. The contacting is effected under conditions which allow immunocomplex formation, wherein a presence of the immunocomplex or level thereof is indicative of the cell presenting the HLA-restricted peptide antigen of interest.

The term “detecting”, as used herein, refers to the act of detecting, perceiving, uncovering, exposing, visualizing or identifying a cell. The precise method of detecting is dependent on the detectable moiety (also referred to herein as identifiable moiety) to which the antibody is attached as further described herein below.

Single cells may be used in accordance with the teachings of the present invention as well as a plurality of cells. For instance the cells may be from any biological sample such as cell-lines, primary (e.g., tumor cultures) and cellular samples, e.g. surgical biopsies including incisional or excisional biopsy, fine needle aspirates and the like. Methods of biopsy retrieval are well known in the art.

The above-mentioned detection method can be harnessed to the diagnosis of diseases which are characterized by above normal presentation or different tissue distribution of the HLA-peptide complex.

As used herein the term “diagnosing” refers to classifying a disease, determining a severity of a disease (grade or stage), monitoring progression, forecasting an outcome of the disease and/or prospects of recovery.

The subject may be a healthy subject (e.g., human) undergoing a routine well-being check up. Alternatively, the subject may be at risk of the disease. Yet alternatively, the method may be used to monitor treatment efficacy.

The TCRL may comprise e.g., attached to an identifiable moiety. Alternatively or additionally, the TCRL (or a complex comprising same) may be identified indirectly such as by using a secondary antibody.

The contacting may be effected in vitro (i.e. in a cell line, primary cells), ex vivo or in vivo.

As mentioned, the method of the present invention is effected under conditions sufficient to form an immunocomplex (e.g. a complex between the antibodies of the present invention and the peptide complexed to the MHC, typically when the cells are not lysed); such conditions (e.g., appropriate concentrations, buffers, temperatures, reaction times) as well as methods to optimize such conditions are known to those skilled in the art, and examples are disclosed herein. According to a specific embodiment the antibody which binds the HLA-A2/Tyrosinase peptide as described herein may be used for the diagnosis of melanoma. According to a specific embodiment the antibody which binds the WT1 restricted peptide as described herein may be used for the diagnosis of hematological malignancies and other malignancies associated with WT1 as described above.

Determining a presence or level of the immunocomplex of the present invention is dependent on the detectable moiety to which the antibody is attached.

Examples of detectable moieties that can be used in the present invention include but are not limited to radioactive isotopes, phosphorescent chemicals, chemiluminescent chemicals, fluorescent chemicals, enzymes, fluorescent polypeptides and epitope tags. The detectable moiety can be a member of a binding pair, which is identifiable via its interaction with an additional member of the binding pair, and a label which is directly visualized. In one example, the member of the binding pair is an antigen which is identified by a corresponding labeled antibody. In one example, the label is a fluorescent protein or an enzyme producing a colorimetric reaction.

Further examples of detectable moieties, include those detectable by Positron Emission Tomagraphy (PET) and Magnetic Resonance Imaging (MRI), all of which are well known to those of skill in the art.

When the detectable moiety is a polypeptide, the immunolabel (i.e. the antibody conjugated to the detectable moiety) may be produced by recombinant means or may be chemically synthesized by, for example, the stepwise addition of one or more amino acid residues in defined order using solid phase peptide synthetic techniques. Examples of polypeptide detectable moieties that can be linked to the antibodies of the present invention using recombinant DNA technology (in which the polynucleotide encoding the TCRL is translationally fused to the detectable moiety) include fluorescent polypeptides, phosphorescent polypeptides, enzymes and epitope tags.

Alternatively, chemical attachment of a detectable moiety to the antibodies of the present invention can be effected using any suitable chemical linkage, direct or indirect, as via a peptide bond (when the detectable moiety is a polypeptide), or via covalent bonding to an intervening linker element, such as a linker peptide or other chemical moiety, such as an organic polymer. Such chimeric peptides may be linked via bonding at the carboxy (C) or amino (N) termini of the peptides, or via bonding to internal chemical groups such as straight, branched or cyclic side chains, internal carbon or nitrogen atoms, and the like. Such modified peptides can be easily identified and prepared by one of ordinary skill in the art, using well known methods of peptide synthesis and/or covalent linkage of peptides. Description of fluorescent labeling of antibodies is provided in details in U.S. Pat. Nos. 3,940,475, 4,289,747, and 4,376,110.

Exemplary methods for conjugating two peptide moieties are described herein below:

SPDP Conjugation:

Any SPDP conjugation method known to those skilled in the art can be used. For example, in one illustrative embodiment, a modification of the method of Cumber et al. (1985, Methods of Enzymology 112: 207-224) as described below, is used.

A peptide, such as an identifiable or therapeutic moiety, (1.7 mg/ml) is mixed with a 10-fold excess of SPDP (50 mM in ethanol) and the antibody is mixed with a 25-fold excess of SPDP in 20 mM sodium phosphate, 0.10 M NaCl pH 7.2 and each of the reactions incubated, e.g., for 3 hours at room temperature. The reactions are then dialyzed against PBS.

The peptide is reduced, e.g., with 50 mM DTT for 1 hour at room temperature. The reduced peptide is desalted by equilibration on G-25 column (up to 5% sample/column volume) with 50 mM KH₂PO₄ pH 6.5. The reduced peptide is combined with the SPDP-antibody in a molar ratio of 1:10 antibody:peptide and incubated at 4° C. overnight to form a peptide-antibody conjugate.

Glutaraldehyde Conjugation:

Conjugation of a peptide (e.g., an identifiable or therapeutic moiety) with an antibody can be accomplished by methods known to those skilled in the art using glutaraldehyde. For example, in one illustrative embodiment, the method of conjugation by G. T. Hermanson (1996, “Antibody Modification and Conjugation, in Bioconjugate Techniques, Academic Press, San Diego) described below, is used.

The antibody and the peptide (1.1 mg/ml) are mixed at a 10-fold excess with 0.05% glutaraldehyde in 0.1 M phosphate, 0.15 M NaCl pH 6.8, and allowed to react for 2 hours at room temperature. 0.01 M lysine can be added to block excess sites. After-the reaction, the excess glutaraldehyde is removed using a G-25 column equilibrated with PBS (10% v/v sample/column volumes).

Carbodiimide Conjugation:

Conjugation of a peptide with an antibody can be accomplished by methods known to those skilled in the art using a dehydrating agent such as a carbodiimide. Most preferably the carbodiimide is used in the presence of 4-dimethyl aminopyridine. As is well known to those skilled in the art, carbodiimide conjugation can be used to form a covalent bond between a carboxyl group of peptide and an hydroxyl group of an antibody (resulting in the formation of an ester bond), or an amino group of an antibody (resulting in the formation of an amide bond) or a sulfhydryl group of an antibody (resulting in the formation of a thioester bond).

Likewise, carbodiimide coupling can be used to form analogous covalent bonds between a carbon group of an antibody and an hydroxyl, amino or sulfhydryl group of the peptide. See, generally, J. March, Advanced Organic Chemistry: Reaction's, Mechanism, and Structure, pp. 349-50 & 372-74 (3d ed.), 1985. By means of illustration, and not limitation, the peptide is conjugated to an antibody via a covalent bond using a carbodiimide, such as dicyclohexylcarbodiimide. See generally, the methods of conjugation by B. Neises et al. (1978, Angew Chem., Int. Ed. Engl. 17:522; A. Hassner et al. (1978, Tetrahedron Lett. 4475); E. P. Boden et al. (1986, J. Org. Chem. 50:2394) and L. J. Mathias (1979, Synthesis 561). The level of immunocomplex may be compared to a control sample from a non-diseased subject, wherein an up-regulation of immunocomplex formation is indicative of melanoma. Preferably, the subject is of the same species e.g. human, preferably matched with the same age, weight, sex etc. It will be appreciated that the control sample may also be of the same subject from a healthy tissue, prior to disease progression or following disease remission.

According to a specific embodiment, the detection is effected by FACS.

As mentioned the antibodies of the present invention can also be used in therapeutics where the antibody comprises a therapeutic moiety.

The therapeutic moiety can be an integral part of the antibody e.g., in the case of a whole antibody, the Fc domain, which activates antibody-dependent cell-mediated cytotoxicity (ADCC). ADCC is a mechanism of cell-mediated immune defense whereby an effector cell of the immune system actively lyses a target cell, whose membrane-surface antigens have been bound by specific antibodies. It is one of the mechanisms through which antibodies, as part of the humoral immune response, can act to limit and contain infection. Classical ADCC is mediated by natural killer (NK) cells; macrophages, neutrophils and eosinophils can also mediate ADCC. For example, eosinophils can kill certain parasitic worms known as helminths through ADCC mediated by IgE. ADCC is part of the adaptive immune response due to its dependence on a prior antibody response.

Alternatively or additionally, the antibody may be a bispecific antibody in which the therapeutic moiety is a T cell engager for example, such as an anti CD3 antibody or an anti CD16a alternatively the therapeutic moiety may be an anti immune checkpoint molecule (anti PD-1).

Alternatively or additionally the antibody may be attached to a heterologous therapeutic moiety (methods of conjugation are described hereinabove). The therapeutic moiety can be, for example, a cytotoxic moiety, a toxic moiety, a cytokine moiety, a drug.

The antibody may be in a soluble or insoluble form.

Insoluble forms may be those in which a molecule comprising the antibody's

CDRs is anchored to or expressed by a cell or a particle (the latter can be used for therapeutic as well as diagnostic applications).

Examples of such cells include immune cells, T cells, B cells, dendritic cells, CIK, NKT, NK cells (autologous, allogeneic, xenogeneic).

According to a specific embodiment, the antibody (or actually CDRs thereof) form a CAR (as explained above) or an artificial T Cell Receptor. Thus a polynucleotide coding for such a molecule is transduced in a cell of interest.

According to some embodiments of the invention, the cell is a T cell, a natural killer cell, a cell that exerts effector killing function on a target cell, a cell that exerts a suppressive effect on effector T cells, an engineered cell with an effector killing function or an engineered cell with a suppressive function.

According to some embodiments of the invention, the cell is a T cell, or αβT cell, or γδT cell.

According to some embodiments of the invention, the cell is a natural killer (NK) cell.

According to some embodiments of the invention, the natural killer cell is used to target cancer, viral and/or immune cells such as in an autoimmune disease.

According to some embodiments of the invention, the natural killer cell is used to treat a pathology caused by or associated with a viral infection, or cancer.

According to some embodiments of the invention, the T cell is a cytotoxic T cell (effector T cell).

According to some embodiments of the invention, the cytotoxic T cell (effector T cell) is used to target cancer, and/or viral antigens.

According to some embodiments of the invention, the cytotoxic T cell is used to treat a pathology caused by or associated with a viral infection, or cancer.

According to some embodiments of the invention, the T cell comprises a Treg (T regulatory cell).

According to some embodiments of the invention, the Treg is used to target autoimmune antigen(s).

According to some embodiments of the invention, the Treg is used to treat an autoimmune disease.

According to some embodiments of the invention, the T cell comprises a CD3 T cell.

According to some embodiments of the invention, the T cell comprises a CD4 T cell.

According to some embodiments of the invention, the T cell comprises a CD8 T cell.

According to some embodiments of the invention, the antigen binding domain comprises a single chain Fv (scFv) molecule.

The cytoplasmic domain (also referred to as “intracellular signaling domain”) of the CAR molecule of the invention is responsible for activation of at least one of the normal effector functions of the immune cell in which the CAR has been placed in.

The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR molecule of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs (ITAMs).

Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the invention comprises a cytoplasmic signaling sequence derived from CD3 zeta.

In a preferred embodiment, the cytoplasmic domain of the CAR can be designed to comprise the CD3-zeta signaling domain by itself or combined with any other desired cytoplasmic domain(s) useful in the context of the CAR of the invention. For example, the cytoplasmic domain of the CAR can comprise a CD3 zeta chain portion and a costimulatory signaling region. The costimulatory signaling region refers to a portion of the CAR comprising the intracellular domain of a costimulatory molecule. A co-stimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. Thus, while the invention in exemplified primarily with 4-1BB as the co-stimulatory signaling element, other costimulatory elements are within the scope of the invention.

According to some embodiments of the invention, the intracellular domain comprises, a co-stimulatory signaling region and a zeta chain portion. The co-stimulatory signaling region refers to a portion of the CAR molecule comprising the intracellular domain of a co-stimulatory molecule. Co-stimulatory molecules are cell surface molecules other than antigen receptors or their ligands that are required for an efficient response of lymphocytes to antigen.

“Co-stimulatory ligand,” as the term is used herein, includes a molecule on an antigen presenting cell [e.g., an aAPC (artificial antigen presenting cell), dendritic cell, B cell, and the like] that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class 1 molecule, BTLA and a Toll ligand receptor.

A “co-stimulatory signal”, as used herein, refers to a signal, which in combination with a primary signal, such as TCR/CD3 ligation, leads to T cell proliferation and/or upregulation or down regulation of key molecules.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter cilia, an MHC Class I molecule loaded with a peptide, an anti-CD3 antibody, a superagonist anti-CD28 antibody, and a superagonist anti-CD2 antibody.

With respect to the cytoplasmic domain, the CAR molecule of some embodiments of the invention can be designed to comprise the CD28 and/or 4-1BB signaling domain by itself or be combined with any other desired cytoplasmic domain(s) useful in the context of the CAR molecule of some embodiments of the invention. In one embodiment, the cytoplasmic domain of the CAR can be designed to further comprise the signaling domain of CD3-zeta. For example, the cytoplasmic domain of the CAR can include but is not limited to CD3-zeta, 4-1BB and CD28 signaling modules and combinations thereof.

According to some embodiments of the invention, the intracellular domain comprises at least one, e.g., at least two, at least three, at least four, at least five, e.g., at least six of the polypeptides selected from the group consisting of: CD3 (CD247, CD3z), CD28, 41BB, ICOS, OX40, and CD137.

According to some embodiments of the invention, the intracellular domain comprises the CD3ζ-chain [CD247 molecule, also known as “CD3-ZETA” and “CD3z”; GenBank Accession NOs. NP_000725.1 and NP_932170.1], which is the primary transmitter of signals from endogenous TCRs.

According to some embodiments of the invention, the intracellular domain comprises various co-stimulatory protein receptors to the cytoplasmic tail of the CAR to provide additional signals to the T cell (second generation CAR). Examples include, but are not limited to, CD28 [e.g., GenBank Accession Nos. NP_001230006.1, NP_001230007.1, NP_006130.1], 4-1BB [tumor necrosis factor receptor superfamily, member 9 (TNFRSF9), also known as “CD137”, e.g., GenBank Accession No. NP_001552.2], and ICOS [inducible T-cell co-stimulator, e.g., GenBank Accession No. NP_036224.1]. Preclinical studies have indicated that the second generation of CAR designs improves the antitumor activity of T cells.

According to some embodiments of the invention, the intracellular domain comprises multiple signaling domains, such as CD3z-CD28-41BB or CD3z-CD28-OX40, to further augment potency. The term “OX40” refers to the tumor necrosis factor receptor superfamily, member 4 (TNFRSF4), e.g., GenBank Accession No. NP_003318.1 (“third-generation” CARs).

According to some embodiments of the invention, the intracellular domain comprises CD28-CD3z, CD3z, CD28-CD137-CD3z. The term “CD137” refers to tumor necrosis factor receptor superfamily, member 9 (TNFRSF9), e.g., GenBank Accession No. NP_001552.2.

According to some embodiments of the invention, when the CAR molecule is designed for a natural killer cell, then the signaling domain can be CD28 and/or CD3. The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

According to some embodiments of the invention, the transmembrane domain comprised in the CAR molecule of some embodiments of the invention is a transmembrane domain that is naturally associated with one of the domains in the CAR. According to some embodiments of the invention, the transmembrane domain can be selected or modified by amino acid substitution to avoid binding of such domains to the transmembrane domains of the same or different surface membrane proteins to minimize interactions with other members of the receptor complex.

According to some embodiments, between the extracellular domain and the transmembrane domain of the CAR molecule, or between the cytoplasmic domain and the transmembrane domain of the CAR molecule, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

According to an aspect of some embodiments of the invention, there is provided a method of treating a pathology in a subject in need thereof, comprising administering to the subject the TCRL antibody (soluble or insoluble e.g., TCR/CAR), thereby treating the pathology in the subject.

Also provided is a use of the TCRL antibody (soluble or insoluble e.g., TCR/CAR) as defined herein in the manufacture of a medicament for treating a pathology e.g., cancer, viral infection or autoimmune disease.

The selection of the TCRL will naturally depend on its presentation in the pathology. Exemplary TCRLs and their association with pathologies are provided in the Tables hereinabove.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, preferably human beings at any age which suffer from the pathology.

The pathology can be, but is not limited to, cancer, viral infection and/or an autoimmune disease.

According to some embodiments of the invention, the pathology is cancer.

The term “cancer” as used herein is defined as disease characterized by the rapid and uncontrolled growth of aberrant cells. Cancer cells can spread locally or through the bloodstream and lymphatic system to other parts of the body.

The cancer may be a hematological malignancy, a solid tumor, a primary or a metastasizing tumor. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, Chronic Lymphocytic Leukemia (CLL), leukemia, lung cancer and the like. Additional non-limiting examples of cancers which can be treated by the method of some embodiments of the invention are provided in Table 1, above.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the Antibodies of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

According to some embodiments of the invention, the pathology is a solid tumor.

According to some embodiments of the invention, the medicament resultant of the method of some embodiments of the invention has an anti-tumor effect.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the medicament of the invention in prevention of the occurrence of tumor in the first place.

According to some embodiments of the invention, the pathology is a viral infection.

Non-limiting examples of viral infections which can be treated by the antibodies of some embodiments of the invention are described in Table 2 above.

According to some embodiments of the invention, the pathology is an autoimmune disease.

Non-limiting examples of autoimmune diseases which can be treated by the method and medicament of some embodiments of the invention include Addision's disease, alopecia greata, ankylosing spondylitis, autoimmune hepatitis, autoimmune parotitis, Celiac (Coeliac), Crohn's disease, diabetes (Type 1), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, stroke, among others.

The antibodies of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the antibody accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (TCRL-antibody) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays.

For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1). Dosage amount and interval may be adjusted individually to provide TCRL (the TCRL tissue) levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit (diagnostic or therapeutic), which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to a specific embodiment the TCRL of the present invention does not comprise the CDRs of the following antibodies appearing in Table 3 below where each is considered a separate embodiment.

TABLE 3

| | | | HLA | TCRL | |
|---|---|---|---|---|---|
| # | Antigen | peptide | type | name | process |
| 1 | hCG-beta | TMTRVLQGV | HLA-A02 | 3F9 | hybridoma |
| 2 | hCG-beta | GVLPALPQV | HLA-A02 | 1610 | hybridoma |
| 3 | MAGE-A3 | FLWGPRALV | HLA-A02 | 7D4 | hybridoma |
| 4 | NY-ESO-1 | SLLMWITQV | HLA-A02 | 3M4E5; | phage display |
| 5 | WT1 | RMFPNAPYL | HLA-A02 | ESK1 | phage display |
| 6 | PR1 | VLQELNVTV | HLA-A02 | 8F4 | hybridoma |
| 7 | MAGE-A1 | EADPTGHSY | HLA-A01 | G8; Hyb3 | phage disp. |
| 8 | p68 | YLLPAIVHI | HLA-A02 | RL6A | hybridoma |
| 9 | HBVEnv ₁₃₈₋₁₉₁ | FLLTRILTI | HLA-A02 | | hybridoma |
| 10 | West Nile | SSVWNATTA(I) | H-2Db | RL36A | hybridoma |
| | Virus NS4B | | | | |
| 11 | HA-1 H | VLHDDLLEA | HLA-A02 | #131 | immunized |
| | | | | | phage display |
| | | | | | library |
| 12 | chorionic | GVLPALPQV | HLA-A02 | 3.2G1 | hybridoma |
| | gonadotropin | | | | |
| | beta | | | | |
| 13 | chorionic | GVLPALPQV | HLA-A02 | RL4B | hybridoma |
| | gonadotropin | | | | |
| | beta | | | | |
| 14 | HBV Env | FLLTRILTI | HLA-A02 | Env183/A2 | hybridoma |
| | | | | MAb | |
| 15 | ovalbumin | SIINFEKL | H-2Kb | 25-D1.16 | hybridoma |
| 16 | Her2 | KIFGSLAFL | HLA-A02 | RL1B | hybridoma |
| 17 | EBV EBNA1, | FMVFLQTHI, | HLA-A02 | LMP1#226 | hybridoma, |
| | LMP1, | YLLEMLWRL, | | | immunomagnetic |
| | LMP2A | CLGGLLTMV | | | enrichment |
| 18 | GP100 | ITDQVPFSV | HLA-A02 | GPA7 | |
| 19 | Multi-MAGE- | YLEYRQVPG | HLA-A02 | Fab AHS | |
| | A | | | | |
| 21 | West Nile | SLFGQRIEV | HLA-A02 | RL14C | |
| 22 | Virus | SVGGVFTSV | HLA-A02 | RL15A | |
| | epitopes | | | | |
| | | | Company or | | |
| # | Isotype | Affinity | group | pub year | ref |
| 1 | hIgG1 | | ReceptorLogic | 2008 | Neethling et al., 2008 |
| 2 | hIgG1 | | ReceptorLogic | 2008 | Neethling et al., 2008 |
| 3 | | 2.4 nM | INSERM | 2005 | Bernardeau et al., 2005 |
| | | | V601, Nantes | | |
| | | | univ, (france) | | |
| 4 | Fab | 2-4 nM | Renner. | 2009 | |
| | | | Wellcome | | |
| | | | Trust Centre, | | |
| | | | U Oxford; U | | |
| | | | Hospital | | |
| | | | Zurich | | |
| 5 | hIgG1 | 100 pM | Sloan | 2013 | US 2014/0271644 |
| | | | Kettering | | US 2014/0294841 |
| 6 | mouse | 9.9 nM | Texas univ. | 2011 | Sergeeva et al., 2011 |
| | IgG2a | | and Bio | | |
| | | | Scientific | | |
| | | | Corporation, | | |
| | | | Austin, TX | | |
| 7 | Fab | 250 nM; | U | 2000, 2008 | Chames et al., 2000 |
| | | 14 nM | Maastricht; U | | |
| | | | Rotterdam; | | |
| | | | Dyax | | |
| 8 | | 0.42 nM | Texas Tech U; | 2010 | Verma et al., 2010 |
| | | | Receptor | | |
| | | | Logic | | |
| 9 | | | | 2011 | Sastry et al., 2011 |
| 10 | | 234 nM | Receptor | 2014 | |
| | | | Logic; | | |
| | | | Washington | | |
| | | | University | | |
| | | | School of | | |
| | | | Medicine | | |
| 11 | scFv | 14.9 nM | Fujita Health | 2014 | |
| | CAR | | University, | | |
| | | | Toyoake, | | |
| | | | Japan | | |
| 12 | IgG2a | | Receptor | 2007 | Weidanz et al., 2007 |
| | | | Logic; Texas | | |
| | | | Tech | | |
| | | | University | | |
| 13 | IgG | 1.5 nM | Receptor | | |
| | | | Logic; Texas | | |
| | | | Tech | | |
| | | | University | | |
| 14 | IgG1 | | Singapore | 2011 | |
| | Kappa | | Institute for | | |
| | | | Clinical | | |
| | | | Sciences | | |
| 15 | IgG1 | 70 nM | NIH | 1997 | |
| 16 | | 2.69 nM | Texas Tech | 2013 | US 2014/0065708 |
| | | | University | | |
| | | | (and Receptor | | |
| | | | Logic) | | |
| 17 | | 1.85-6.98 nM | Singapore | 2013 | US 2012/0294874 |
| | | | universities | | |
| 18 | | | Chinese | | |
| | | | universities | | |
| 19 | | | | | US 2014/0120090 |
| | | | | | EP2658873 |
| | | | | | WO 2012/091564 |
| | | | | | APO-T B.V |
| 21 | | 6.96 nM | Receptor | | US 2014/0065708 |
| 22 | | | logic | | |

It is expected that during the life of a patent maturing from this application many relevant TCRLs will be developed and the scope of the term TCRLs is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Production of Biotinylated Single-Chain MHC-Peptide Complexes

Single-chain MHC (scMHC)³-peptide complexes were produced by in vitro refolding of inclusion bodies produced in Escherichia coli upon isopropyl β-D-thiogalactoside (IPTG) induction. Briefly, a scMHC, which contains the β₂-microglobulin and the extracellular domains of the HLA-A2 gene connected to each other by a flexible linker, was engineered to contain the BirA recognition sequence for site-specific biotinylation at the C terminus (scMHC-BirA). In vitro refolding was performed in the presence of peptides as described. Correctly folded MHC-peptide complexes were isolated and purified by anion exchange Q-Sepharose chromatography (Pharmacia), followed by site-specific biotinylation using the BirA enzyme (Avidity). A more detailed description for the production of single chain-MHC peptide complexes is provided in Denkberg, et al. (2002) PNAS. 99:9421-9426.

Flow Cytometry

T-B hybrid T2 cells were washed with serum-free RPMI 1640 medium and incubated overnight with medium containing 10⁻⁴-10⁻⁵M tyrosinase₃₆₉₋₃₇₇YMDGTMSQV (SEQ ID NO: 1)/WT1₁₂₆₋₁₃₄ (RMFPNAPYL, SEQ ID NO: 141) peptide/MAGE-A4₂₃₀₋₂₃₉ SEQ ID NO: 176/MAGE-A9₂₂₃₋₂₃₁ 203/PAP₁₁₂₋₁₂₀ SEQ ID NO: 230 peptide or relevant control peptides (listed in the Table 15 below). Peptide loading efficiency was verified by using the ratio between MFI of HLA-A2-binding antibody BB7.2 on peptide-loaded T2 cells and MFI of unloaded T2 cells (>1) data not shown.

T2 or primary cells or cell lines (10⁶) were incubated with 10 μg/ml of specific Ab (with or without biotinylation) for 1 h at 4° C., followed by incubation with PE-labeled anti-mouse/human/steptavidin Ab for 45 min at 4° C. It will be appreciated that the work with anti mouse secondary antibody or with streptavidin gave similar results for D11 and B47B6. Cells were finally washed and analyzed by:

### FACS 1:

Machine: BD FACS calibur
Analysis software: CELLQuest

### FACS 2:

### Machine: Beckman Coulter NAVIOS

Analysis software: Kaluza version 1.3

Production of TCR-Like Antibodies to HLA-A2/Tyrosinase369-377/WT1₁₂₆₋₁₃₄/MAGE-A4₂₃₀₋₂₃₉/MAGE-A9₂₂₃₋₂₃₁/PAP₁₁₂₋₁₂₀ using the hybridoma technique

HHD mice were immunized by 5-6 injections of HLA-A2-peptide complex 50 μg/mouse. 2-3 first injections were administrated s.c with addition of QuilA adjuvant. Hybridoma clones were generated by fusion of splenocytes isolated from mice immunized with the above complex (as previously described e.g., Weidanz et al. 2011 Int. Rev. Immunol. 30:328-340) with NSO myeloma cells and were screened and isolated by differential ELISA assays as described below. For example, for Tyrosinase TCRLs selection the relevant TyrD369-377 peptide HLA-A2 complexes were used and compared to the non relevant p68-DDX5 control peptide (SEQ ID NO: 2 YLLPAIVHI) HLA-A2 complexes. ELISA with purified HLA-A2-Tyr complexes as well as with control HLA-A2 complex displaying other HLA-A2-restricted peptide (Table 15) was used to select specific clones Isolated hybridoma clones were sub-cloned and were sequenced. Two clones 906-11-D11 (termed D11, FIG. 68) and 905-2-D7 (termed D7, FIG. 69) were characterized.

Hybridomas are were to >80% confluency in HAT DMEM or serum free DCCM2 medium and supernatant was collected. Purified IgG was isolated from culture supernatant by affinity chromatography using Protein A column. SDS-PAGE analysis of the purified protein revealed homogenous, pure IgG with the expected molecular mass of ˜150 kDa.

Construction of whole IgG Ab

The H and L Fab genes (only for MC1) were cloned for expression as human IgG1 κ Ab into the eukaryotic expression vectors the eukaryotic expression vectors pOptiVEC and pcDNA3.3-TOPO respectively. Each shuttle expression vector carries a different gene selection (for pOptiVEC the DHFR/HT- and for pcDNA3.3 Geneticin). Expression was facilitated by co-transfection of the two constructs into the dihydrofolate reductase (DHFR)-deficient, Chinese hamster ovary (CHO)-derived DG44 cells in suspension culture by using the FreeStyle MAX reagent (Invitrogen). After co-transfection, cells were grown on selective medium. Clones that reacted specifically with JY T2 cells pulsed with tyrosinase 369-377 peptide were adapted to growth in 0.5% serum and were further purified using protein A affinity chromatography. SDS-PAGE analysis of the purified protein revealed homogenous, pure IgG with the expected molecular mass of ˜150 kDa.

ELISA with Supernatant or Purified Abs

The binding specificities of individual supernatant or purified TCRL antibodies were determined by ELISA using biotinylated scMHC-peptide complexes. Maxi sorp 96 wells ELISA plates (Nunc #442404) were coated overnight with BSA-biotin (1 μg/well). After having been washed, the plates were incubated (1 h, RT) with streptavidin (1 μg/well), washed extensively, and further incubated (1 h, RT) with 0.25 μg of MHC/peptide complexes. The plates were blocked for 30 min at RT with PBS/2% skim milk and subsequently were incubated for 1 h at RT with 1 μg/well supernatant or purified TCRL antibodies. After having been washed, the plates were incubated with HRP-conjugated/anti-human or mouse Ab. Detection was performed using TMB tetramethylbenzidine reagent (DAKO, S1599). The HLA-A2-restricted peptides used for specificity studies of the purified supernatant or purified TCRL antibodies.

Proteon XPR36 Surface Plasmon Resonance (SPR) Binding Analysis

Immobilization of IgG TCR-like antibody was performed on a GLM (General Layer Medium) chip (Bio-Rad Laboratories, Hercules, Calif., USA) at 25° C. in the vertical orientation and the continuous running buffer was PBST (10 mM Na-phosphate, 150 mM NaCl, and 0.005% Tween 20, pH 7.4). Five channels were activated with 50 μl of a mixture of 0.04 M N-ethyl-N′-(3-dimethylaminopropyl) carbodiimide (EDC) and 0.01 M sulfo-N-hydroxysuccinimide (Sulfo-NHS) at a flow rate of 30 μl/min. The anti-mouse or human IgG/NeutrAvidin was diluted in 10 mM sodium acetate buffer pH 4.5 to a final concentration of 25 μg/ml and 150 μl were injected followed by an injection of 150 μl of 1 M ethanolamine-HCl pH 8.5. The IgG TCRL antibody/purified biotinylated single-chain recombinant HLA-A2/Tyrosinase/WT1/MAGE-A4/MAGE-A9/PAP complex ligand was diluted in PBST to 5-10 μg/ml and 90 μl were injected in the vertical orientation with a flow rate of 30 μl/min. The sixth channel remained empty to serve as a reference. The analyte purified single-chain recombinant HLA-A2/Tyrosinase/WT1/MAGE-A4/MAGE-A9/PAP complex/Fab TCRL antibody was injected (75 μl at 50 μl/min) in the horizontal orientation of the ProteOn using five different concentrations (1000, 500, 250, 125 and 62.5 nM). Running buffer was injected simultaneously in the sixth channel for double referencing to correct for loss of the captured antibodies from the chip sensor surface during the experiment. All binding sensorgrams were collected, processed and analyzed using the integrated ProteOn Manager (Bio-Rad Laboratories, Hercules, USA) software. Binding curves were fitted using the Langmuir model describing 1:1 binding stoichiometry, or with the Langmuir and mass transfer limitation model.

Functional Assays

LDH-Release Assay

Bispecific TCRL redirected target cell killing was measured in a non-radioactive cytotoxicity assay using CytoTox96® (Promega). This assay quantitatively measures lactate dehydrogenase (LDH), an enzyme that is released upon cell lysis. Released LDH in culture supernatants is measured with a 10 minute coupled enzymatic assay, which results in the conversion of a tetrazolium salt (INT) into a red formazan product. The amount of color produced is proportional to the number of lysed cells.

Specifically, target cells and effector cells were washed, counted and resuspended in cRPMI medium (1% FBS) without phenol red. Target cells were adjusted to a cell density of 2.5×10⁵ cells per ml and the effector cells at a cell density of 2.5×10⁶ cells per ml. 40 μl (1×10⁴ cells) of target cells were cultured in a 96-well V-shaped plate. A 5 times concentrated stock of the Bispecific TCRL test reagent was prepared at the highest test concentration, which was serially diluted 1 in 10 in medium without phenol red in a separate plate to obtain other test concentrations. The Bispecific TCRL was then added to the target cells in the assay plate at 20 μl per well to give the final indicated titrated amounts. The assay plate containing the target cells mixed with the Bispecific TCRL was then incubated for 20 minutes at 37° C./5% CO₂. Following the incubation, 40 μl effector cells (1×10⁵ cells) were added to each well resulting in an effector to target (E:T) ratio of 10:1. Control wells were set up with effector cells alone to calculate effector spontaneous release, target cells alone to calculate target spontaneous release, and target cells with 80 μg/ml digitonin final to calculate maximum release. Each condition was assayed in triplicates in a final volume of 100 μl. The plate was incubated at 37° C./5% CO₂ for 24 hours. Following the incubation period, the plate was centrifuged at 700×g for 5 minutes and 50 μl transferred from each well to the corresponding well in a 96-well flat bottomed Maxisorb plate (Nunc). The CytoTox96® substrate mix was reconstituted using CytoTox96® assay buffer, as per manufacturer's instructions, and 50 μl added to each well of the plate. The plate was covered with aluminum foil and incubated at room temperature for 10 minutes. Then absorbance recorded at 490 nm on a plate reader. Percentage cytotoxicity was then calculated using the following equation: Specific lysis=[(Experimental−Effector Spontaneous−Target Spontaneous)/(Target Maximum−Target Spontaneous)]×100. PBMCs for killing assays are isolated from healthy volunteers and with all regulatory IRBs approvals and written consents. Effector PBMCs are isolated using the Lymphoprep procedure.

Tumor Cell Lines and Normal Primary Cells

Cells lines A375 (melanoma), U2OS (osteosarcoma), TCCSUP (bladder carcinoma) and Fib (fibroblasts) were cultured in complete DMEM supplemented with 10% FBS (all supplied by GIBCO). 501A, SKMe15, Mewo and 1938 (melanoma), Saos2 (osteosarcoma), Panc1 (pancreatic carcinoma), J82 and UMUC3 (bladder), H1703 (non-small cell lung adenocarcinoma), JVM2 (Mantle cell lymphoma), IM9 (multiple myeloma), U266 (myeloma) and SW620 (colorectal adenocarcinoma) were cultured in complete RPMI supplemented with 10% FBS (all supplied by GIBCO). Malme3m (melanoma), JEKO1 (mantle-cell lymphoma), SET2 (essential thrombocythemia) and BV173 (B cell precursor leukemia) were cultures in complete RPMI supplemented with 20% FBS (all supplied by GIBCO). THP-1 (AML) were cultured in complete RPMI supplemented with 10% FBS (all supplied by GIBCO) and 0.05 mM beta-mercaptoethanol (supplied by Thermo-fisher). OVCAR-3 (ovary adenocarcinoma) were cultured in complete RPMI supplemented with 20% FBS (all supplied by GIBCO) and 0.01 mg/ml bovine insulin (supplied by Sigma). All cell lines were maintained at 37° C. in a humidified atmosphere of 7.5% CO₂ and were purchased from American Type Culture Collection.

Normal primary hepatocytes, cardiac myocytes, osteoblasts, astrocytes, bronchial epithelial cells, colonic smooth muscle cells, urothelial cells and renal epithelial cells were obtained from Sciencell and cultured according to the manufacturer's instructions. All cell lines were maintained at 37° C. in a humidified atmosphere of 7.5% CO₂.

Expression and Purification of Soluble Recombinant Fab Abs in Expi293 System

The VH-CH1 and VL-CL genes of Tyr D11 and D7, MAGE-A4 C106B9, WT1 B47B6 and ESK1 IgGs were cloned for expression as Fab in the eukaryotic expression vector pcDNA3.4. His-tag was connected to the C-terminus of the CH1 region.

Expression was facilitated by co transfection of the two constructs (heavy and light chains) into the Expi293F human cells in Expi293 expression medium (both are components of the Expi293 expression system) by the Fectamine transfection reagent (Life technologies). Following co-transfection, cells were grown for 6 days. A fter 6 days cells were centrifuged at 700×g for 5 minutes. Following centrifugation, the supernatant containing the D11, D7, C106B9, B47B6 or ESK1 Fab was removed from cells and filtered through 0.22μ filter. The supernatant was then dialyzed overnight against PBS.

The D11, D7, C106B9, B47B6 or ESK1 Fab recombinant protein was purified by metal affinity column (Talon) and dialyzed overnight against PBS. The purified D11, D7, C106B9, B47B6 or ESK1 Fab were analyzed on reduced and non-reduced SDS-PAGE.

Construction, Expression and Purification of Bispecific TCRLs in Expi293 System

The VH-CH1 and VL-CL genes of Tyr D11 and D7, WT1 B47B6 and ESK1 and MAGE-A4 C106B9, IgGs were cloned for expression as bispecific (BS) in the eukaryotic expression vector pcDNA3.4 (sequences are shown in FIGS. 68-70, sequences of ESK1 is available from WO 2015/070061). For the light chain vector of Tyr D11, WT1 B47B6 and ESK1 and MAGE-A4 C106B9, anti CD3 (clone UCHT1) scFv was connected to the N-terminus of the VL region (BS format 3, #F3). For the heavy chain vector, His-tag was connected to the C-terminus of the CH1 region. For Tyr D7, anti CD3 (clone UCHT1) scFv was connected to the N-terminus of the VH region of the heavy chain (BS format 1, #F1) and His-tag was connected to the C-terminus of the CH1 region.

Expression was facilitated by co transfection of the two constructs into the Expi293F human cells in Expi293 expression medium (both are components of the Expi293 expression system) by the Fectamine transfection reagent (Life technologies). After co-transfection, cells were grown for 6 days. Following 6 days cells were centrifuged at 700×g for 5 minutes. Following centrifugation, the supernatant containing the TCRL bispecific antibodies were removed from cells and filtered through 0.22 μm filter. The supernatant was then dialyzed overnight against PBS.

The BS-TCRLs recombinant proteins were purified by two steps of metal affinity (Talon) and size exclusion chromatography (Superdex 200 10/300 GL GE). The purified BS-TCRLs were analyzed on SDS-PAGE.

In Vivo Assays

For 501A Melanoma Cell Line (ATCC, Manassas Va., USA)]

Cells were cultured in RPMI1640 growth medium (GIBCO, Waltham Mass., USA) supplemented with 10% fetal bovine serum (GIBCO, Waltham Mass., USA). Human peripheral blood mononuclear cells (PBMC) were prepared from healthy donors by using SepMate™-50 tubes (Stemcell).

At day 0, eight to ten weeks old female NOD/SCID mice (Envigo, Israel; n=6-8) were inoculated subcutaneously (s.c.) in a single flank with 5×10⁶ 501 A melanoma cells +/−25×10⁶ PBMCs (Effector:Tumor cell ratio 5:1) in a final volume of 0.25 ml phosphate-buffered saline (PBS); D7 bispecific TCRL (0.1 mg/kg) or vehicle (PBS) were administered i.v. one hour after the s.c. inoculation in a final volume of 0.2 ml, with 4 additional doses administered every 24 hours.

For A375 Melanoma Cell Line (ATCC, Manassas Va., USA)]

Cells were cultured in RPMI1640 growth medium (GIBCO, Waltham Mass., USA) supplemented with 10% fetal bovine serum (GIBCO, Waltham Mass., USA). Activated CD8 T-cells were prepared from human peripheral blood mononuclear cells (PBMC) using a rapid expansion protocol (REP). Naïve PBMCs were produced from healthy donor's peripheral blood using SepMate™-50 tubes (Stemcell), following CD8 T cells enrichment using Dynabeads® Untouched™ Human CD8 T Cells kit (Invitrogen). Activation of the purified CD8 T cells was performed in flasks pre-coated with monoclonal antibodies against CD3 (OKT3) and CD28 for 72 hrs in media supplemented with 10% FBS and 100 IU/mL of human IL-2. Activated cells were expanded over the period of 14 days in media supplemented with 10% FBS, 3000 IU/ml IL-2, 30 ng/ml OKT3 and 2×10⁸ irradiated PBMCs.

At day 0, eight to ten weeks old female NOD/SCID mice (Envigo, Israel; n=6-8) were inoculated subcutaneously (s.c.) in a single flank with 5×10⁶ A375 melanoma cells +/−10×10⁶ REP CD8 T-cells (Effector: Tumor cell ratio 2:1) in a final volume of 0.25 ml phosphate-buffered saline (PBS); MAGE-A4 C106B9 bispecific TCRL (0.1 mg/kg), WT1 B47B6 bispecific TCRL (0.1 mg/kg) or vehicle (PBS) were administered i.v. one hour after the s.c. inoculation in a final volume of 0.2 ml, with 4 additional doses administered every 24 hours.

In both cases (501A and A375) tumors were measured two times per week with calipers in two perpendicular dimensions and tumor volumes were calculated with the following formula:

$$width x {( \frac{length}{2} )}^{2} \times 3.14$$

Other TCRL Antibodies Used in the Present Study

The generation of MC1 is described in WO2008/120202.

The generation of ESK1 (Dao T, Yan S, Veomett N, Pankov D, Zhou L, Korontsvit T, Scott A, Whitten J, Maslak P, Casey E, Tan T, Liu H, Zakhaleva V, Curcio M, Doubrovina E, O'Reilly R J, Liu C, Scheinberg D A. Targeting the intracellular WT1 oncogene product with a therapeutic human antibody. Sci Transl Med. 2013 Mar. 13; 5(176):176ra33). ESK1 was thus generated by synthetic gene synthesis according to the published sequence WO 2015/070061 ESK1 full VH—SEQ ID NO:128 and ESK1 full VL—SEQ ID NO: 130 in the sequence listing of WO 2015/070061. The antibody was produced in HEK293 cells as IgG using the Expi293 system as described above and was purified from culture supernatants using protein A affinity chromatography.

Extraction of Nucleic Acids

Total RNA was extracted from 1*10⁶-5*10⁶ cells cultured cells with RNeasy Plus Mini (Qiagen) according to the manufacturer's instructions.

cDNA Synthesis cDNA was synthesized from 1-5 μg RNA, using a combination of oligo dT and random hexamer (1:1) with SuperScript® III First-Strand Synthesis System (Invitrogen) according to the manufacturer's instructions. F or quantitative PCR, cDNA was diluted 1:5 with H₂O.

Conventional PCR (PCR)

The PCR cycling conditions were 95° C. for 2 minutes, followed by 40 cycles of 95° C. for 20 s, 60° C. for 1 min and 72° C. for 1 min. The PCR was ended with a final extension of 72° C. for 10 min. Reactions were performed with KAPA HiFi PCR Kit (Kapa Biosystems) according to the manufacturer's instructions.

Following primers were used:

| (SEQ ID NO: 3) | |
|---|---|
| TYR S: | TTAGCAAAGCATACCATCA |
| and | |
| (SEQ ID NO: 4) | |
| TYR AS: | CCAGACAAAGAGGTCATAA |

for tyrosinase expression (expected product size: 117 bp) and WT1_S: AGGCTGCAATAAGAGATA (SEQ ID NO: 5) and WT1_AS: TTCGCTGACAAGTTTTAC (SEQ ID NO: 6) for WT1 expression (expected product size: 188 bp).

To visualize the amplified products, 10 μL of samples were mixed with 2 μL of 6× loading buffer (New England Biolabs) and subjected to electrophoresis on 1.5% agarose gels stained with ethidium bromide with DNA markers (New England Biolabs). The presence and intensity of the PCR product bands was determined on an ImageQuant LAS 4000 (GE Healthcare Life Sciences).

Quantitative PCR (qPCR)

Quantitative PCR was carried out using TaqMan Gene Expression Master Mix on a ABI 7300 instrument (Applied Biosystems), according to the manufacturer's instructions. The cycle conditions for real-time PCR were 95° C. for 10 min, followed by 40 cycles of 95° C. for 15 sec, and 60° C. for 1 min. Probes for real-time PCR were purchased from Applied Biosystems; at the 5′ end, they were conjugated to the fluorochrome FAM. Following assays (primers and probes) were used: for TYR (cat# Hs00165976), for MAGE A4 (cat# Hs00751150), and for WT1 (cat# Hs01103751). Beta-actin was used as a housekeeping gene for normalization (cat# Hs99999903).

Peptides used in the present study

TABLE 4
Ala Scan - TyrD

| Peptide name | Peptide-HLA-A2 sequence | SEQ ID NO: |
|---|---|---|
| TyrD-A1 | AMDGTMSQV | 104 |
| TyrD-A2 | YADGTMSQV | 105 |
| TyrD-A3 | YMAGTMSQV | 106 |
| TyrD-A4 | YMDATMSQV | 107 |
| TyrD-A5 | YMDGAMSQV | 108 |
| TyrD-A6 | YMDGTASQV | 109 |
| TyrD-A7 | YMDGTMAQV | 110 |
| TyrD-A8 | YMDGTMSAV | 111 |
| TyrD-A9 | YMDGTMSQA | 112 |

TABLE 5
Similar peptides - TyrD

| | Peptide-HLA- | | |
|---|---|---|---|
| Peptide name | A2 sequence | SEQ ID NO: | Similar to |
| Tyrosinase D | YMDGTMSQV | 113 | |
| (Tyrosinase | | | |
| peptide) | | | |
| Tyrosinase N | YMNGTMSQV | 114 | |
| *KIAA0355 | YMDNVMSEV | 115 | TyrD |
| KPNA1 | VMDSKIVQV | 116 | TyrD |
| GPLD1 | LMNGTLKQV | 117 | TyrD |
| TyrD-S1 | SQDGTRSQV | 118 | TyrD |
| TyrD-S2 | VMDTTKSQV | 119 | TyrD |
| TyrD-S3 | GMDGTQQQI | 120 | TyrD |
| TyrD-S4 | GMVGTMTEV | 121 | TyrD |
| TyrD-S5 | MMDATFSAV | 122 | TyrD |
| TyrD-S6 | QMDPTGSQL | 123 | TyrD |
| *TyrD-S7 | SMDGSMRTV | 124 | TyrD |
| TyrD-S8 | WMDGIASQI | 125 | TyrD |
| TyrD-S9 | YLEGILSQV | 126 | TyrD |
| TyrD-S10 | YMAIKMSQL | 127 | TyrD |
| TyrD-S11 | YMDAVVSLV | 128 | TyrD |
| TyrD-S12 | YMDGTNRRI | 129 | TyrD |
| TyrD-S13 | YMDPSTYQV | 130 | TyrD |
| TyrD-S14 | YMLGTNHQL | 131 | TyrD |
| TyrD-S15 | YMPGTASLI | 132 | TyrD |
| TyrD-S16 | YMRETRSQL | 133 | TyrD |
| *TyrD-S17 | MMDGAMGYV | 134 | TyrD |
| *TyrD-S18 | NMDSFMAQV | 135 | TyrD |
| *TyrD-S19 | QMDFIMSCV | 136 | TyrD |
| *TyrD-S20 | YEDLKMYQV | 137 | TyrD |
| *TyrD-S21 | YMDTIMELV | 138 | TyrD |
| *TyrD-S22 | YTDLAMSTV | 139 | TyrD |
| *TyrD-S23 | YVDFVMSSV | 140 | TyrD |

*Ala-based similar peptides

TABLE 6
Similar peptides - WT1

| | Peptide-HLA-A2 | | |
|---|---|---|---|
| Peptide name | sequence | SEQ ID NO: | Similar to |
| WT1 (WT1 | RMFPNAPYL | 141 | |
| peptide) | | | |
| WT1-S1 | LDFPNLPYL | 142 | WT1 |
| *WT1-S2 | RCFPNCPFL | 143 | WT1 |
| WT1-S3 | LMFENAAYL | 144 | WT1 |
| WT1-S4 | RMFPNKYSL | 145 | WT1 |
| WT1-S5 | RLFPNAKFL | 146 | WT1 |
| *WT1-S6 | RLFPNLPEL | 147 | WT1 |
| *WT1-S7 | RMFPTPPSL | 148 | WT1 |
| WT1-S8 | RMVPRAVYL | 149 | WT1 |
| WT1-S9 | RMFFNGRYI | 150 | WT1 |
| WT1-S10 | RMLPHAPGV | 151 | WT1 |
| WT1-S11 | YMFPNAPYL | 152 | WT1 |
| WT1-S12 | AMDPNAAYV | 153 | WT1 |
| WT1-S13 | ICFPNAPKV | 154 | WT1 |
| WT1-S14 | NMFENGCYL | 155 | WT1 |
| WT1-S15 | NMPPNFPYI | 156 | WT1 |
| WT1-S16 | REMTQAPYL | 157 | WT1 |
| WT1-S17 | RMAPRAPWI | 158 | WT1 |
| WT1-S18 | RMEPRAPWI | 159 | WT1 |
| WT1-S19 | RMEPRAPWV | 160 | WT1 |
| WT1-S20 | RMFLNNPSI | 161 | WT1 |
| WT1-S21 | RMFQQTFYL | 162 | WT1 |
| WT1-S22 | RMNPNSPSI | 163 | WT1 |
| WT1-S23 | RQFPNASLI | 164 | WT1 |
| WT1-S24 | RQFPNKDAL | 165 | WT1 |
| WT1-S25 | RVFPWASSL | 166 | WT1 |
| WT1-S26 | RLFPWGNKL | 167 | WT1 |

*Ala-based similar peptides

TABLE 7
Ala Scan - WT1

| Peptide name | Peptide-HLA-A2 sequence | SEQ ID NO: |
|---|---|---|
| WT1-A1 | AMFPNAPYL | 168 |
| WT1-A2 | RAFPNAPYL | 169 |
| WT1-A3 | RMAPNAPYL | 170 |
| WT1-A4 | RMFANAPYL | 171 |
| WT1-A5 | RMFPAAPYL | 172 |
| WT1-A7 | RMFPNAAYL | 173 |
| WT1-A8 | RMFPNAPAL | 174 |
| WT1-A9 | RMFPNAPYA | 175 |

TABLE 8
Similar peptides - MAGE-A4

| | Peptide-HLA- | | |
|---|---|---|---|
| Peptide name | A2 sequence | SEQ ID NO: | Similar to |
| MAGE-A4 | GVYDGREHTV | 176 | |
| (MAGE-A4 | | | |
| peptide) | | | |
| MAGE-A4-S1 | GLADGRTHTV | 177 | MAGE-A4 |
| MAGE-A4-S2 | GVSDGRWHSV | 178 | MAGE-A4 |
| MAGE-A4-S4 | GVYDGEEHSV | 179 | MAGE-A4 |
| MAGE-A4-S5 | GLYDGMEHL | 180 | MAGE-A4 |
| MAGE-A4-S6 | GVSDGQWHTV | 181 | MAGE-A4 |
| MAGE-A4-S9 | GVYAGREHFL | 182 | MAGE-A4 |
| MAGE-A4-S10 | GLYDGMEHLI | 183 | MAGE-A4 |
| MAGE-A4-S12 | ASYDGTEVTV | 184 | MAGE-A4 |
| MAGE-A4-S13 | AVLDGRELRV | 185 | MAGE-A4 |
| MAGE-A4-S15 | GLYDGIEHFM | 186 | MAGE-A4 |
| MAGE-A4-S16 | GLYDGPVHEV | 187 | MAGE-A4 |
| MAGE-A4-S17 | GVCAGREHFI | 188 | MAGE-A4 |
| MAGE-A4-S18 | GVYAGRPLSV | 189 | MAGE-A4 |
| MAGE-A4-S19 | TVYDLREQSV | 190 | MAGE-A4 |
| MAGE-A4-S20 | VVDDGVEHTI | 191 | MAGE-A4 |
| MAGE-A4-S21 | GVFDGLHTV | 192 | MAGE-A4 |

TABLE 9
Ala Scan - MAGE-A4

| Peptide name | Peptide-HLA-A2 sequence | SEQ ID NO: |
|---|---|---|
| MAGE-A4-A1 | AVYDGREHTV | 193 |
| MAGE-A4-A2 | GAYDGREHTV | 194 |
| MAGE-A4-A3 | GVADGREHTV | 195 |
| MAGE-A4-A4 | GVYAGREHTV | 196 |
| MAGE-A4-A5 | GVYDAREHTV | 197 |
| MAGE-A4-A6 | GVYDGAEHTV | 198 |
| MAGE-A4-A7 | GVYDGRAHTV | 199 |
| MAGE-A4-A8 | GVYDGREATV | 200 |
| MAGE-A4-A9 | GVYDGREHAV | 201 |
| MAGE-A4-A10 | GVYDGREHTA | 202 |

TABLE 10
Similar peptides - MAGE-A9

| | Peptide-HLA- | | |
|---|---|---|---|
| Peptide name | A2 sequence | SEQ ID NO: | Similar to |
| MAGE-A9 | ALSVMGVYV | 203 | |
| (MAGE-A9 | | | |
| peptide) | | | |
| MAGE-A9S1 | ALSVLGVMV | 204 | MAGE-A9 |
| MAGE-A9S3 | ALSRKGIYV | 205 | MAGE-A9 |
| MAGE-A9S4 | ALSVMYSYL | 206 | MAGE-A9 |
| MAGE-A9S6 | AVSHMGVLV | 207 | MAGE-A9 |
| MAGE-A9S7 | LLSLMGVLV | 208 | MAGE-A9 |
| *MAGE-A9S8 | VLSIMGVYA | 209 | MAGE-A9 |
| MAGE-A9S10 | ALQVRKVYV | 210 | MAGE-A9 |
| MAGE-A9S11 | ALQVYGVEV | 211 | MAGE-A9 |
| MAGE-A9S13 | ALSVAGGFV | 212 | MAGE-A9 |
| MAGE-A9S14 | ALSVLGKVV | 213 | MAGE-A9 |
| MAGE-A9S15 | ALSVMIPAV | 214 | MAGE-A9 |
| MAGE-A9S16 | DLSVCSVYV | 215 | MAGE-A9 |
| MAGE-A9S17 | ILGVMGVDV | 216 | MAGE-A9 |
| MAGE-A9S20 | LLSVNGVSV | 217 | MAGE-A9 |
| MAGE-A9S23 | SLSPMGRYV | 218 | MAGE-A9 |
| MAGE-A9S24 | ALSAVMGVTL | 219 | MAGE-A9 |
| MAGE-A9S25 | AILLVMGVDV | 220 | MAGE-A9 |
| MAGE-A9S26 | ALSDHHVYL | 221 | MAGE-A9 |

*Ala-based similar peptides

TABLE 11
Ala Scan - MAGE-A9

| Peptide name | Peptide-HLA-A2 sequence/ | SEQ ID NO: |
|---|---|---|
| MAGE-A9-A2 | AASVMGVYV | 222 |
| MAGE-A9-A3 | ALAVMGVYV | 223 |
| MAGE-A9-A4 | ALSAMGVYV | 224 |
| MAGE-A9-A5 | ALSVAGVYV | 225 |
| MAGE-A9-A6 | ALSVMAVYV | 226 |
| MAGE-A9-A7 | ALSVMGAYV | 227 |
| MAGE-A9-A8 | ALSVMGVAV | 228 |
| MAGE-A9-A9 | ALSVMGVYA | 229 |

TABLE 12
Similar peptides - PAP

| | Peptide-HLA- | | |
|---|---|---|---|
| Peptide name | A2 sequence | SEQ ID NO: | Similar to |
| PAP | TLMSAMTNL | 230 | |
| (PAP peptide) | | | |
| PAP(TLM)S1 | TLMSAEANL | 231 | PAP |
| PAP(TLM)S2 | QLCSAMTQL | 232 | PAP |
| PAP(TLM)S3 | RLMSALTQL | 233 | PAP |
| PAP(TLM)S4 | GLMSLTTNL | 234 | PAP |
| PAP(TLM)S5 | GLMSMATNL | 235 | PAP |
| PAP(TLM)S6 | GLMSMTTNL | 236 | PAP |
| PAP(TLM)S7 | LLMSISTNL | 237 | PAP |
| PAP(TLM)S8 | QLPSTMTNL | 238 | PAP |
| PAP(TLM)S9 | TLASSMGNL | 239 | PAP |
| PAP(TLM)S10 | TLFSALTGL | 240 | PAP |
| PAP(TLM)S11 | TLGSATTEL | 241 | PAP |
| PAP(TLM)S12 | TLMRAMTDC | 242 | PAP |
| PAP(TLM)S13 | TLMSMVANL | 243 | PAP |
| PAP(TLM)S14 | TLPSAETAL | 244 | PAP |
| PAP(TLM)S15 | TLPSRMTVL | 245 | PAP |
| PAP(TLM)S18 | RLMSALTQV | 246 | PAP |
| PAP(TLM)S19 | SIHSQMTNL | 247 | PAP |
| PAP(TLM)S20 | SIMFAMTPL | 248 | PAP |
| PAP(TLM)S21 | TIVAAMSNL | 249 | PAP |
| PAP(TLM)S22 | TLITAMEQL | 250 | PAP |
| PAP(TLM)S23 | TLTSNMSQL | 251 | PAP |

TABLE 13
Ala Scan - PAP

| Peptide name | Peptide-HLA-A2 sequence | SEQ ID NO: |
|---|---|---|
| PAP A1 | ALMSAMTNL | 252 |
| PAP A3 | TLASAMTNL | 253 |
| PAP A4 | TLMAAMTNL | 254 |
| PAP A6 | TLMSAATNL | 255 |
| PAP A7 | TLMSAMANL | 256 |
| PAP A8 | TLMSAMTAL | 257 |
| PAP A9 | TLMSAMTNA | 258 |

TABLE 14
Similar peptides found in normal essential tissues by MS.

| | Peptide | | |
|---|---|---|---|
| | sequence/SEQ ID | | Normal tissue in which |
| Peptide name | NO: | Gene | peptide was found by MS |
| KPNA1 | VMDSKIVQV/259 | KPNA1, KPNA5, KPNA6 | Adrenal, bladder, brain |
| | | | cerebellum, brain cerebral |
| | | | cortex, brain cerebrum, colon, |
| | | | heart, intestine, kidney, liver, |
| | | | lung, mesothelial, nerve, |
| | | | pituitary, retina, spinal cord |
| | | | cervical, adipose, breast, |
| | | | duodenum, esophagus, |
| | | | gallbladder, ovary, pancreas, |
| | | | prostate, skin, spleen, stomach, |
| | | | testis, uterus |
| WT1-S10 | RMLPHAPGV/260 | HDAC1, HDAC2 | Adrenal, bladder, brain |
| | | | cerebellum, brain cerebral |
| | | | cortex, brain cerebrum, colon, |
| | | | heart, intestine, kidney, liver, |
| | | | lung, mesothelial, nerve, |
| | | | pituitary, retina, spinal cord |
| | | | cervical, adipose, breast, |
| | | | duodenum, esophagus, |
| | | | gallbladder, ovary, pancreas, |
| | | | prostate, skin, spleen, stomach, |
| | | | testis, uterus |
| WT1-S12 | AMDPNAAYV/261 | SERPINA6 | Liver |
| WT1-S22 | RMNPNSPSI/262 | ERH | Colon, intestine, kidney, lung, |
| | | | duodenum, gallbladder, uterus |
| MAGE-A4-S1 | GLADGRTHTV/263 | THBS3 | Colon, endothelium, intestine, |
| | | | kidney, mesothelial, nerve, |
| | | | pituitary, duodenum, stomach |
| MAGE-A4-S16 | GLYDGPVHEV/264 | DPYSL4 | Brain cerebellum, brain |
| | | | cerebrum, intestine, lung, |
| | | | prostate, spleen |
| MAGE-A4-S21 | GVFDGLHTV/265 | BTD | Brain cerebral cortex, intestine, |
| | | | kidney, liver, lung, |
| | | | mesothelial, retina, breast, |
| | | | duodenum, stomach, testis, |
| | | | uterus |
| MAGE-A9-S26 | ALSDHHVYL/266 | ALDOC | Adrenal, bladder, brain |
| | | | cerebellum, brain cerebral |
| | | | cortex, brain cerebrum, colon, |
| | | | endothelium, heart, intestine, |
| | | | kidney, liver, lung, |
| | | | mesothelial, nerve, pituitary, |
| | | | retina, spinal cord cervical, |
| | | | breast, duodenum, esophagus, |
| | | | prostate skin, spleen, stomach, |
| | | | testis, uterus |
| PAP-S3 | RLMSALTQL/267 | DAB2IP | Brain cerebellum, brain |
| | | | cerebral cortex, brain |
| | | | cerebrum, colon, heart, |
| | | | intestine, kidney, lung, |
| | | | mesothelial, nerve, retina, |
| | | | spinal cord cervical, adipose, |
| | | | breast, duodenum, prostate, |
| | | | spleen, uterus |
| PAP-S18 | RLMSALTQV/268 | RASAL2 | Bladder, brain cerebellum, |
| | | | brain cerebral cortex, brain |
| | | | cerebrum, colon, endothelium, |
| | | | heart, intestine, kidney, liver, |
| | | | lung, mesothelial, nerve, |
| | | | pituitary, retina, spinal cord |
| | | | cervical, adipose, breast, |
| | | | duodenum, esophagus, |
| | | | gallbladder, ovary, prostate, |
| | | | skin, spleen, stomach, testis, |
| | | | uterus |

TABLE 15
Control peptides

| | | SEQ |
|---|---|---|
| | | ID |
| Peptide | Peptide-HLA-A2 sequence | NO: |
| MART1(26) | ELAGIGILTV | 269 |
| CMV | NLVPMVATV | 270 |
| Gag | SLYNTVATL | 271 |
| Tyrosinase D | YMDGTMSQV | 272 |
| WT-1 | RMFPNAPYL | 273 |
| MAGE-A4 | GVYDGREHTV | 274 |
| PAP | TLMSAMTNL | 275 |
| MAGE-A9 | ALSVMGVYV | 276 |
| SSX-2 | KASEKIFYV | 277 |
| NY-ESO | SLLMWITQC | 278 |
| UHRF1 | TLFDYEVRL | 279 |

### Example I

### TCR-Like Antibodies for HLA-A2/Tyrosinase

### Isolation of Abs with TCR-Like Specificity to HLA-A2/Tyrosinase369-377

Generation of MHC-TyrD369-377 Complex—

Previous studies performed by the present inventors have shown the generation of recombinant antibodies with peptide-specific, HLA-A2-restricted specificity to tumor and viral T cell epitopes using large antibody phage libraries. These molecules are termed TCR-like antibodies. To generate antibodies with a specificity to the HLA-A2/TyrD369-377 complex, recombinant peptide-HLA-A2 complexes were generated that present the Tyrosinase peptide (tyrosinase₃₆₉₋₃₇₇YMDGTMSQV, SEQ ID NO: 1) using a single chain MHC construct. HHD mice were immunized by 5-6 injections of HLA-A2-peptide complex 50 μg/mouse. 2-3 first injections were administrated s.c with addition of QuilA adjuvant. Hybridoma clones were generated by fusion of splenocytes isolated from immunized mice (as previously described e.g., Weidanz et al. 2011 Int. Rev. Immunol. 30:328-340) with NSO myeloma cells and were screened and isolated by differential ELISA assays as described above using TyrD369-377 peptide and HLA-A2 complexes folded with p68-DDX5 control peptide. ELISA with purified HLA-A2-Tyr complexes as well as with control HLA-A2 complex displaying other HLA-A2-restricted peptide was used to select specific clones Isolated hybridoma clones were sub-cloned and were sequenced. Two clones 906-11-D11 (termed D11, FIG. 69) and 905-2-D7 (termed D7, FIG. 68) were characterized.

Characterization of TCR-Like Antibodies with Specificity to HLA A2/Tyrosinase 369-377

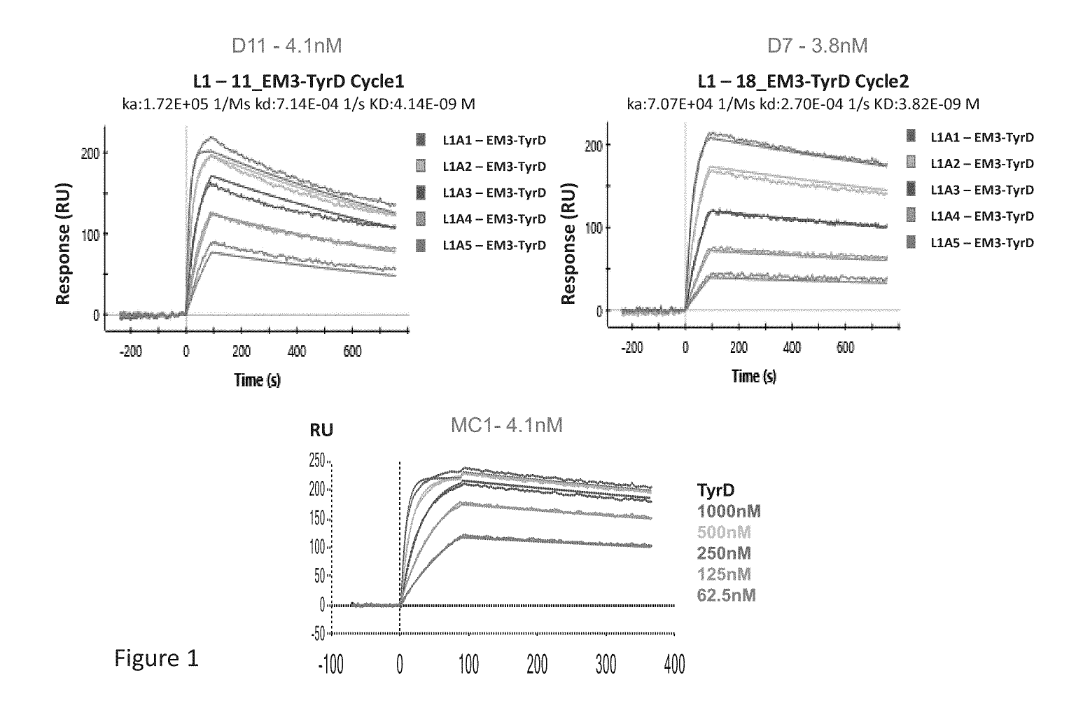

To determine the apparent affinity of isolated TCR-like antibodies, surface plasmon resonance (SPR) binding analysis was used in which the isolated purified IgG TCR-like antibody was immobilized to the SPR sensor chip by using anti-mouse IgG to indirectly immobilize the TCR-like antibodies on the chip surface. The analyte is the purified single-chain recombinant HLA-A2/Tyrosinase complex used at various concentrations. As shown in FIG. 1, the sensorgrams of SPR analysis revealed similar affinity for the HLA-A2/Tyrosinase specific TCR-like antibody clones MC1, D11, and D7 with corresponding affinity of 4.1 nM for MC1 and D11 and 3.8 nM for D7. These results indicate that all three TCR-like antibody clones exhibited similar high affinity of 4 nM towards the specific HLA-A2/peptide complex.

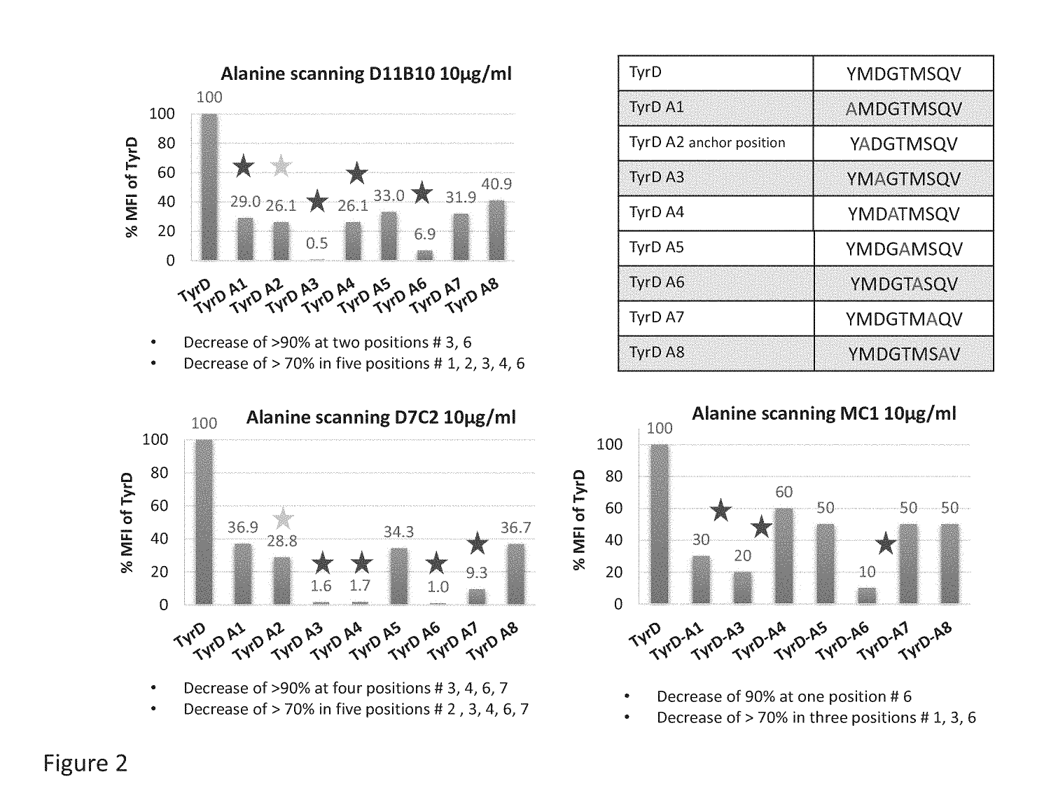

To investigate the fine peptide epitope specificity of the isolated TCR-like antibodies towards the Tyrosinase 369-377 peptide alanine scanning was performed in which specific residues in the peptide were mutated to alanine and the binding of the TCR-like antibodies to Ala mutated peptides was tested by their loading onto T2 antigen presenting cells. Binding was monitored by flow cytometry and extent of binding of TCR-like antibodies to the mutated presented peptides as measured by mean fluorescence intensity (MFI) was compared in comparison to T2 APCs loaded with the native unmutated Tyrosinase peptide. The proper loading of the various Ala mutated peptides (described in FIG. 2) was monitored by flow cytometry using BB7.2 a monoclonal antibody for HLA-A2.

All Ala mutated peptides were efficiently loaded onto T2 cells in comparison to the native un-mutated Tyrosinase peptide (data not shown). Peptide loading efficiency is verified using the ratio between MFI of HLA-A2-binding Ab BB7.2 on peptide-loaded T2 cells and MFI of unloaded T2 cells (>1).). As shown in FIG. 2, all three TCR-like antibodies exhibited peptide dependency binding as specific mutations affected the binding and induced a decrease in the binding intensity of the TCR-like antibody upon introduction of Ala at specific peptide positions. These results indicate that all three TCR-like antibodies exhibited peptide-specific and restricted binding in the context of HLA-A2 loaded with various Ala mutated Tyrosinase peptides, indicating that these antibodies are TCR-like in their binding properties, thus, they bind the MHC-peptide complex with MHC-restricted and peptide-specific manner.

However, the three TCR-like antibodies differ in their fine specificity and peptide-dependent reactivity with the number of positions in the peptide that were sensitive to Ala mutation and affected binding sensitivity. As MC1 exhibited a marked decrease of 90% in binding to a single Ala mutated peptide at one position #6, D11 and D7 exhibited a decrease of >90% at two positions #3, 6 for D11 and a decrease of >90% for D7 binding at four positions #3, 4, 6, 7. A milder but highly significant decrease of >70% in three positions #1, 3, 6 was further observed for MC1 binding to Ala mutated peptides while D11 and D7 exhibited significant decrease in binding of >70% when 5 peptide residues were mutated to Ala (positions #1, 2, 3, 4, 6 for D11 and positions #2, 3, 4, 6, 7 for D7).

Overall, the Alanine scanning analysis reveals that D11 and D7 are more influenced and sensitive to Ala mutations compared to MC1 as observed by the ability of the various Ala mutated Tyr peptide to bind properly the Tyr specific TCR-like antibodies. According to the data presented in FIG. 2, D11 and D7 are more peptide restricted and sensitive in their binding properties compared to MC1; they are sensitive (not including anchor positions) to Ala mutations in 4 out of 9 peptide residues while MC1 only to 3 positions. D11 and D7 are even sensitive in their binding properties in a 5^{th} position 7, and 5, respectively. Specifically, D11 decrease the binding in 68% at position #7, 67% position #5, 59% position #8; D7 decrease the binding in 66% at position #5, 63% position #1, 63% position #8.

It is concluded that Ala scanning can be used as a measure to determine the selectivity and fine specificity of TCR-like antibodies. As more sensitivity to Ala mutations is exhibited the more specific and peptide-dependent binding will be observed. This strategy can be used to filter and select for the optimal TCR-like antibodies that exhibited the higher and optimized selectivity and specificity properties as MHC-restricted peptide-specific binders.

Binding Selectivity and Specificity of TCR-Like Antibodies Towards HLA-A2/Tyrosinase

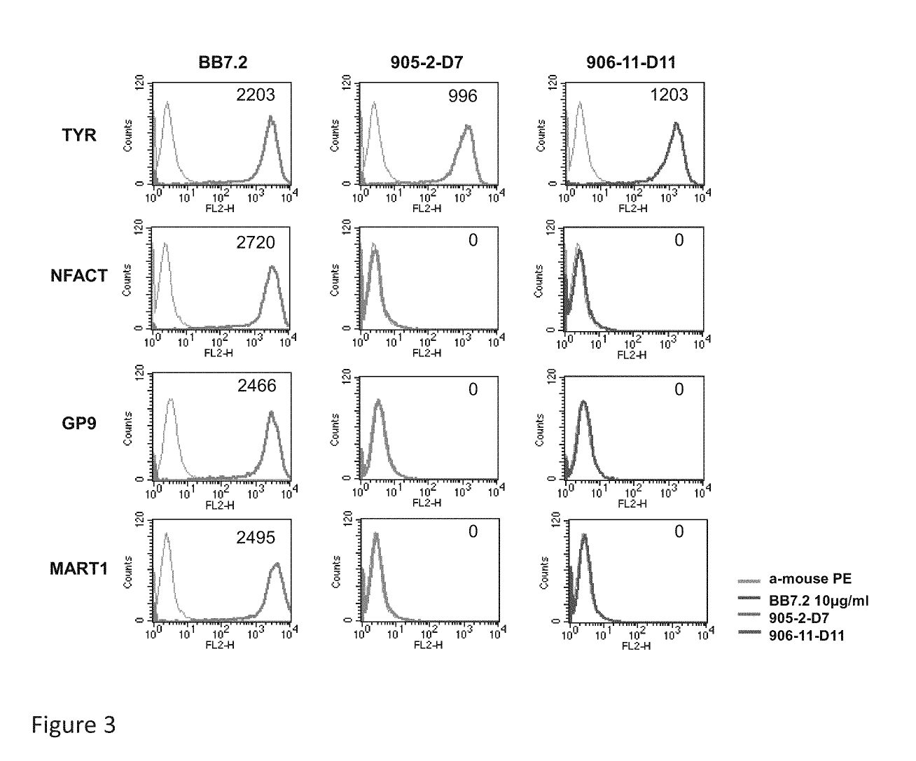

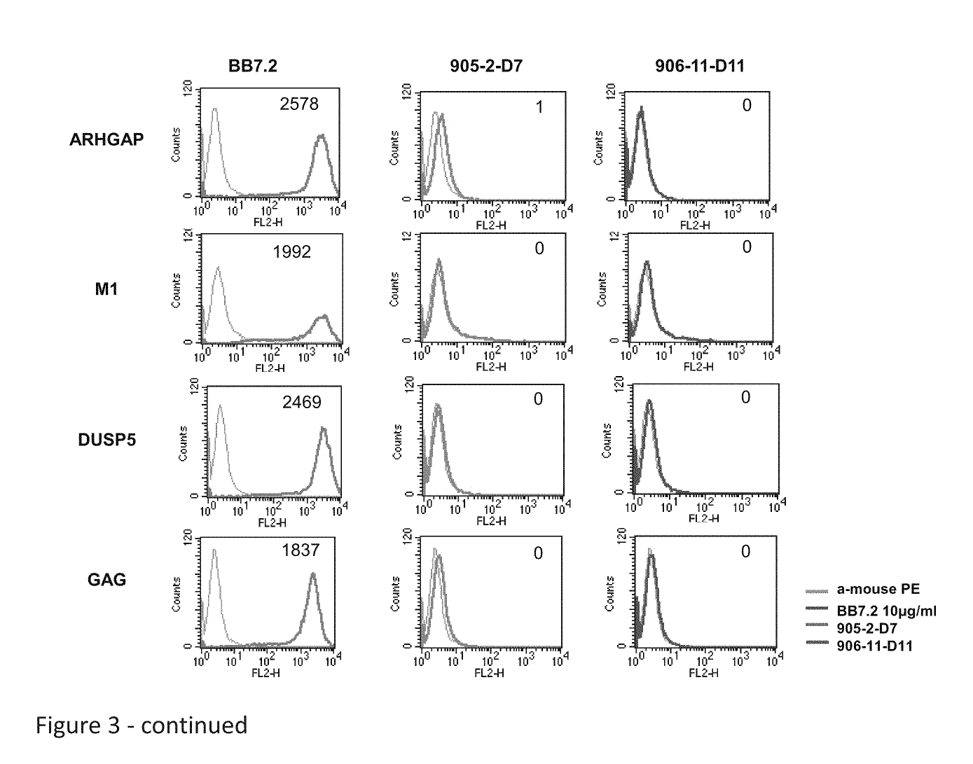

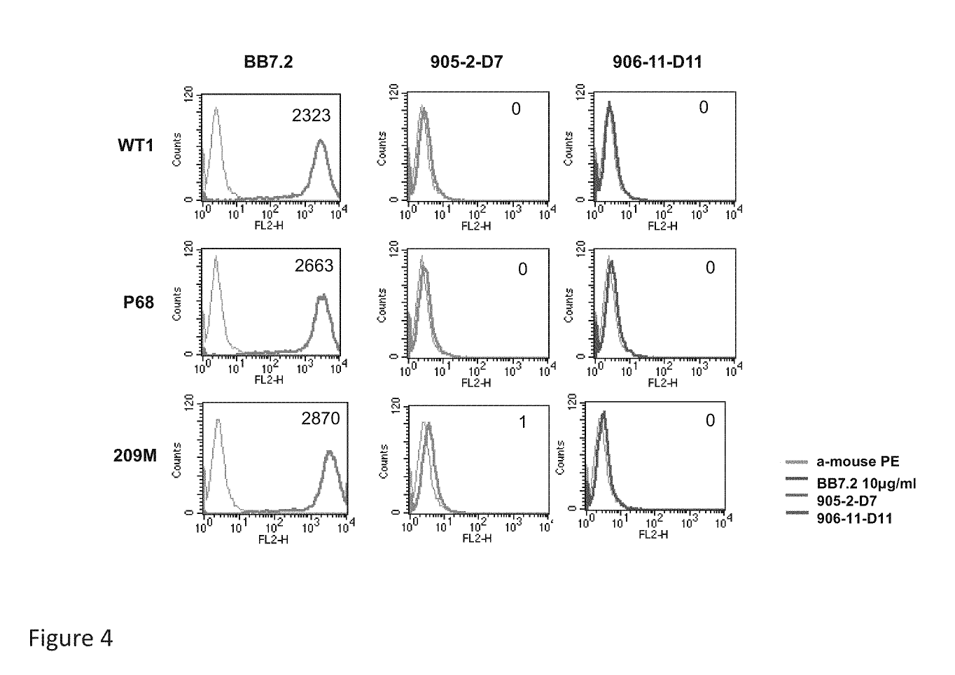

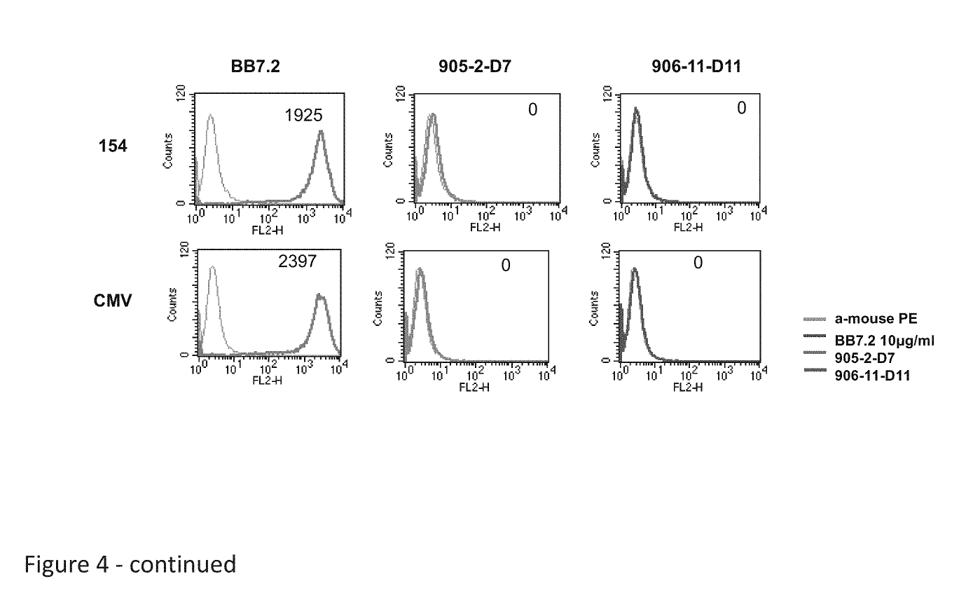

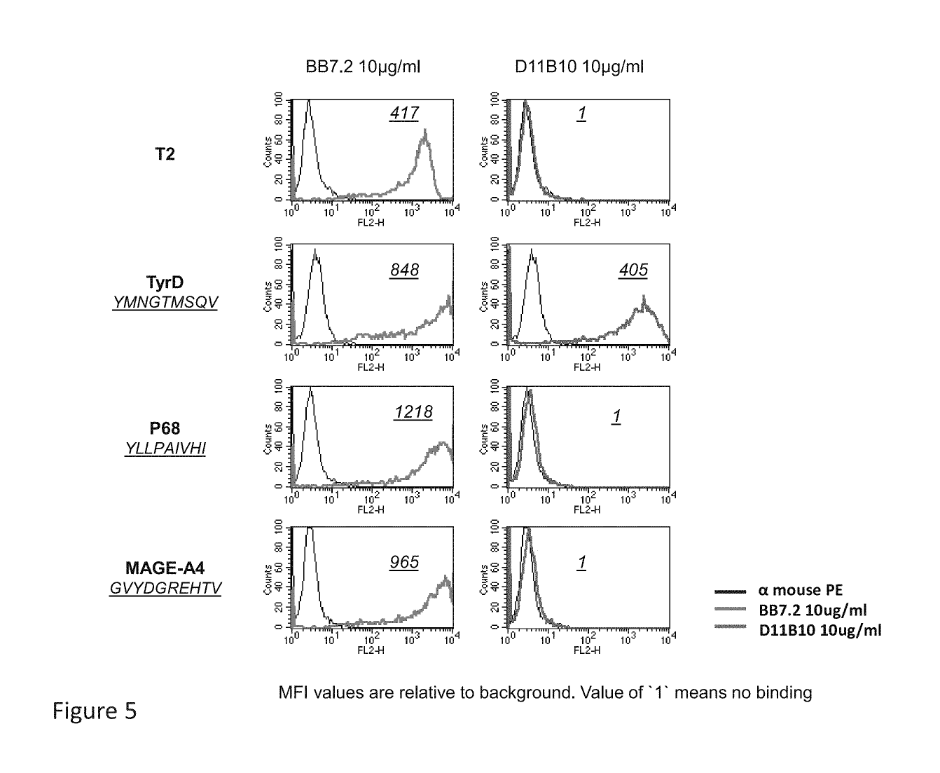

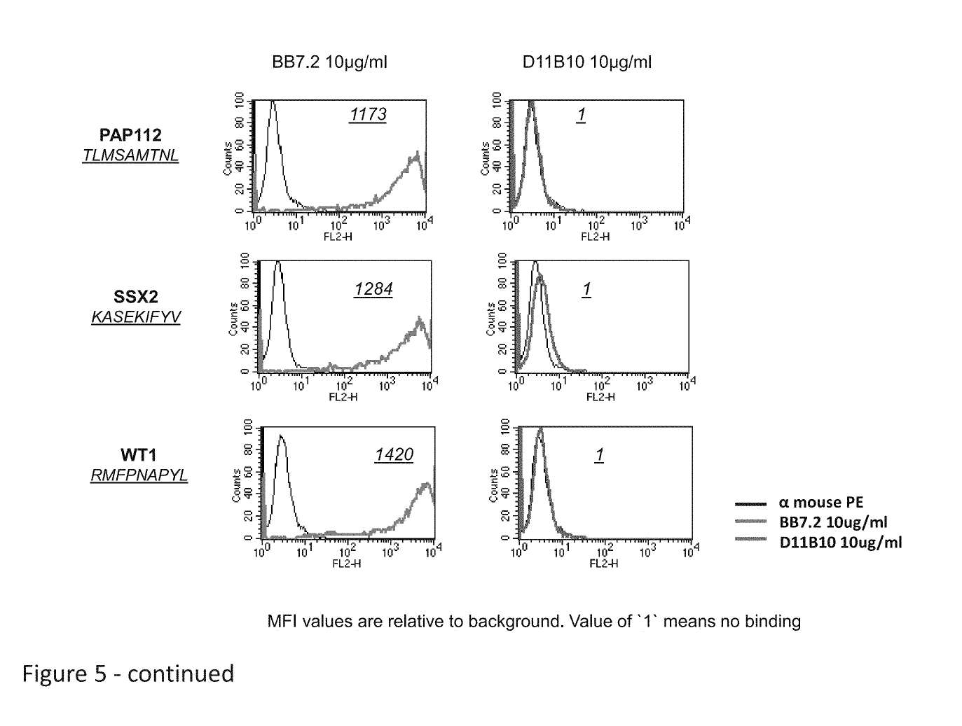

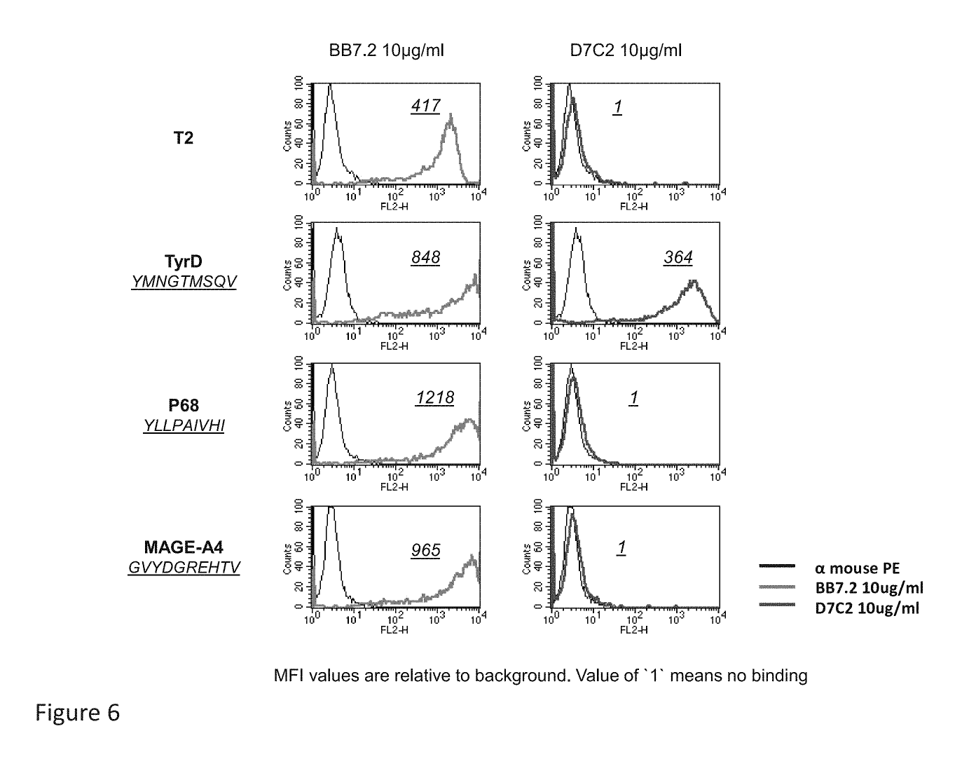

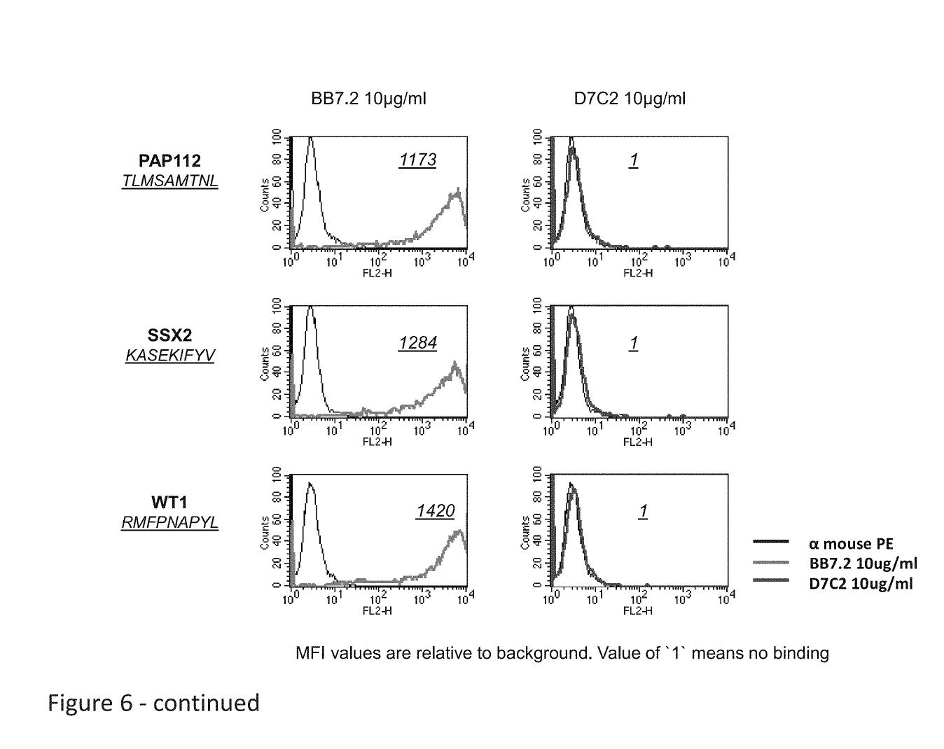

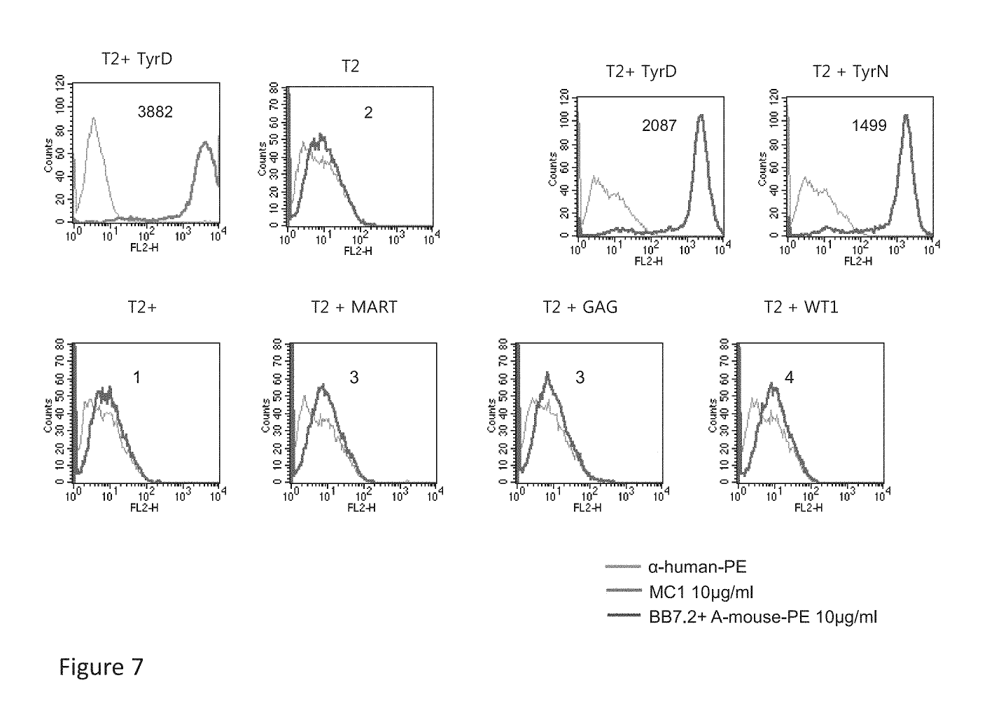

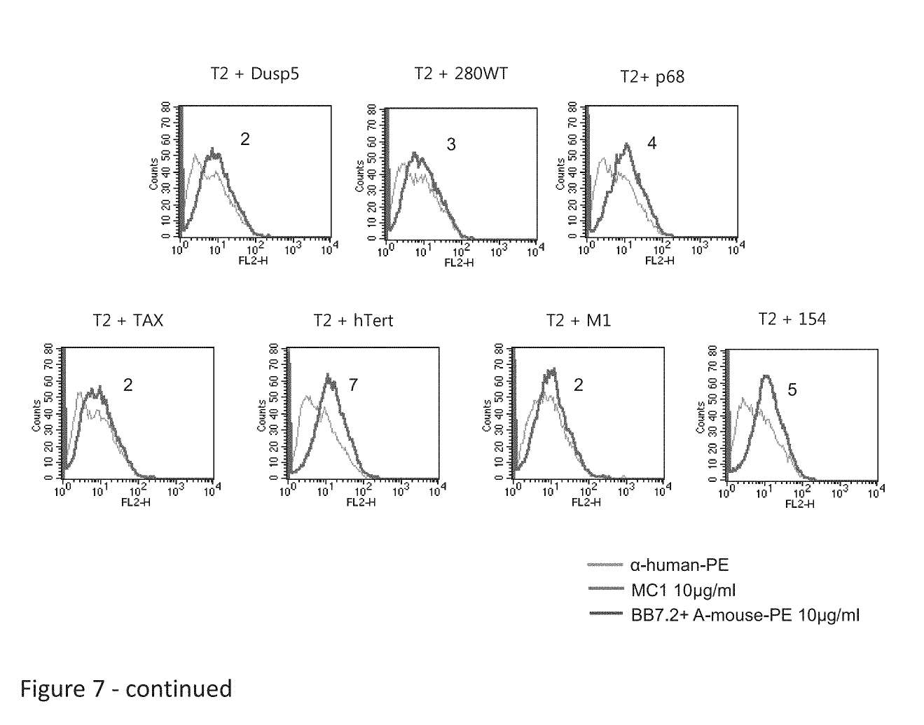

To characterize the binding specificity of the isolated TCR-like antibodies the reactivity and specificity of the purified IgGs were assessed by flow cytometry. T2 APCs were loaded with specific or control peptides and incubated with the Ab, followed by incubation with PE-labeled anti-human or mouse. Ab. As shown in FIGS. 3-7, the MC1 (FIG. 7), D11, and D7 (FIGS. 3-6) IgGs bound T2 cells loaded with the tyrosinase peptide but did not bind significantly to cells loaded with control peptides (Table 15). Very low background binding was observed on control peptides with MFIs ratio of 3-7 for MC1 (FIG. 7) while D11 and D7 did not exhibit any background binding (FIG. 3-6). The extent of loaded peptide presentation was monitored by binding of MAb BB7.2 which binds all HLA-A2 peptide complexes. These results indicate that all three TCR-like antibodies exhibited HLA-A2-restricted peptide-specific binding as they bound only to cells presenting the Tryosinase but no other HLA-A2 restricted peptides.

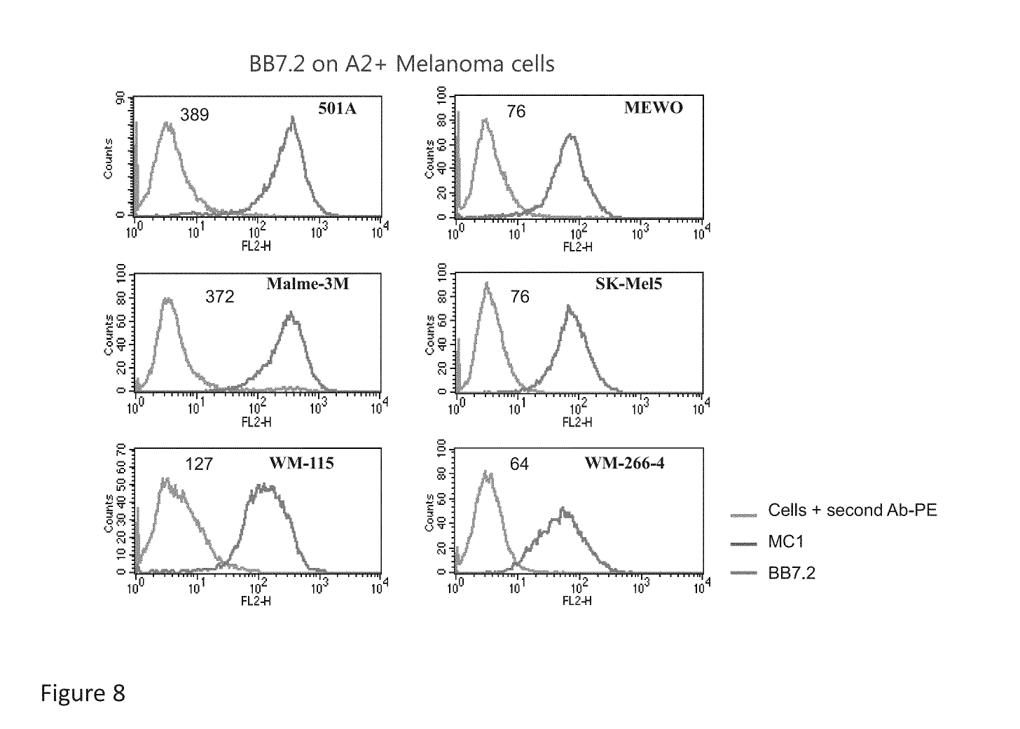

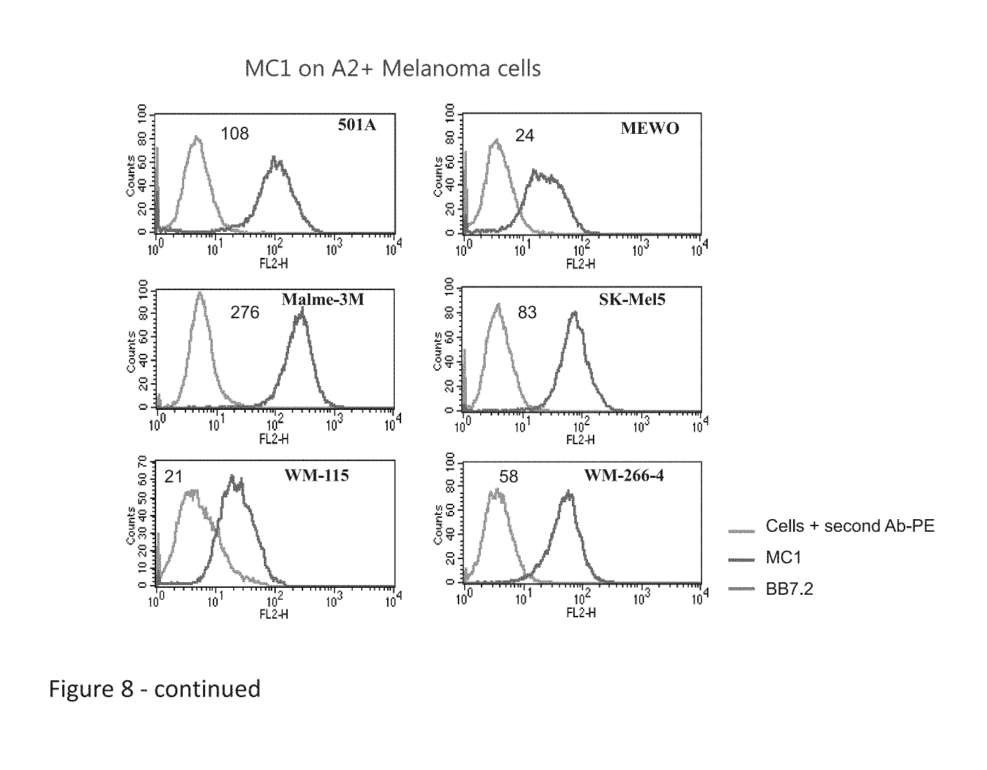

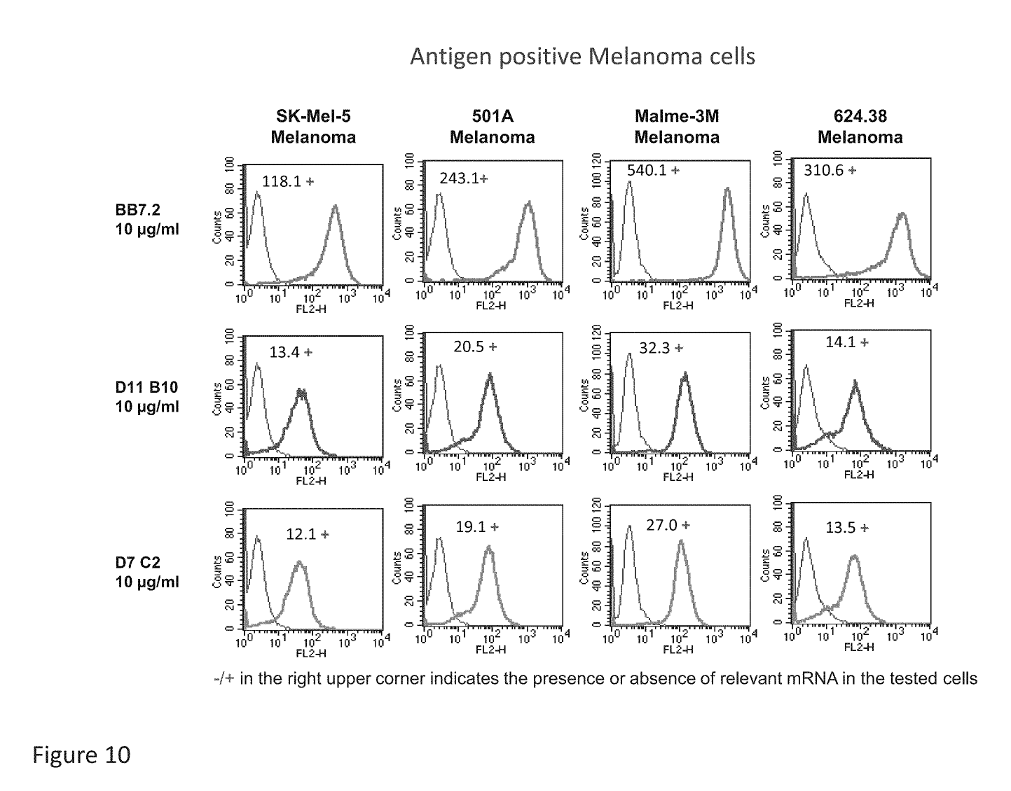

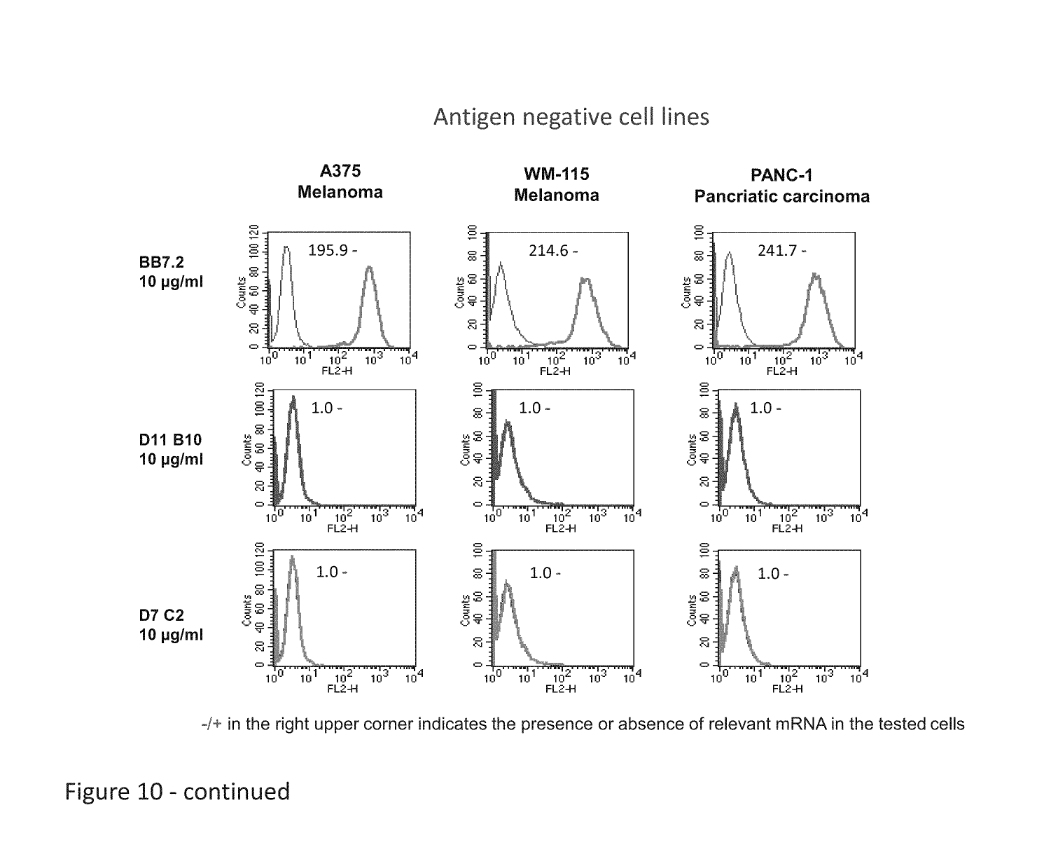

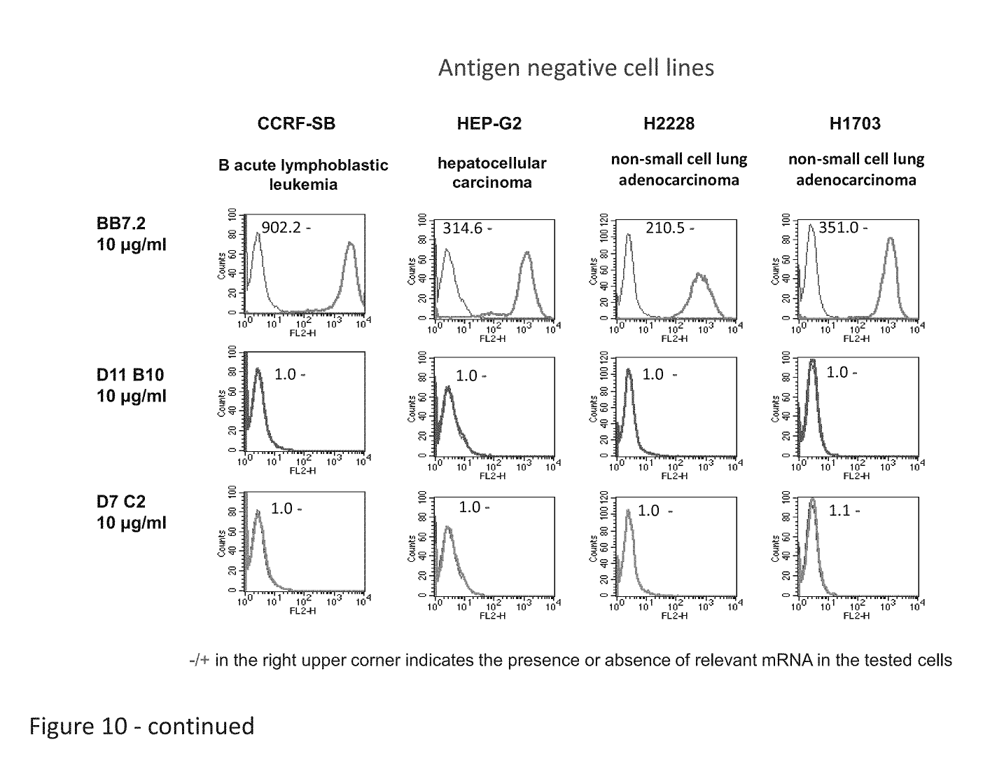

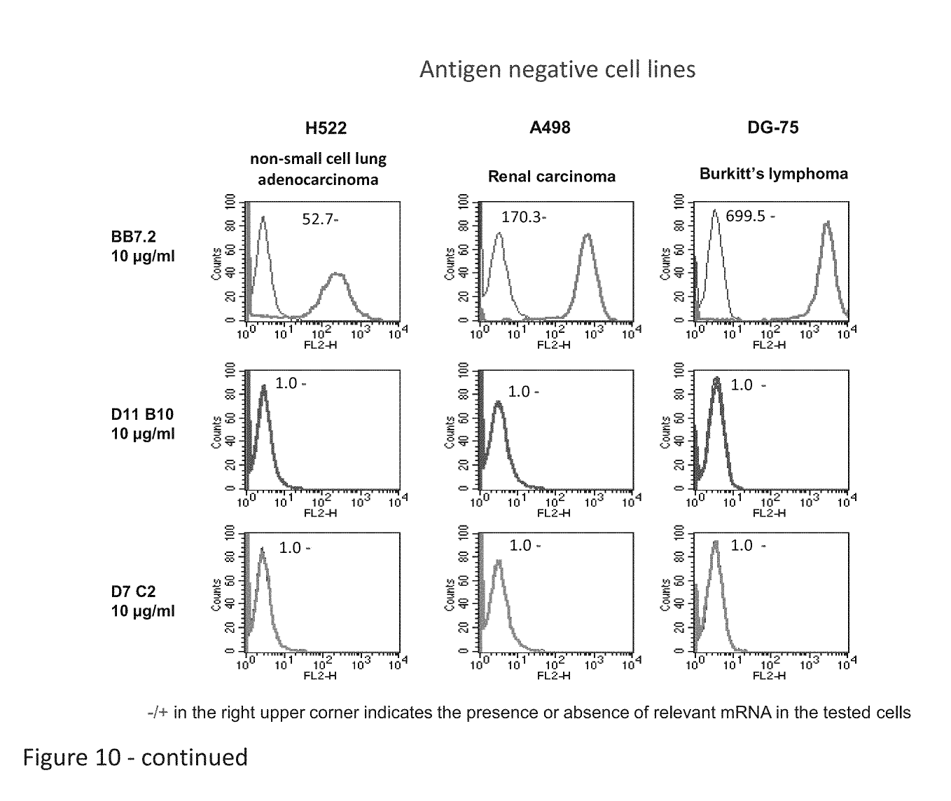

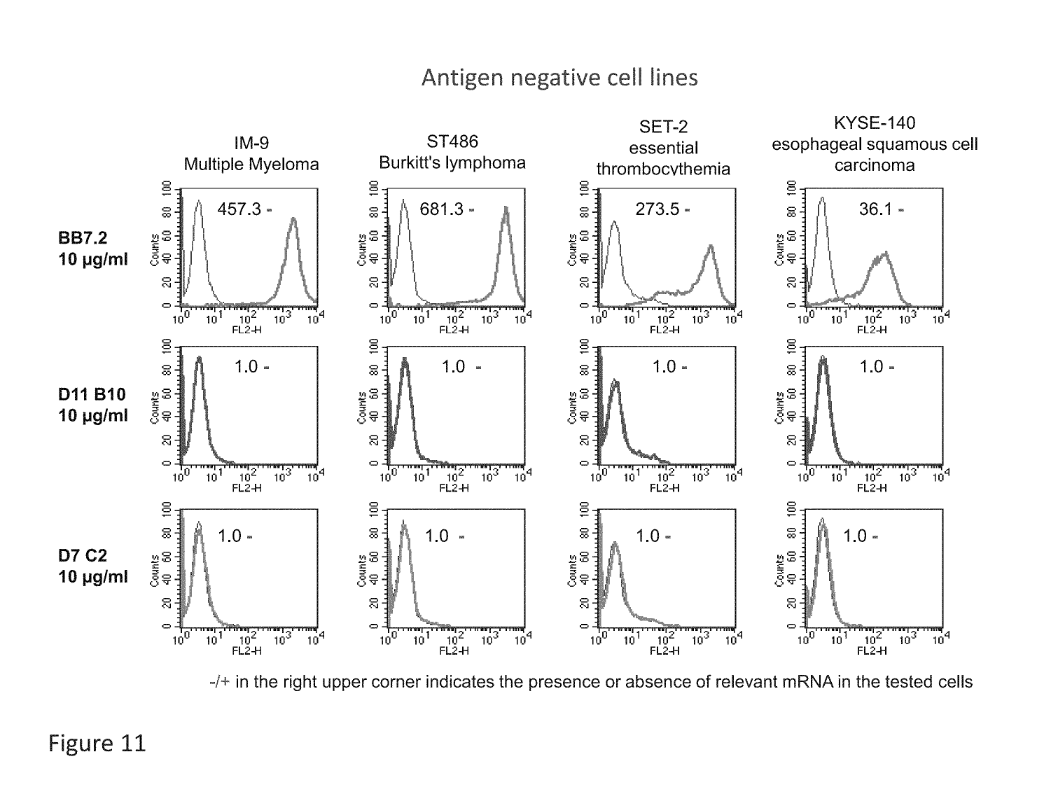

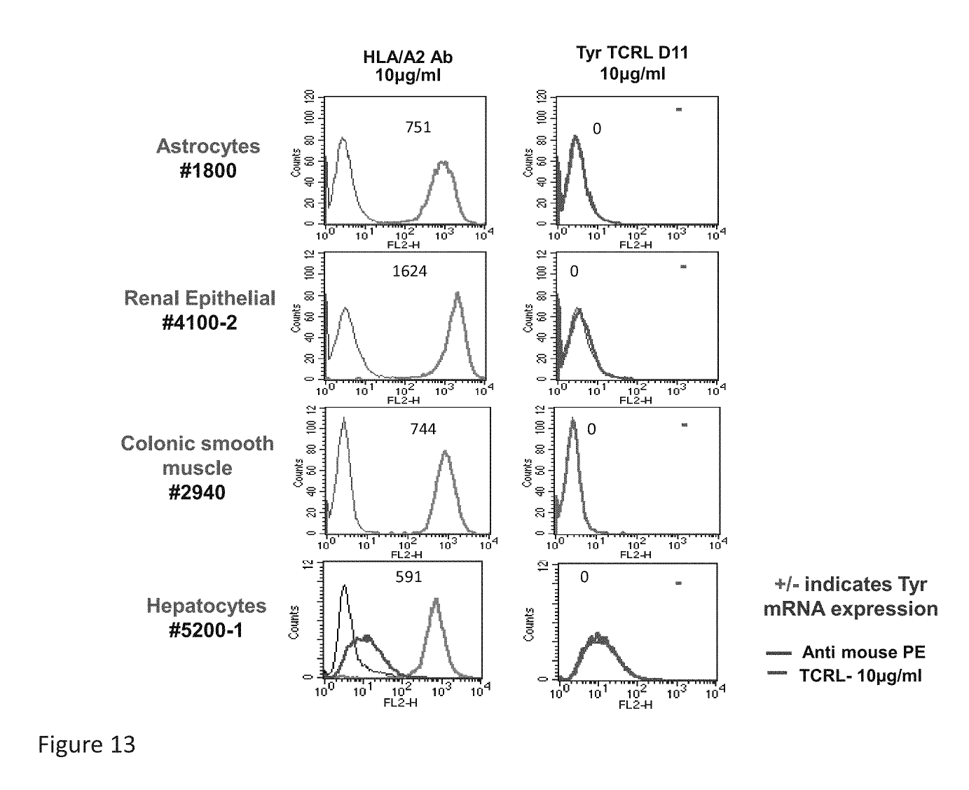

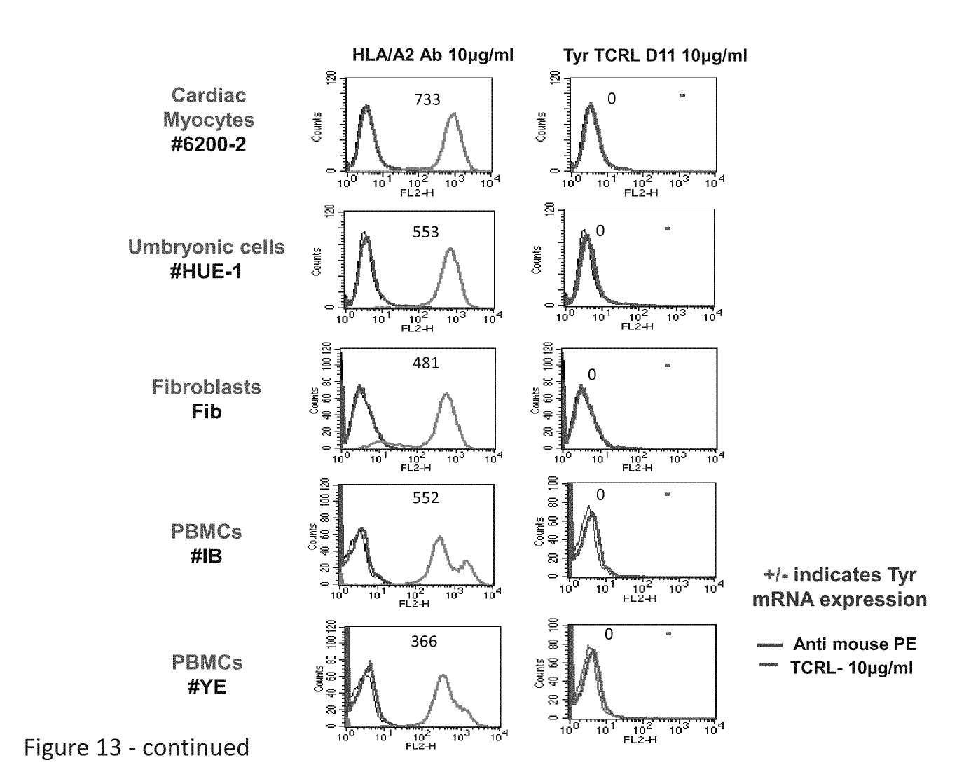

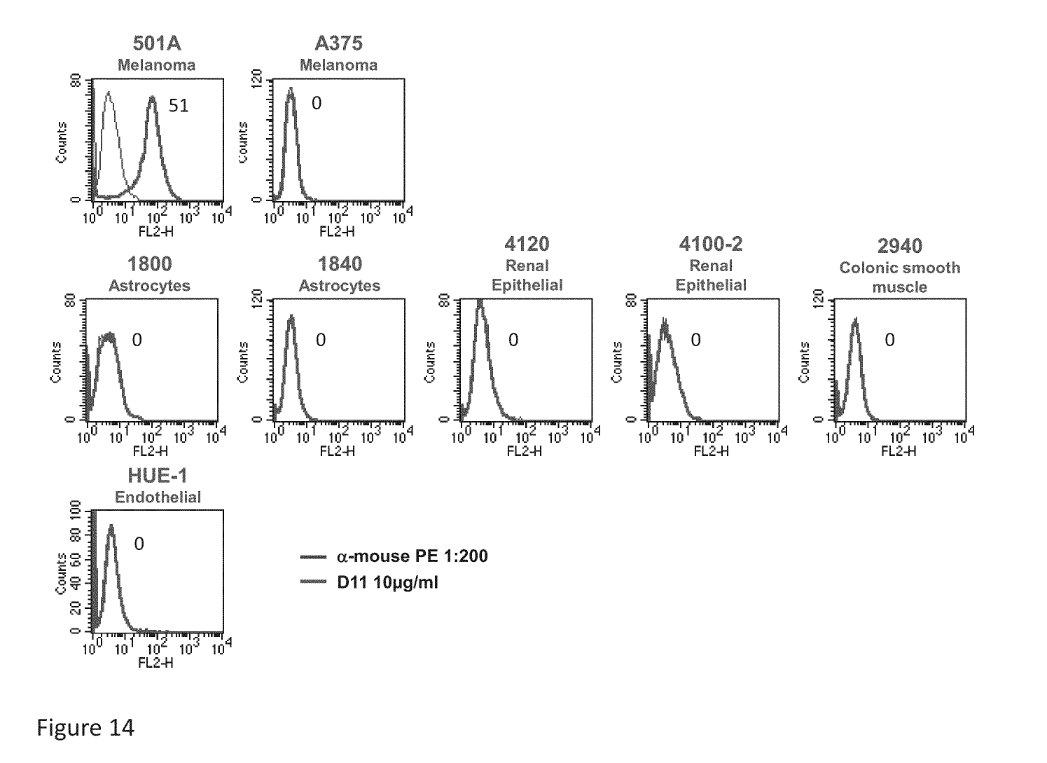

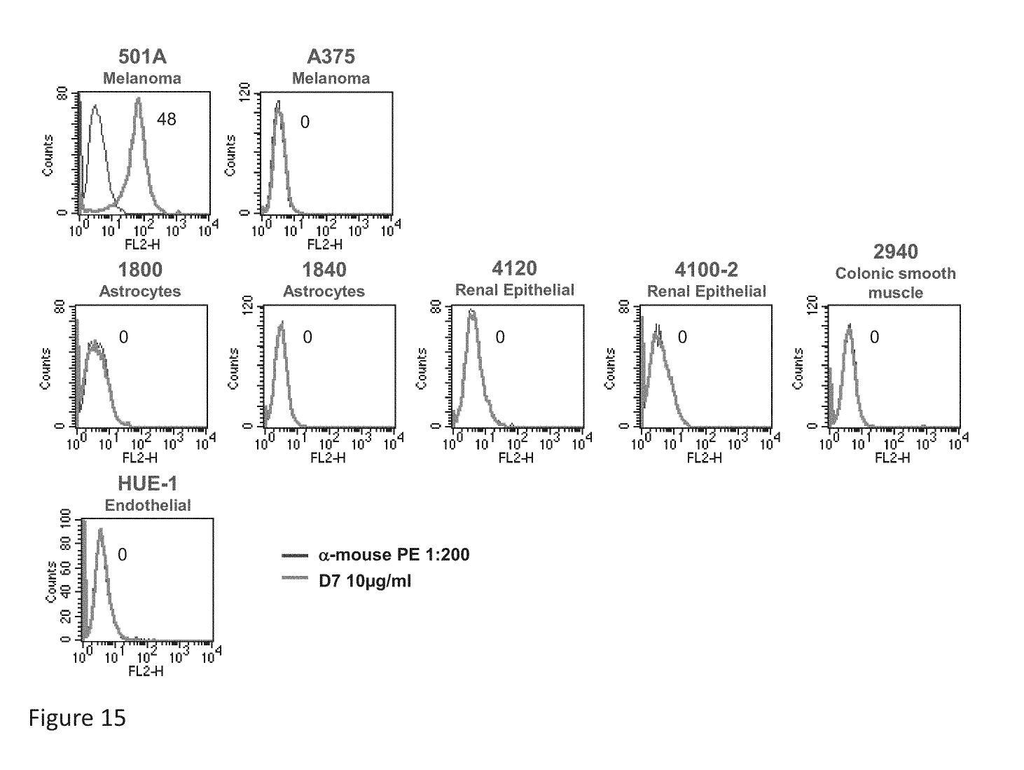

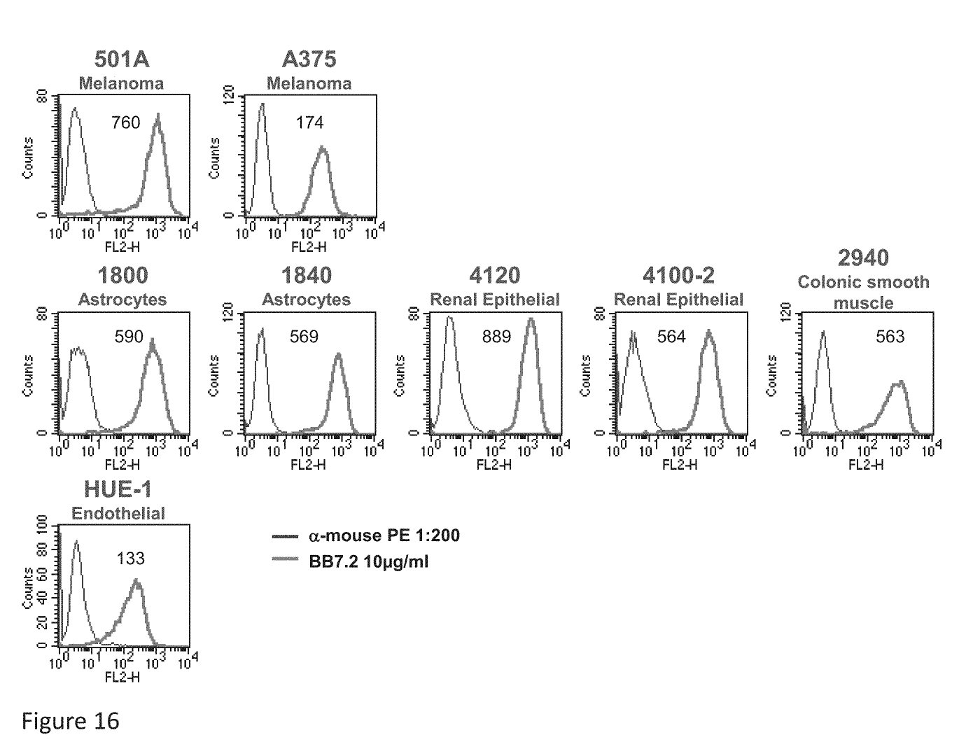

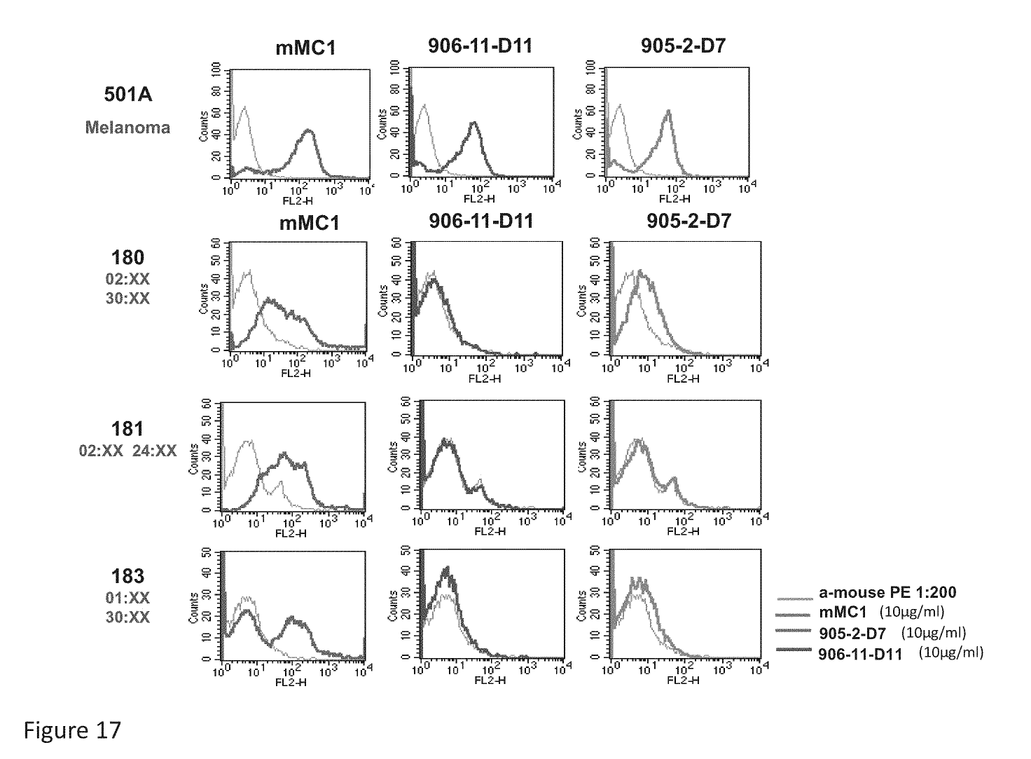

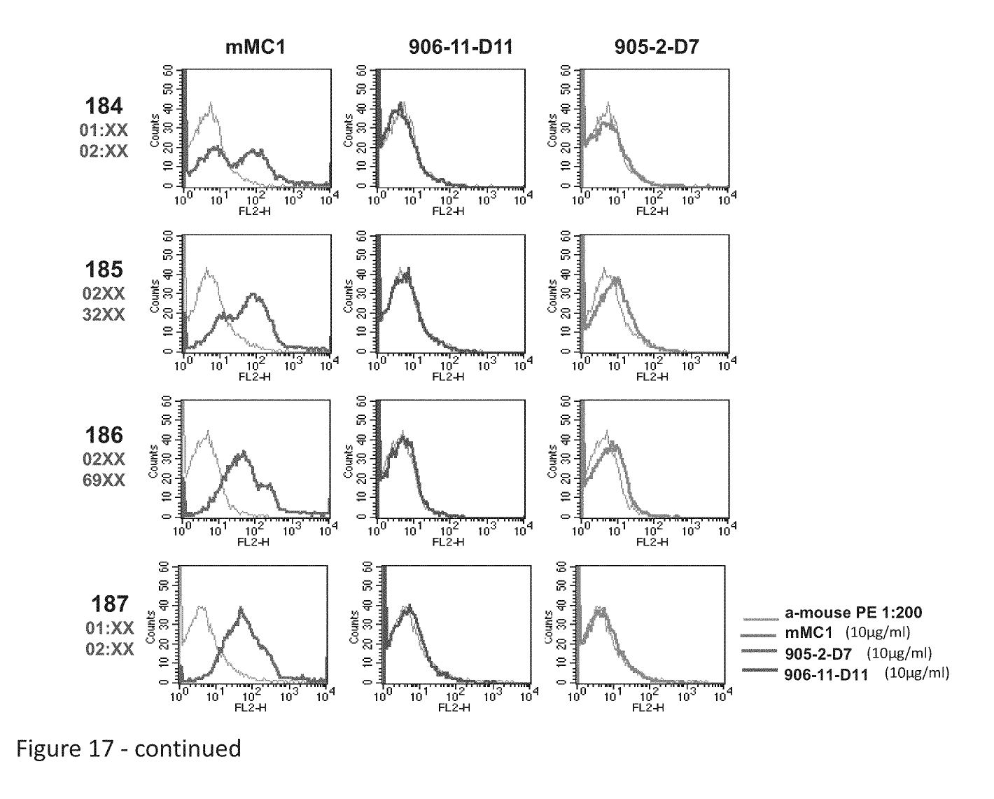

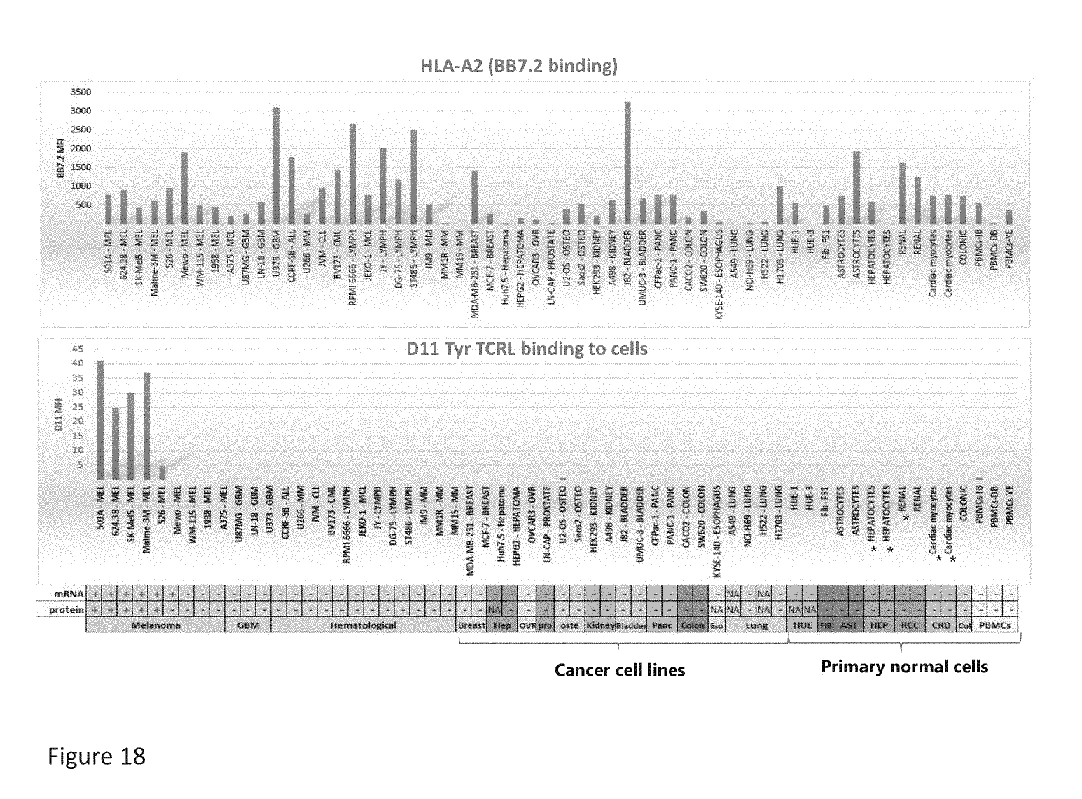

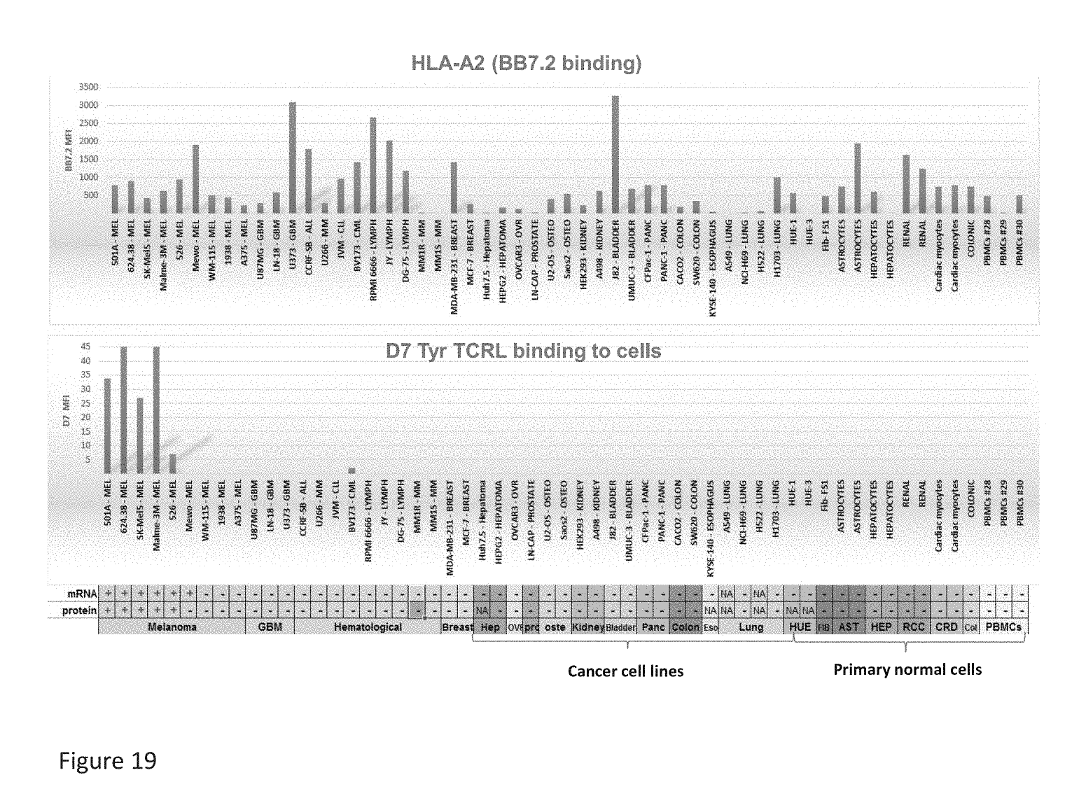

To explore whether the HLA-A2/tyrosinase TCR-like Abs are capable of binding endogenously derived MHC-tyrosinase complexes on the surface of tumor cells, flow cytometry analysis was done on lines derived from melanoma patients. Cells were incubated with anti-tyrosinase 369-377/HLA-A2 TCR-like antibodies Ab followed by incubation with PE-labeled anti-human or anti-mouse Ab. As shown in FIGS. 8-12 the TCR-like antibodies recognized tyrosinase-positive and HLA-A2-positive cells with a very high intensity. As shown this indicates that large numbers of HLA-A2-tyrosinase complexes are presented on the surface of the melanoma cells. The staining with the TCR-like antibodies was very homogeneous; intracellular staining of these melanoma cells (for example 624.38, and 501A) with Ab against the tyrosinase protein revealed that ˜95% of the cells in each line tested express the tyrosinase protein (data not shown). No reactivity was detected with tyrosinase-negative or HLA-A2-negative cells. The specificity of the anti-tyrosinase/HLA-A2 TCR-like Abs was verified by extensive flow cytometry analysis of multiple cell lines of various histological origins which are HLA-A2 positive and Ag (tyrosinase) negative. This analysis is shown in FIGS. 10-12. D11 and D7 reactivity was tested also on a panel of normal primary cells including endothelial cells, fibroblasts, astrocytes, hepatocytes, renal cells, cardiac myocytes, colonic muscle, and PBMCs (FIGS. 13-17). No binding to these HLA-A2+ and Tyr− normal primary cells was observed while background binding was observed when MC1 was tested on PBMCs (FIG. 17). Summary of the analysis of D11 and D7 reactivity with HLA-A2+/Tyrosinase+ melanoma cells as well as extensive panel of HLA-A2+/Tyrosinase− cells of various histological origins including the normal primary cells is presented in FIGS. 18-19. D11 and D7 TCR-like antibodies reactivity looks extremely specific only to melanoma cells expressing HLA-A2 and the antigen tyrosinase.

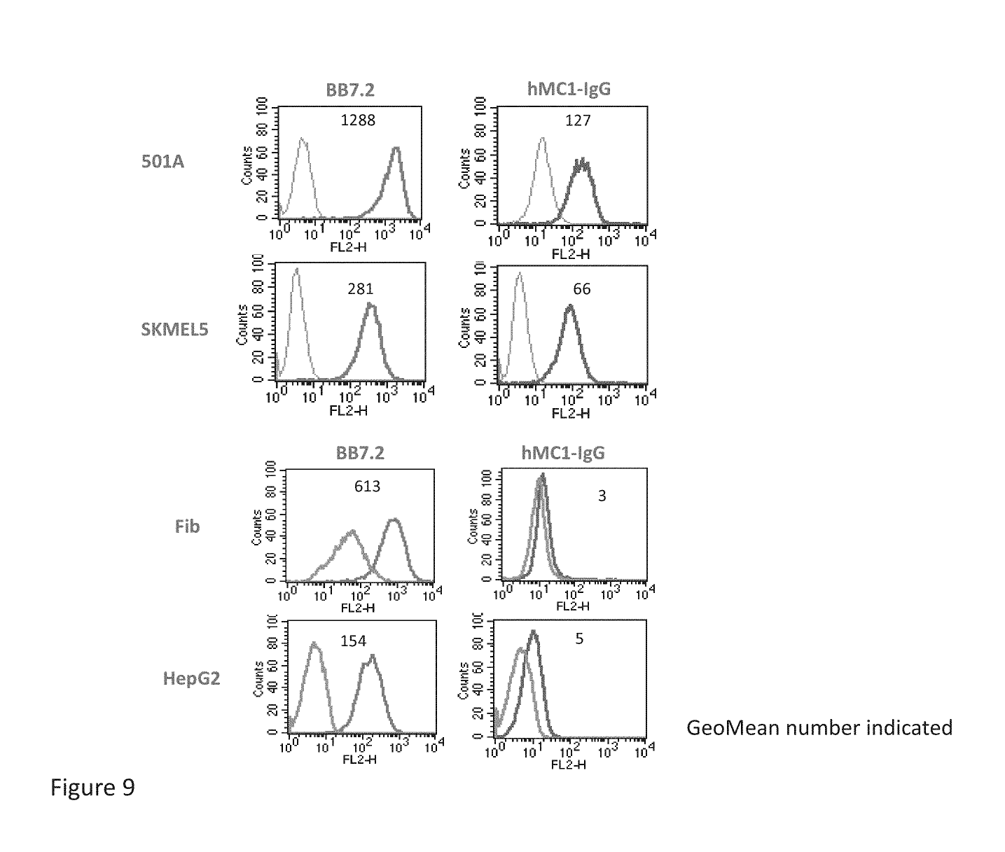

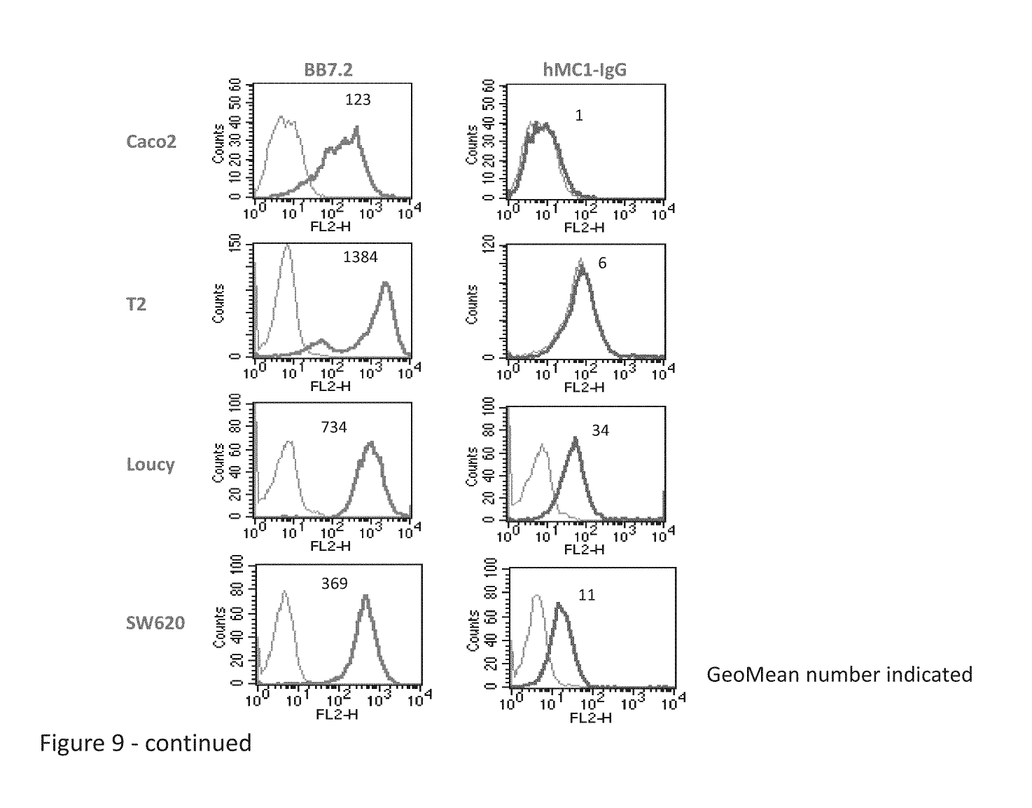

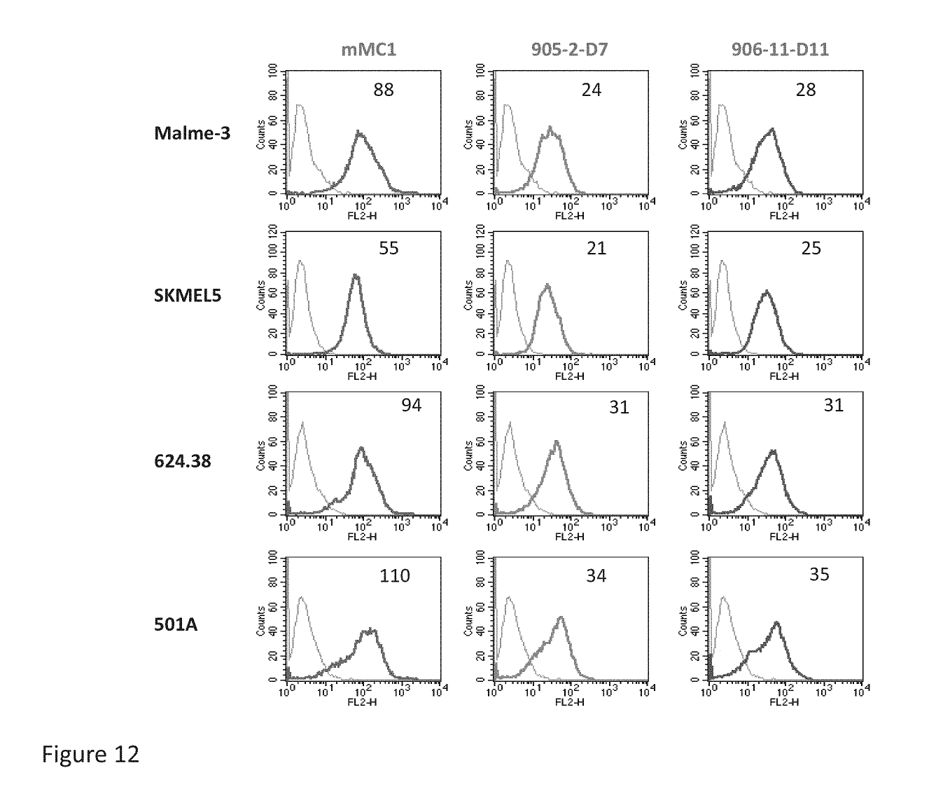

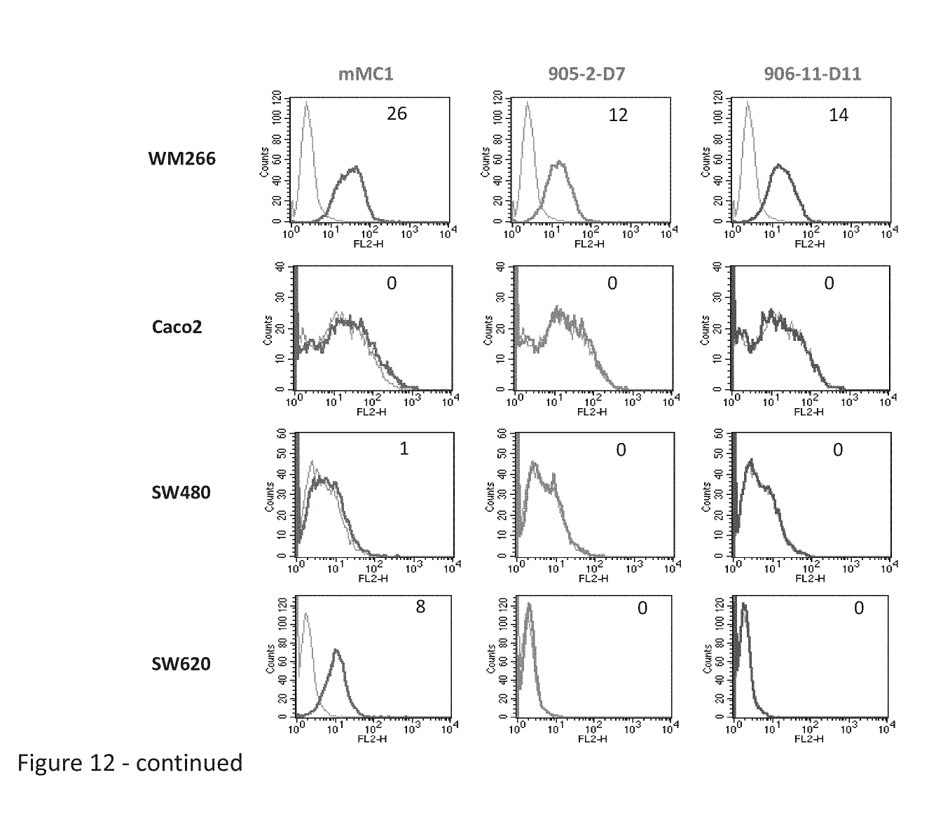

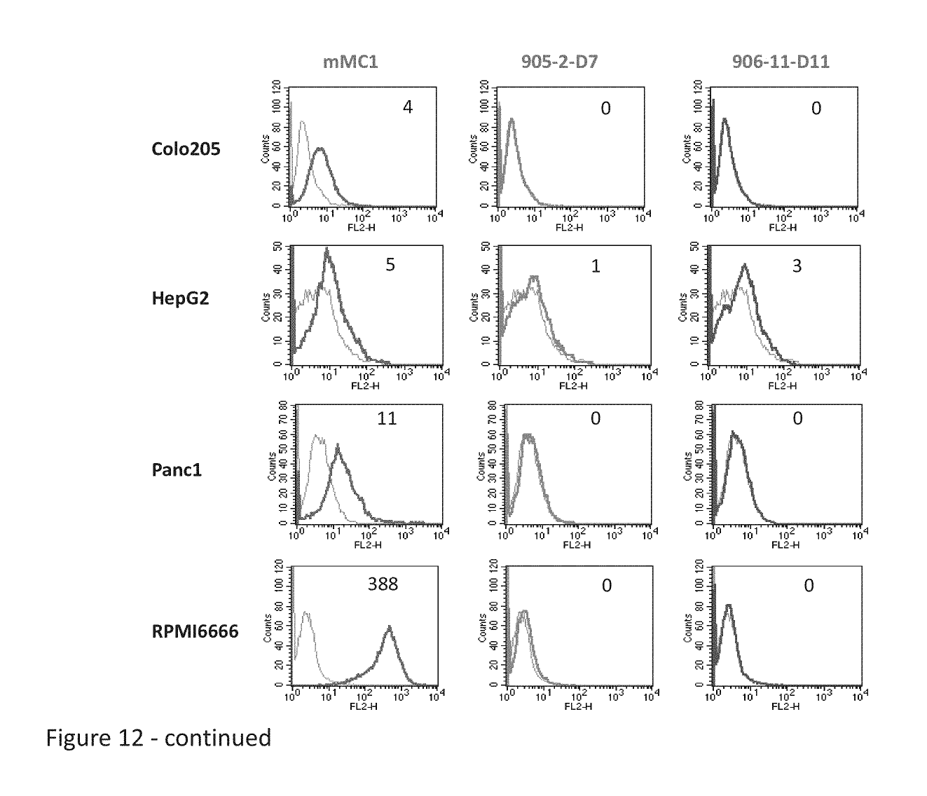

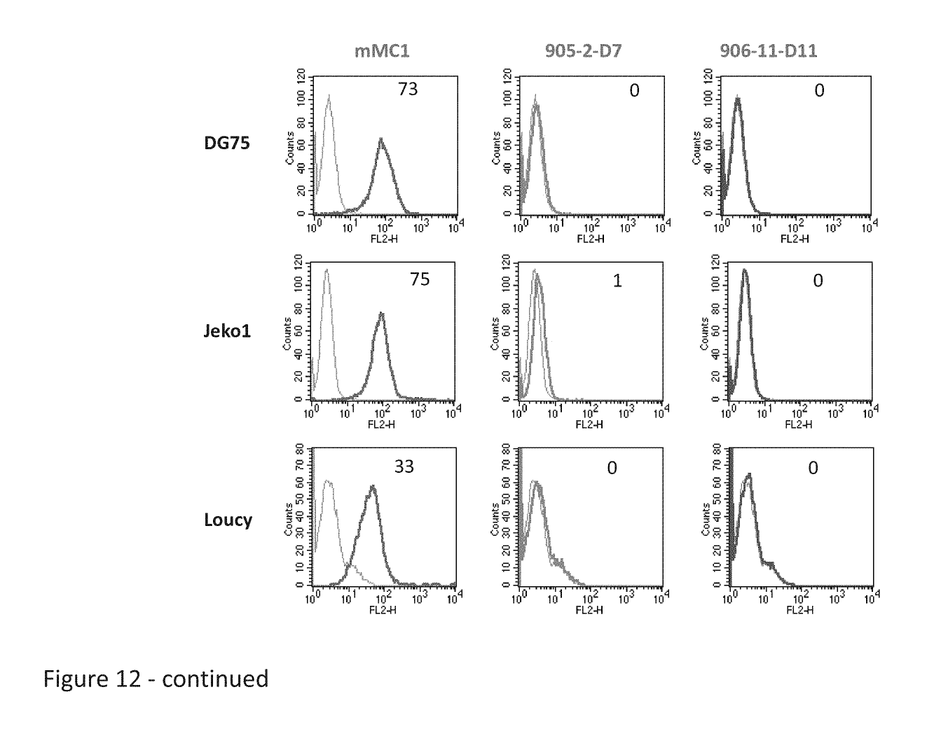

The overall conclusion from these studies is that the TCR-like Abs are specific and they recognize only the specific peptide-MHC complex presented on the cell surface when the adequate combination of HLA allele and Ag exist. However, careful evaluation of flow cytometry data exhibited results that demonstrate differential selectivity of MC1 compared to D11 and D7. For example, analysis of binding of MC1 to HLA-A2+ and Tyr− cell lines HepG2, SW620, and Loucy as shown in FIG. 9 reveals background binding as measured by MFI, however, similar analysis of D11 and D7 on these cells revealed no binding (FIGS. 10 and 12). Side by side comparison of the three TCR-like antibodies on these and additional cells (FIG. 12) revealed that MC1 exhibited significant binding to HLA-A2+/Tyr+ melanoma cells but had background binding on a variety of HLA-A2+/Tyr− cells (SW620, Colo205, HepG2, Panc1, RPMI, DG75, Jeko1, and Loucy) while D11 and D7 did not exhibit any background binding to these cells.

It may thus be concluded that D11 and D7 are more specific and selective compared to MC1 and that comprehensive flow cytometry studies as well as other assays, for example, functional assays utilizing a large panel of cells of different histological origins that express the appropriate HLA allele and are positive or negative for the antigen are useful tools to evaluate the selectivity of TCR-like antibodies.

To further evaluate the fine specificity of the Tyrosinase specific TCR-like antibodies their reactivity with peptides that exhibit sequence similarity to the native tyrosinase was evaluated (Table 5).

Thus, another round of similar peptides selection is performed when Alanine/Glycine scanning data are available as described above for a particular TCR-like antibody. Based on alanine scanning the contribution of each amino acid residue in the peptide antigen to TCRL binding is measured and evaluated. Similar peptides that preserve the critical positions are identified by the above described tools and are assigned higher priority. These peptides are synthesized and used for fine specificity evaluation as described above.

The strategy described here combines in silico analysis of peptide sequence similarity combined with Mass spectroscopy analysis of eluted HLA peptides, peptide data bases and alanine scanning provides a tool box to fully control peptide search parameters, more than other tools such as BLAST or ScanProsite provide. Additional parameters are employed including the range of allowed peptide lengths, the maximum allowed number or differences in sequence, and the requirement for HLA binding score. The tool also applies the ability to define certain amino acids as equivalent. Most important is the ability to highlight peptides that have been found by mass spectrometry or by peptide databases.

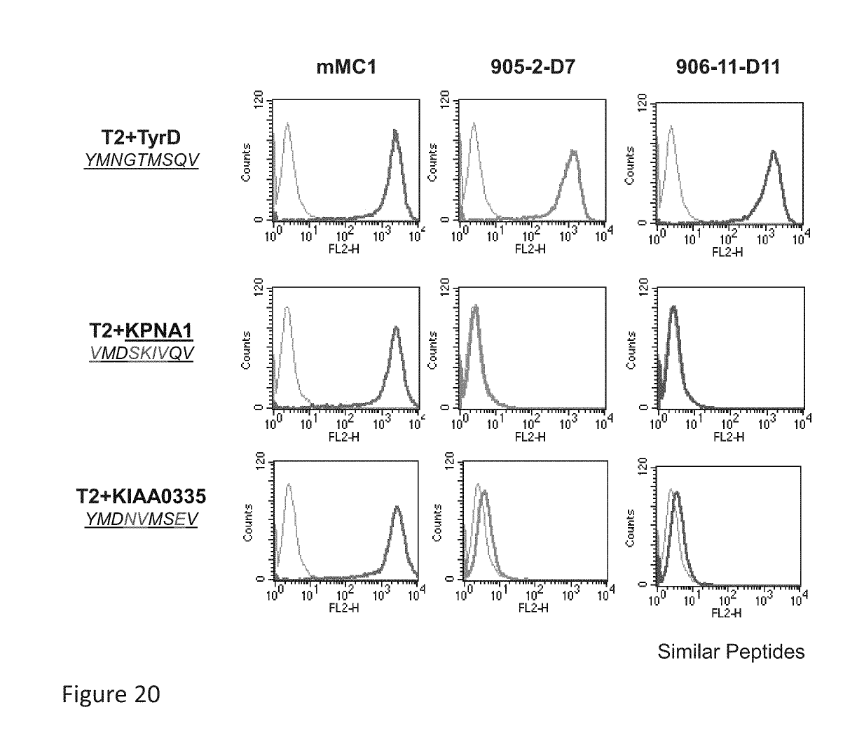

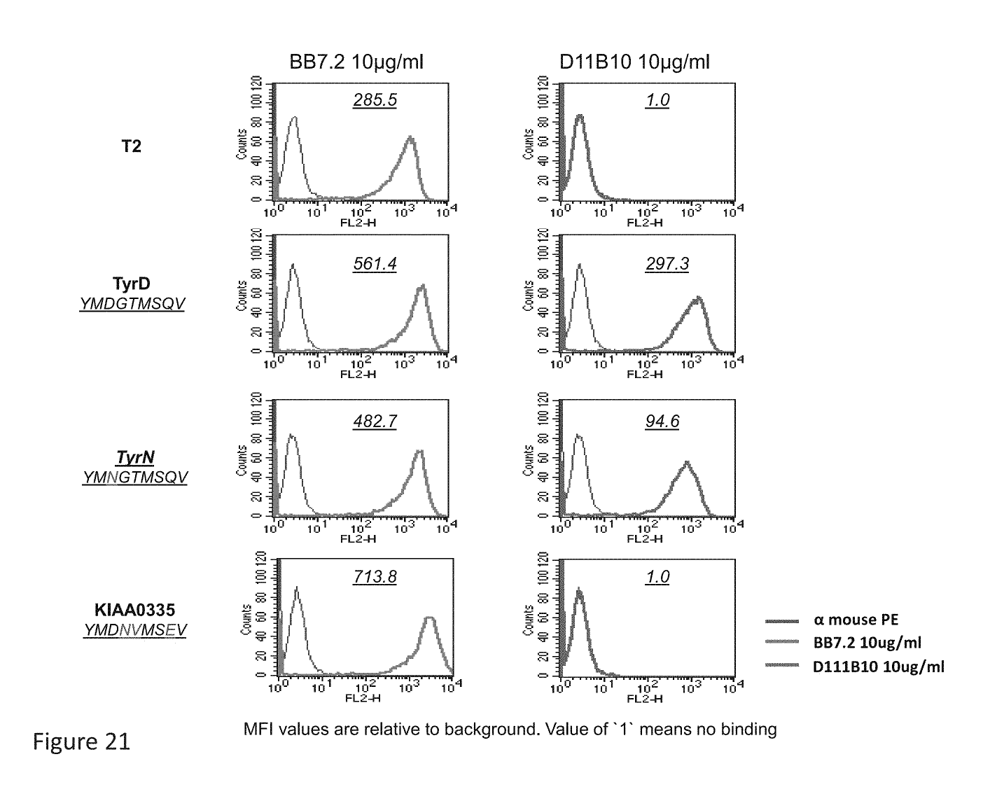

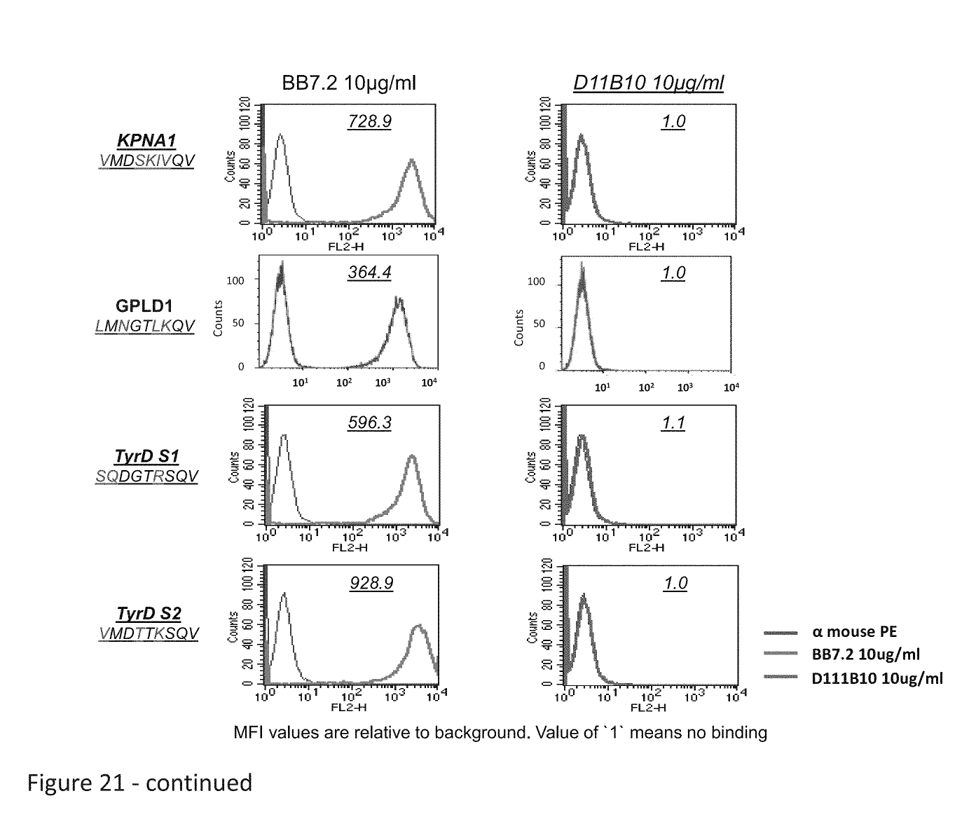

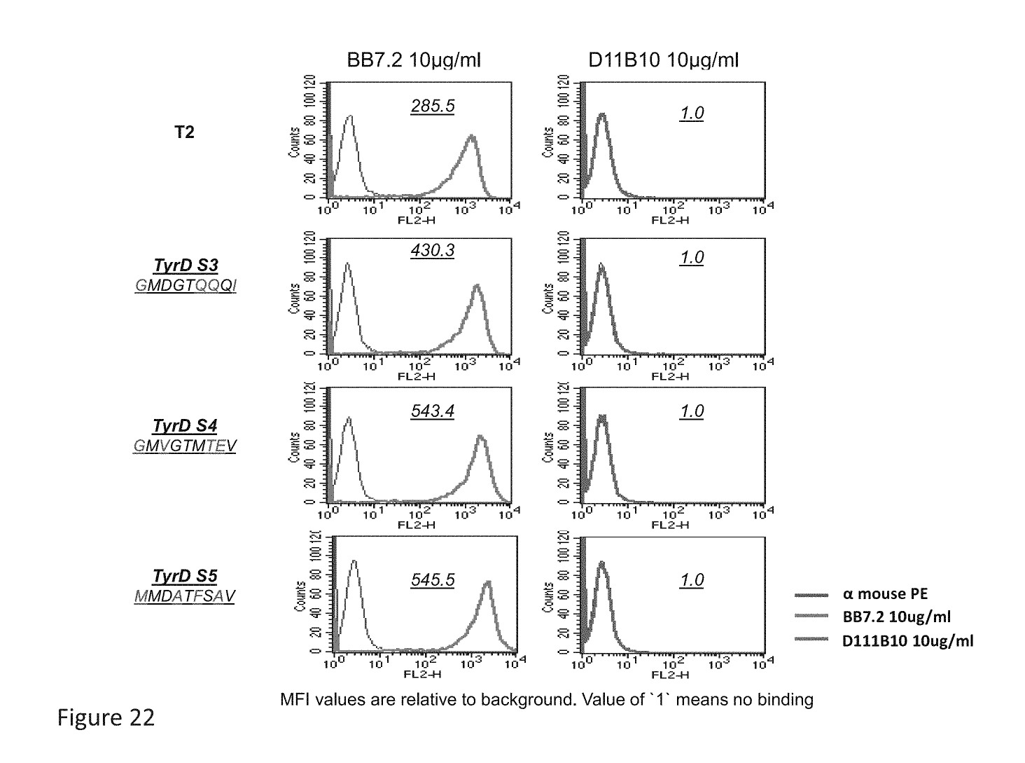

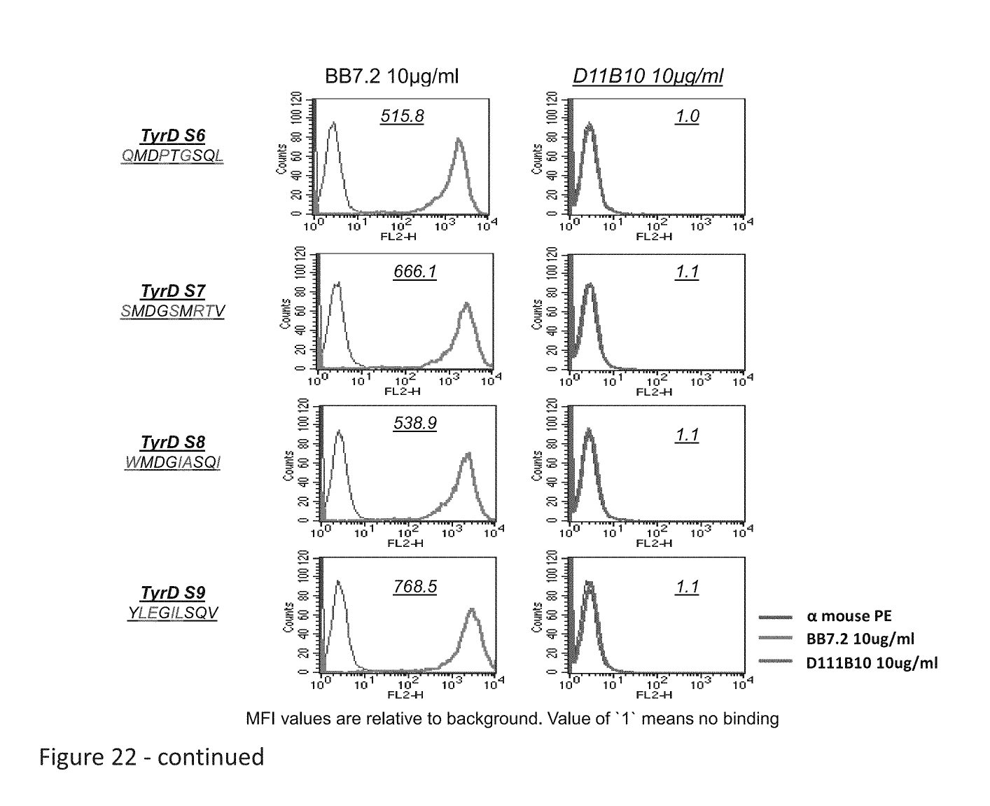

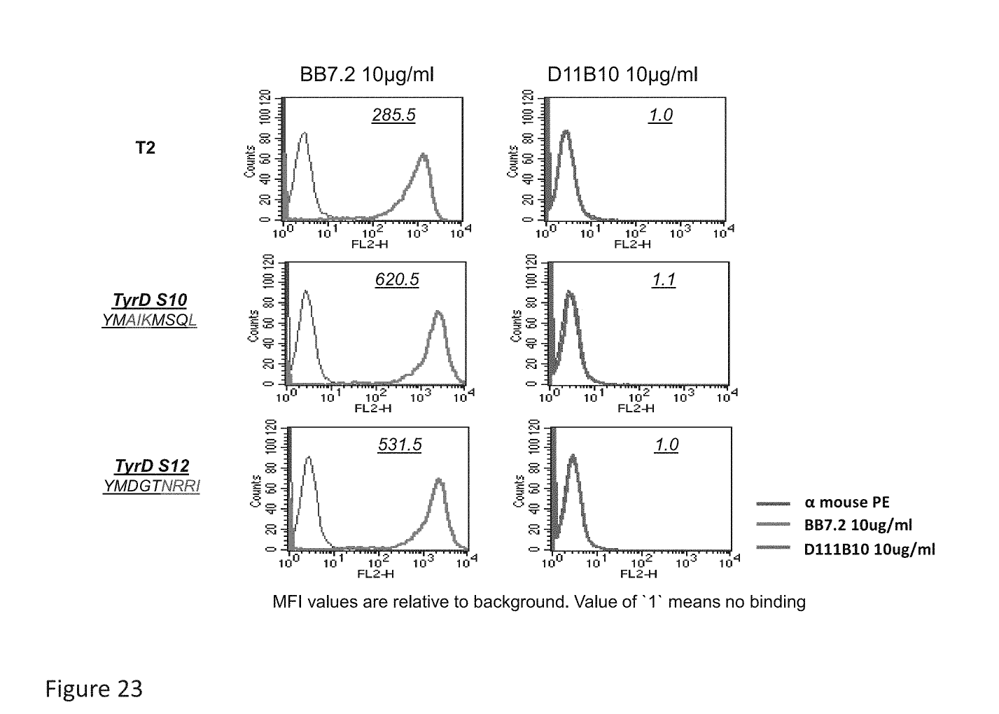

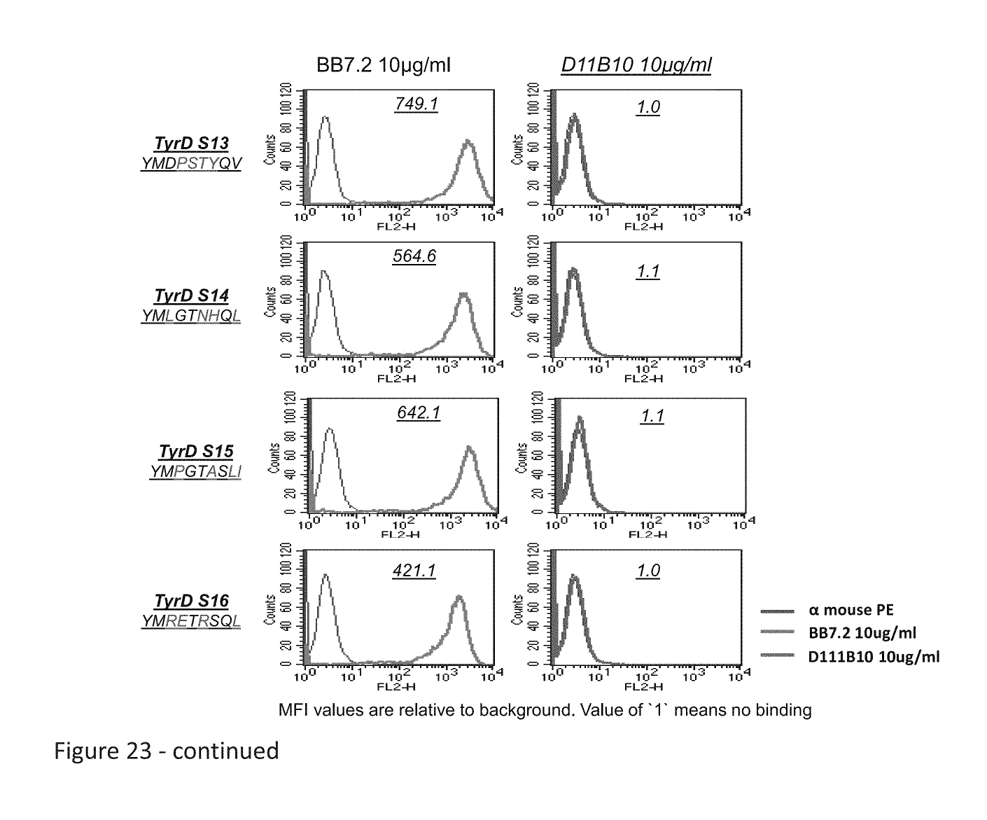

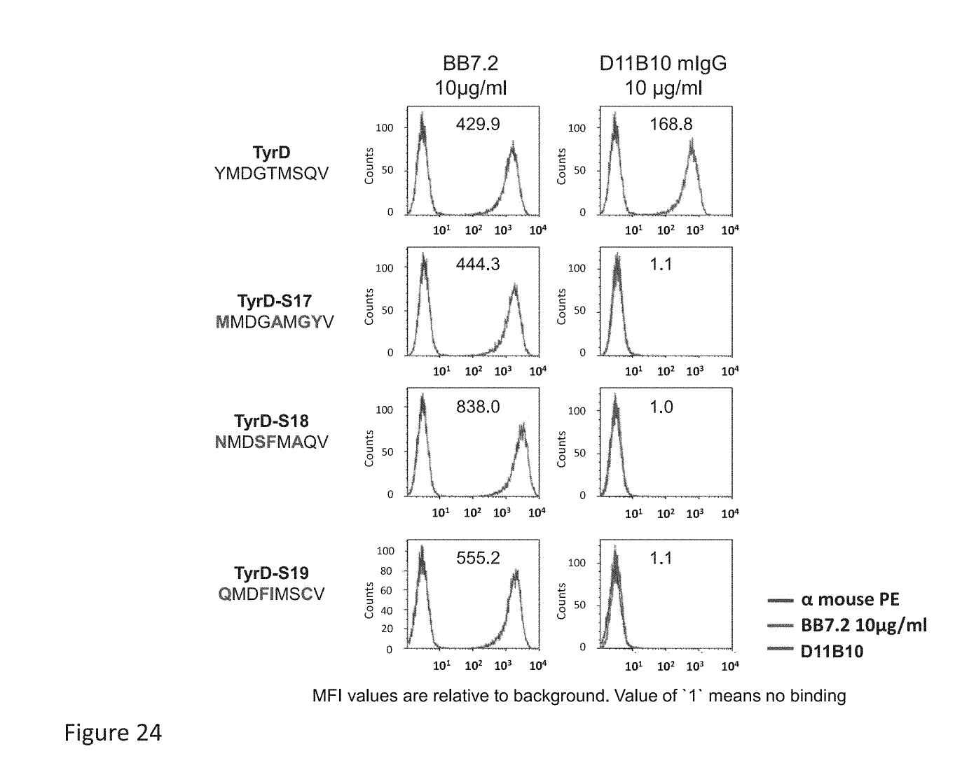

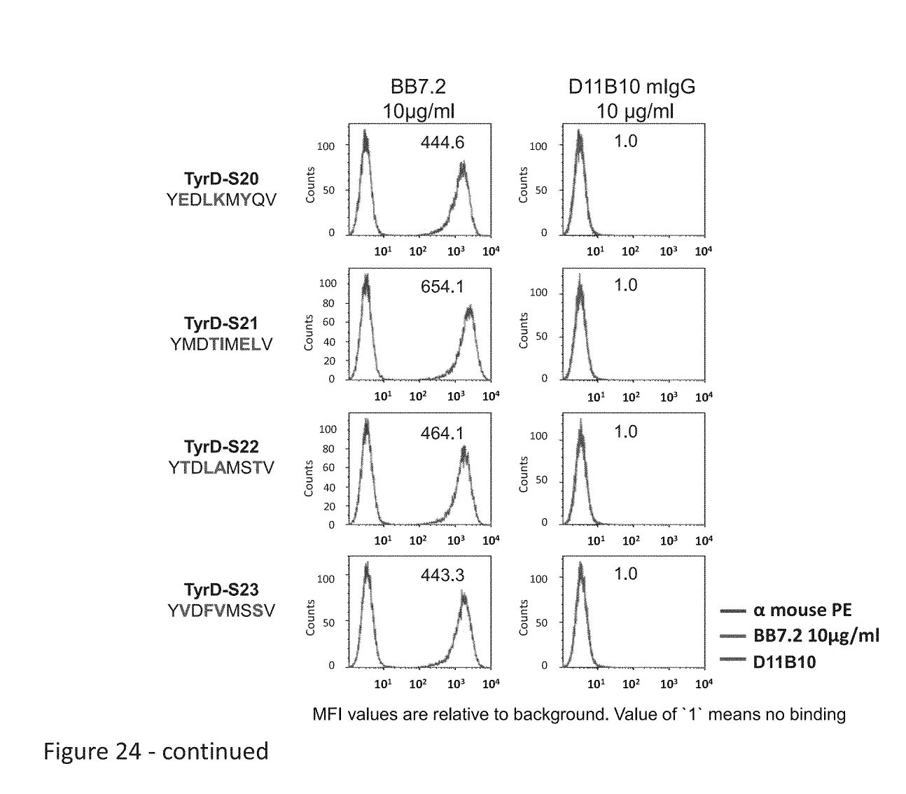

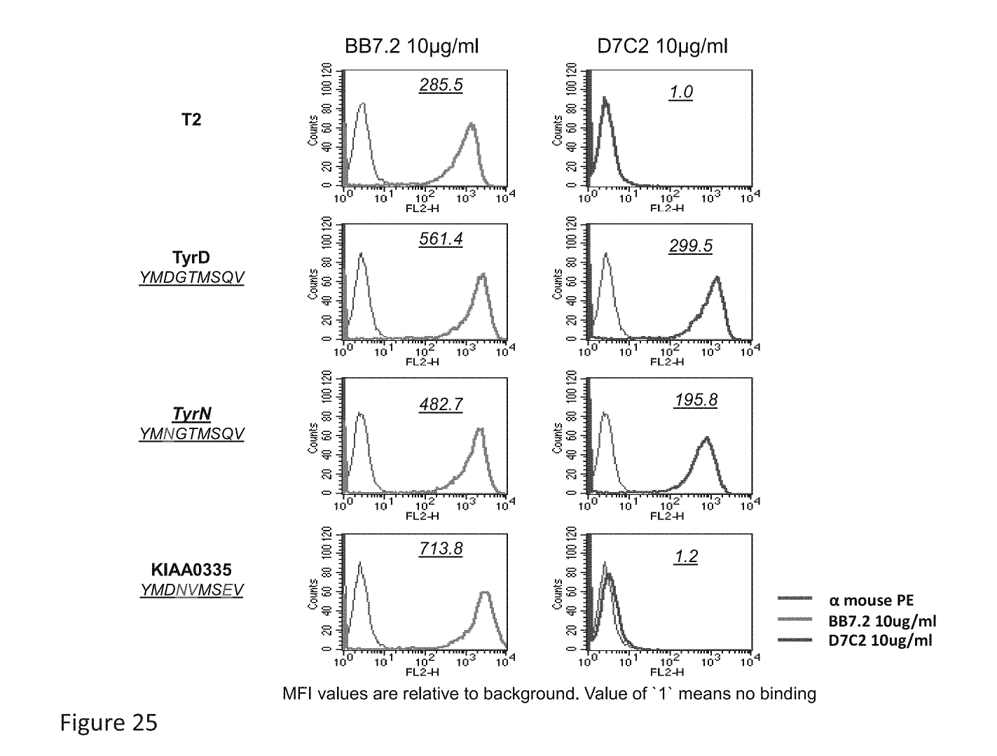

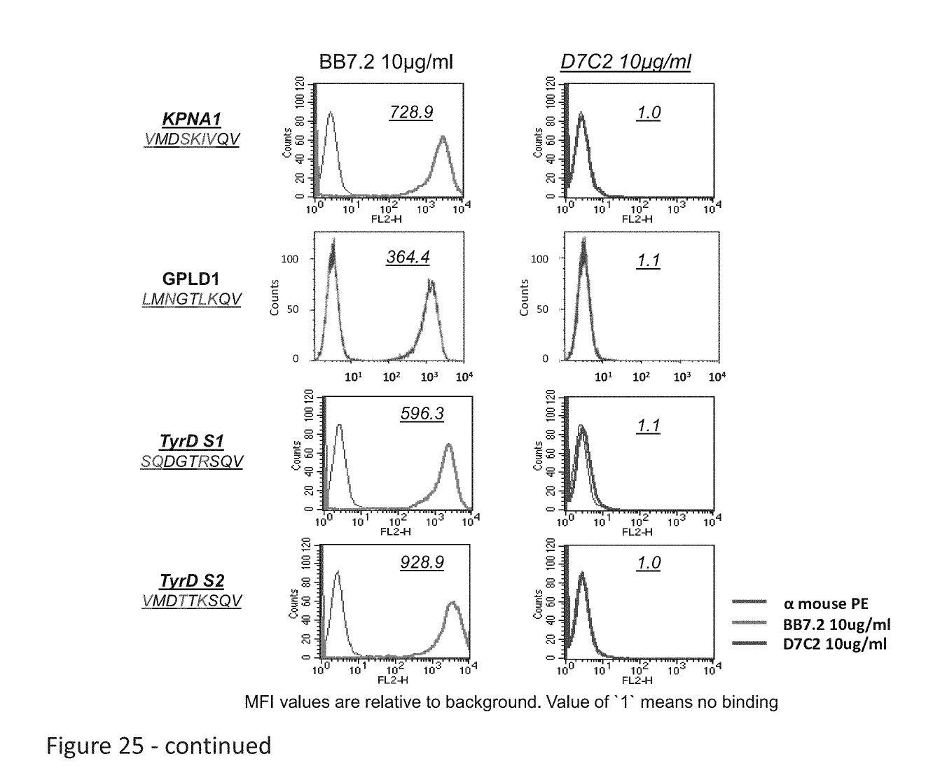

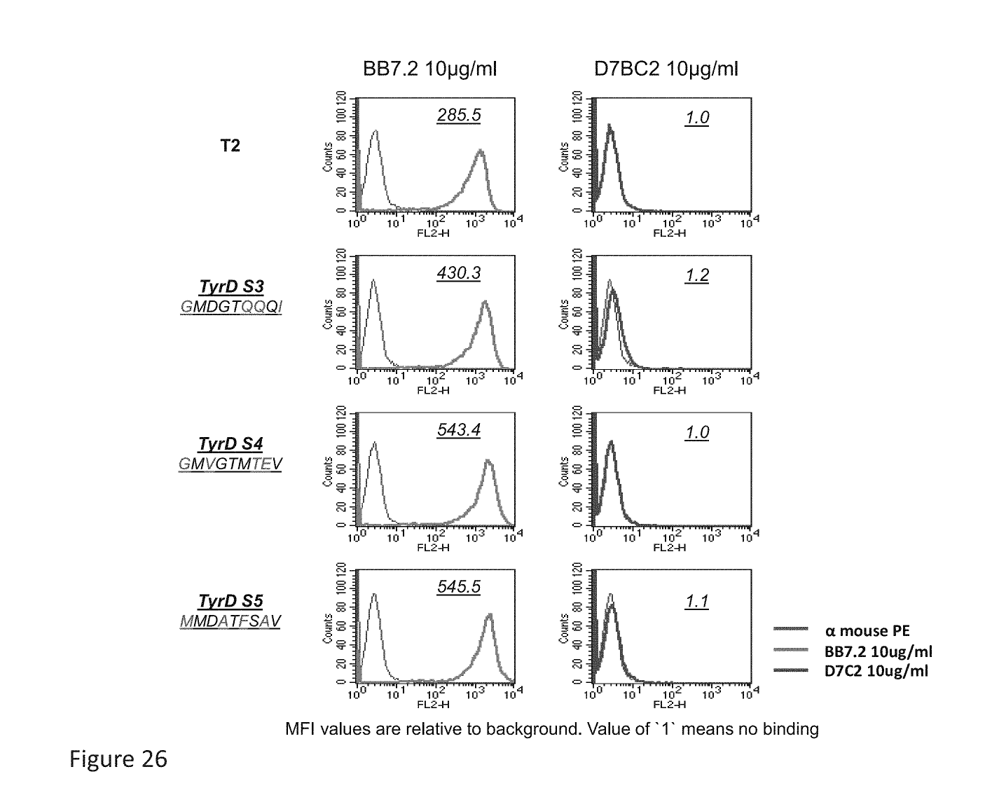

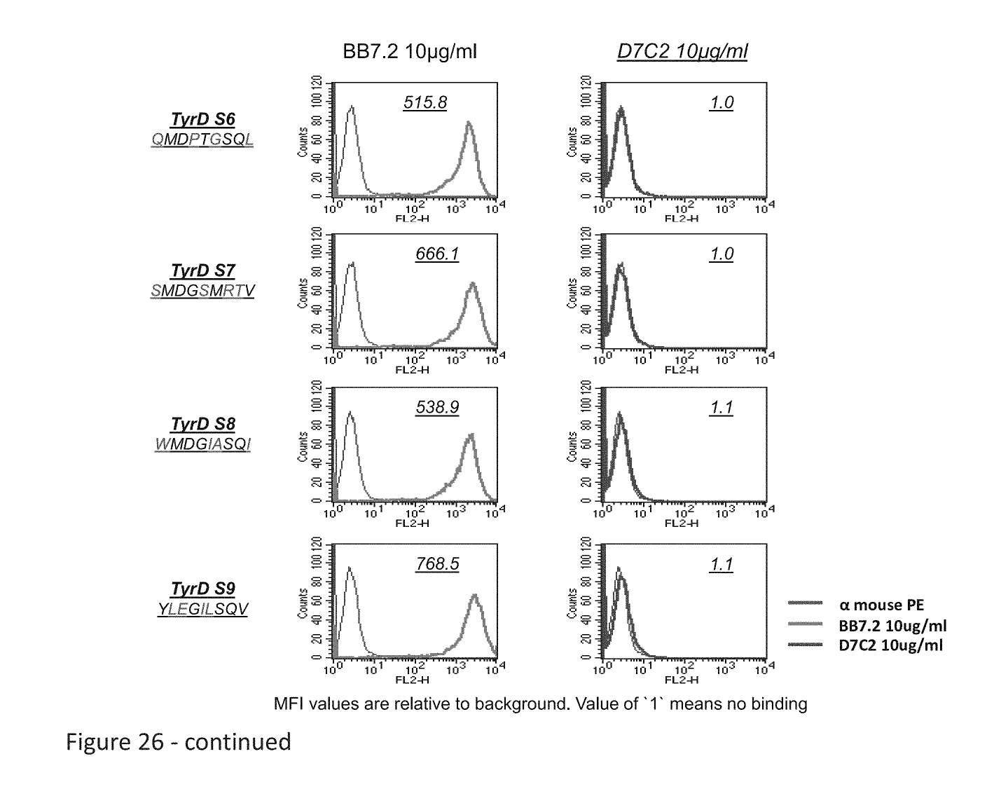

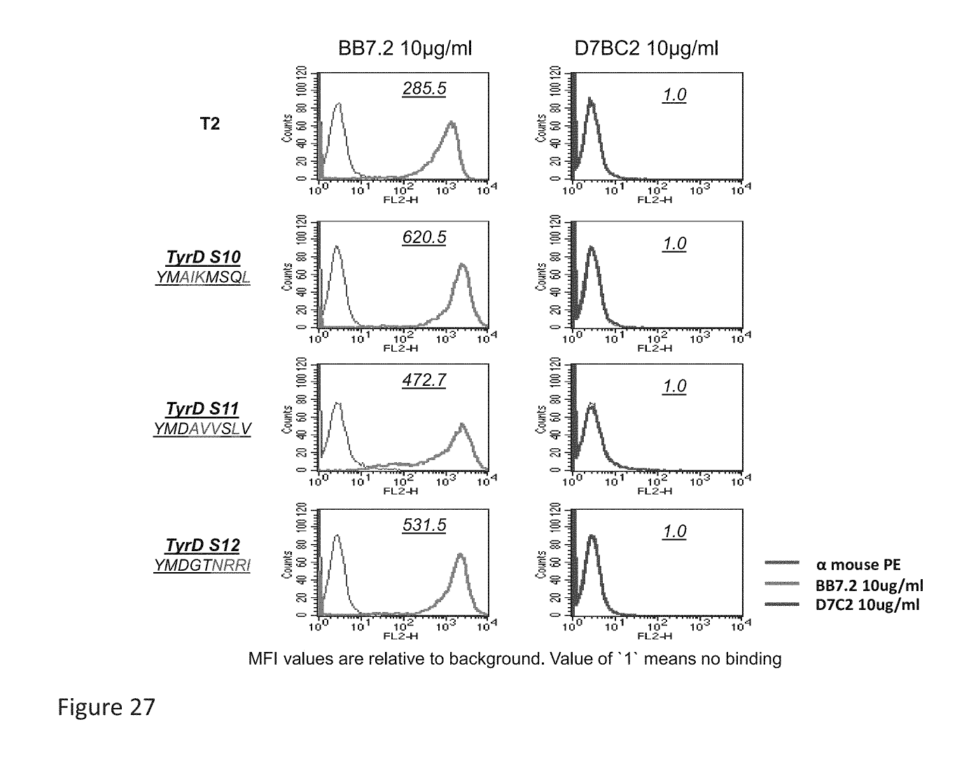

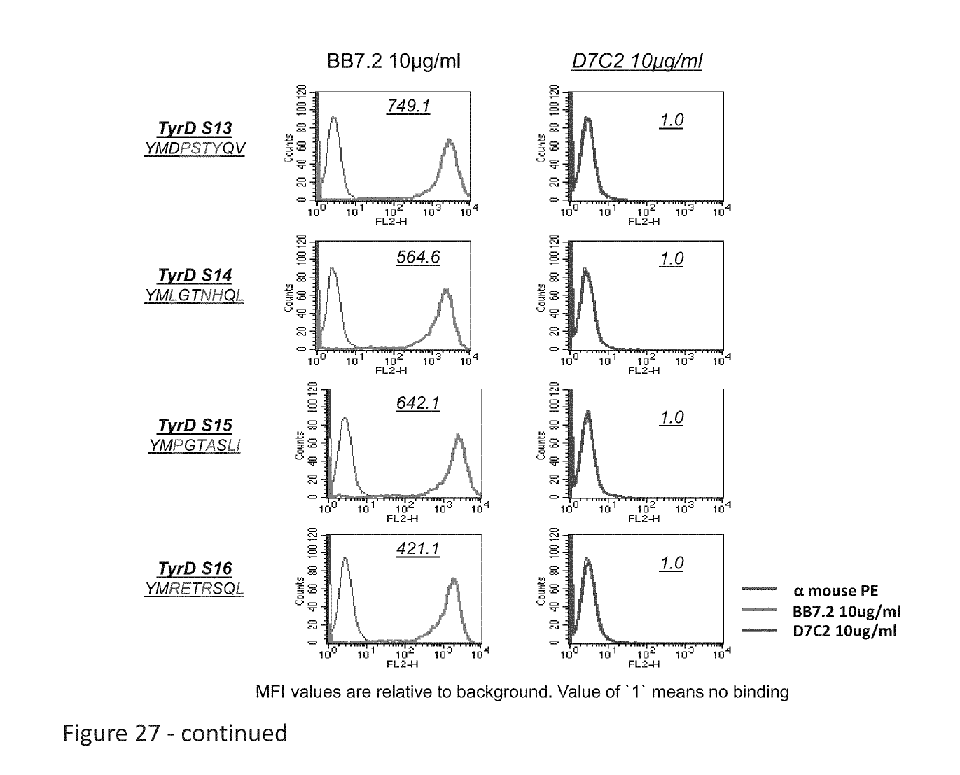

Applying the above tools, the fine specificity of the three TCR-like antibodies was evaluated by synthesizing a large panel of similar peptides that have been selected for evaluation according to the criteria described herein (Table 5). These similar peptides have been loaded on T2 APCs and the reactivity of the TCR-like antibodies was tested. As shown in FIG. 20, when MC1 was tested on a panel of similar peptides in comparison with binding to native tyrosinase peptide it was observed that it exhibits background binding to peptides with sequence similarity to Tyrosinase such as KIAA0335 and KPNA1. However, as shown in FIGS. 21-28, the D11 and D7 TCR-like antibodies did not bind any similar peptide from a large panel of such that were analyzed by peptide loading including no recognition of the KIAA0335 and KPNA1 peptides that exhibited background binding with MC1. These data demonstrate the superior selectivity and fine specificity of D11 and D7 in comparison to MC1 and demonstrates the usefulness of the similar peptide approach and tools developed as described above as important tools to evaluate the selectivity and fine specificity hierarchy when evaluating a panel of TCR-like antibodies for the best and optimal candidate for further evaluation.

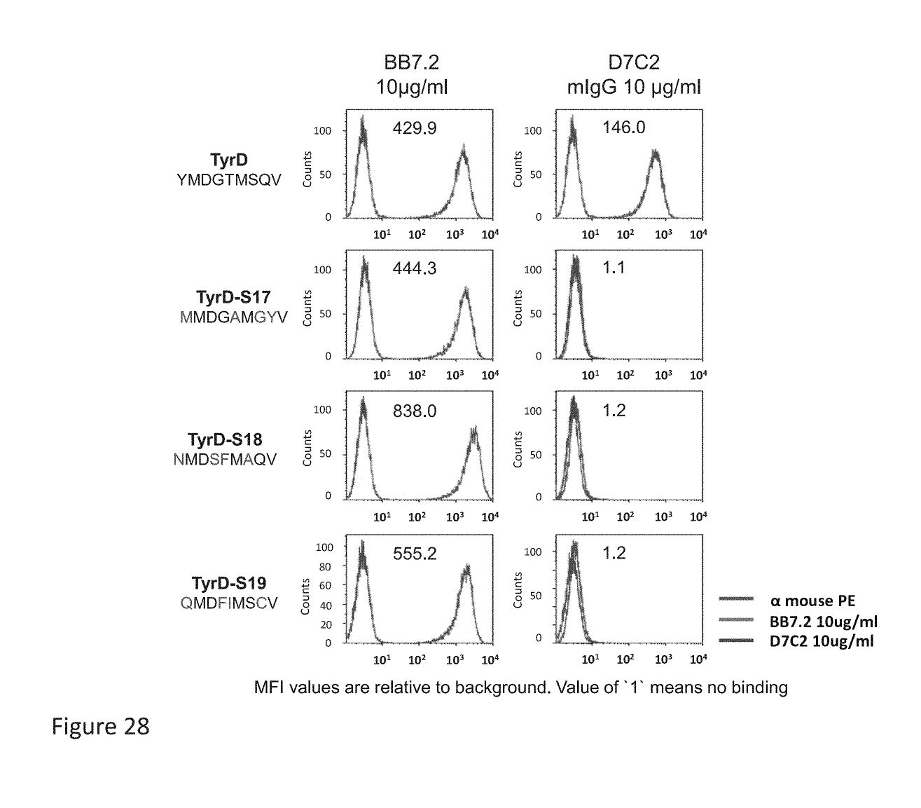

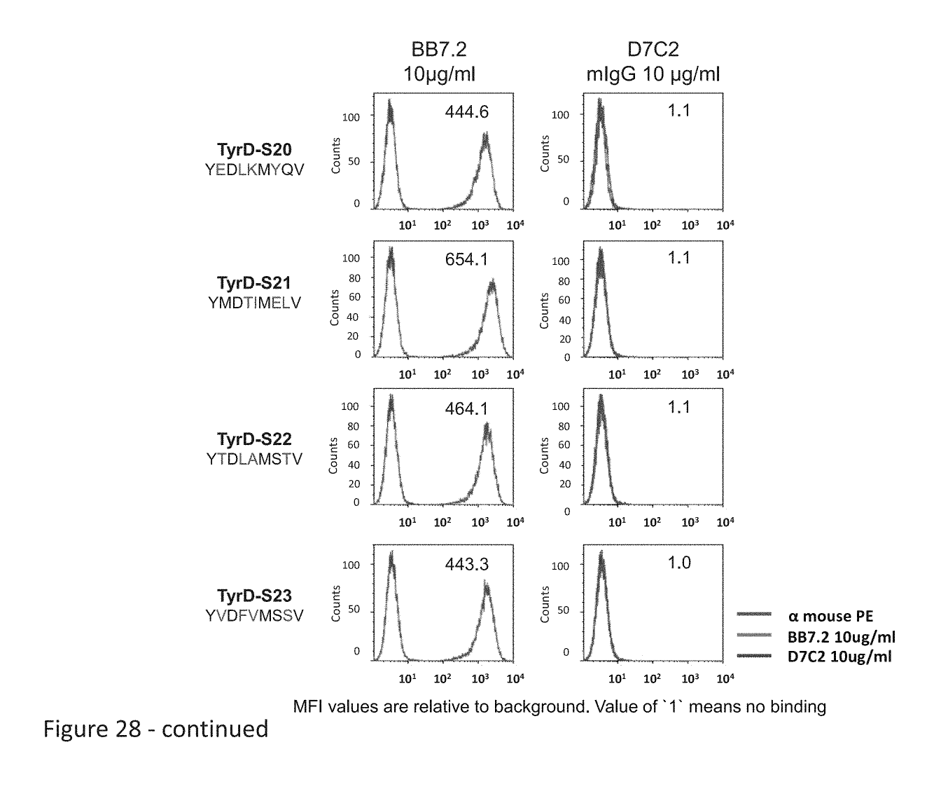

Moreover, after alanine scanning of TCR-like antibodies additional similar peptides have been selected and tested. Since each amino acid within the TyrD peptide sequence is unlikely to contribute equally to Tyr TCRL binding, the peptide residues critical for recognition by the Tyr TCRL were identified. A set of synthetic peptides were produced in which each amino acid of the TyrD 9-mer was sequentially replaced by alanine. The ability of Tyr TCRL to bind cells pulsed with each of these alanine-substituted peptides was determined by FACS analysis and the binding results was compared to those obtained with the non-mutated peptide. The residue at position that alanine substitution result in a large decrease in binding compared to the non-mutated peptide, was considered critical. A directed in-silico search was then carried out to identify protein sequences that contain only the critical positions motif. These peptides were also utilized for specificity evaluation of Tyr TCRLs (Table 5 S17-S23). These alanine scanning analysis-derived similar peptides were synthesized and loaded onto T2 APCs cells and the reactivity of D11 and D7 was tested. As show in FIG. 28, no binding to these peptides was observed, thereby further confirming and strengthening the fine specificity and selectivity of these TCR-like antibodies.

### Example Ia

### Characterization of TCR-Like Antibodies for HLA-A2/Tyrosinase

Comparison of the Fine Specificity of Abs with TCR-Like Specificity to HLA-A2/Tyrosinase369-377

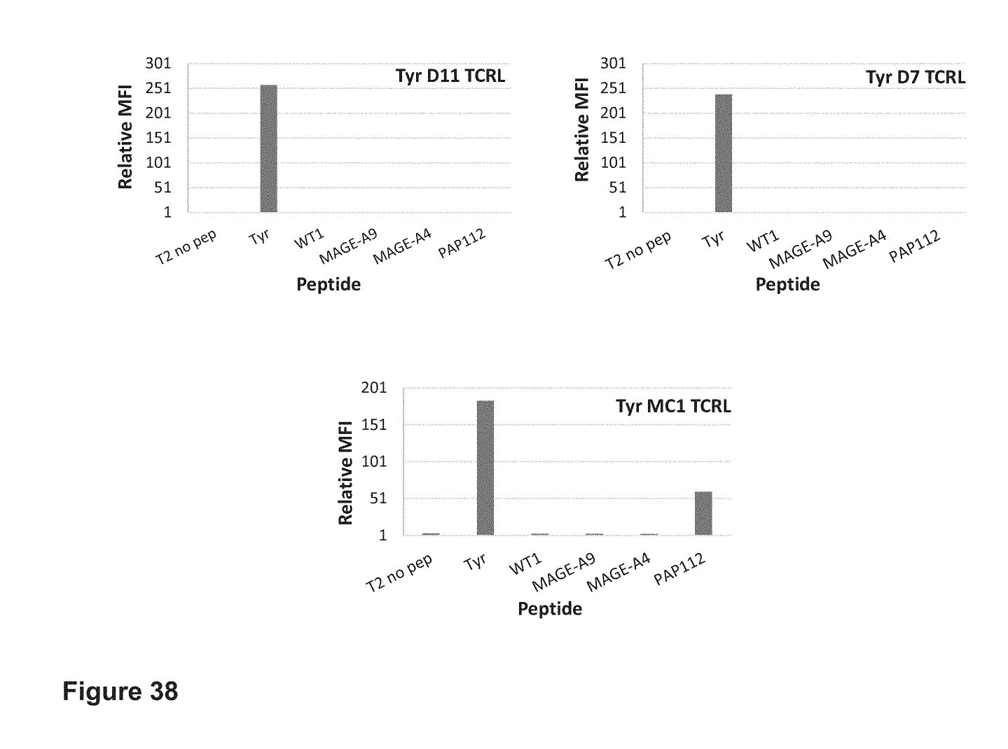

To characterize the binding specificity of the isolated TCR-like antibodies the reactivity and specificity of the purified IgGs (with or without biotinylation) were assessed by flow cytometry. T2 APCs were loaded with Tyrosinase peptide or control peptides (Table 15) and incubated with the Ab (D7, D11 or MC1), followed by incubation with PE-labeled streptavidin or PE-labeled anti mouse Abs. As shown in FIG. 38, D11, and D7 TCRLs bound T2 cells loaded with the tyrosinase peptide but showed no binding to cells loaded with control peptides. In contrast, MC1 TCRL showed binding to T2 cells loaded with both the Tyrosinase peptide and with the irrelevant peptide used as control.

To further evaluate the specificity of the D7 and D11 TCR-like antibodies their reactivity with peptides that exhibit sequence similarity to the tyrosinase peptide was evaluated. The peptides are shown in Table 5.

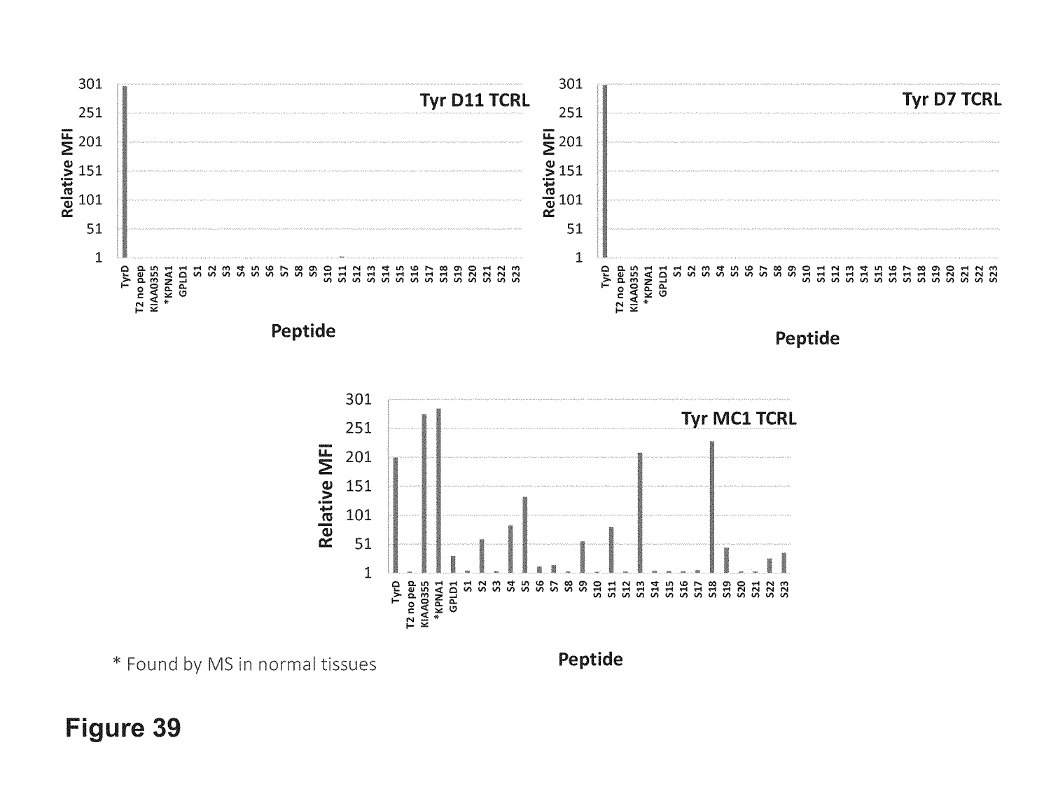

As shown in FIG. 39 MC1 TCRL exhibits readily detectable binding to various peptides with sequence similarity to Tyrosinase peptide such as KIAA0335 and KPNA1 (Table 14) as well as to peptides marked as S2, S4, S5, S9, S11, S13, S18, (S19, S22 and S23). D11 and D7 TCR-like antibodies did not bind any of the peptides from this same panel of similar peptides. These data demonstrate the superior selectivity and fine specificity of D11 and D7 TCRLs compared to MC1 TCRL and demonstrates the usefulness of the similar peptide approach and tools developed as described above to evaluate the selectivity and fine specificity hierarchy of TCRLs.

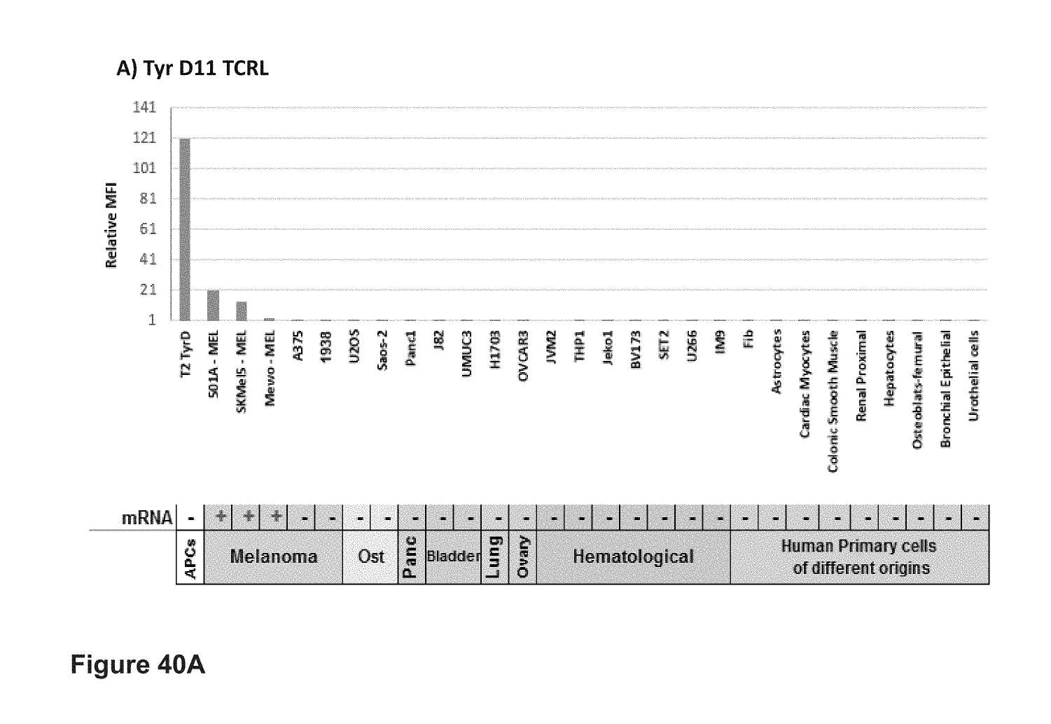

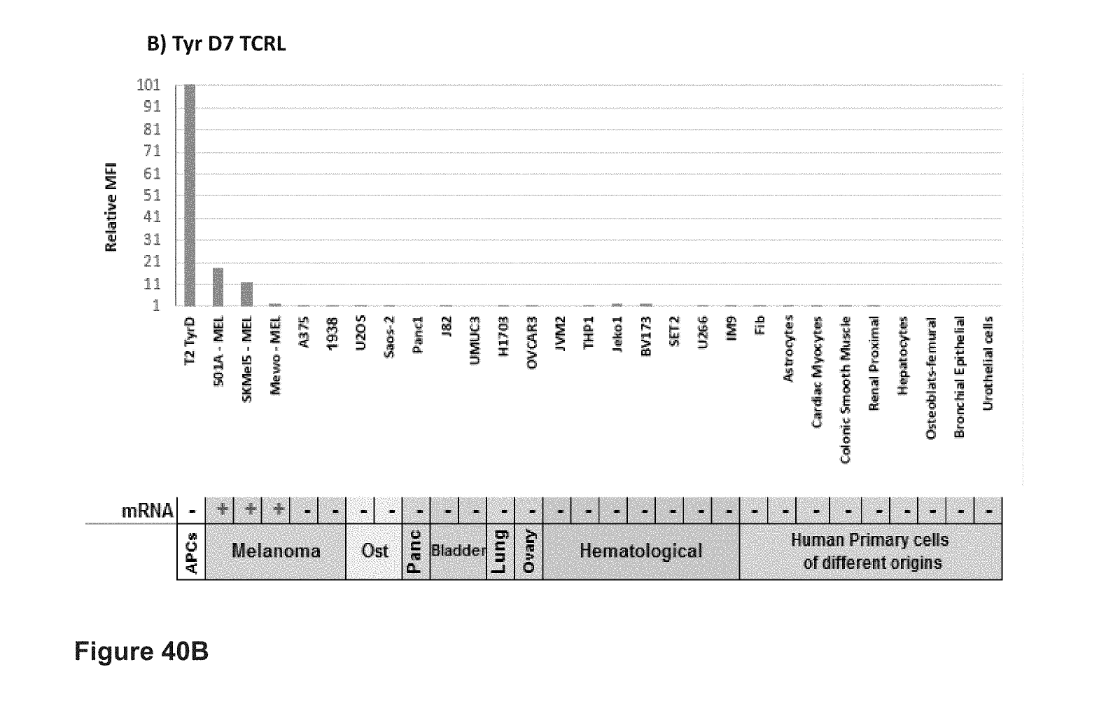

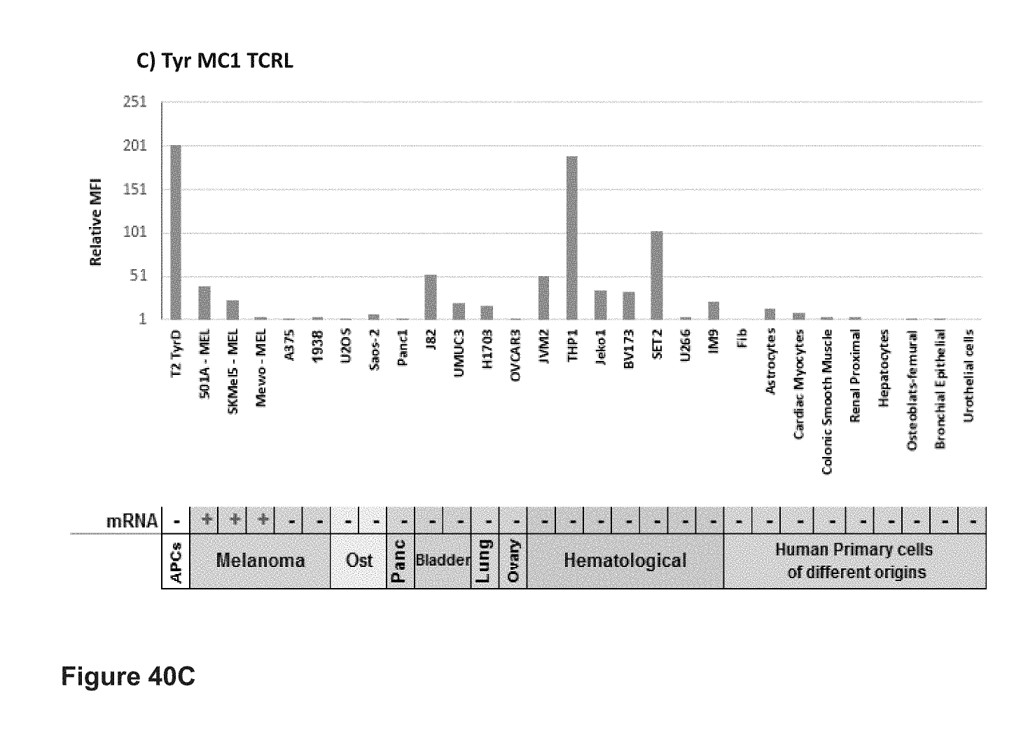

The present inventors explored binding specificity of the HLA-A2/tyrosinase TCR-like Abs to MHC-tyrosinase peptide complexes endogenously displayed on the surface of melanoma cell lines. Cells were incubated with anti-tyrosinase 369-377/HLA-A2 TCR-like antibodies Ab (with or without biotinylation) followed by incubation with PE-labeled streptavidin or anti-mouse Abs. A panel of tumor cells and normal primary cells that have been characterized for HLA-A2 (positive) and Tyrosinase (positive or negative) expression was used to compare the binding of the TCR-like antibodies. As shown in FIG. 40A-C, the TCR-like antibodies recognized tyrosinase-positive and HLA-A2-positive cells. The TCR-Like antibodies were tested on multiple HLA-A2-positive cell lines of various origin that do not show Tyr RNA expression (Tyr-negative). As shown in FIGS. 40A-B, D11 and D7 TCRLs did not bind any of these cells while MC1 readily stained various HLA-A2+/Tyr− cells. D7 and D11 TCRLs did not exhibit any binding to normal primary cells, while MC1 displayed detectable binding to some of them (FIG. 40C).

Overall, D7 and D11 TCRLs demonstrated superior specificity and selectivity recognizing tyrosinase peptide presented by HLA-A2 compared to MC1 TCRL.

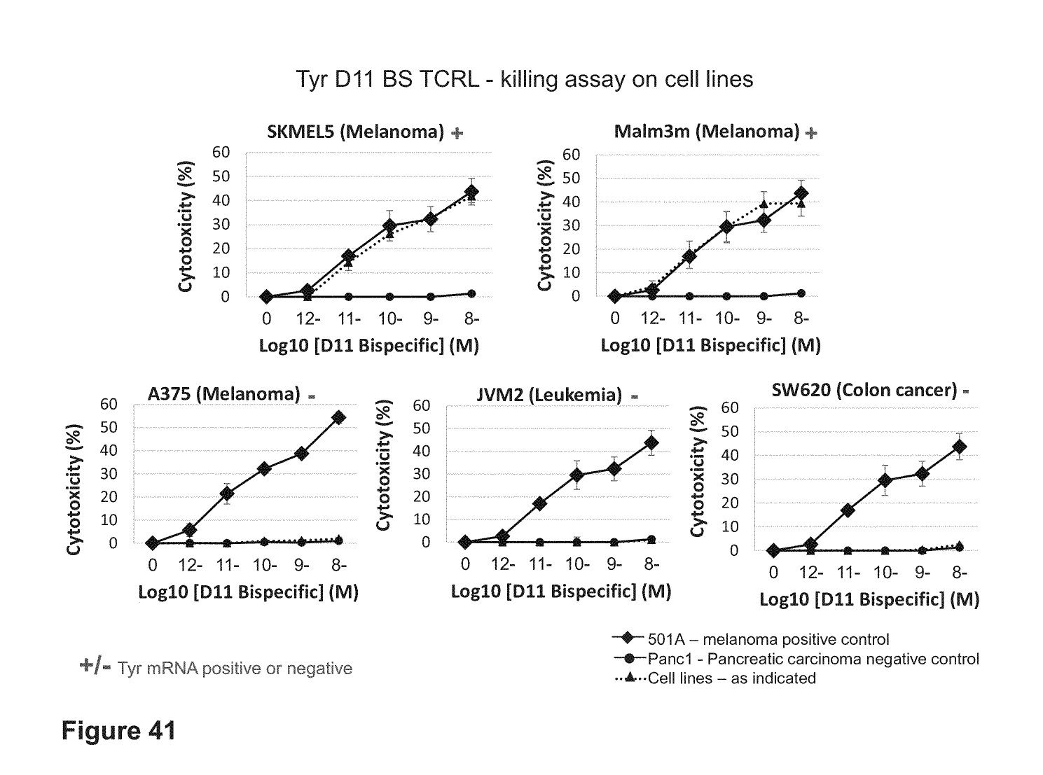

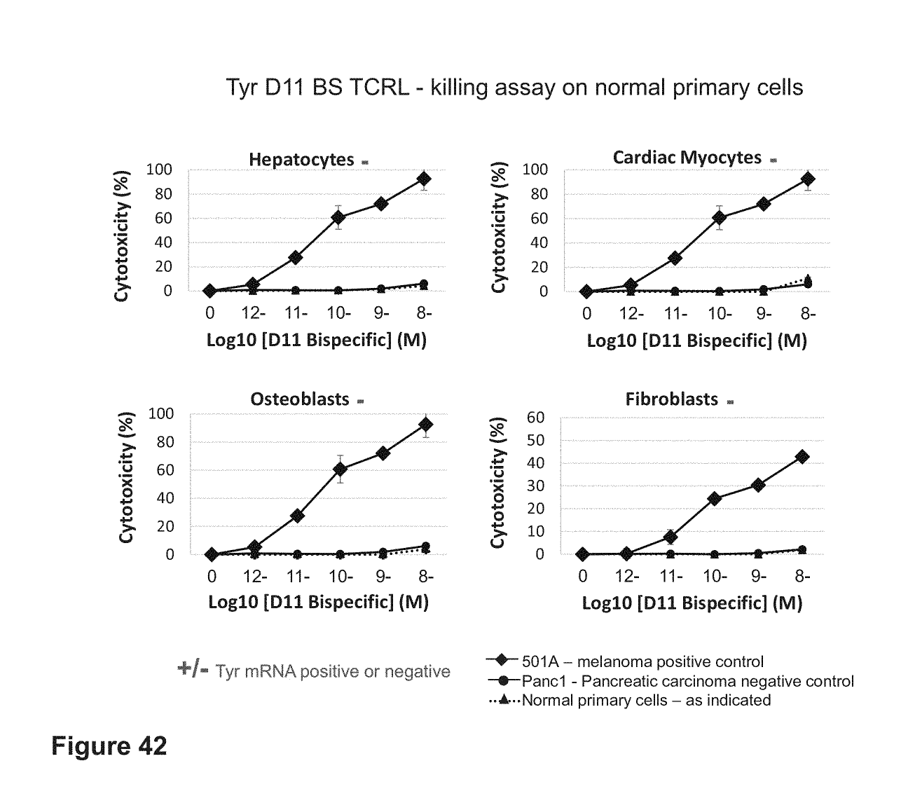

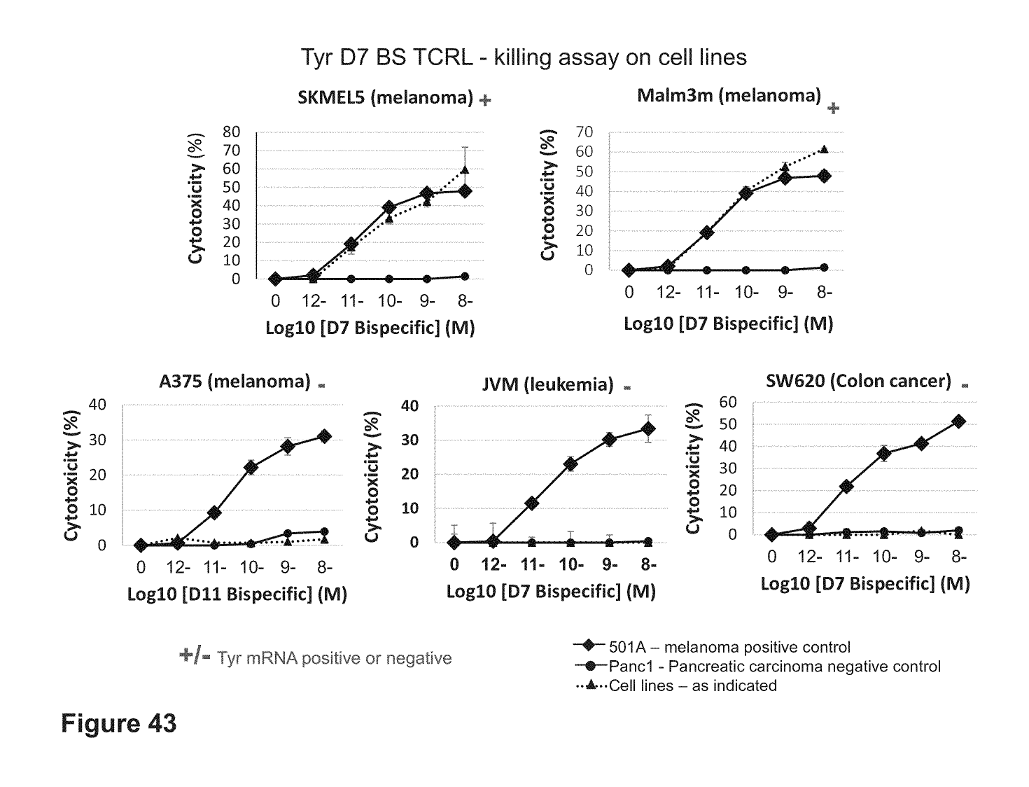

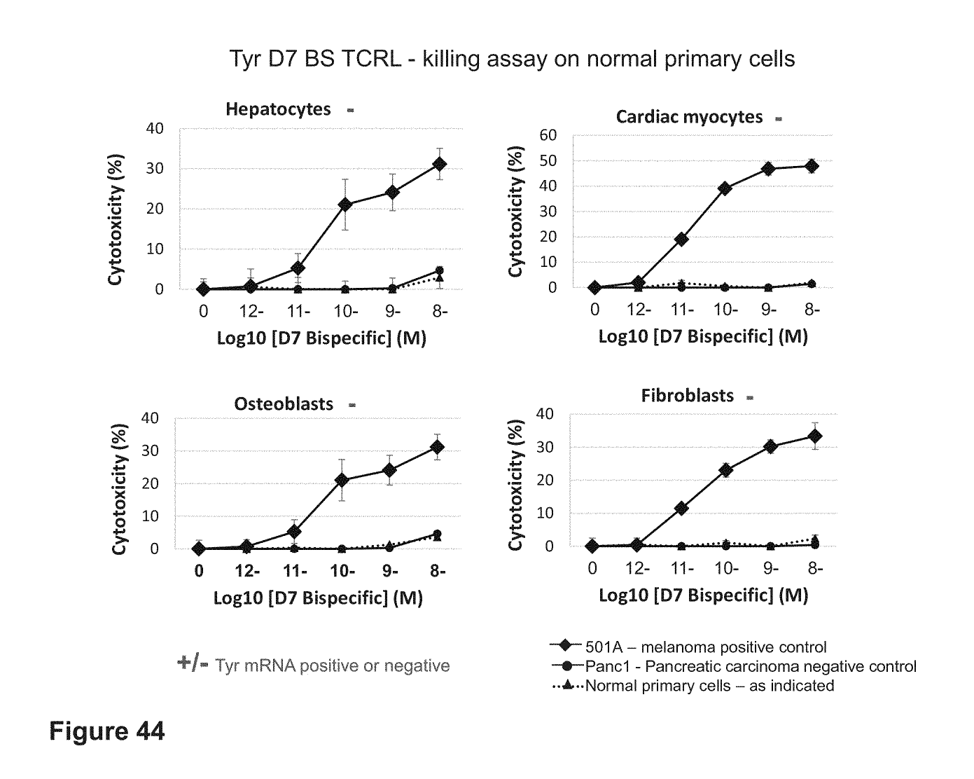

Functional assays were used to further characterize the D7 and D11 TCR-like antibodies. TCRLs variable regions were fused to an anti-CD3 scFv which can re-target effector T cells to kill tumor target cell in a of bi-specific format. As shown in FIGS. 41-44, D7 and D11 CD3 Bi-specific TCR-like antibody constructs showed robust cytotoxicity against melanoma 501A cells in vitro in the presence of human PBMCs. Panc-1, Tyrosinase negative cell line served as negative control and demonstrated no cytotoxocitiy. No cytotoxicity was detected against a panel of HLA-A2+/Tyr− normal human primary cells with D7 and D11 TCRLs confirming their selectivity.

### Example Ib

### In Vivo Efficacy of D7 BS TCRL in s.c. 501A Melanoma Tumor Formation Model in NOD/SCID Mice

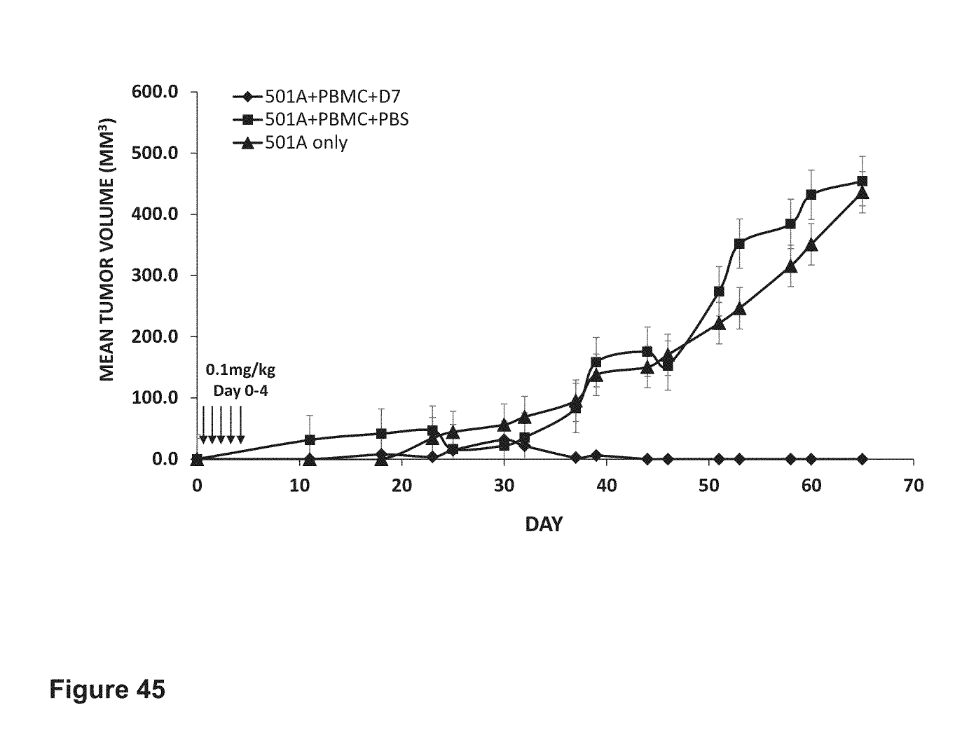

FIG. 45 shows in vivo efficacy of D7 BS TCRL in S.C. 501A melanoma tumor formation model in NOD/SCID mice. Clearly, administration of the bispecific antibody completely inhibited tumor formation over 65 days of the experiment, as evidenced by tumor volume. The results support the use of variable sequences of the TCRLs described herein in clinical settings.

### Example II

### TCR-Like Antibodies for HLA-A2/WT1

Isolation and Characterization of Abs with TCR-Like Specificity to HLA-A2/Wt1

To generate such antibodies with a specificity to the HLA-A2/WT1 complex, recombinant peptide-HLA-A2 complexes were generated that present the WT1 peptide

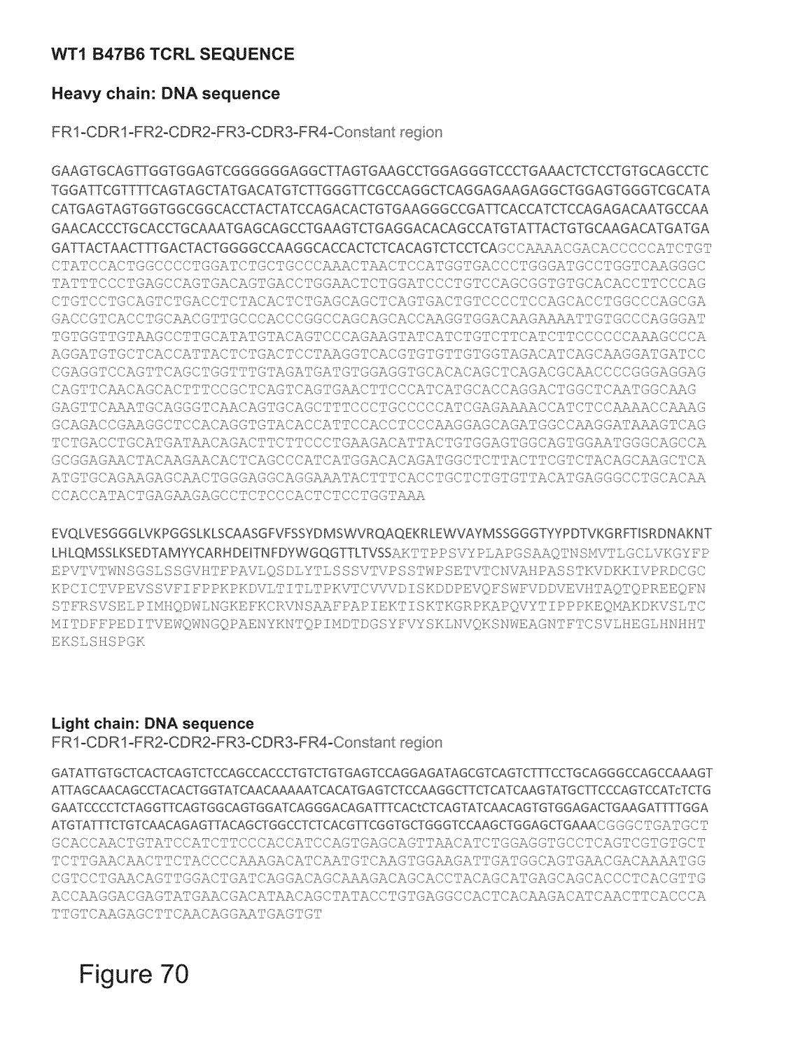

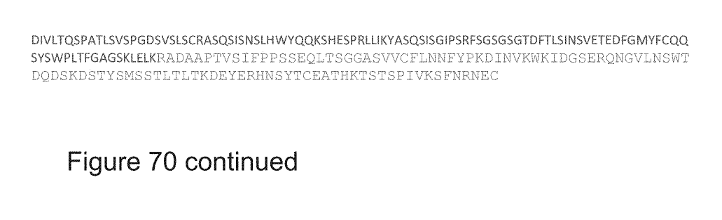

(RMFPNAPYL, SEQ ID NO: 151) using a single chain MHC construct. The generation of antibodies was as described in the general materials and methods as well as in Example I above, A TCR-like specific clone termed B47 (also referred to as B47B6) was isolated and characterized (FIG. 70).

As a comparison for TCR-like antibody binding selectivity, a TCR-like antibody termed ESK1 Dao T, Yan S, Veomett N, Pankov D, Zhou L, Korontsvit T, Scott A, Whitten J, Maslak P, Casey E, Tan T, Liu H, Zakhaleva V, Curcio M, Doubrovina E, O'Reilly R J, Liu C, Scheinberg D A.

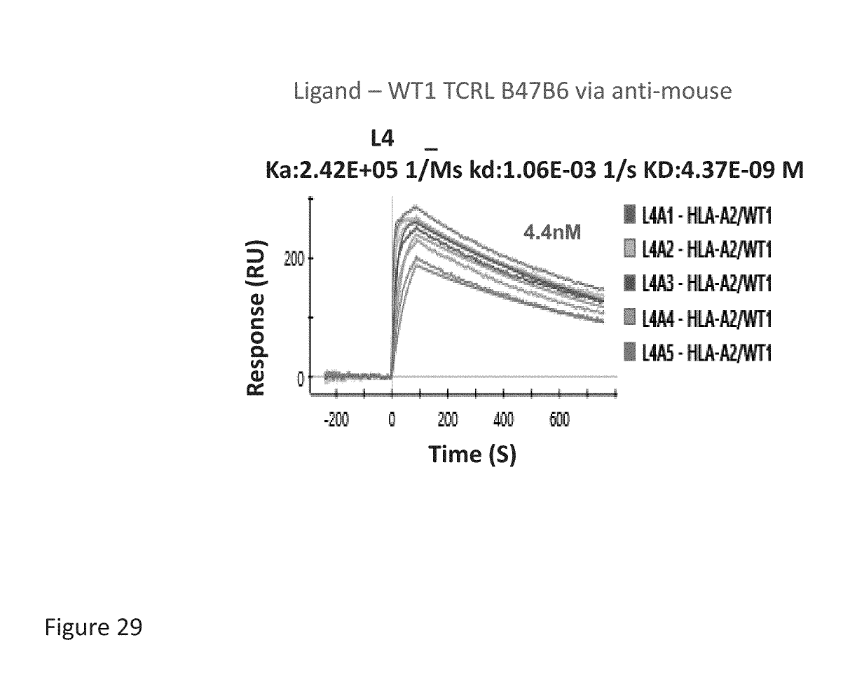

The binding affinity of B47 was evaluated by surface plasmon resonance (SPR) binding analysis in which the isolated purified IgG TCR-like antibody was immobilized to the SPR sensor chip by using anti-mouse IgG to indirectly immobilize the TCR-like antibodies on the chip surface. The analyte is the purified single-chain recombinant HLA-A2/WT1 complex used at various concentrations. As shown in FIG. 29, the sensorgrams of SPR analysis revealed an affinity for the HLA-A2/WT1 specific TCR-like antibody clone B47 of 4.4 nM.

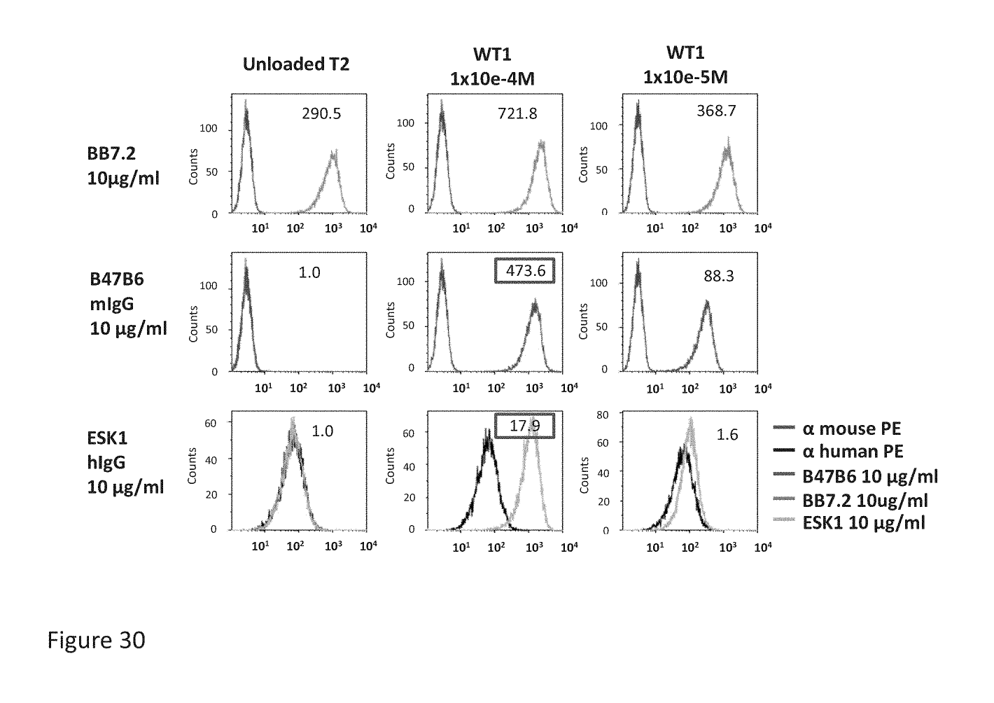

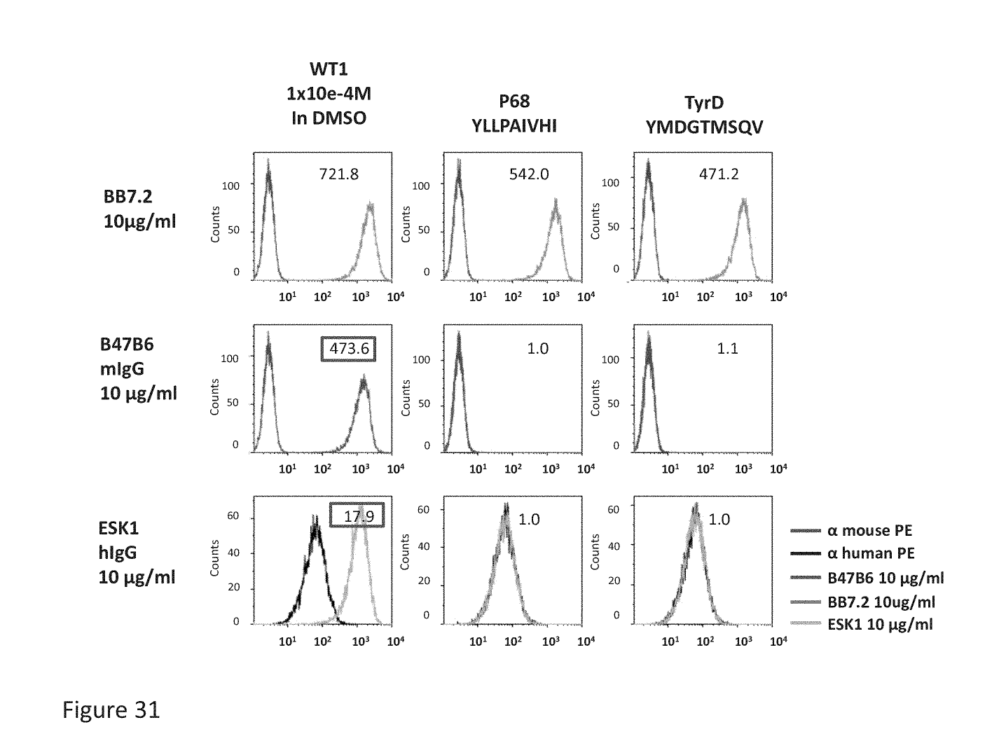

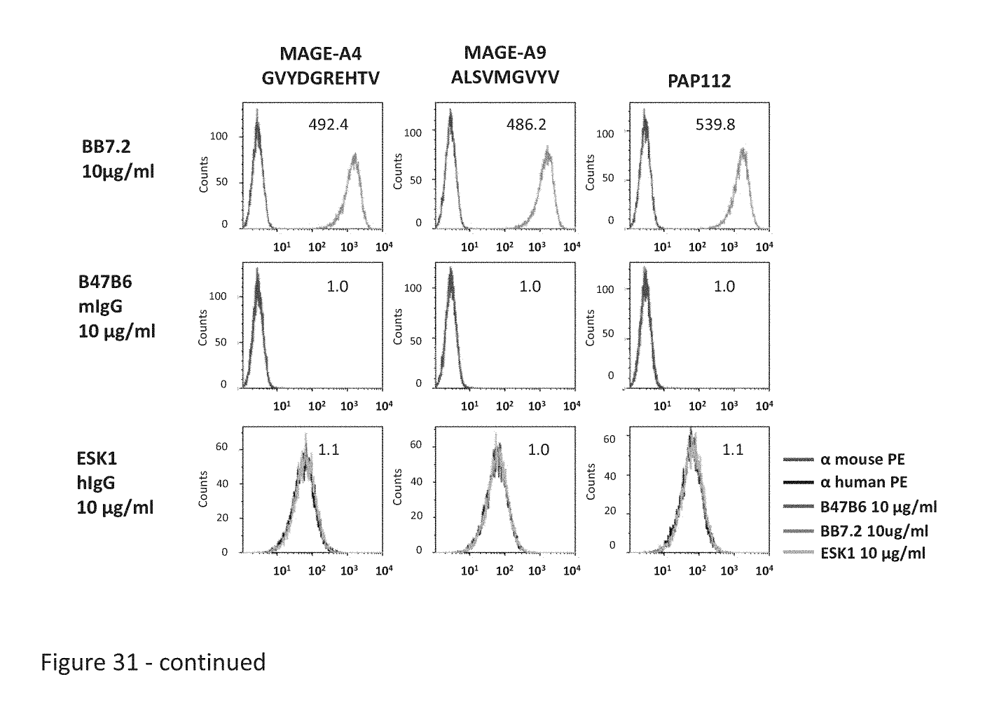

To characterize the binding specificity of the isolated TCR-like antibodies the reactivity and specificity of the purified IgGs were assessed by flow cytometry. T2 APCs were loaded with specific or control peptides (Table 15) and incubated with the Ab, followed by incubation with PE-labeled anti-human or mouse Ab. As shown in FIGS. 30 and 31, B47 and ESK1 bound T2 cells loaded with the WT1 peptide (FIG. 30) but did not bind to cells loaded with control peptides (FIG. 31). Of significance difference was the binding intensity observed for B47 and ESK1. While B47 bound intensely to T2 cells loaded with 10⁻⁴-10⁻⁵M peptide, ESK1 bound much weaker to T2 cells loaded with 10⁻⁴M WT1 peptide (MFI 18 for ESK1 compared with 474 for B47). At peptide concentration of 10⁻⁵M B47 still bound significantly (MFI 88) while binding of ESK1 was almost undetectable or very low (FIG. 30). These results indicated marked differences in the affinity and binding sensitivity of B47 compared to ESK1 with sharp decrease in the binding intensity of ESK1 compared to B47 with 10× decreases in peptide concentration. B47 and ESK1 did not bind T2 APCs loaded with control HLA-A2 restricted peptides (FIG. 31). These results indicate that both TCR-like antibodies exhibited HLA-A2-restricted peptide-specific binding as they bound only to cells presenting the WT1 but no other HLA-A2 restricted peptides.

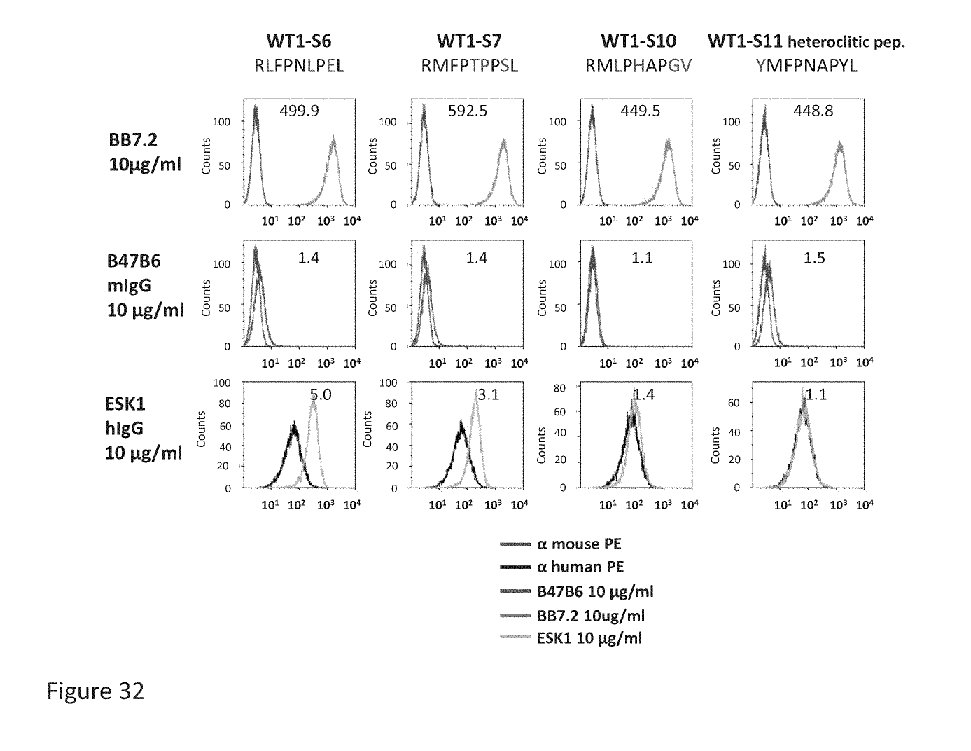

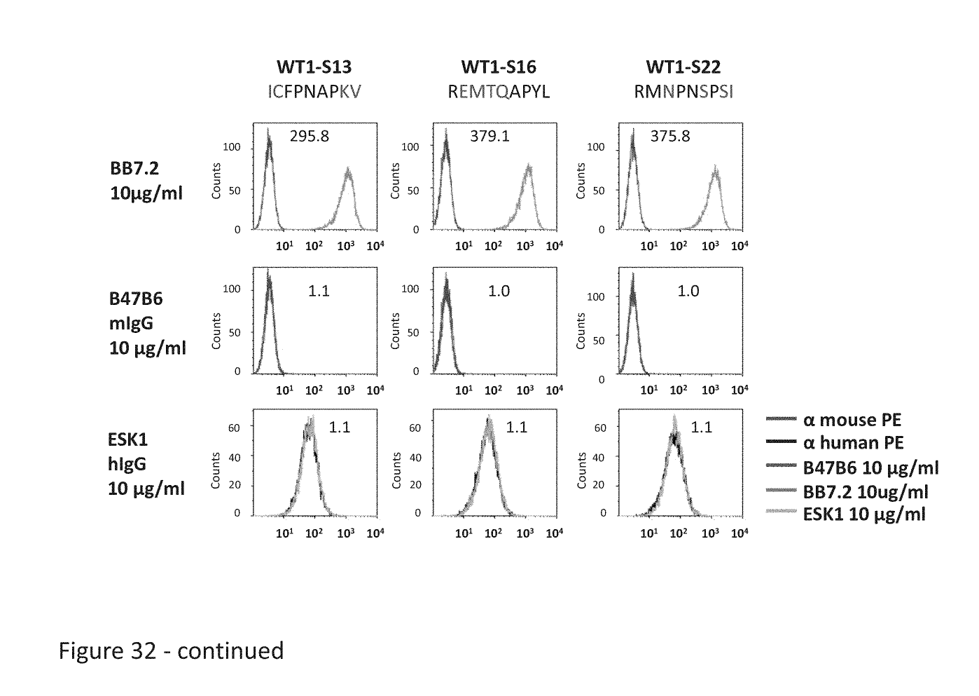

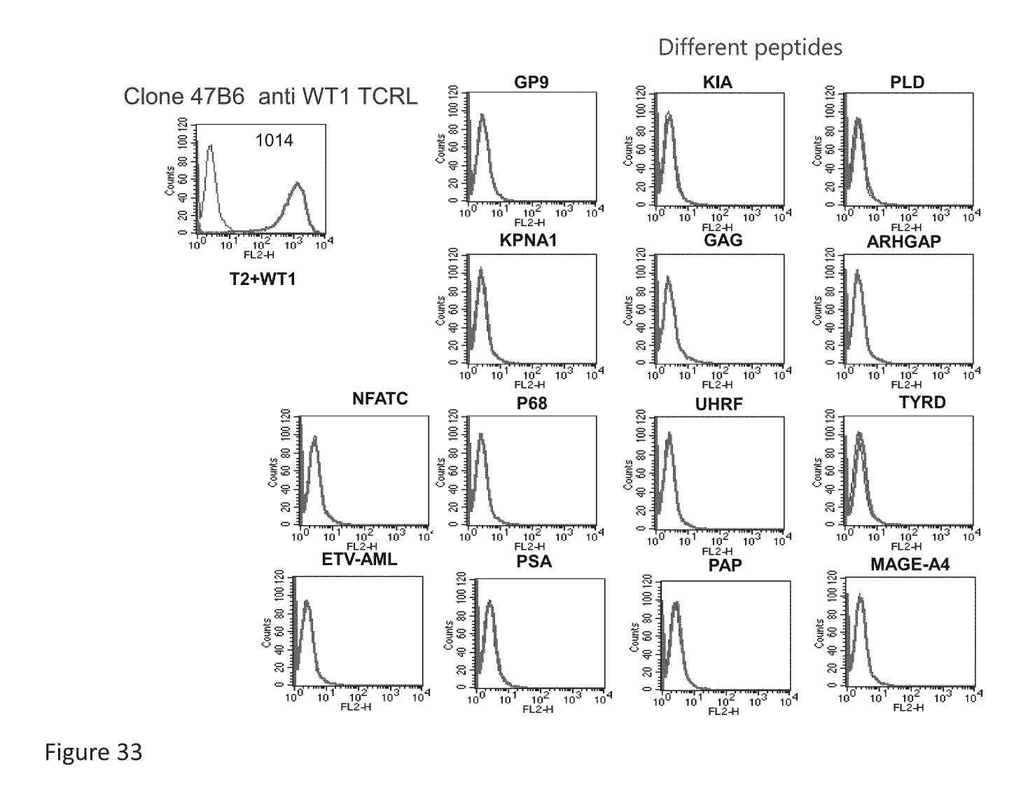

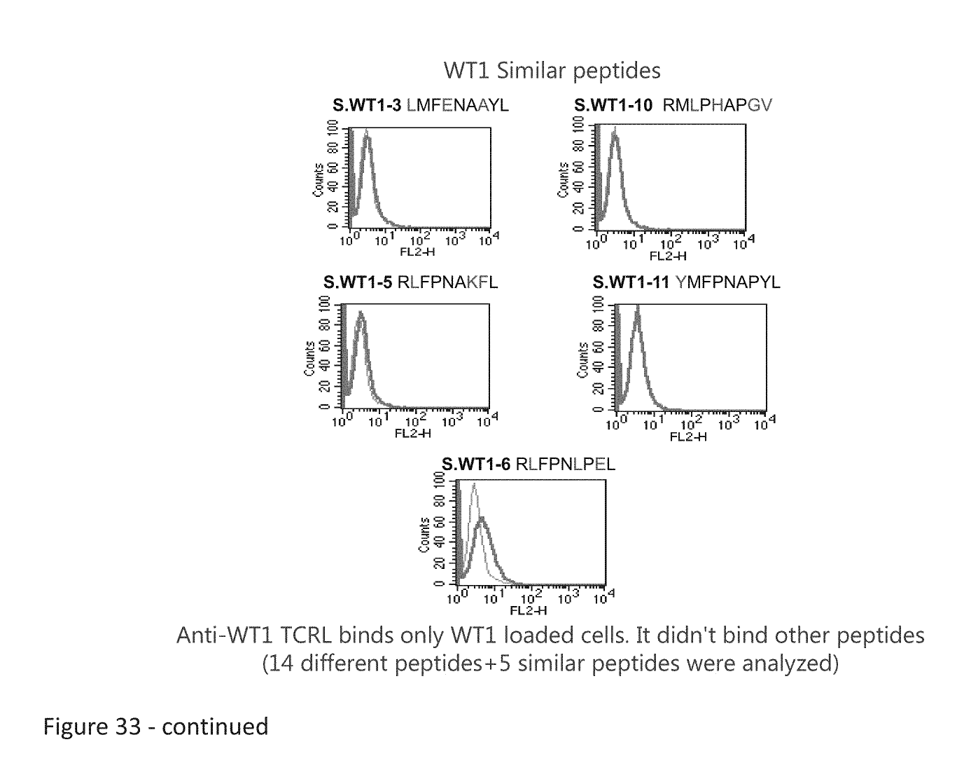

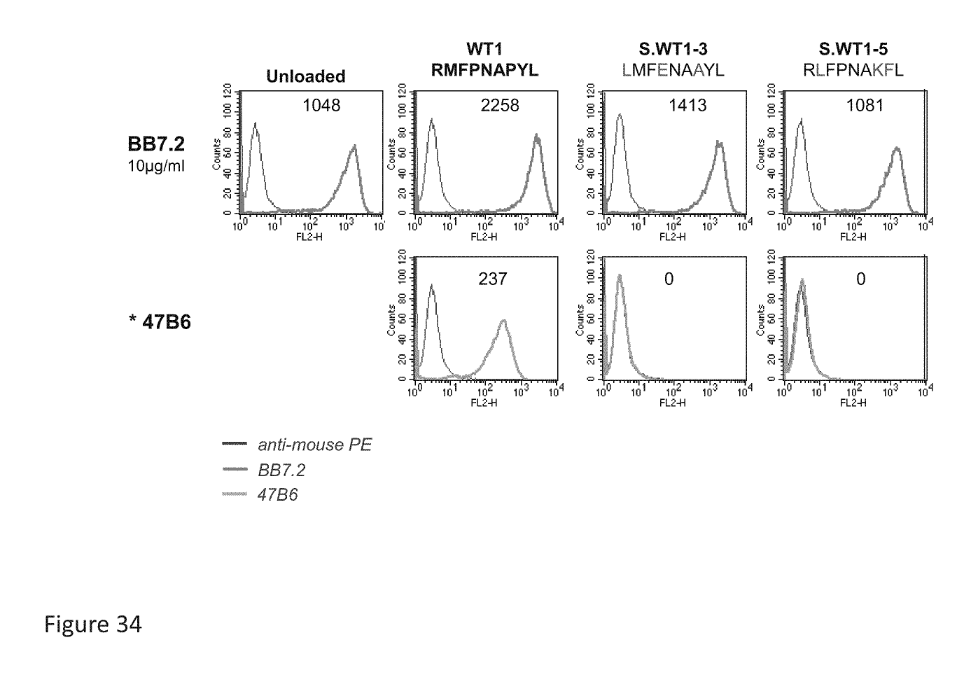

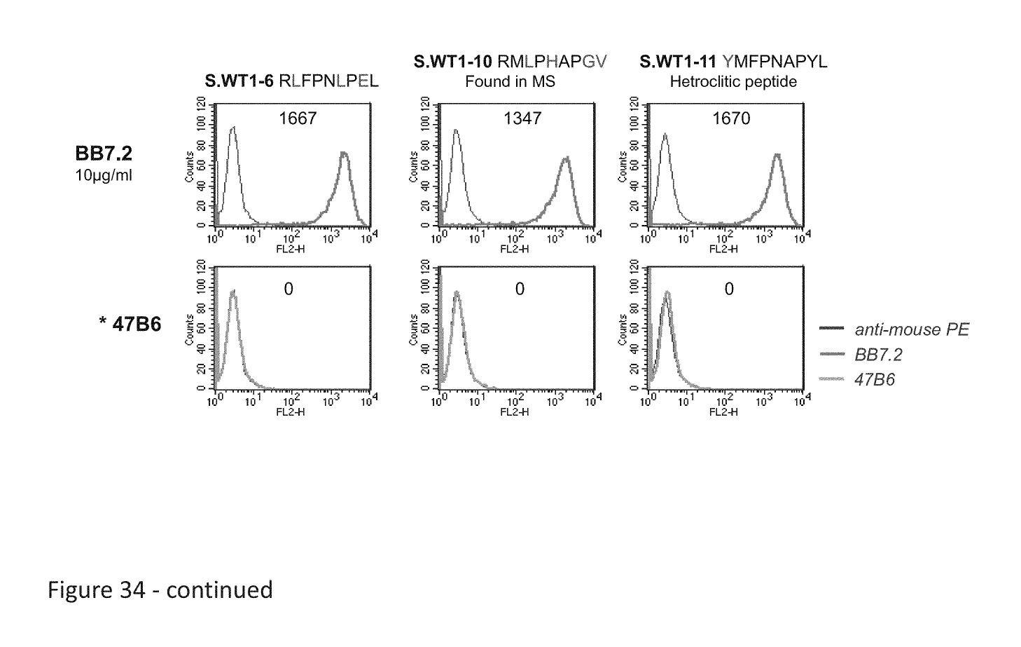

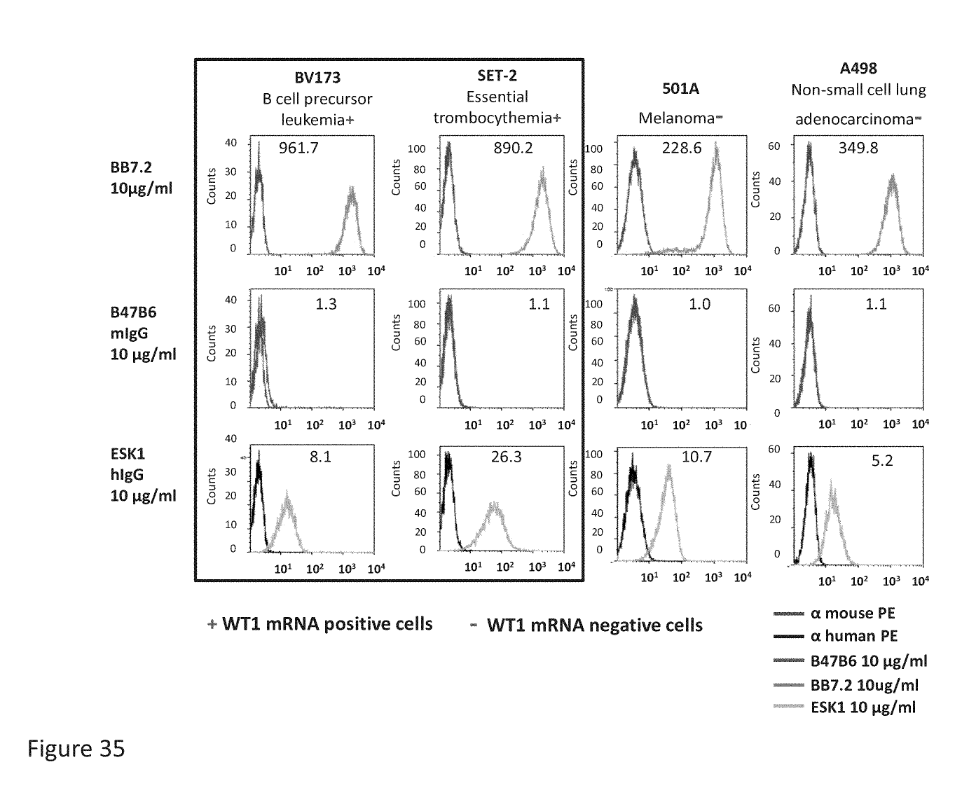

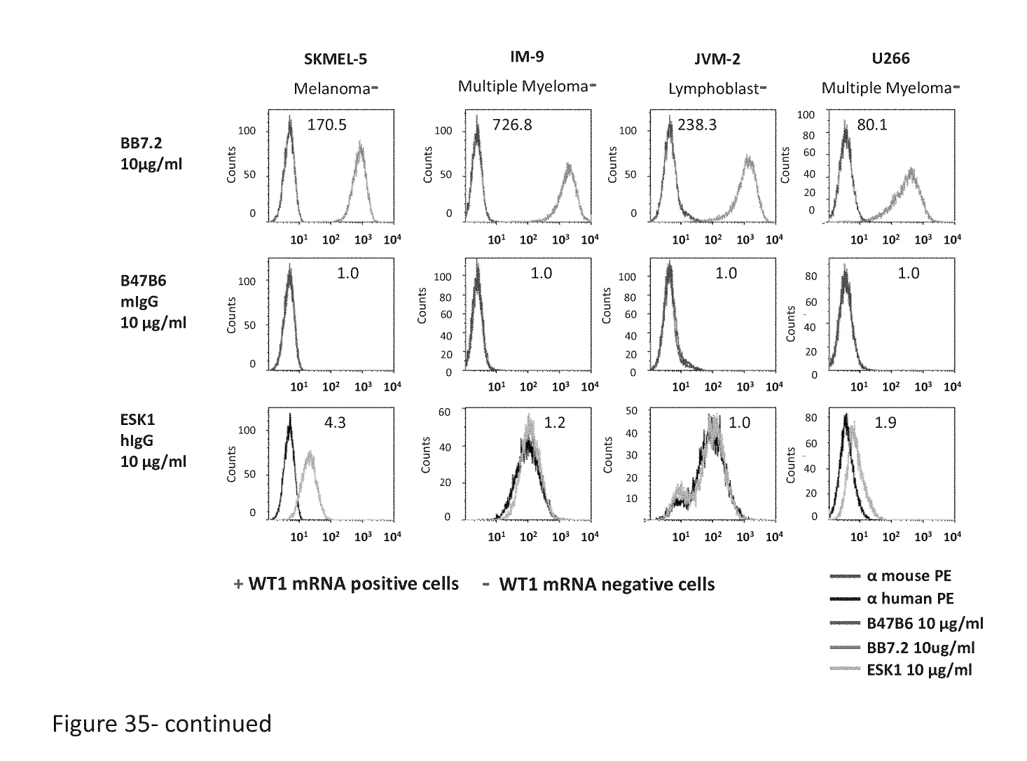

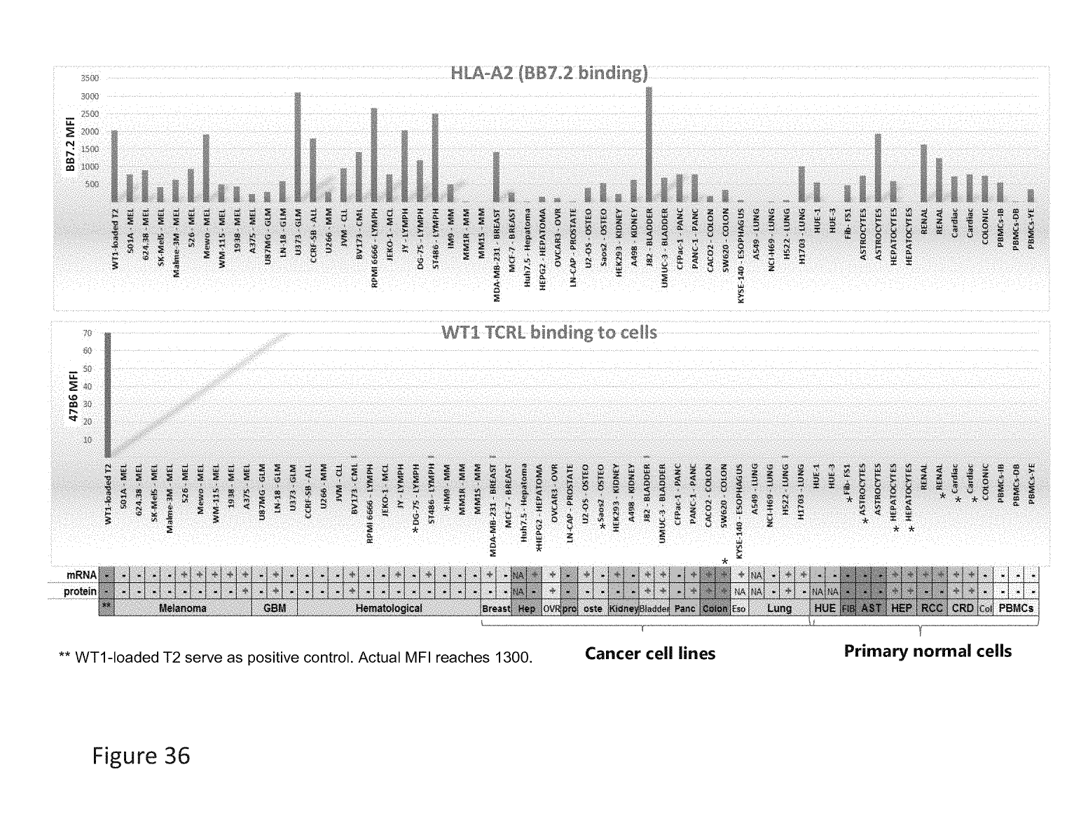

To further investigate the WT1 TCR-like antibodies fine specificity evaluation of binding to similar peptides identified in silico with the strategy described above was performed. As shown in FIGS. 32 and 33, B47 did not bind any similar peptide from a designed panel (Table 6). However, as shown in FIG. 32, ESK1 exhibited low background binding with two similar peptides. B47 was evaluated on additional control peptides and similar peptides (FIG. 34). Further analysis of these TCR-like antibodies was performed by flow cytometry using tumor cells that are HLA-A2 and express or not the WT1 antigen. As shown in FIG. 35, the ESK1 WT1 TCR-like antibody bound intensely to HLA-A2+/WT+BV173 and SET2 cells however B47 did not exhibit any binding to these cells to the level of flow cytometry sensitivity. To further investigate specificity the reactivity of ESK1 and B47 was evaluated on cells that are HLA-A2 but do not express the WT1 gene as evaluated by PCR. As shown B47 did not bind to any of these cells while ESK1 bound to 501, A498, and SKMEL cells that were found to be WT1 negative. Other WT1 negative cells were not bound by ESK1. The level of HLA-A2 expression was monitored with MAb BB7.2 which recognizes all HLA-A2/peptide molecules on the cell surface. A summary of binding data for B47 WT-specific TCR-like antibody is shown in FIG. 36.

To further investigate the conflicting data of the binding of ESK1 and B47 to HLA-A2+/WT1+BV173 and SET2 cells, i.e binding could be detected significantly by ESK1 but not B47 we employed direct biochemical means to evaluate actual WT1 presentation on these cells. We employed HLA peptide elution strategies from various tissues as well from BV173 and SET2 cells followed by MS analysis of eluted peptides. The data of these experiments indicate that the WT1 peptide has not been detected in any of the MS runs of clinical tissues or cell lines. In depth analysis of the BV173 or SET-2 cell lines (mRNA WT1-positive) failed to detect the peptide (Orbitrap or Q Exactive MS instruments). The WT1 peptide was detected by OrbiTrap MS following direct elution from T2 peptide-loaded cells. These T2 cells were loaded with various WT1 peptide concentrations of 10⁻⁵, 10⁻⁷, 10⁻⁹ M and the peptide was detected by the MS in elutions from T2 APCs loaded with peptide concentration of 10⁻⁵ and 10⁻⁷M. Detecting the peptide from T2 cells loaded at 10⁻⁷M peptide by the MS corresponds to actual presentation of ˜250 sites/cell (using the Orbitrap MS).

These data exemplifies the usefulness of the described binding tools towards peptide loaded cells that display similar peptides and cells of various histological origins to evaluate the specificity and selectivity of TCR-like antibodies.

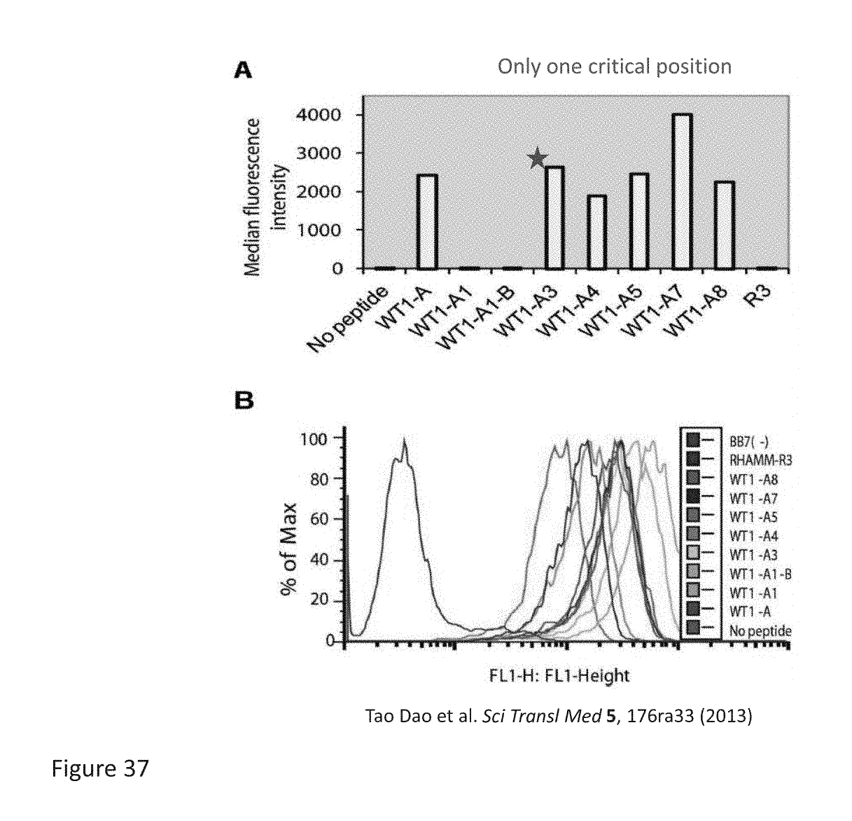

To further investigate epitope specificity, alanine scanning mutagenesis was performed on the WT1 peptide sequence. As shown in FIG. 37 which demonstrates that only mutation in position 1 of the WT1 peptide influenced the binging intensity of ESK1 indicating that the binding selectivity and fine specificity of ESK1 is limited compared to B47 as also observed for the specificity pattern as observed for similar peptides and for cells that are HLA-A2+/WT1−/These data suggest that the selectivity and fine specificity of B47 is superior compared to ESK1 and that the tool box presented herein is a valuable tool to evaluate the selectivity and fine specificity of TCR-like antibodies in the process of their selection, characterization, and pre-clinical development.

### Example Iia

### TCR-Like Antibodies for HLA-A2/WT1

Comparison of Fine Specificity of Abs with TCR-Like Specificity to HLA-A2/WT1

The selectivity of TCR-like antibodies B47 and ESK1 both recognizing WT1 peptide was compared (Dao et al. Sci Transl Med. 2013 Mar. 13; 5(176):176ra33)

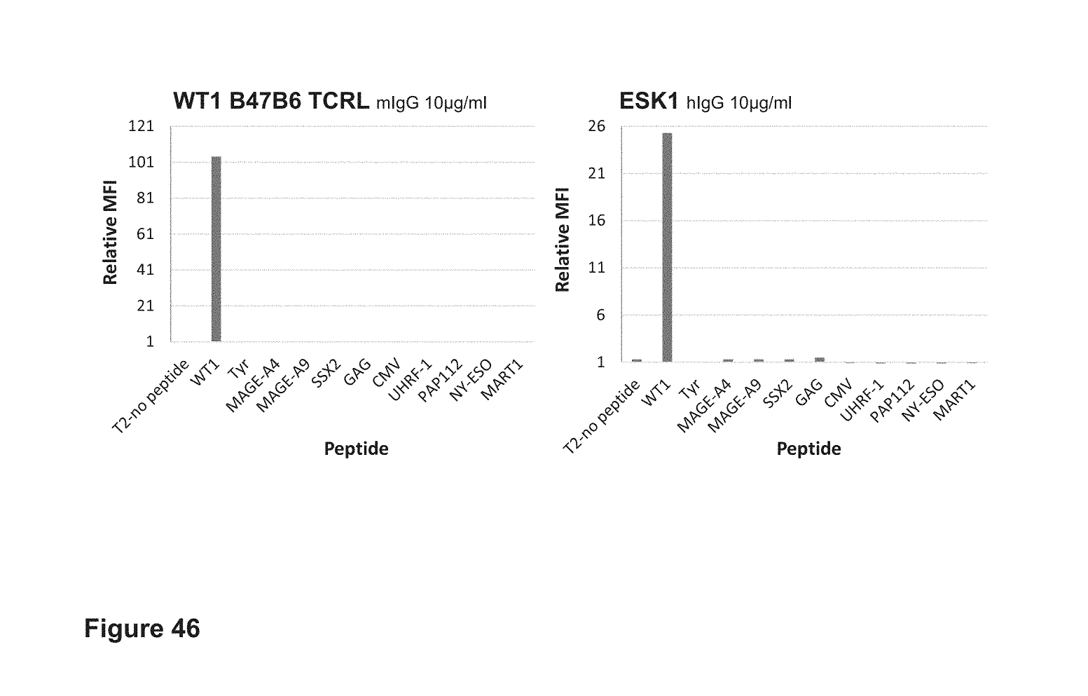

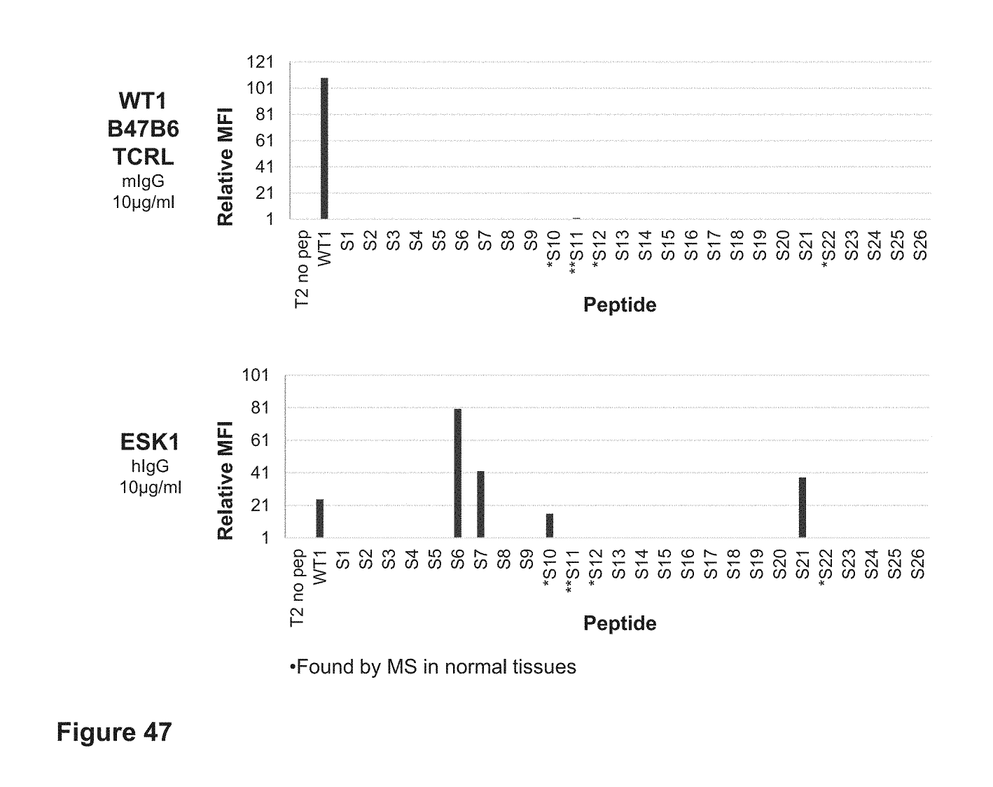

T2 APCs were loaded with specific (WT1, SEQ ID NO: 141) or control peptides (Table 15) and incubated with the B47 and ESK1 antibodies, followed by incubation with PE-labeled streptavidin or anti-mouse Abs. Both B47 and ESK1 TCRLs bound T2 cells loaded with the WT1 peptide but did not bind to cells loaded with control peptides (FIG. 46). A panel of similar peptides (Table 6) was synthesized to further characterize specificity of the WT1 TCRLs. The B47 TCRL did not bind to any of the similar peptides loaded onto T2 cells while ESK1 showed detectable binding to several similar peptides (FIG. 47). ESK1 TCRL showed binding to a similar peptide derived from HDAC2 (Histone deacetylase 2, Table 14) that is ubiquitously presented by many normal cells. WT1-S10 (SEQ ID NO: 151) is presented in normal tissues as evidenced by mass spectrometry in brain, cerebral cortex, heart, kidney, liver, lung, and other normal tissues (Table 14).

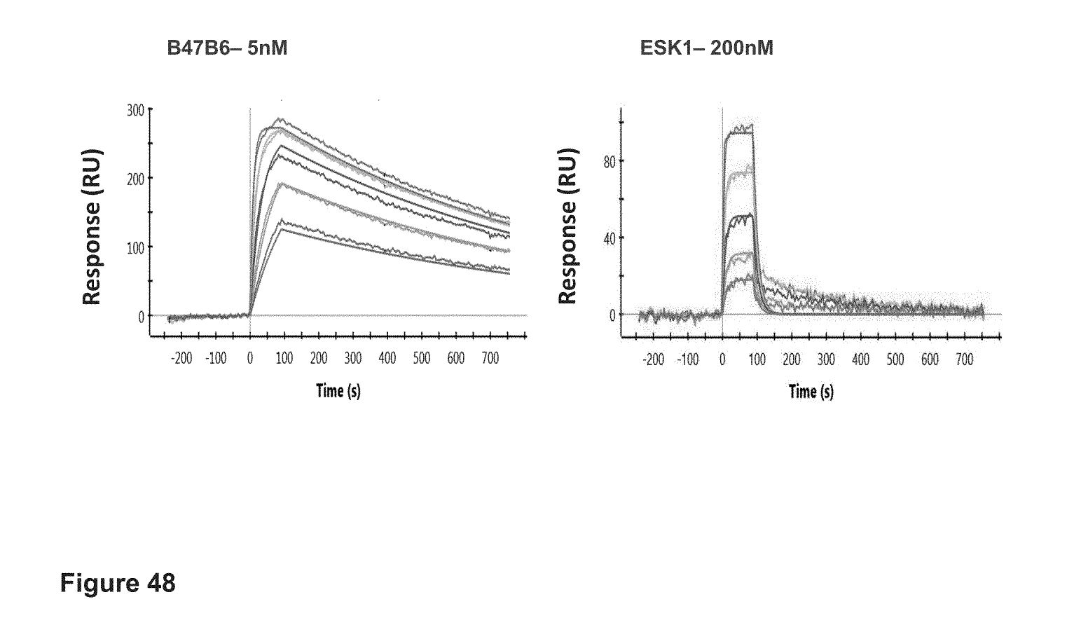

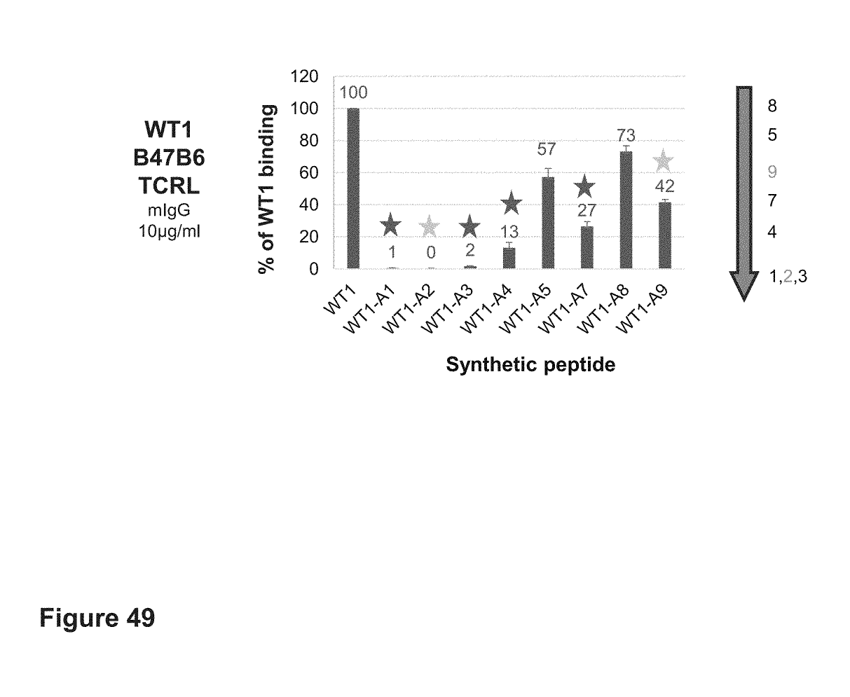

Further characterization of binding of B47 and ESK1 TCRLs by SPR showed that affinity of B47 (5 nM) is much stronger than that of ESK1 (200 nM) mainly due to faster dissociation rate of ESK1 and MHC-WT1 peptide complexes (FIG. 48). Additional alanine scanning mutagenesis of the WT1 peptide was performed to refine peptide epitope specificity of B47 TCR-like antibodies (FIG. 49). The mutant peptides were loaded onto T2 cells and binding assay was performed as described above. The loading of the various Ala mutants was monitored by flow cytometry using BB7.2 monoclonal antibody against HLA-A2.

As shown in FIG. 49, substitutions to Ala at some positions significantly affected B47 binding to the mutated peptides. B47 TCRL exhibited greater sensitivity to positional substitutions (as compared to ESK1, FIG. 37). The B47 TCR-like antibody lost >73% of its binding to presented peptide with when 4 residues in the peptide were mutated to Alanine (positions 1, 3, 4, and 7). A 5^{th} position sensitivity can be attributed to position number 5. For both B47 and ESK1 TCRLs position 2 was critical as it is expected to serve as anchor position for the peptides in the HLA-A2 peptide binding groove.

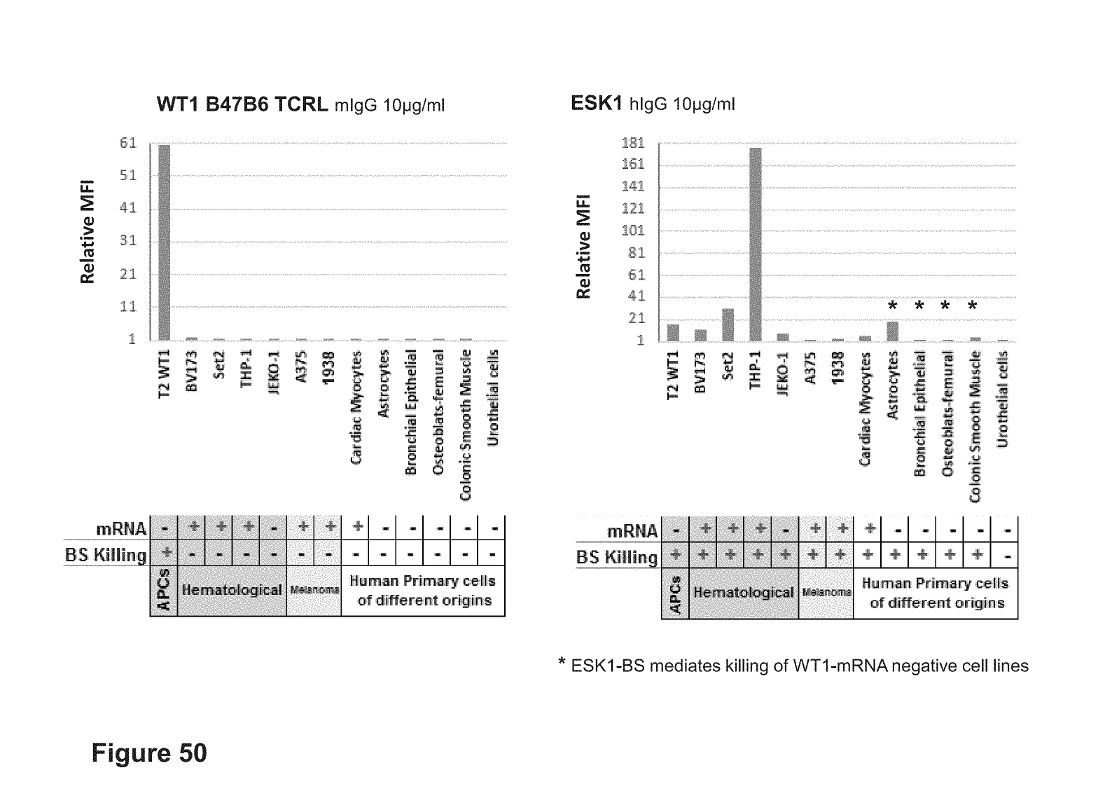

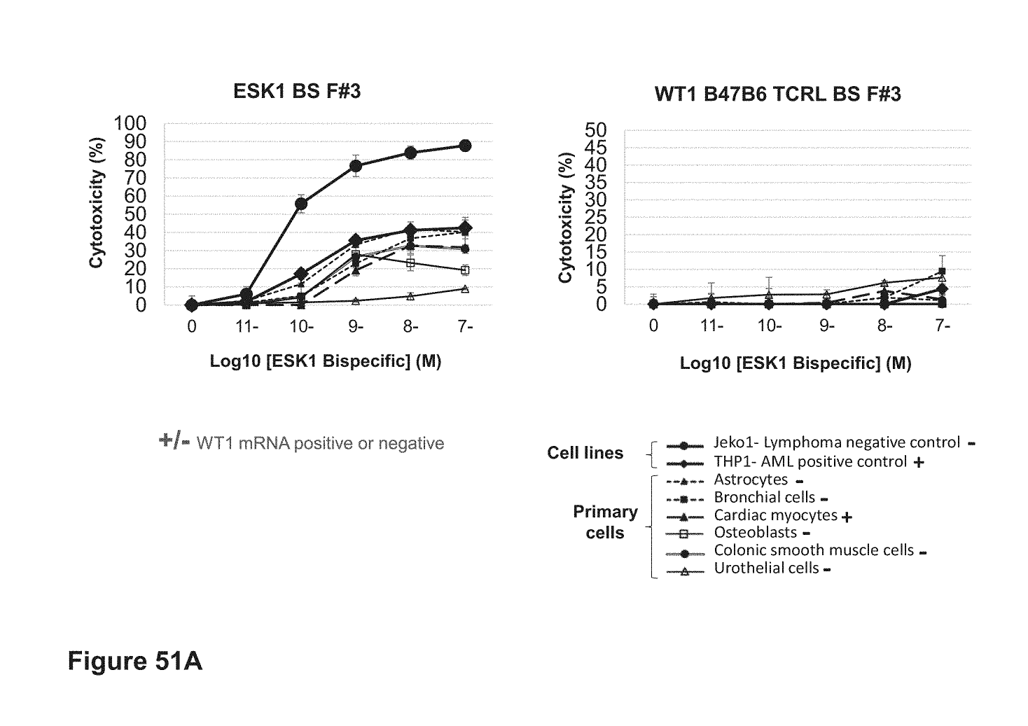

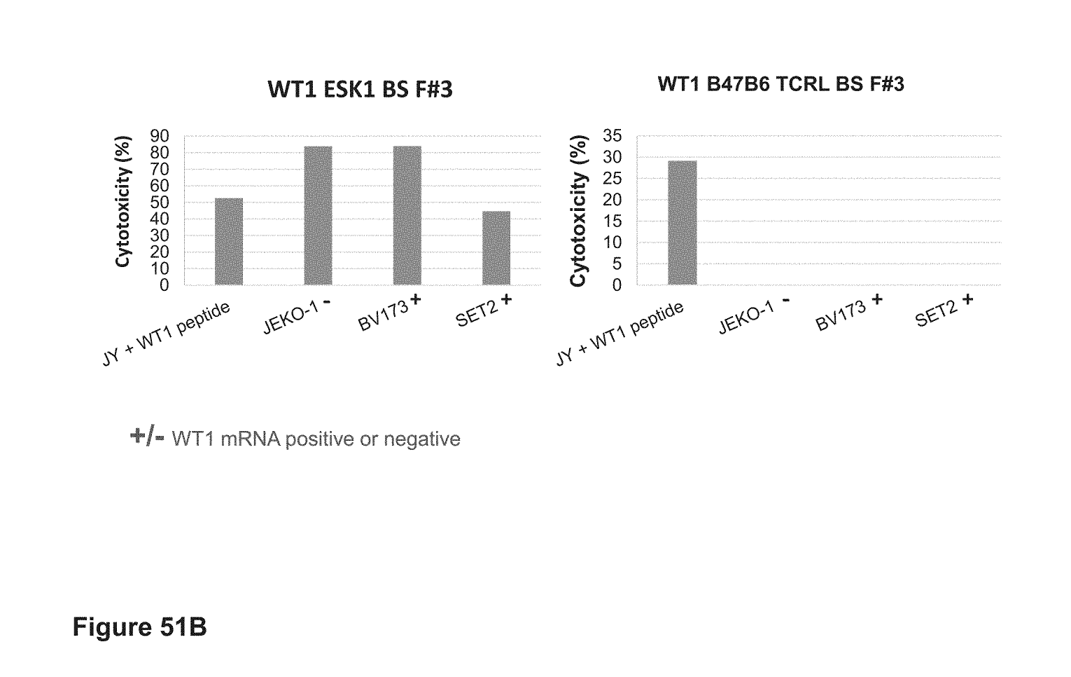

Further characterization and comparison between B47 and ESK1 TCRLs was done on tumor cell lines and primary cells of various origins. As shown in FIG. 50, B47 did not bind to a panel of cells that were all HLA-A2 positive and WT1 mRNA positive or negative cells. In contrast, ESK1 TCRL bound to a number of both tumor and normal primary cells (all HLA-A2+). For example, JVM2 and IM9 (both HLA-A2 positive and WT1 negative) as well as normal primary astrocytes showed binding. Cytotoxicity assays using TCRL-aCD3 bi-specific constructs and human PBMCs showed that B47 TCRL did not induce death of HLA-A2+/WT1+ or HLA-A2+/WT1-cells while ESK1 TCRL-aCD3 was cytotoxic to a number of cells, including WT-1 negative. Thus, B47 TCRL demonstrate superior specificity in both binding and functional activity in the bi-specific format compared to ESK1 that binds to and re-targets CD3 T-cells toward some cells, including normal primary cells, regardless of WT-1 expression.

### Example III

### TCR-Like Antibodies with Specificity to HLA-A2/MAGE-A4

### Example Iiia

### Isolation and Characterization of TCRL with Specificity to HLA-A2/MAG-A4

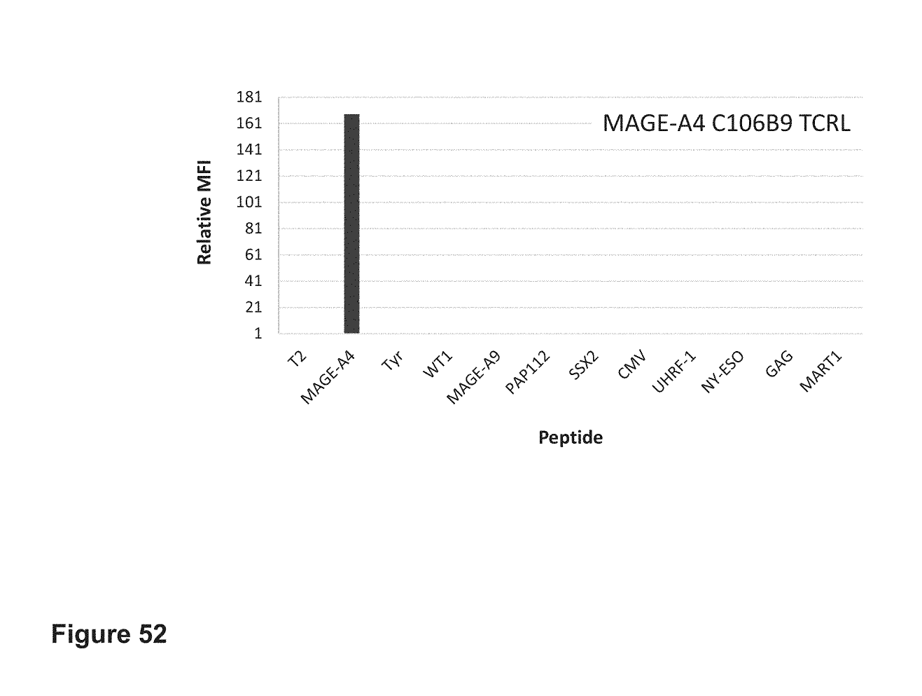

To characterize the binding specificity of the isolated TCR-like antibodies the reactivity and specificity of the purified IgGs were assessed by flow cytometry. T2 APCs were loaded with MAGE-A4 peptide or control peptides (Table 15) and incubated with the TCRL Ab C106B, followed by incubation with PE-labeled streptavidin or PE-labeled anti mouse Abs. As shown in FIG. 52, C106B9 bound T2 cells loaded with the MAGE-A4 peptide but showed no binding to cells loaded with control peptides.

To further evaluate the specificity of the C106B9 TCR-like antibody its reactivity with peptides that exhibit sequence similarity to the MAGE-A4 peptide was evaluated. The peptides are shown in Table 8.

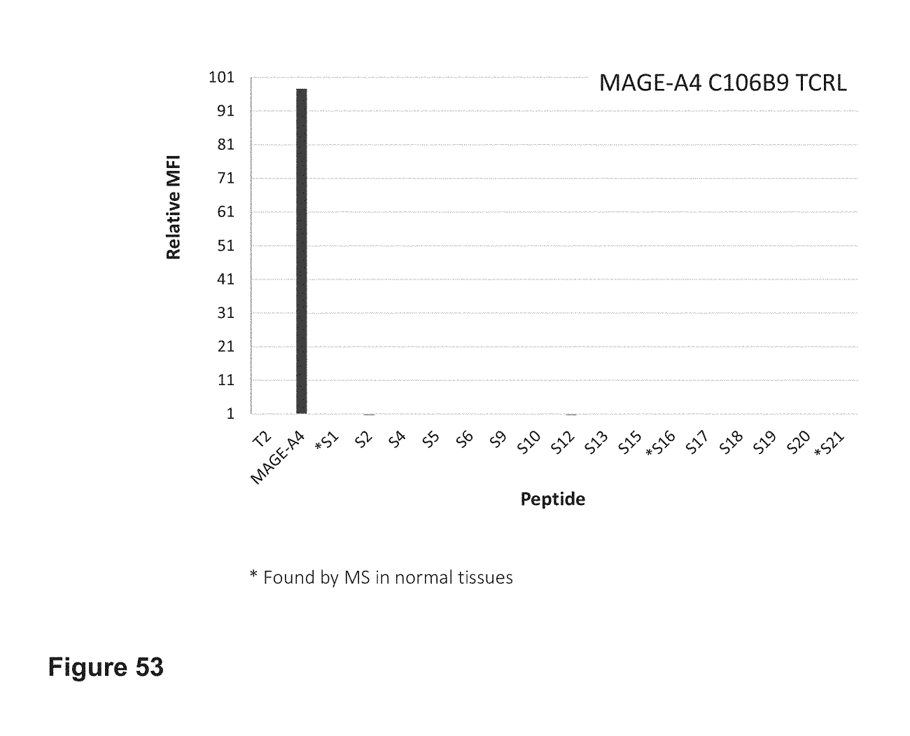

As shown in FIG. 53, C106B9 TCRL did not bind any of the peptides from this panel of similar peptides. These data demonstrate the high selectivity and fine specificity of C106B9 and demonstrates the usefulness of the similar peptide approach and tools developed as described above to evaluate the selectivity and fine specificity of TCRLs.

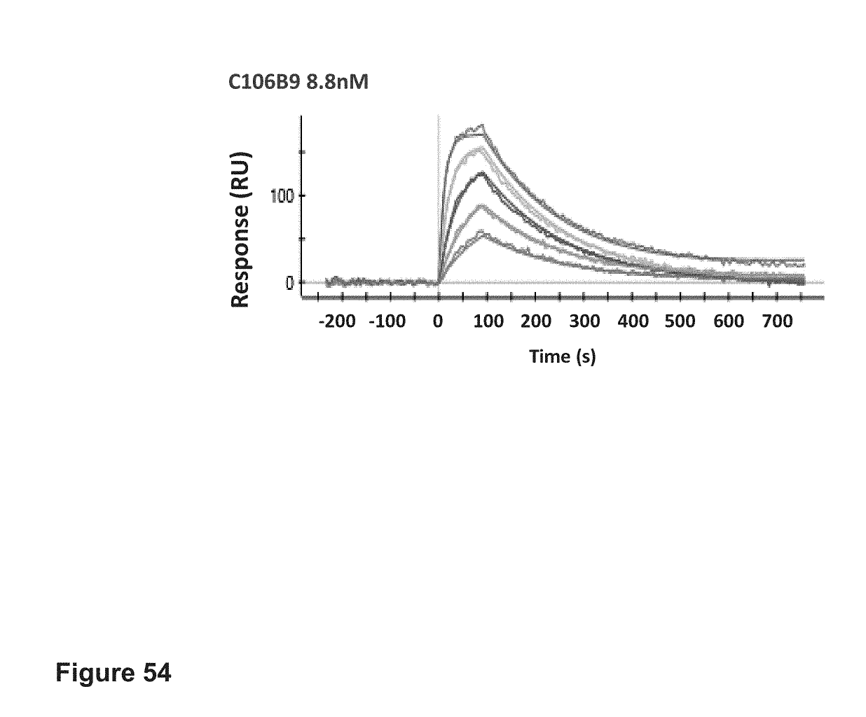

To determine the apparent affinity of the isolated TCR-like antibody, surface plasmon resonance (SPR) binding analysis was used in which the isolated purified IgG TCR-like antibody was immobilized to the SPR sensor chip by using anti-mouse IgG to indirectly immobilize the TCR-like antibodies on the chip surface. The analyte is the purified single-chain recombinant HLA-A2/MAGE-A4 complex used at various concentrations. As shown in FIG. 54, the sensorgrams of SPR analysis revealed affinity for the HLA-A2/MAGE-A4 specific TCR-like antibody clone C106B9 with corresponding affinity of 8.8 nM.

To investigate the fine peptide epitope specificity of the isolated TCR-like antibodies towards the MAGE-A4 peptide alanine scanning was performed in which specific residues in the peptide were mutated to alanine and the binding of the TCR-like antibody to Ala mutated peptides was tested by their loading onto T2 antigen presenting cells (Table 9). Binding was monitored by flow cytometry and extent of binding of TCR-like antibodies to the mutated presented peptides as measured by mean fluorescence intensity (MFI) was compared in comparison to T2 APCs loaded with the native unmutated MAGE-A4 peptide. The proper loading of the various Ala mutated peptides (described in FIG. 2) was monitored by flow cytometry using BB7.2 a monoclonal antibody for HLA-A2.

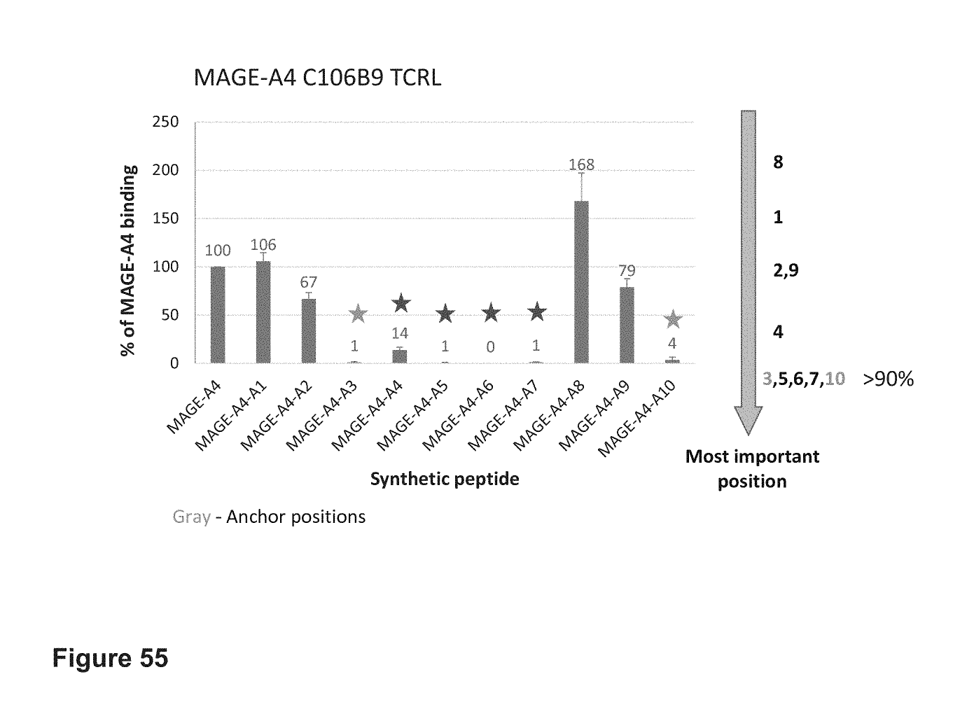

All Ala mutated peptides were efficiently loaded onto T2 cells in comparison to the native un-mutated MAGE-A4 peptide (data not shown). As shown in FIG. 55, The TCR-like antibody exhibited peptide dependent binding as specific mutations affected the binding and induced a decrease in the binding intensity of the TCR-like antibody upon introduction of Ala at specific peptide positions. These results indicate that MAGE-A4 TCR-like antibody exhibited peptide-specific and restricted binding in the context of HLA-A2 loaded with various Ala mutated MAGE-A4 peptides, indicating that this antibody is TCR-like in its binding properties, thus, it binds the MHC-peptide complex with MHC-restricted and peptide-specific manner.

The C106B9 TCR-like antibody exhibited a marked decrease of 90% in binding to Ala mutated peptide at four positions #4, 5, 6, and 7. A 5^{th} position sensitivity can be attributed to position number 2 (decrease of 33%).

Overall, the Alanine scanning analysis reveals that Ala scanning can be used as a measure to determine the selectivity and fine specificity of TCR-like antibodies. As more sensitivity to Ala mutations is exhibited the more specific and peptide-dependent binding will be observed. This strategy can be used to filter and select for the optimal TCR-like antibodies that exhibited the higher and optimized selectivity and specificity properties as MHC-restricted peptide-specific binders.

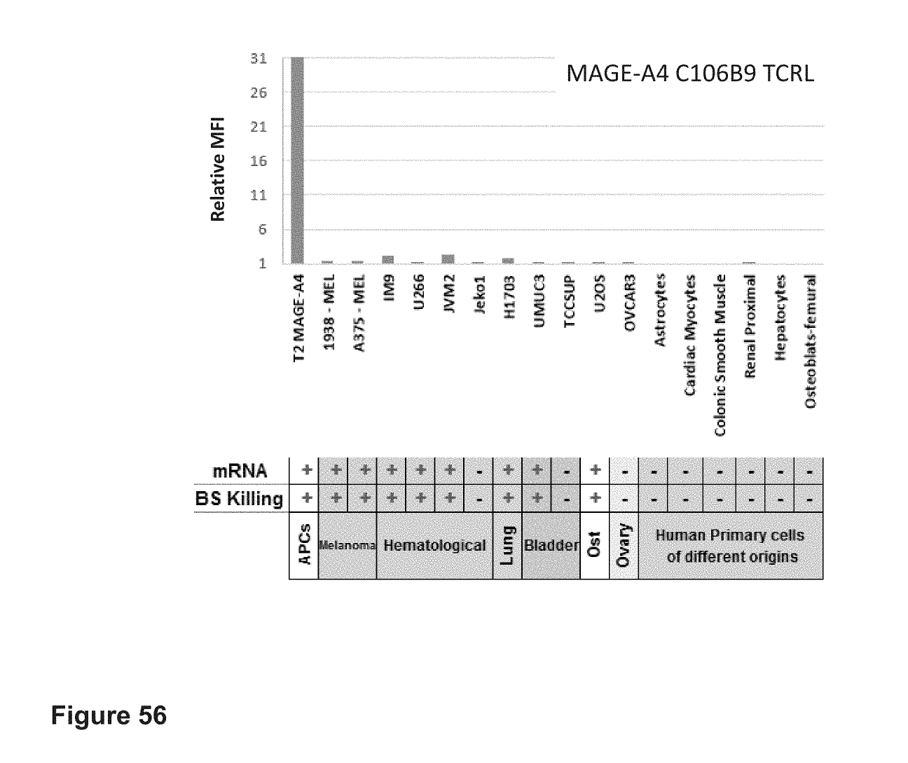

The present inventors explored binding specificity of the HLA-A2/MAGE-A4 TCR-like Ab to MHC-MAGE-A4 peptide complexes endogenously displayed on the surface of tumor cell lines. Cells were incubated with anti-MAGE-A4-HLA-A2 TCR-like antibodies Ab followed by incubation with PE-labeled streptavidin or anti-mouse Abs. A panel of tumor cells and normal primary cells that have been characterized for HLA-A2 (positive) and MAGE-A4 (positive or negative) expression was used to compare the binding of the TCR-like antibodies. As shown in FIG. 56, the TCR-like antibody recognized with low intensity MAGE4-positive and HLA-A2-positive cells. The TCR-Like antibodies were tested on multiple HLA-A2-positive cell lines of various origin that do not show MAGE-A4 RNA expression (MAGE-A4-negative), killing activity of these cells with a MAGE-A4/HLA-A2 TCRL-Bispecific construct was also tested. As shown in FIG. 56, C106B9 TCRL did not bind any of these cells.

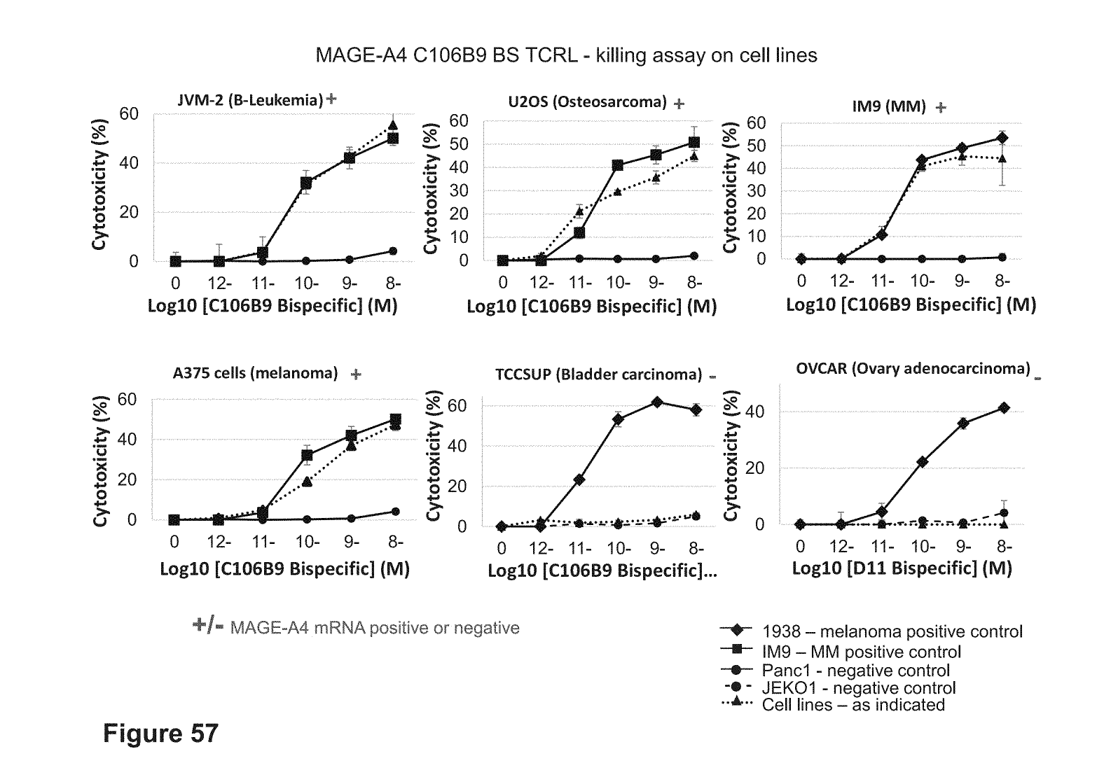

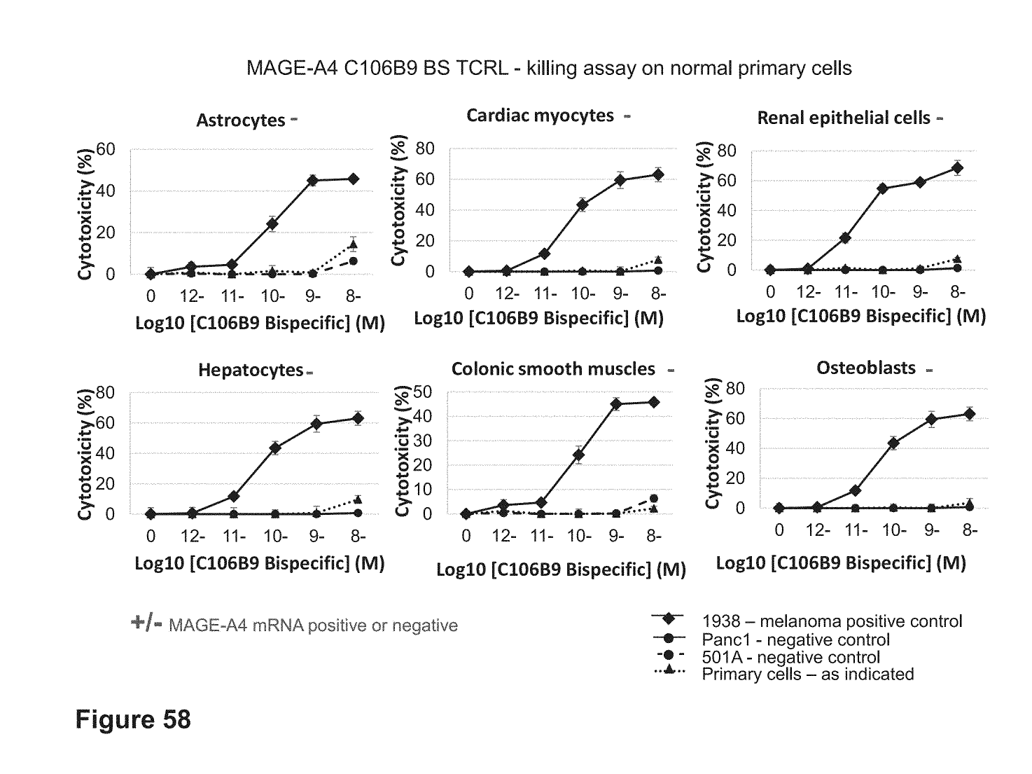

Functional assays were used to further characterize the C106B9 TCR-like antibody. TCRLs variable regions were fused to an anti-CD3 scFv which can re-target effector T cells to kill tumor target cell in a of bi-specific format. As shown in FIG. 57, the C106B9 Bi-specific TCR-like antibody constructs showed robust cytotoxicity against MAGE-A4 positive cells in vitro in the presence of human PBMCs. TCCSUP and OVCAR, MAGE-A4 negative cell line served as negative control and demonstrated no cytotoxicity. As further shown in FIG. 58, No cytotoxicity was detected against a panel of HLA-A2+/MAGE-A4− normal human primary cells with C106B9 TCRL confirming its selectivity.

### Example Iiib

### In Vivo Efficacy of MAGE-A4 C106B9 BS TCRL in s.c. A375 Melanoma Tumor Formation Model in NOD/SCID Mice

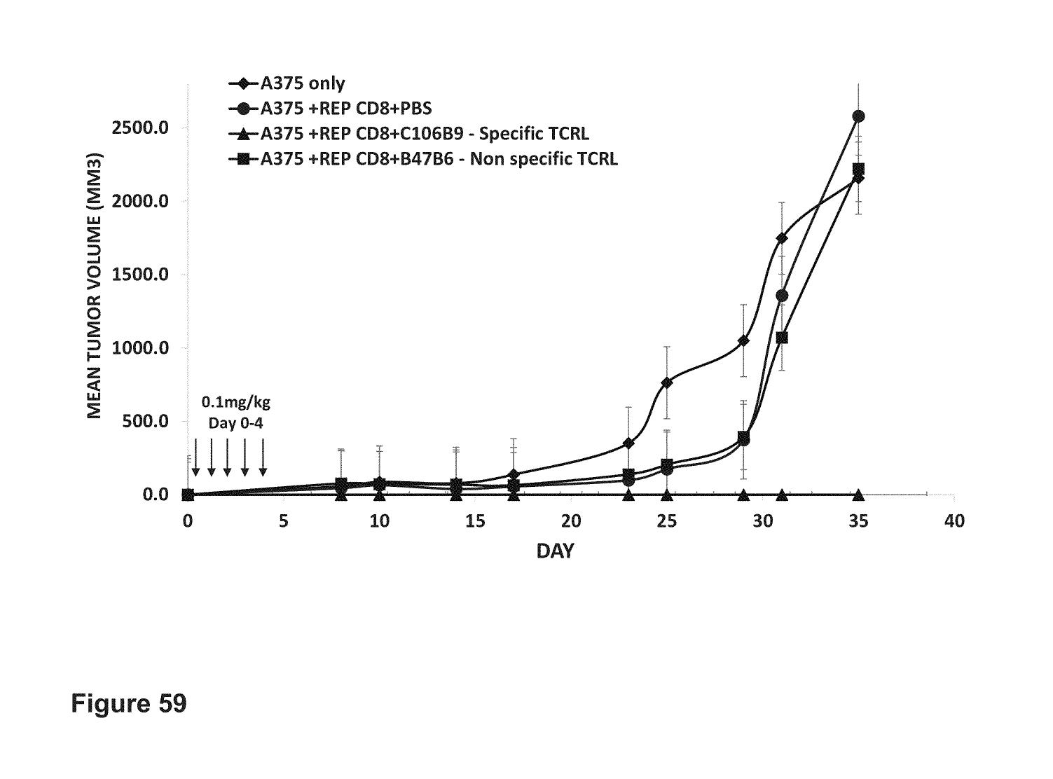

FIG. 59 shows in vivo efficacy of C106B9 BS TCRL in S.C. A375 melanoma tumor formation model in NOD/SCID mice. Clearly, administration of the bispecific antibody completely inhibited tumor formation over 35 days of the experiment, as evidenced by tumor volume. The results support the use of variable sequences of the TCRLs described herein in clinical settings.

### Example IV

### TCR-Like Antibodies with Specificity to HLA-A2/MAGE-A9

Isolation and Characterization of TCRL with Specificity to HLA-A2/MAGE-A9

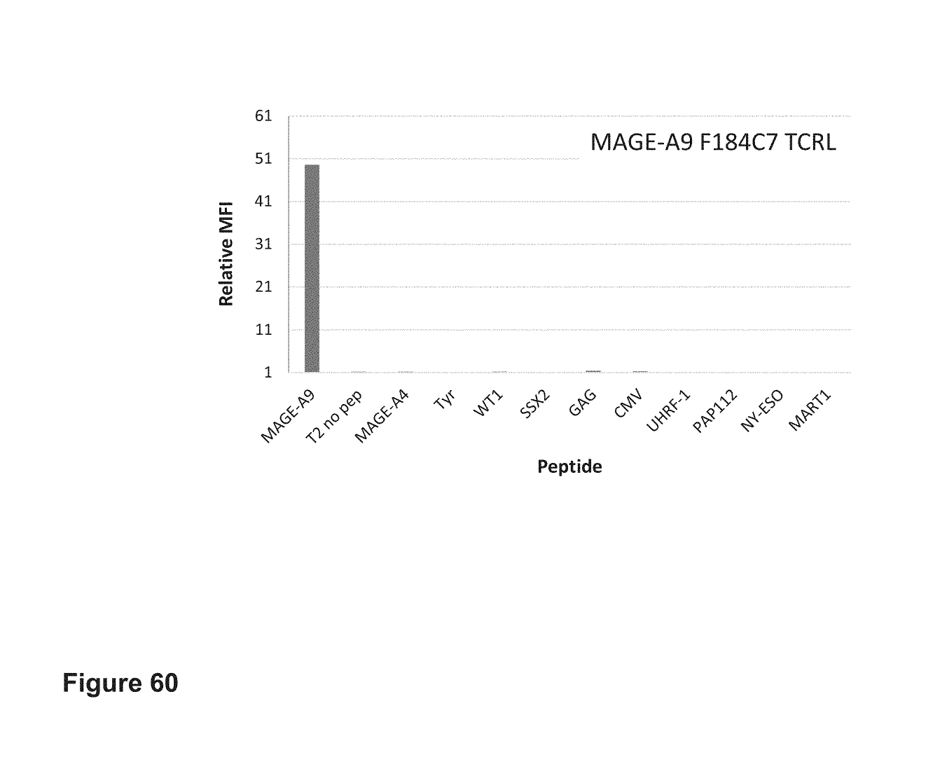

To characterize the binding specificity of the isolated TCR-like antibodies the reactivity and specificity of the purified IgGs were assessed by flow cytometry. T2 APCs were loaded with MAGE-A9 peptide or control peptides and incubated with the TCRL Ab F184C7, followed by incubation with PE-labeled streptavidin or PE-labeled anti mouse Abs. As shown in FIG. 60, F184C7 bound T2 cells loaded with the MAGE-A9 peptide but showed no binding to cells loaded with control peptides.

To further evaluate the specificity of the F184C7 TCR-like antibody its reactivity with peptides that exhibit sequence similarity to the MAGE-A9 peptide was evaluated. The peptides are shown in Table 10.

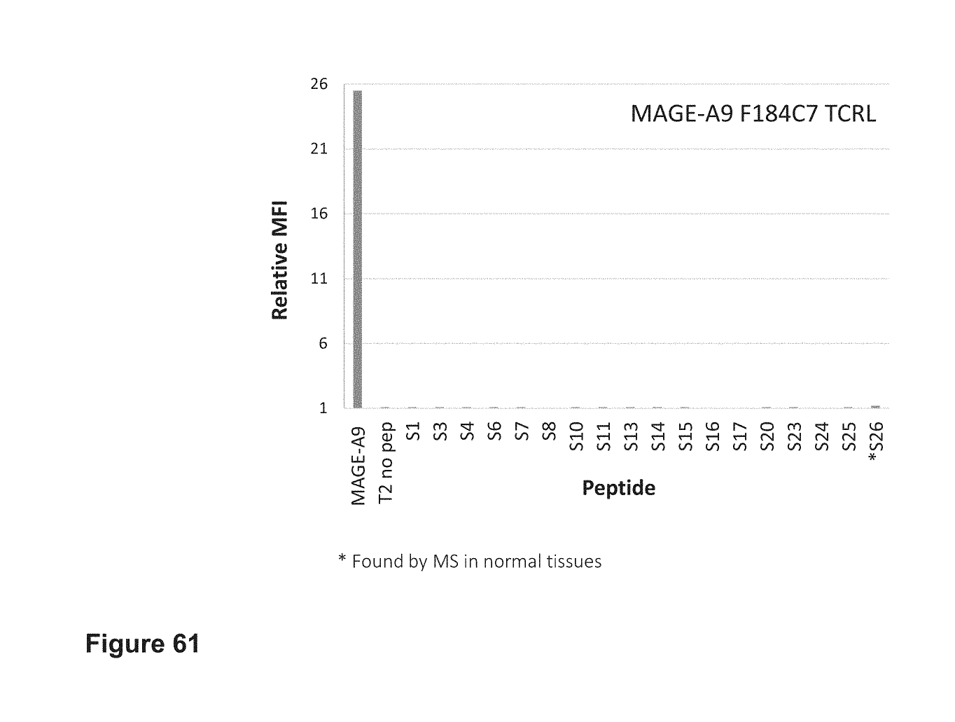

As shown in FIG. 61, F184C7 TCRL did not bind any of the peptides from this panel of similar peptides. These data demonstrate the high selectivity and fine specificity of F184C7 and demonstrates the usefulness of the similar peptide approach and tools developed as described above to evaluate the selectivity and fine specificity of TCRLs.

To investigate the fine peptide epitope specificity of the isolated TCR-like antibodies towards the MAGE-A9 peptide alanine scanning was performed in which specific residues in the peptide were mutated to alanine and the binding of the TCR-like antibody to Ala mutated peptides was tested by their loading onto T2 antigen presenting cells (Table 11). Binding was monitored by flow cytometry and extent of binding of TCR-like antibodies to the mutated presented peptides as measured by mean fluorescence intensity (MFI) was compared in comparison to T2 APCs loaded with the native unmutated MAGE-A9 peptide. The proper loading of the various Ala mutated peptides (described in FIG. 2) was monitored by flow cytometry using BB7.2 a monoclonal antibody for HLA-A2.

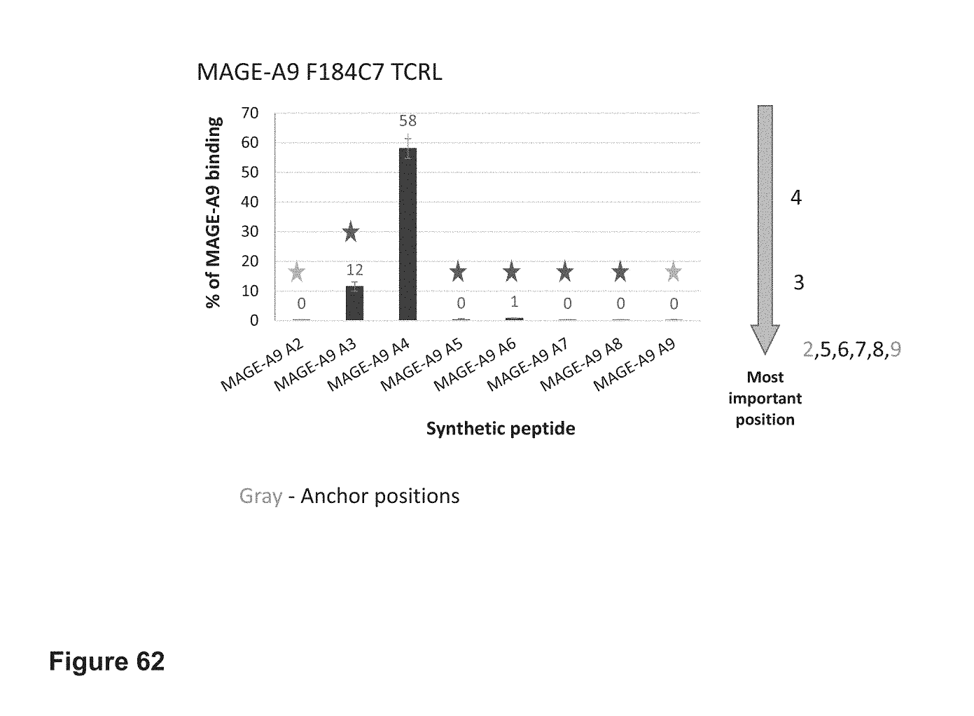

All Ala mutated peptides were efficiently loaded onto T2 cells in comparison to the native un-mutated MAGE-A9 peptide (data not shown). As shown in FIG. 62, The TCR-like antibody exhibited peptide dependency binding as specific mutations affected the binding and induced a decrease in the binding intensity of the TCR-like antibody upon introduction of Ala at specific peptide positions. These results indicate that MAGE-A9 TCR-like antibody exhibited peptide-specific and restricted binding in the context of HLA-A2 loaded with various Ala mutated MAGE-A9 peptides, indicating that this antibody is TCR-like in its binding properties, thus, it binds the MHC-peptide complex with MHC-restricted and peptide-specific manner.

The F184C7 TCR-like antibody exhibited a marked decrease of 90% in binding to five Ala mutated peptide at five positions #3, 5, 6, 7 and 8.

Overall, the Alanine scanning analysis reveals that Ala scanning can be used as a measure to determine the selectivity and fine specificity of TCR-like antibodies. As more sensitivity to Ala mutations is exhibited the more specific and peptide-dependent binding will be observed. This strategy can be used to filter and select for the optimal TCR-like antibodies that exhibited the higher and optimized selectivity and specificity properties as MHC-restricted peptide-specific binders.

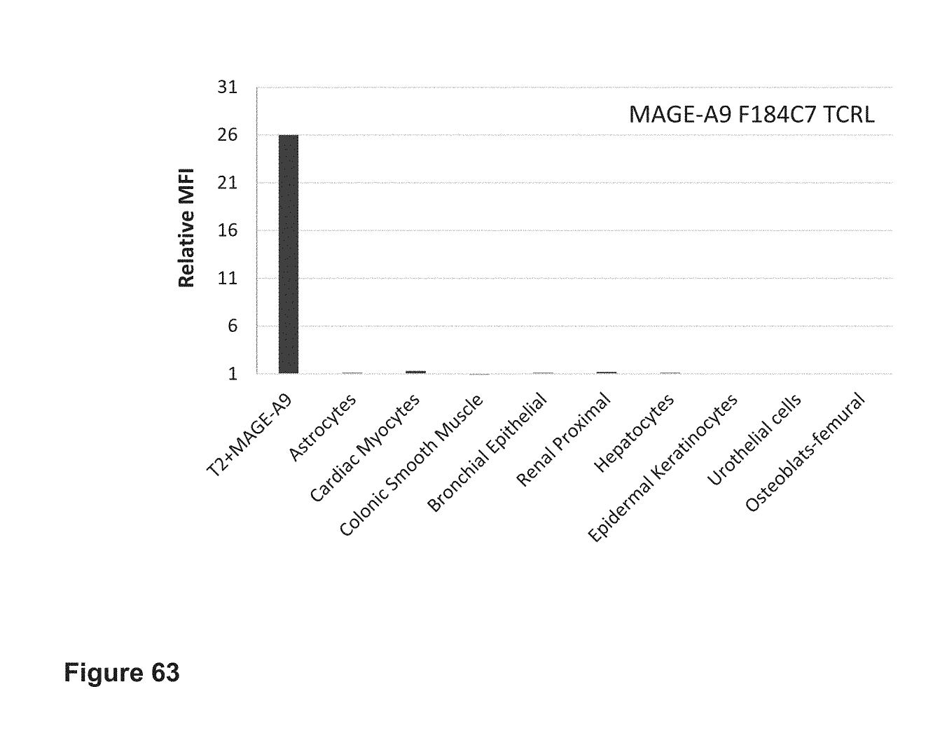

The present inventors explored binding specificity of the HLA-A2/MAGE-A9 TCR-like Ab to a panel of normal primary cells of various origin that do not show MAGE-A9 RNA expression. As shown in FIG. 63, F184C7 TCRL did not bind any of these cells. Positive control was T2 cells loaded with the MAGE-A9 peptide to which F184C7 bound intensely.

### Example V

### TCR-Like Antibodies with Specificity to HLA-A2/PAP

Isolation and Characterization of TCRL with Specificity to HLA-A2/PAP

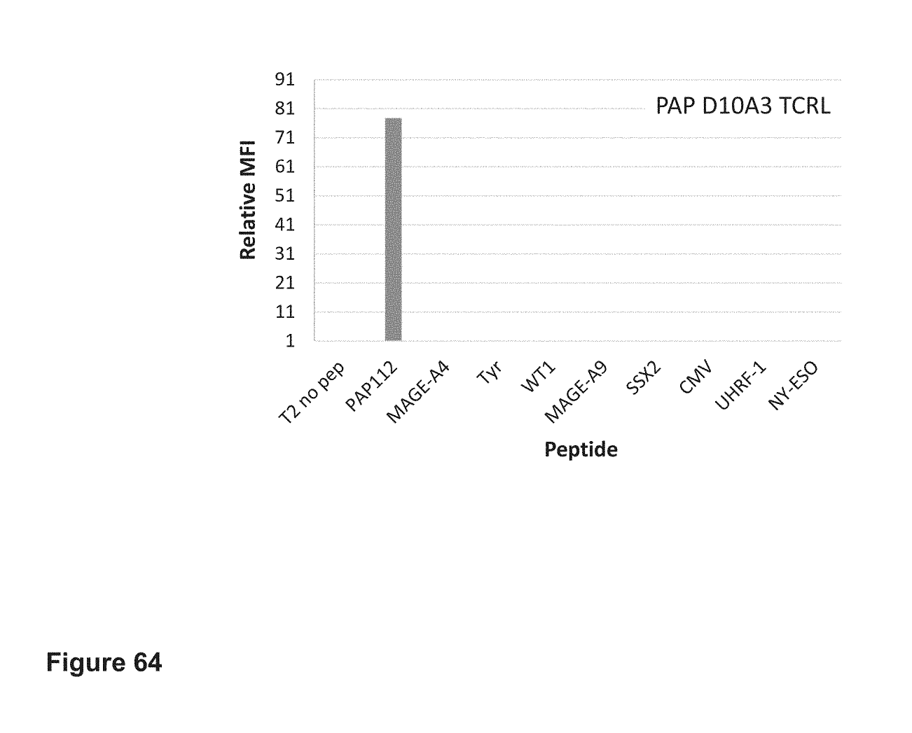

To characterize the binding specificity of the isolated TCR-like antibodies the reactivity and specificity of the purified IgGs were assessed by flow cytometry. T2 APCs were loaded with PAP peptide or control peptides and incubated with the TCRL Ab D10A3, followed by incubation with PE-labeled streptavidin or PE-labeled anti mouse Abs. As shown in FIG. 64, D10A3 bound T2 cells loaded with the PAP peptide but showed no binding to cells loaded with control peptides.

To further evaluate the specificity of the D10A3 TCR-like antibody its reactivity with peptides that exhibit sequence similarity to the PAP peptide was evaluated. The peptides are shown in Table 12.

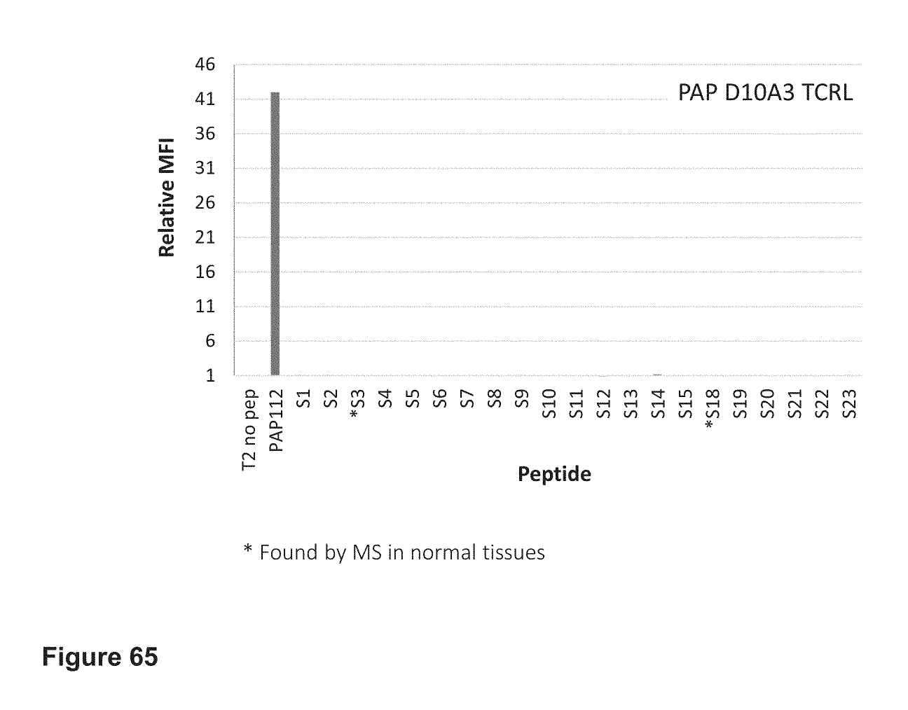

As shown in FIG. 65, D10A3 TCRL did not bind any of the peptides from this panel of similar peptides. These data demonstrate the high selectivity and fine specificity of D10A3 and demonstrates the usefulness of the similar peptide approach and tools developed as described above to evaluate the selectivity and fine specificity of TCRLs.

To investigate the fine peptide epitope specificity of the isolated TCR-like antibodies towards the PAP peptide alanine scanning was performed in which specific residues in the peptide were mutated to alanine and the binding of the TCR-like antibody to Ala mutated peptides was tested by their loading onto T2 antigen presenting cells (Table 13). Binding was monitored by flow cytometry and extent of binding of TCR-like antibodies to the mutated presented peptides as measured by mean fluorescence intensity (MFI) was compared in comparison to T2 APCs loaded with the native unmutated PAP peptide. The proper loading of the various Ala mutated peptides (described in FIG. 2) was monitored by flow cytometry using BB7.2 a monoclonal antibody for HLA-A2.

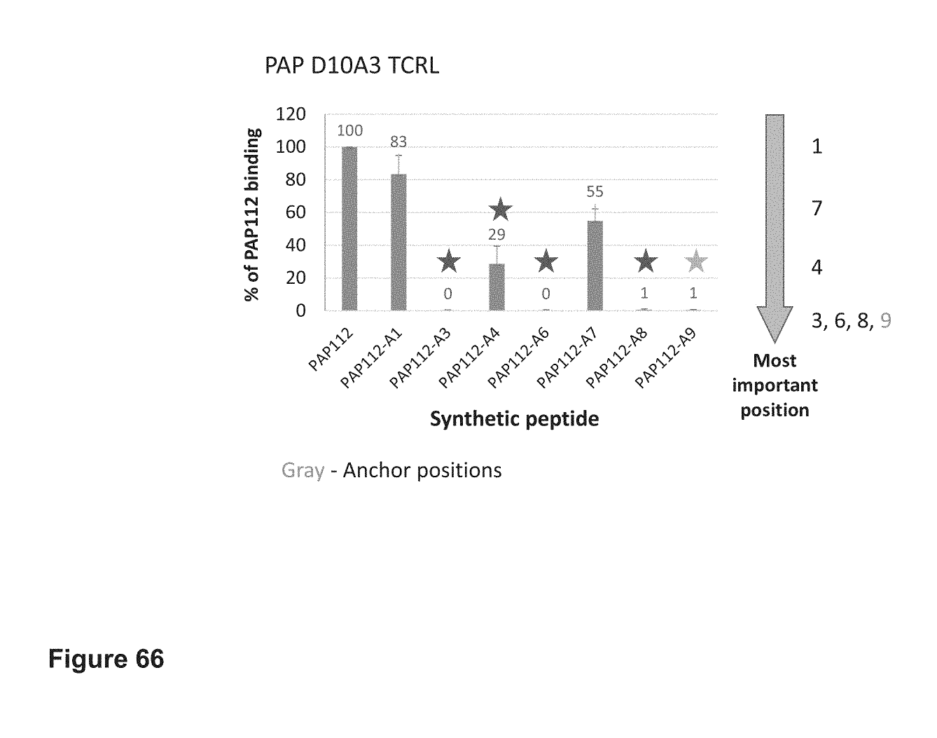

All Ala mutated peptides were efficiently loaded onto T2 cells in comparison to the native un-mutated PAP peptide (data not shown). As shown in FIG. 66, The TCR-like antibody exhibited peptide dependency binding as specific mutations affected the binding and induced a decrease in the binding intensity of the TCR-like antibody upon introduction of Ala at specific peptide positions. These results indicate that PAP TCR-like antibody exhibited peptide-specific and restricted binding in the context of HLA-A2 loaded with various Ala mutated PAP peptides, indicating that this antibody is TCR-like in its binding properties, thus, it binds the MHC-peptide complex with MHC-restricted and peptide-specific manner.

The D10A3 TCR-like antibody exhibited a marked decrease of 90% in binding to three Ala mutated peptide at three positions #3, 6, and 8. Decrease of 70% in binding to one Ala mutated peptide at position #4 was also observed. A 5′ position sensitivity can be attributed to position number 7 (decrease of 45%).

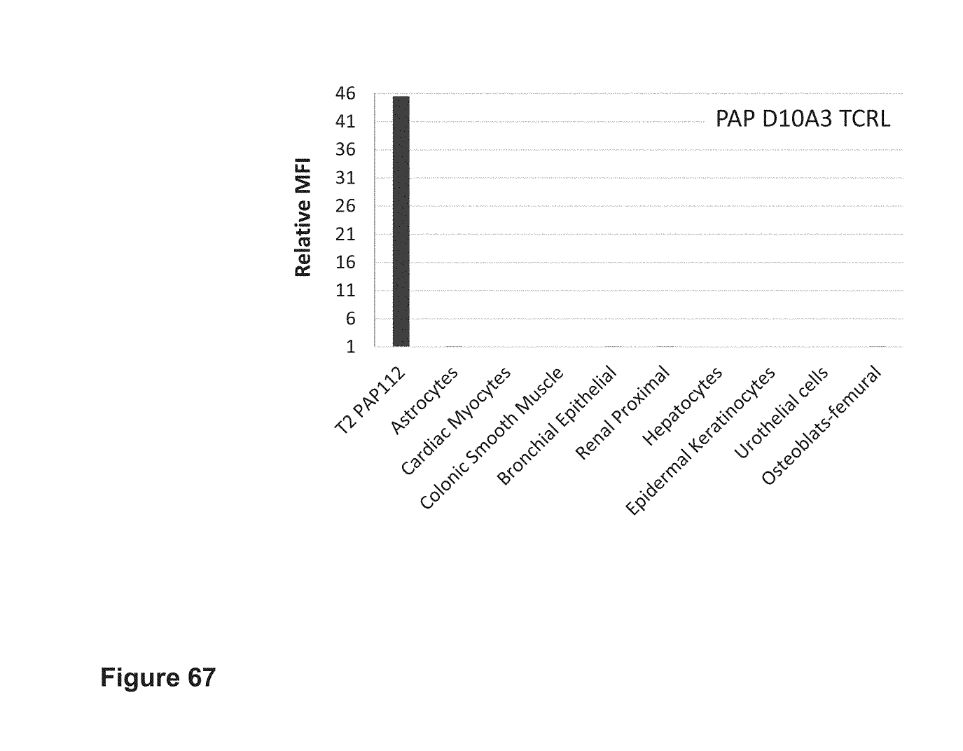

The present inventors explored binding specificity of the HLA-A2/PAP TCR-like Ab to a panel of normal primary cells of various origin that do not show PAP RNA expression. As shown in FIG. 67, D10A3 TCRL did not bind any of these cells. Positive control was T2 cells loaded with the PAP peptide to which D10A3 TCRL bound strongly.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An antibody capable of binding, with a human major histocompatibility complex (MHC)-restricted specificity, a MHC being complexed with an HLA-restricted peptide tumor antigen or autoimmune antigen, said antibody having a binding specificity dictated by at least 4 amino acid residues in said HLA-restricted peptide such that at least 70% reduction in binding of said antibody to said complex is observed when each of said at least 4 amino acid residues is substituted by alanine as determined by FACS of cells loaded with said HLA-restricted peptide comprising said substitution, said at least 4 amino acid residues not being anchor residues.

2. An antibody capable of binding, with a human major histocompatibility complex (MHC)-restricted specificity, a MHC being complexed with an HLA-restricted peptide antigen, said antibody having a binding specificity dictated by at least 4 amino acid residues in said HLA-restricted peptide such that at least 70% reduction in binding of said antibody to said complex is observed when each of said at least 4 amino acid residues is substituted by alanine as determined by FACS of cells loaded with said HLA-restricted peptide comprising said substitution, said at least 4 amino acid residues not being anchor residues and further wherein the antibody does not bind HLA-restricted peptide antigens presented on normal essential tissues, wherein said HLA-restricted peptide antigen is not from LMP-2A polypeptide.

3. An antibody capable of binding, with a human major histocompatibility complex (MHC)-restricted specificity, a HLA-A2/TyrD369-377 peptide complex, said antibody having a binding specificity dictated by at least 4 amino acid residues in said TyrD369-377 peptide such that at least 70% reduction in binding of said antibody to said complex is observed when each of said at least 4 amino acid residues is substituted by alanine as determined by FACS of cells loaded with said peptide comprising said substitution, said at least 4 amino acid residues not being anchor residues.

4. The antibody of claim 3, wherein said at least 4 amino acid residues are selected from X1, X3, X4, X6 and X7 of TyrD 369-377.

5. The antibody of claim 4, wherein said at least 4 amino acid residues are selected from X3, X4, X6 and X7 of TyrD369-377.

6. The antibody of claim 4, wherein said at least 4 amino acid residues are selected from X1, X3, X4 and X6 of TyrD369-377.

7. The antibody of claim 1, wherein said at least 70% reduction in binding is at least 90% reduction in binding observed when at least 1 amino acid residue of said at least 4 amino acid residues comprises said substitution.

8. The antibody of claim 1, wherein said at least 70% reduction in binding is at least 90% reduction in binding observed when each of at least 2 amino acid residues of said at least 4 amino acid residues comprise said substitutions.

9. The antibody of claim 1, wherein said at least 4 amino acid residues comprise 5 amino acid residues such that at least 70% reduction in binding of said antibody to said complex is observed when each of at least 4 amino acid residues of said 5 amino acids residues is substituted by alanine and wherein at least 30% reduction in binding of said antibody to said complex is observed when a fifth amino acid of said 5 amino acids is substituted by alanine.

10. The antibody of claim 1, wherein said antibody does not bind to HLA-presented peptides, which are present in essential tissues as determined by FACS analysis of cells loaded with said HLA-presented peptides, said HLA-presented peptides having at least one amino acid substitution as compared to said HLA-restricted peptide antigen in an amino acid residue which is not one of said at least 4 amino acid residues.

11. An antibody capable of binding, with a human major histocompatibility complex (MHC)-restricted specificity, a MHC being complexed with an HLA-restricted peptide antigen, wherein said antibody does not bind to HLA-presented peptides, which are present in essential tissues as determined by FACS analysis of cells loaded with said HLA-presented peptides, said HLA-presented peptides having at least one amino acid substitution as compared to said HLA-restricted peptide antigen in an amino acid residue not critical for binding said peptide antigen, as determined by FACS of cells loaded with said HLA-restricted peptide comprising an alanine substitution.

12. The antibody of claim 7, wherein said HLA-restricted peptide antigen is not from LMP-2A polypeptide.

13. The antibody of claim 1 having a binding affinity below 20 nanomolar to a single chain human major histocompatibility complex (MHC) complexed with said HLA-restricted peptide antigen, as determined by surface plasmon resonance assay.

14. (canceled)

15. The antibody of claim 1, capable of binding said HLA-restricted peptide antigen when naturally presented on cells, as determined by FACS.

16. The antibody of claim 15, wherein said cells are cancer cells.

17. (canceled)

18. The antibody of claim 1 comprising a therapeutic moiety.

19. The antibody of claim 1 comprising an identifiable moiety.

20. The antibody of claim 11, not binding an in-silico predicted HLA-restricted peptide comprising 1-4 amino acid substitutions as compared to said HLA-restricted peptide tumor antigen or autoimmune antigen.

21. The antibody of claim 2, wherein said HLA-restricted peptide antigen is selected from the group consisting of a tumor HLA-restricted peptide antigen, a viral HLA-restricted peptide antigen and an autoimmune HLA-restricted peptide antigen.

22. The antibody of claim 1, wherein said MHC is class I MHC.

23. (canceled)

24. The antibody of claim 18, wherein said therapeutic moiety comprises a CD3.

25. The antibody of claim 1, being a bispecific antibody.

26-27. (canceled)

28. The antibody of claim 18, forming a CAR.

29. The antibody of claim 1, wherein said HLA-restricted peptide antigen is derived from tyrosinase.

30. The antibody of claim 1, wherein said HLA-restricted peptide antigen is derived from WT1.

31. The antibody of claim 1, wherein said HLA-restricted peptide antigen is derived from MAGE-A4.

32. The antibody of claim 1, wherein said HLA-restricted peptide antigen is derived from MAGE-A9.

33. The antibody of claim 1, wherein said HLA-restricted peptide antigen is derived from PAP.

34. The antibody of claim 11, wherein said at least one amino acid substitution comprises 1-4 amino acid substitutions.

35. (canceled)

36. An expression vector comprising a polynucleotide comprising a nucleic acid sequence encoding the antibody of claim 1 operably linked to a cis-acting regulatory element.

37. A cell comprising the expression vector of claim 36.

38. A method for selecting highly selective TCR-like antibody with the optimal fine specificity towards a specific MHC being complexed with an HLA-restricted peptide antigen, the method comprising:
(a) providing an antibody capable of binding, with a human major histocompatibility complex (MHC)-restricted specificity, a MHC being complexed with an HLA-restricted peptide antigen, wherein said binding is with a predetermined affinity;
(b) determining binding of the antibody to peptides similar to said HLA-restricted peptide antigen having been mutated with alanine/glycine/valine/leucine in amino acids other than anchor residues so as to identify amino acids which are critical for binding of said antibody to said HLA-restricted peptide;
(c) determining binding of the antibody to in silico-predicted and/or validated HLA-presented peptides which are present on at least one normal essential tissue, said HLA-presented peptides comprising 1-4 amino acid substitutions as compared to said HLA-restricted peptide;
wherein said determining binding of said antibody to said peptides of (a) and (b) is by FACS analysis of cells loaded with said peptides or by functional assay, said antibody being qualified if said binding of (b) is undetectable by said FACS analysis.

39. The method of claim 38, wherein said HLA-presented peptides which are present on at least one essential tissue have at least one amino acid substitution as compared to said HLA-restricted peptide antigen in an amino acid residue not critical for binding said peptide antigen.

40. The method of claim 38, further comprising determining binding of the antibody to normal cells, which do not present said HLA-restricted peptide but are positive for said HLA.

41. An antibody which qualifies the selection criteria of claim 38.

42. The method of claim 38, wherein said predetermined affinity is below 20 nM.
